(19) United States
(12) Patent Application Publication
(10) Pub. No.: US 2004/0006787 A1
(43) Pub. Date: Jan. 8, 2004

(54) Plant defense-related genes regulated in response to plant-pathogen interactions and methods of use

(76) Inventors: Gregory B. Martin, Ithaca, NY (US); Kiran Kumar Mysore, Ardmore, OK (US); Oswald R. Crasta, Clinton, CT (US); Otto Folkerts, Guilford, CT (US); Peter Swirsky, Branford, CT (US)

Correspondence Address: BROWN & MICHAELS, PC, 400 M & T BANK BUILDING, 118 NORTH TIOGA ST, ITHACA, NY, 14850, US

(21) Appl. No.: 10/341,961

(22) Filed: Jan. 14, 2003

Related U.S. Application Data
(60) Provisional application No. 60/348,792, filed on Jan. 14, 2002.
(60) Provisional application No. 60/390,249, filed on Jun. 20, 2002.

(51) Int. Cl. A01H 1/00; C12N 15/82

(52) U.S. Cl. 800/279

(57) ABSTRACT

Compositions and methods of use for the expression of plant pathogen defense-related and signaling genes are provided. The compositions include nucleotide sequences identified from tomato that show specific patterns of gene expression associated with the Avr-Pto mediated defense response in tomato plants resistant to bacterial speck disease caused by Pseudomonas syringae pathovar tomato [strain T1(A)]. The nucleotide sequences are specific to genes that are up-regulated or down-regulated in response to the interaction of AvrPto and Pto in the presence of Prf. These compositions have agricultural utility for increasing resistance to a variety of biotic and abiotic stresses, including plant pathogens.

### REFERENCE TO RELATED APPLICATIONS

This application claims an invention which was disclosed in Provisional Application No. 60/348,792, filed Jan. 14, 2002, entitled “PLANT DEFENSE RELATED GENES AND METHODS OF USE”; and Provisional Application No. 60/390,249, filed Jun. 20, 2002, entitled “PLANT DEFENSE RELATED GENES REGULATED IN RESPONSE TO PLANT-PATHOGEN INTERACTIONS AND METHODS OF USE”. The benefit under 35 USC § 119(e) of the United States provisional applications is hereby claimed, and the aforementioned applications are hereby incorporated herein by reference.

### ACKNOWLEDGEMENT OF GOVERNMENT SUPPORT

This invention was made with Government support under Grant No. IBN-9872617 and Grant No. IBN-0109633, awarded by the National Science Foundation. The government has certain rights in the invention.

### BACKGROUND OF THE INVENTION

1. Field of the Invention

The invention pertains to the field of genetic engineering of plants. More particularly, the invention pertains to the genetic manipulation of plants, through transformation and\or breeding, by expressing or manipulating the regulation of expression of certain polynucleotides that enhance plant disease resistance.

2. Description of the Related Art

Biotic stress is major cause of loss in yield and quality of crop produce. It is caused by infection by plant pathogens, infestation by insects and other pests and parasitism by another organism. Developing plants that can resist or tolerate biotic and/or abiotic stress impacts positively on the quality and yield of crop plants. Understanding of the molecular mechanisms used by plants to resist, tolerate or avoid biotic and/or abiotic stress provides new strategies of crop improvement for stress tolerance.

Plants possess defense mechanisms, which enable the plants to prevent or limit the spread of plant pathogens. For example, in response to pathogen infection, plants trigger a cascade of signal transduction pathways that result in the activation of a defense response. Two types of relatively well-characterized defense responses are: 1) localized defense response, called the hypersensitive (HR) response, and 2) systemic response induced by signaling molecules moving from a location of infection to uninfected parts of a plant (systemic acquired resistance) or systemic induction of proteinase inhibitors (systemic wound response). The products of such induction of defense responses are known to enhance resistance against a broad range of biotic and/or abiotic stresses. Polynucleotides encoding such gene products can be used to enhance resistance to biotic and/or abiotic stresses, through various strategies, which are well known in the art, such as, for example, genetic engineering (e.g., transformation by, for example, particle bombardment or Agrobacterium-type vectors) and\or marker assisted breeding methods.

Resistance to bacterial speck disease is mediated by the dominant Pto gene in tomato and the corresponding avirulence gene avrPto in Pseudomonas syringae (Ps) pathovar tomato [strain T1(A)]. Pto, isolated by a mapped-based cloning strategy (Martin et al. 1992), encodes a protein kinase (Loh and Martin 1995), which is the molecular receptor for the bacterial avirulence factor encoded by the avrPto gene (Tang et al. 1996). Yeast two hybrid screens indicate that Pto interacts with and phosphorylates another protein kinase (Ptil; Zhou et al. 1995) as well as transcription factors (Pti 4/5/6) that directly bind to an element in the promoter region of defense-related genes (Zhou et al. 1997). The insecticide fenthion elicits an HR-like response, similar to that induced by avirulent bacteria, with this phenotype being conditioned by the highly similar and closely linked gene Fen (Martin et al. 1994). Expression of both sensitivity to fenthion and resistance to Pseudomonas strain T1(A) requires the action of another tomato gene, Prf, indicating a potential convergence of signal transduction pathways for the two phenotypes (Salmeron et al., 1996). Rapid and genotype-differential defense gene expression can be detected after both treatments within 4 hr (Halterman, 1999; Jia and Martin, 1999). A model of response to bacterial speck disease and fenthion treatment has been described by Zhou, et al. (1997).

### SUMMARY OF THE INVENTION

Methods and compositions for expression in plants, or manipulation of expression of plant pathogen defense-related and signaling genes are provided. The compositions comprise nucleotide sequences identified from tomato that show specific patterns of gene expression associated with the Avr-Pto mediated defense response in tomato plants resistant to bacterial speck disease caused by Pseudomonas syringae pathovar tomato [strain T1(A)]. Some of the nucleotide sequences are specific to 227 genes that are induced in response to the interaction of AvrPto and Pto in the presence of Prf, while others are specific to 129 genes that are down-regulated in response to the interaction of AvrPto and Pto in the presence of Prf. The compositions find use in agriculture, especially for increasing resistance of plants to a variety of plant pathogens, methods of modifying the signaling pathways and methods of inducing specific genes or decreasing expression of specific genes, resulting in a favorable resistance response in the plants. Methods for regulating the expression of a gene in response to a stimulus also are provided. The methods comprise stably transforming the genome of plants with the nucleotide sequences of the invention operably linked to a suitable promoter that drives the expression in a plant cell, and decreasing expression of the nucleotide sequences of the invention. Methods for marker assisted selection in breeding strategies also are provided.

### BRIEF DESCRIPTION OF THE DRAWING

FIGS. 1A and 1B show an illustration of trace indexing, as used for identification of specific nucleotides at a specific position immediately following a restriction-site of the cDNA fragment. See Materials and Methods for details on the trace indexing process. The color of the trace indicates the last nucleotide of the unlabelled primer used in the competitive PCR reaction (blue=A, Red=C, Green=G and black=T). FIG. 1A—Identification of the nucleotides in R2 position. FIG. 1B—Identification of the nucleotides in J2 position. The detected nucleotides of the corresponding cDNA fragments are indicated next to each peak in the digital images.

FIG. 2 shows a chart depicting the various experimental treatments used to treat leaves.

FIG. 3 shows a chart depicting a Pseudomonas syringae disease response model.

FIG. 4 shows a chart depicting the CuraGen GeneCalling procedure.

FIG. 5 shows a cartoon depicting the GV2260 Agro transient assay.

FIG. 6 shows a graphic map of the pQuiet2 vector used for VIGS.

FIG. 7 shows a graphic representation of the PCR method, illustrating how the VIGS library was normalized to reduce the frequency of highly expressed genes.

FIG. 8 shows a graphic representation of the “VIGS strategy” used for library screening.

FIG. 9 shows a graph of the resistance response for induced or up-regulated genes.

FIG. 10 shows a graph of the resistance response for suppressed or down-regulated genes.

FIG. 11 shows the resistance response for an induced gene in the jasmonic acid synthesis pathway.

FIG. 12 shows Northern blot hybridization data using clones for a subset of the polynucleotide sequences of the invention. Plant samples from PtoR (R), PtoS (S) and Prf-3 were used in the RNA-gel blots at 0, 2, 4 and 8 hours after infection by the pathogen, Pseudomonas syringae pathovar tomato [strain T1(A)].

FIGS. 13 A-S shows the phenotypes of several plants wherein VIGS was used to silence specific genes modulated in the resistance response.

### DETAILED DESCRIPTION OF THE INVENTION

Materials and Methods

1. Plant Material and Treatments

Tomato seeds were placed on Whatman paper in Petri plates to ensure uniform germination. Germinated seeds (8 for each treatment, 96 total,) were planted singly in 1″ cells and grown for ˜2 weeks. Eight uniform plants per treatment were transplanted into 5″ pots, with 2 plants in each pot (4 pots per treatment). This planting schedule was repeated 3 consecutive weeks to allow for repetition of the entire experiment if necessary. However, all plant material processed was result from a single experimental cohort. Approximately 2 weeks after repotting, plants were subjected to several different treatments. Three tomato genotypes were infiltrated with the avirulent bacteria T1(A). Rio Grande Pto-R (RG Pto-R) responds with a macroscopic HR after ˜8-12 hr. The ptoS genotype has a single nucleotide mutation in Pto, causing these plants to respond with visible disease symptoms after ˜18 hr. Finally, plants with the prf-3 mutation have a ˜1 kb deletion in the Prf gene, disrupting the Pto pathway and resulting in susceptibility. The ptoS and prf-3 mutations are in the RG Pto-R genotype and the plants are thus isogenic. Plants were vacuum infiltrated as described (Jia and Martin, 1999) and brought back to the greenhouse for 4 hours. Control plants were infiltrated with buffer only. RG Pto-R were also vacuum infiltrated with a solution of fenthion (Halterman, 1999). As a control to identify expression induced by fenthion (but not related to the Pto pathway), the prf-3 mutant line was also be infiltrated with fenthion. At the appropriate time point following each treatment, leaf tissue from the two plants in a pot (avoiding both the youngest and oldest leaflet) was harvested together, placed in liquid nitrogen, and stored at −80 C. This was repeated for three (of the four total) pots for each treatment, resulting in three replicates. The fourth pot was saved so that the expected phenotype can be confirmed. RNA was then be isolated from 3-5 grams of tissue by the hot phenol method (Jia and Martin, 1999). The quality of the RNA preparation was assessed by formaldehyde gel-electrophoresis and spectrophotometry, and then stored in 70% ethanol.

TABLE 1
Experimental samples and treatments

| Tomato | | | | Treatment |
|---|---|---|---|---|
| genotype | Treatment | Time point | Replicates | Notation |
| RG Pto_R | T1(A) | 4 hr post infiltration | 3 | PtoR_T1A |
| | 2.2 × 10⁷ | | | |
| ptoS | T1(A) | 4 hr post infiltration | 3 | ptoS_T1A |
| | 2.2 × 10⁷ | | | |
| prf_3 | T1(A) | 4 hr post infiltration | 3 | prf3_T1A |
| | 2.2 × 10⁷ | | | |
| RG Pto_R | fenthion | 4 hr post infiltration | 3 | PtoR_Fn |
| prf_3 | fenthion | 4 hr post infiltration | 3 | prf3_Fn |
| RG Pto_R | vacuum | 4 hr post infiltration | 3 | PtoR_Ctrl |
| | infiltration | | | |
| | (control) | | | |

2. RNA Profiling Analysis

RNA profiling studies were conduced using GeneCalling. GENECALLING™ is a differential expression analysis, RNA profiling technology, which is described in U.S. Pat. No. 5,871,697 and in Shimkets et al., Nature Biotechnology 17:798-803 (1999), the complete disclosures both of which are hereby incorporated herein by reference in their entirety. Poly-A mRNA was isolated from each of the RNA samples and double-stranded cDNA was synthesized from each of the poly-A mRNA samples (Shimkets, et al., 1999). Each cDNA sample was subjected to digestion with 96 pairs of restriction endonucleases. The digested product was ligated to two specific adapters at each end and subjected to quantitative PCR amplification using two primers labeled with FAM (J-primer) and Biotin (R-primer) specific to the two adapters (Bruce et al., 2000). After electrophoresis of the PCR-amplified product, the relative intensity of the cDNA fragments was measured by laser excitation of the FAM-label. DNA fragments of known sizes (sizing-ladder) were simultaneously electrophoresed to accurately (±0.1 base pairs) determine the sizes of the cDNA fragments. The electrophoresis data were stored as digital gel images with the relative intensity of the cDNA fragments in the Y-axis and the fragment size in base pairs in the X-axis. The RNA profiling was repeated three times for each cDNA sample.

Pair-wise in silico comparisons were made between the treatments using the digital trace images to identify cDNA fragments modulated at least 2-fold. The replicated data were used to calculate the statistical significance of the difference, as described in prior publications (Shimkets, et al., 1999; Bruce et al., 2000). Secondary comparisons were made using the data from specific pair-wise comparisons to identify specific gene expression patterns associated with a biological response.

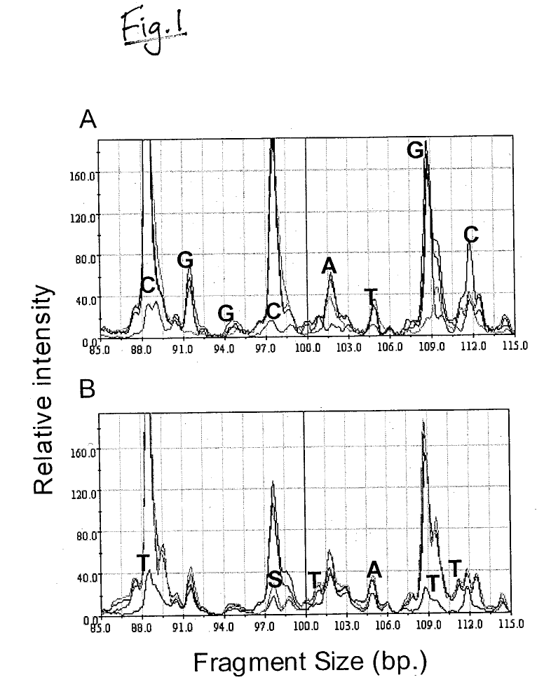

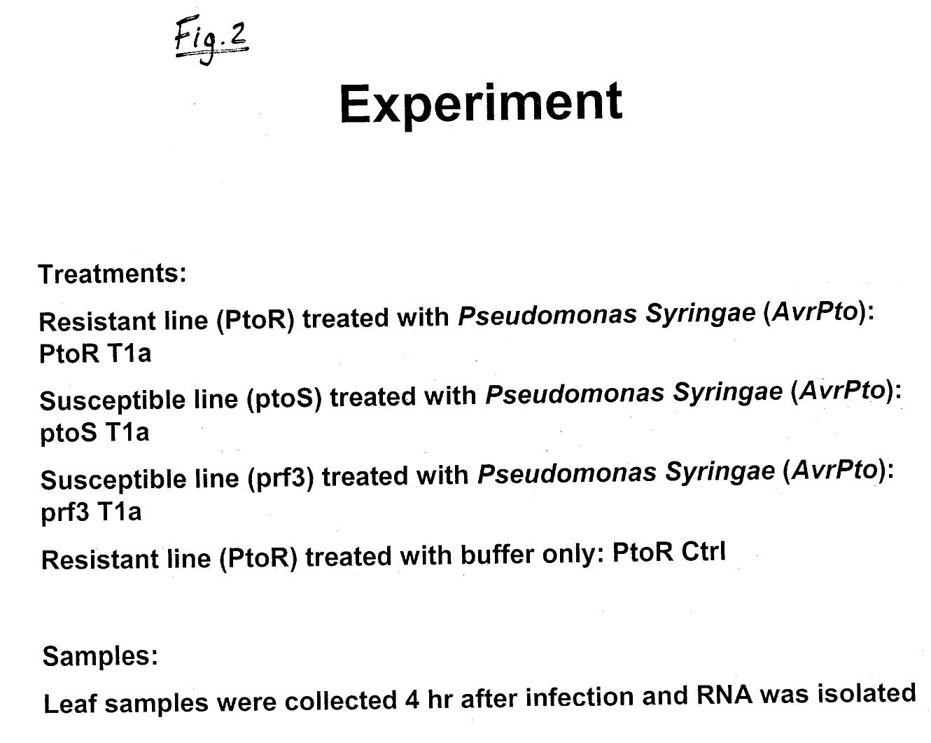

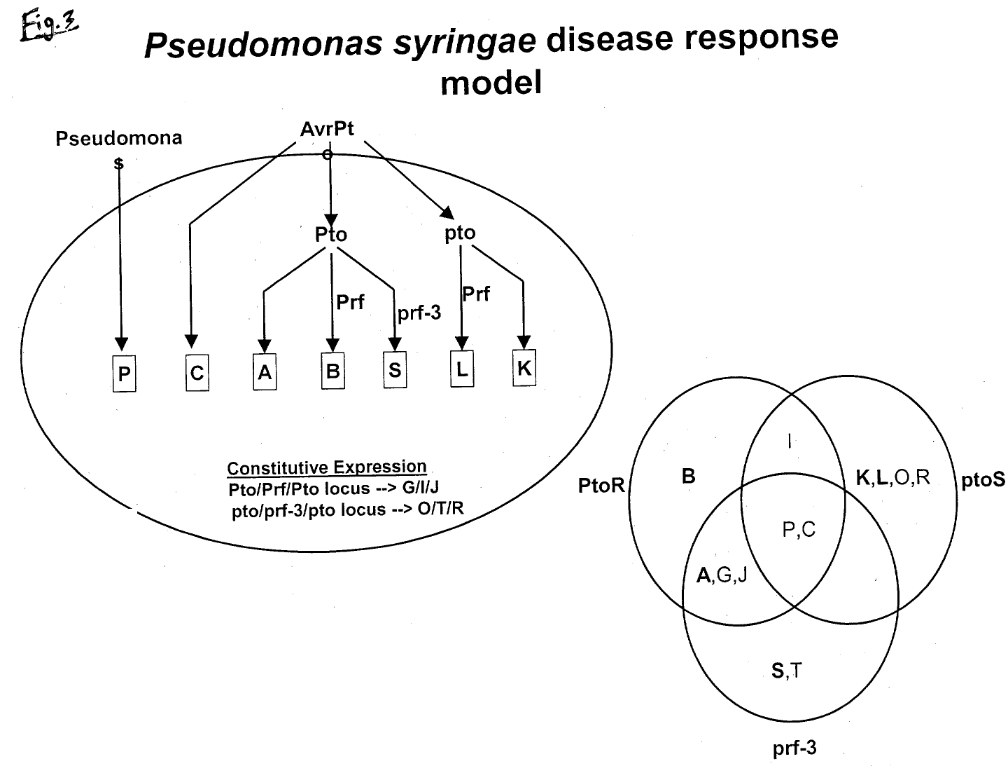

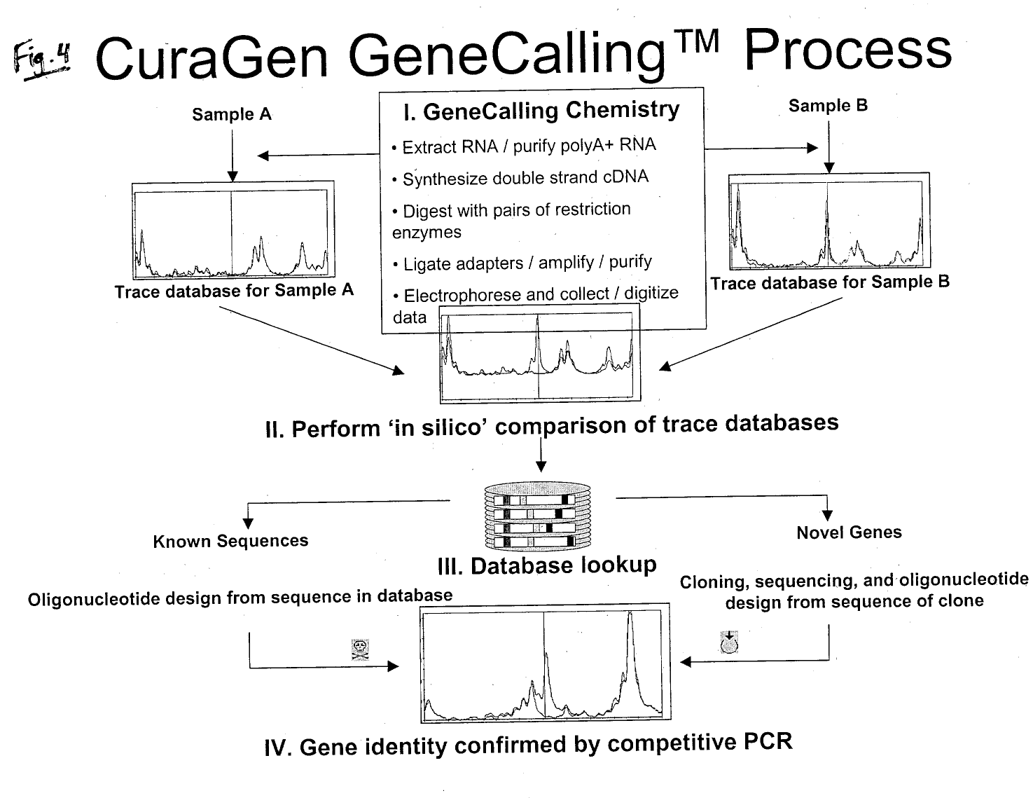

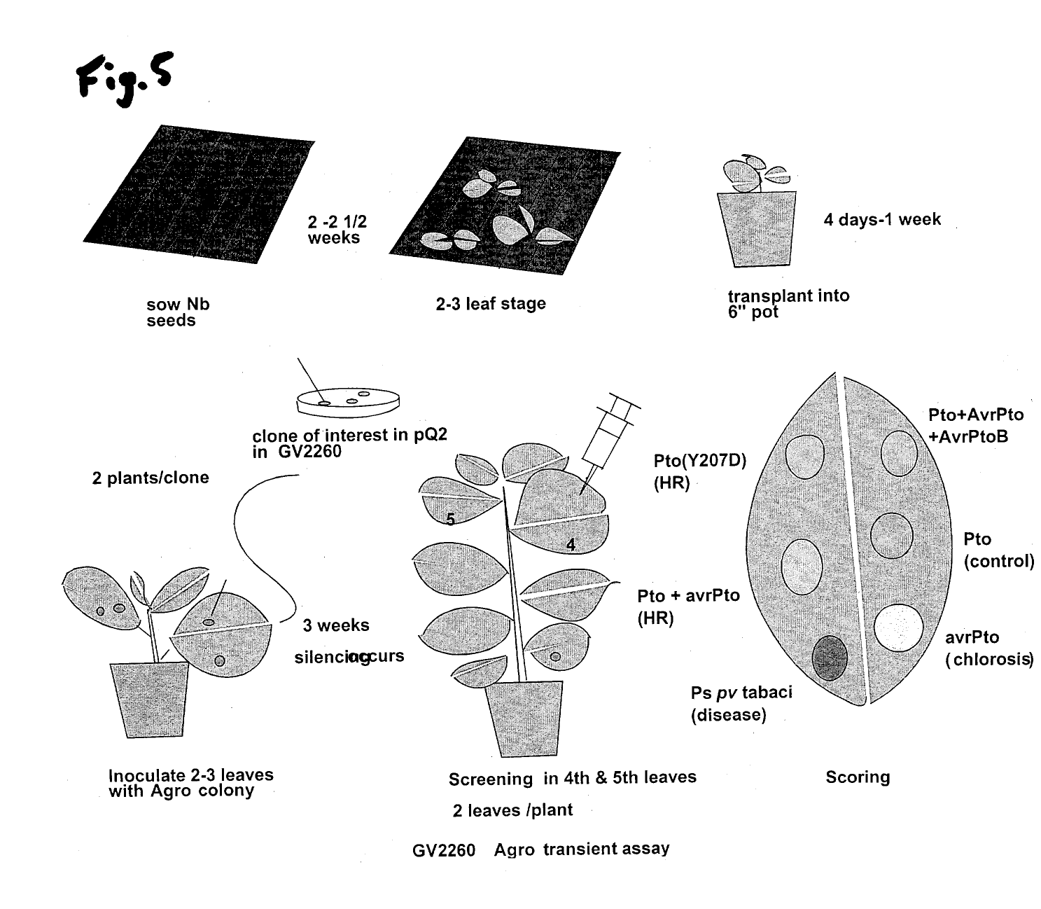

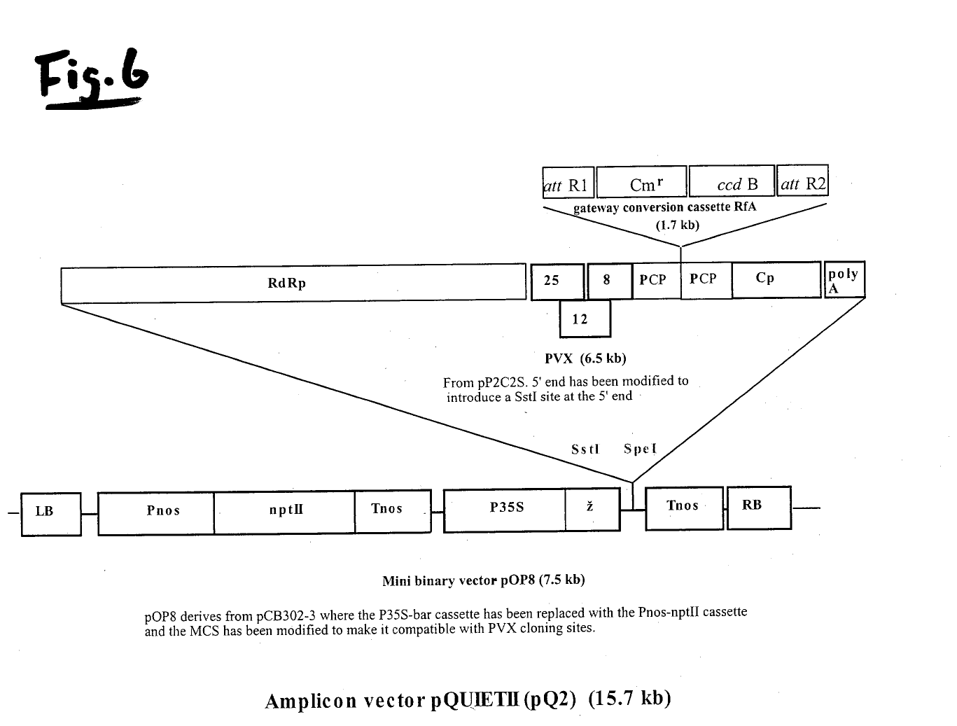

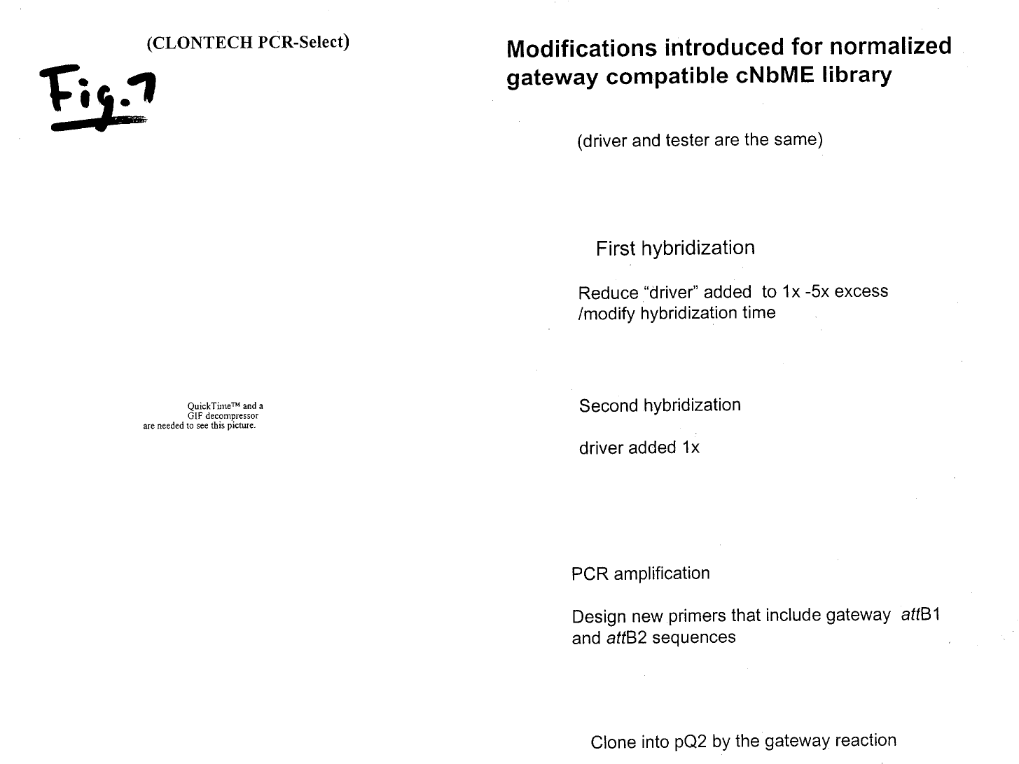

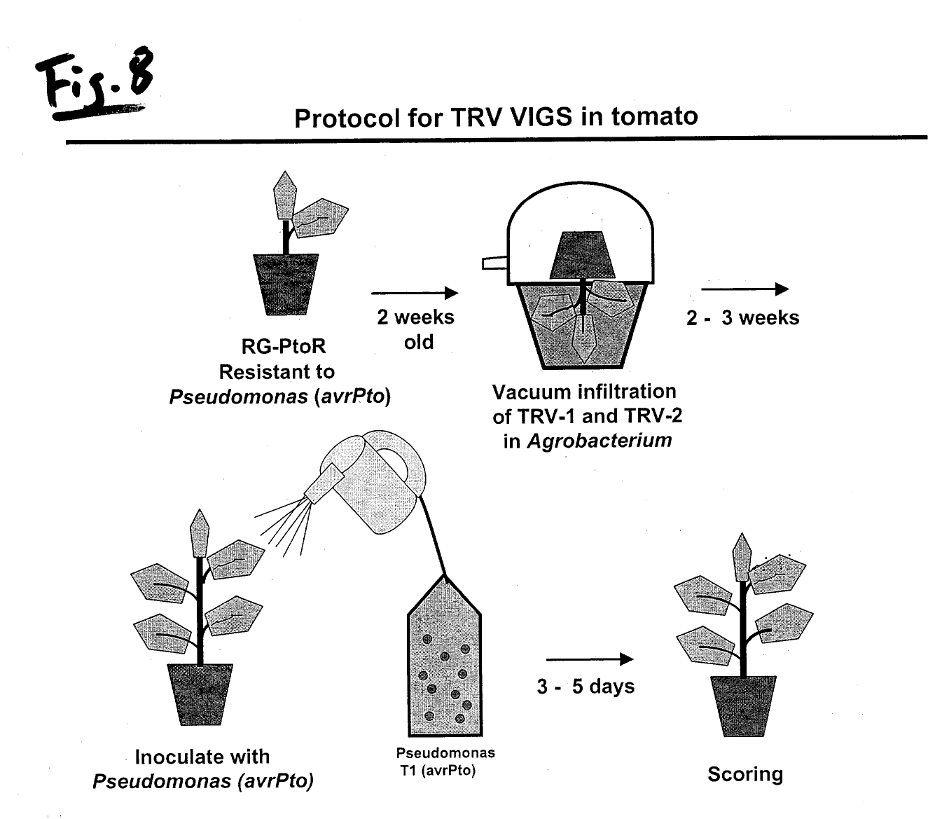

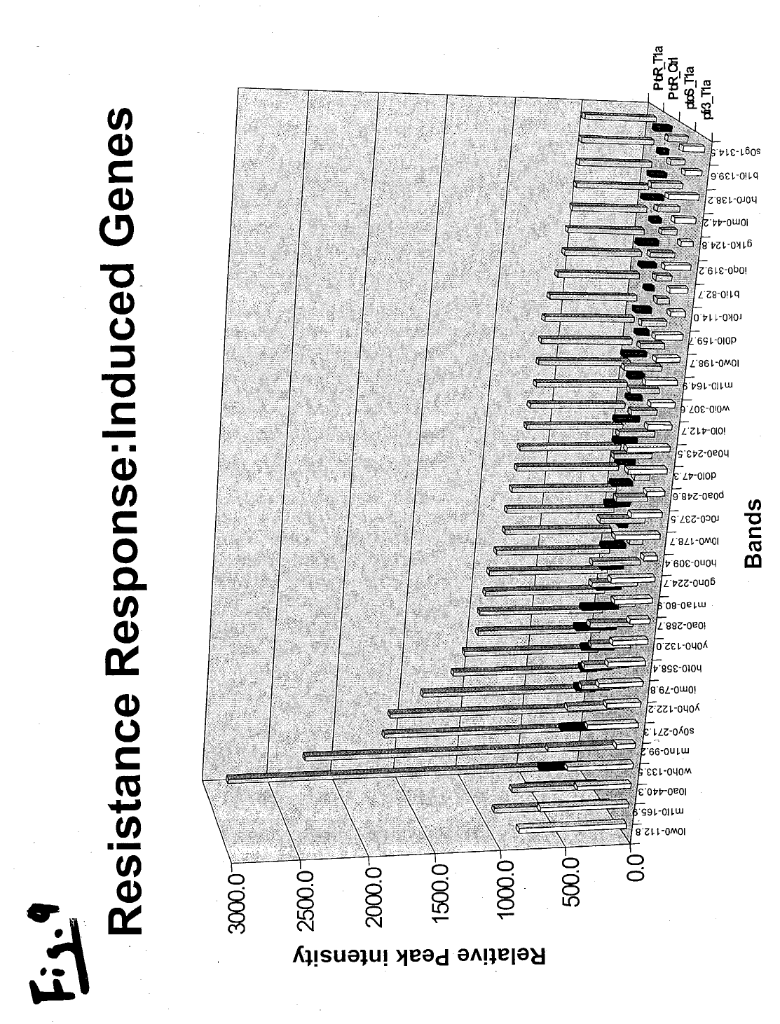

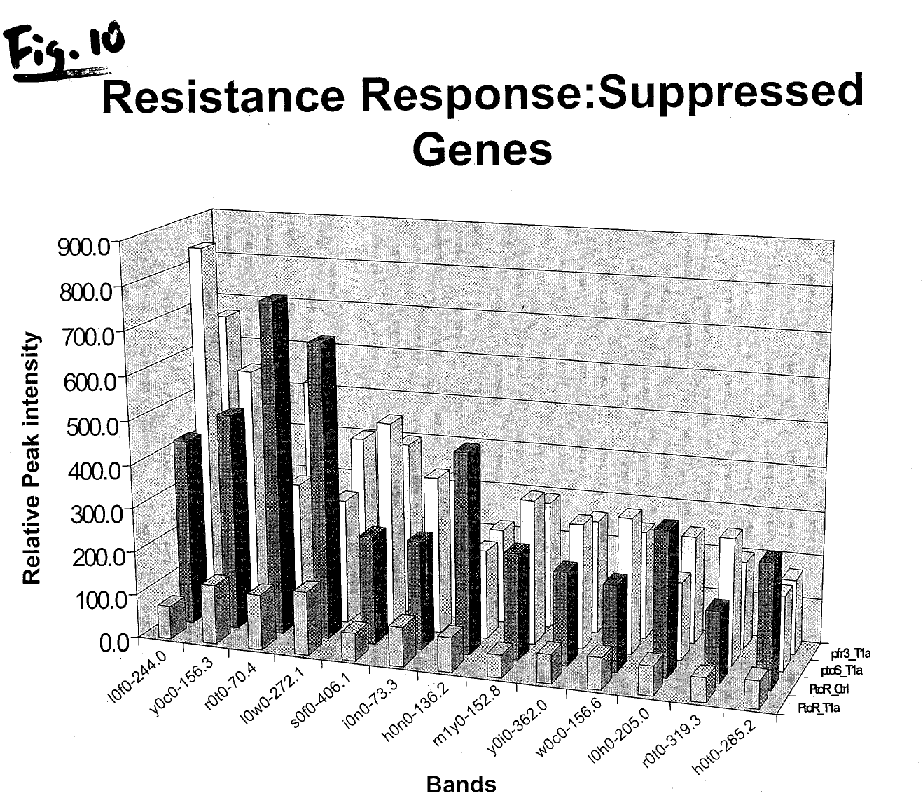

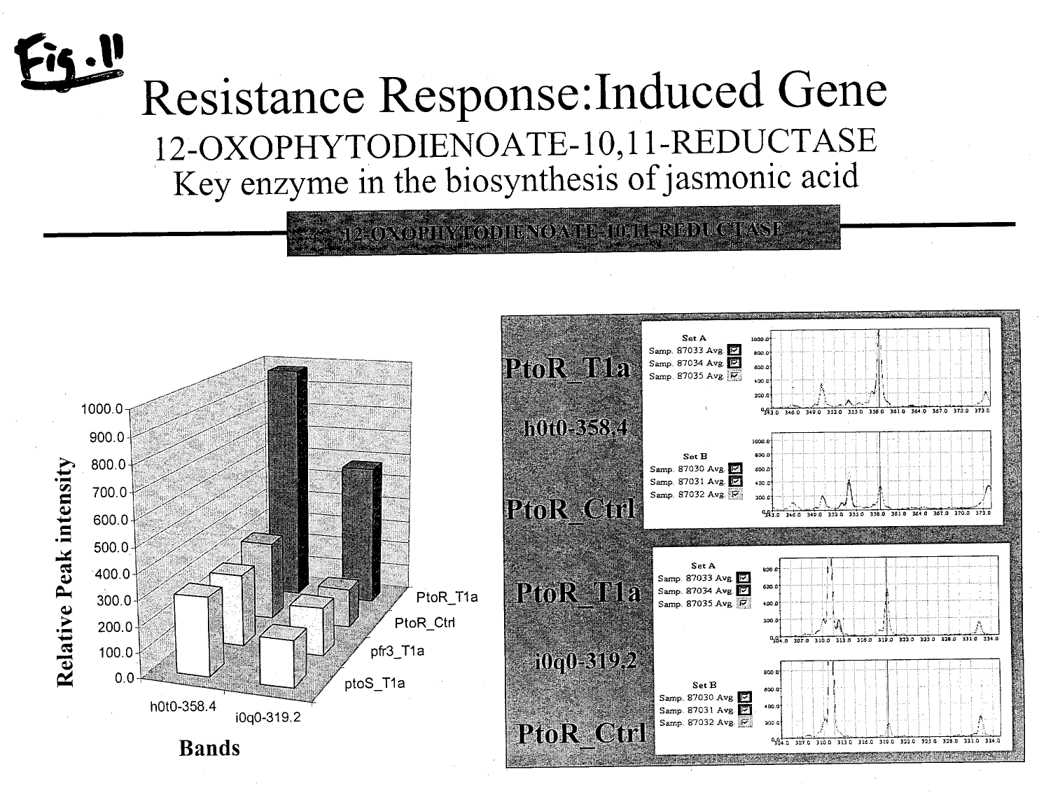

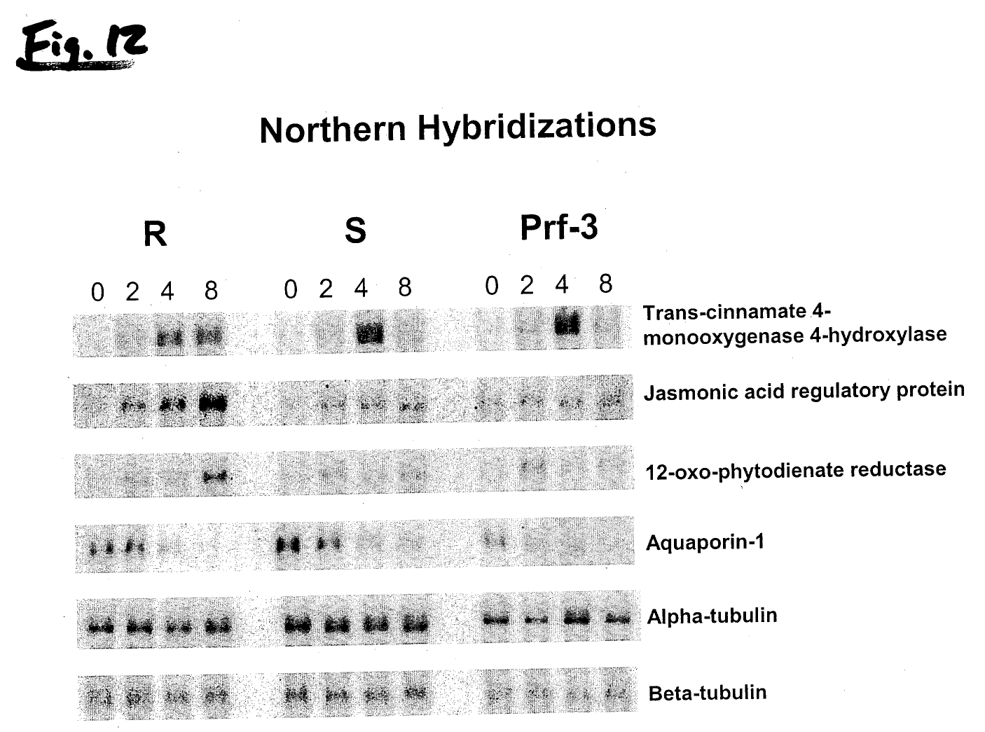

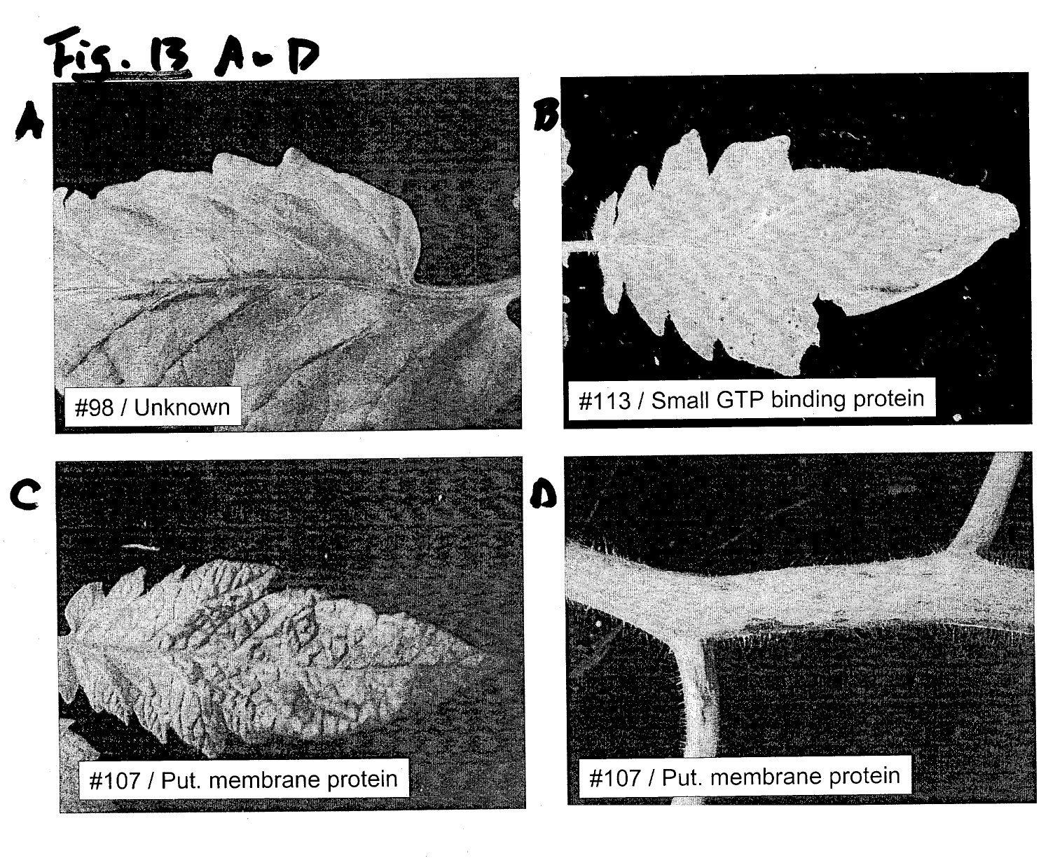

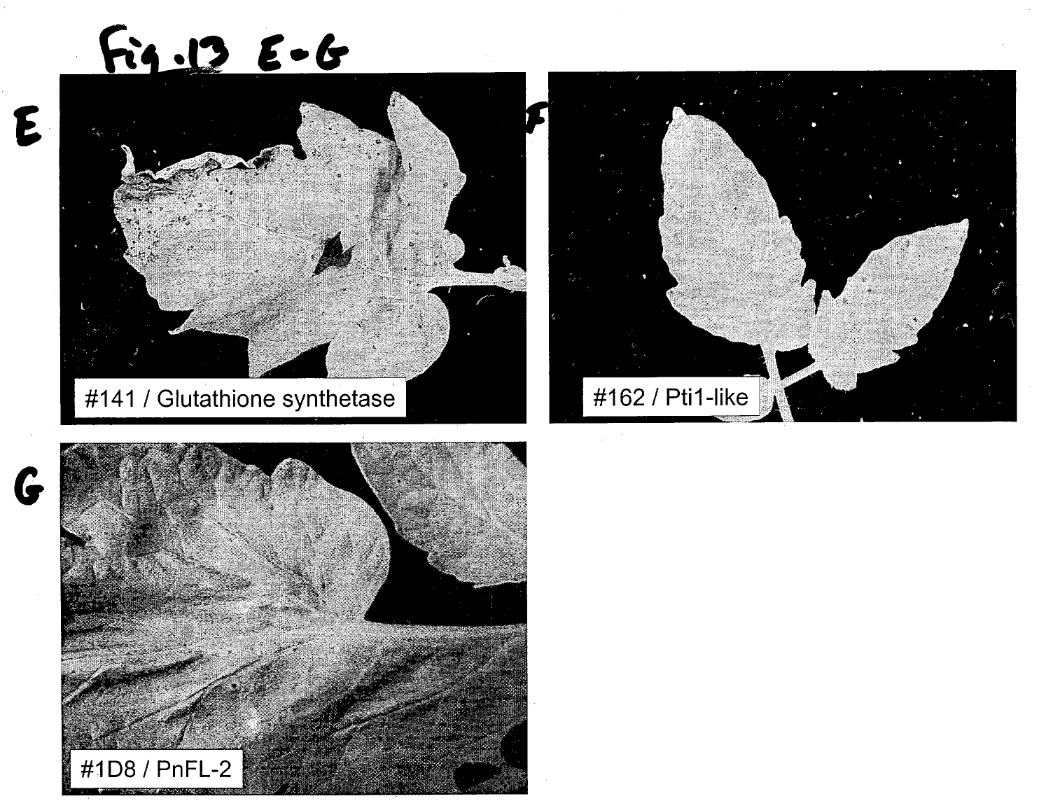

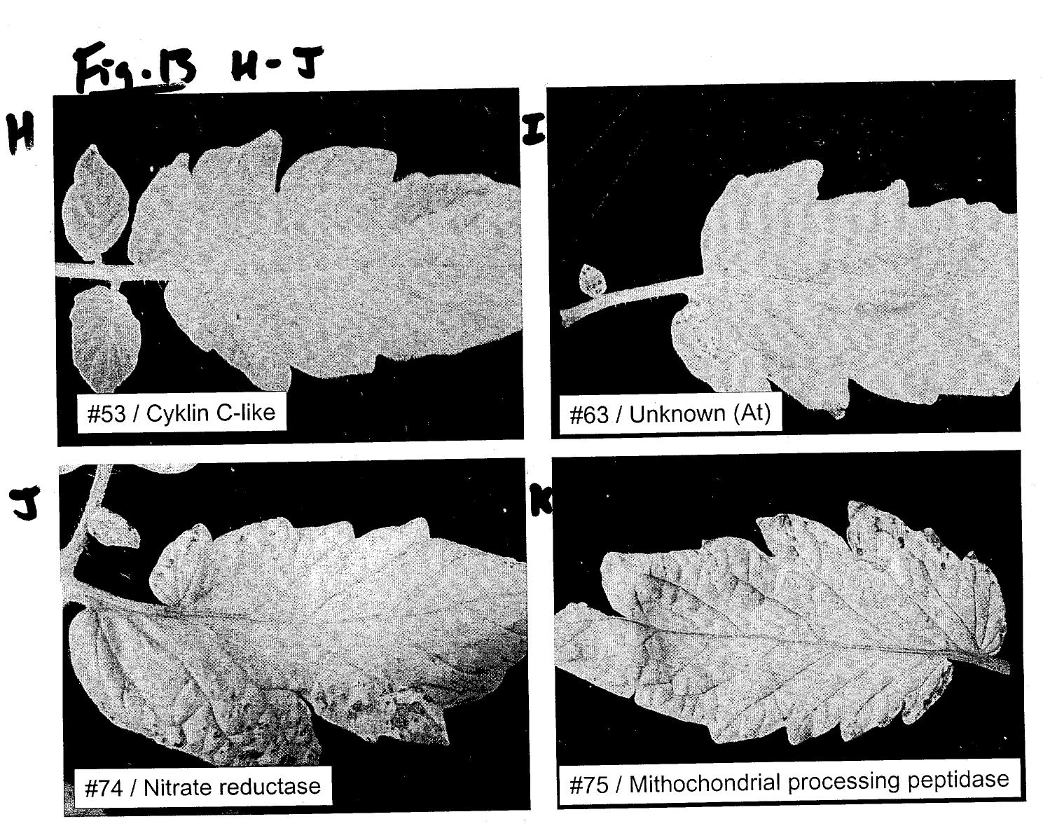

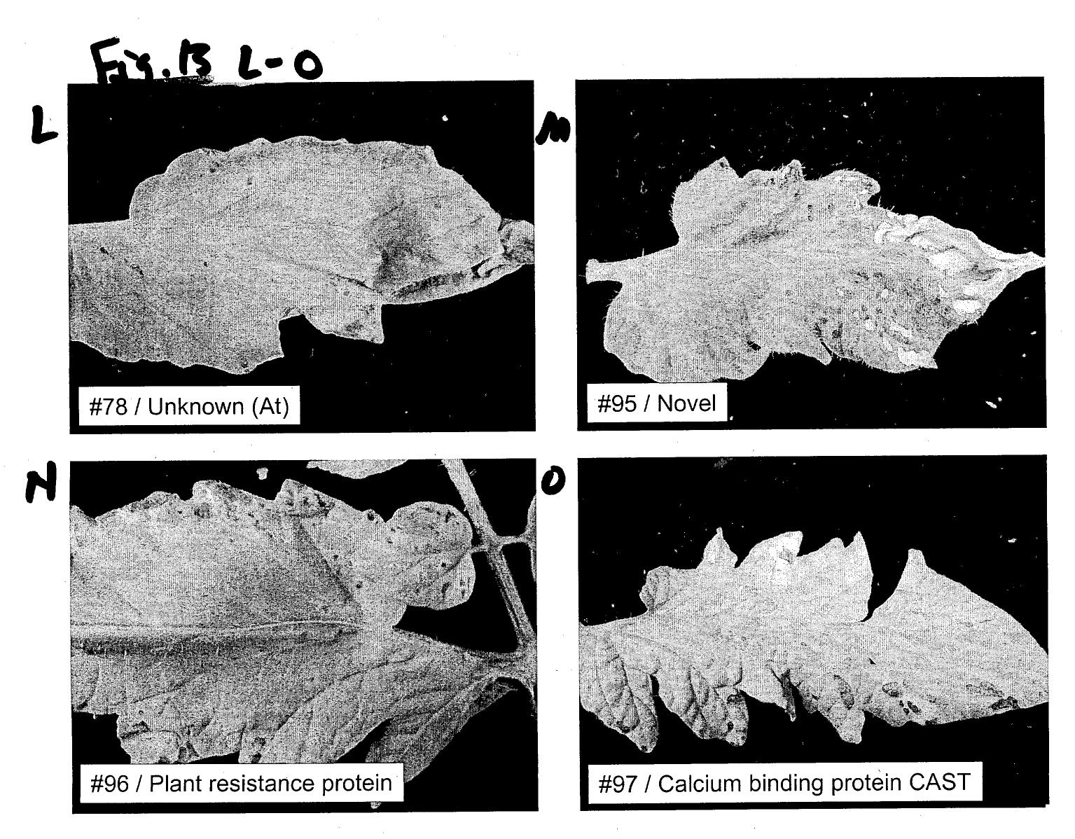

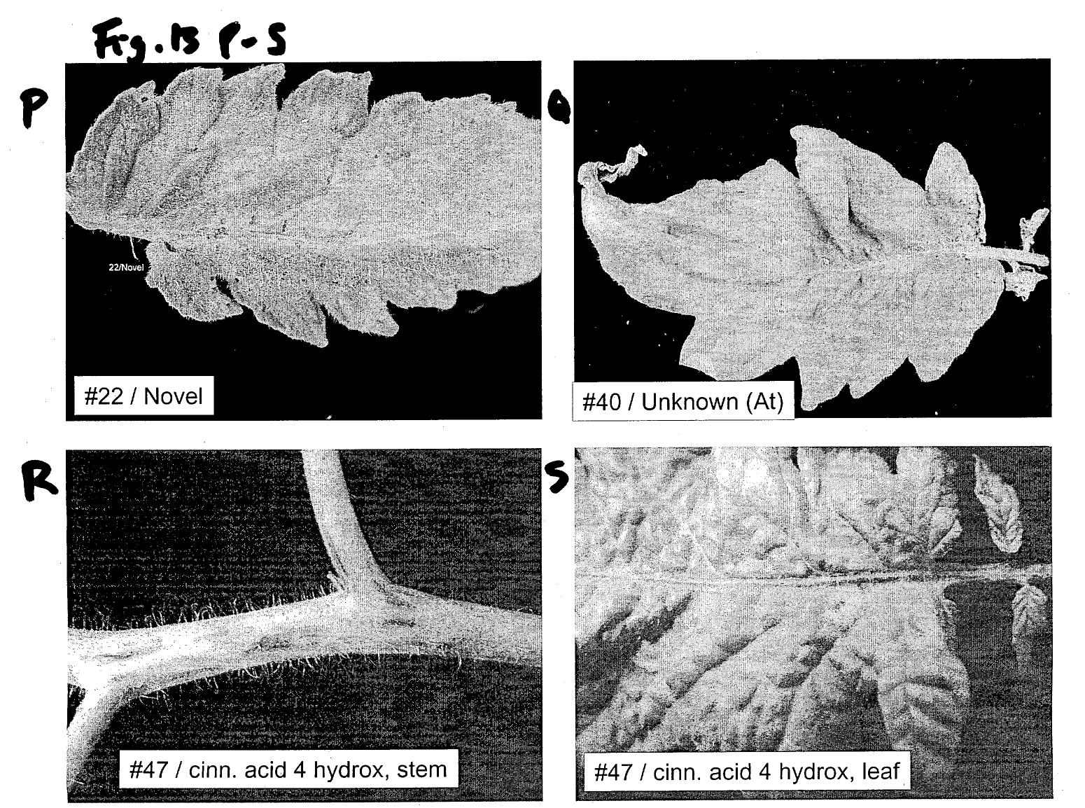

The PCR-amplified product from the four treatments was subjected to Trace Indexing (see FIG. 1) to obtain additional nucleotide sequences of the cDNA fragments. Trace Indexing was done using a competitive PCR procedure (Shimkets et al., 1999 and Bruce et al., 2000), which involved re-amplification of the PCR product from RNA-profiling under same conditions, except with the addition of an unlabeled oligonucleotide primer. The unlabeled primers were designed such that each group of primers were identical to the labeled primers (J- and R- primers) except that the 3′ end of the primers were extended to include one (A, T, G or C) or two (NA, NT, NG or NC) nucleotides immediately following the unique restriction-site at each end (J and R). A total of four groups of such unlabeled primers were designed (J1, J2, R1 and R2). The digital trace-images obtained by 4 different unlabelled primers within a group were superimposed to detect the specific nucleotides at a specific position immediately following the restriction site of the cDNA fragments, by observing which of the four primers oblate the peak intensity of a given band in a trace. Likewise, the bands in a given digital trace of a treatment were indexed for up to four additional nucleotides, based on the oblation of the intensities of the corresponding fragments by specific unlabeled primers.

The differentially modulated cDNA fragments were classified into known and novel genes by querying the available GenBank tomato sequence database using the data on fragment size (base pairs) and the restriction-site specific sequences for every cDNA fragment. The list of genes thus identified was further evaluated using the data from Trace Indexing. The confidence on the association of the known gene with the cDNA fragments was derived from the total number of additional nucleotides matched between the gene and all associated cDNA fragments, using the nucleotide data from trace indexing. The genes with maximum number of nucleotide matches from all associated cDNA fragments were selected as putative defense-related genes. The cDNA fragments without a significant association with known genes were annotated as putative novel genes and were cloned and sequenced. The association of a cDNA fragment with known or novel sequence was finally confirmed by the competitive PCR-method with an unlabeled oligonucleotide primer containing 20 to 23 nucleotides of sequence specific to the known or novel sequence associated with the cDNA fragment. The confirmation that the cDNA fragment is part of the sequence was obtained when the unlabeled oligonucleotide successfully competed with the labeled primer and oblated the intensity of the cDNA fragment of interest, while the intensities of the remaining cDNA fragments in the digital gel image were the same as those in the original gel image (Shimkets et al., 1999 and Bruce et al., 2000).

3. Virus-Induced Gene Silencing (VIGS)

One very powerful method for studying gene function is post-transcriptional gene silencing [PTGS; also known as RNA interference (RNAi)]. We used virus induced transient gene silencing (VIGS) to determine the role of differentially modulated genes, identified through GeneCalling, in disease resistance. Transient VIGS, based on potato virus X (PVX), is a technically simple and rapid method to assess function of genes in the Solanaceae. This method relies on the construction of a PVX derivative that contains the plant gene of interest (e.g., differentially modulated gene fragment obtained through GeneCalling) targeted for silencing. When the transgenic virus infects a plant, a double-stranded RNA species is formed which triggers sequence-specific gene silencing. PVX-based gene silencing has been shown to be especially effective in Nicotiana benthamiana. N. benthamiana is an excellent surrogate species for studying defense responses of tomato because its large fleshy leaves lend themselves to infection by many of the same pathogens that attack tomato (e.g., Pseudomonas, Phytophthora). Another advantage of N. benthamiana is that the tomato Pto gene is functional in this species, and therefore the Pto-AvrPto interaction can be easily studied using an Agrobacterium-based transient assay. Hence, we performed our VIGS assays in N. benthamiana.

In order to genetically dissect the Pto-mediated disease resistance pathway, a Fast-Forward genetics strategy based on Virus-Induced Gene Silencing (VIGS) was developed. We constructed a Nicotiana benthamiana (Nb) cDNA library in a PVX-based vector. We chose Nb instead of tomato for two simple reasons: silencing is more straightforward and the Pto/avrPto signaling pathway is conserved in this species. The Nb Mixed Elicitor cDNA (cNbME) library was made with Nb tissue treated with different abiotic elicitors (Salicylic Acid, Jasmonic Acid, ethylene) and undergoing Pto/avrPto mediated hypersensitive response (HR), Ps pv. tomato T1-mediated non-host HR, disease caused by Ps p.v tabaci and tissue expressing transiently avrPto (to analyze avrPto virulence-response).

Two time points for each treatment were included, as well as control or untreated tissue. RNA samples were extracted, analyzed for RNA quality control and pooled for mRNA purification. The cDNA library was constructed following the SSH method (Clontech, PCR-Select kit) with some modifications: the cDNA population was normalized instead of subtracted, size selected (0.3-1. Kb) and the inserts were made compatible with the gateway cloning system. The library was cloned in a PVX-based vector, which includes a mini-binary vector with the PVX cDNA sequence cloned within the T-DNA region and under control of the CaMV 35S promoter. NbME cDNAs were cloned within the PVX cDNA between the triple block and the Coat Protein via Gateway Reaction (yielding a chimeric virus) and the library was transformed into Agrobacterium tumefaciens (GV2260 strain).

Biotic treatments used to induce defense responses in leaf tissue include: AvrPto-Pto HR and AvrPto-dependent virulence (transient assay using EHA105); Agro (35S::avrPto)+ Agro (35S::Pto); Agro (35S::avrPto). Each Agro culture was diluted to OD600=0.2, mixed with the same volume and hand infiltrated with a syringe. Time points taken were 24 and 48 hours postinfection. For the non-host HR treatment, P. syringae pv. tomato T1 (titer 10 8) time points were 4 and 18 hours post-infiction, and P. syringae pv. tabaci and P. syringae pv. tabaci (avrPto) (titer 10 6) time points were 24 and 72 hours postinfection. Tissue silenced with PVX::PP2A was also included.

Elicitor treatments included INA 1.3 mM: 4, 24 h, (foliar spray application, incubation in Petri dish with lid at lab bench [INA=CGA41396 from CIBA]; Jasmonic acid 100 μM: 4, 12 hours (foliar spray application, incubation in closed chamber); Ethylene 50 ppm: 2, 12 hours (sealed chamber incubation). An equal amount of tissue from each time point/treatment was pooled and mRNA was obtained.

Amplicon vector pQ2 includes a 5′ modified PVX vector derived from pP2C2S (obtained from D. Baulcombe) in pOP8 mini-binary vector and converted into the gateway system. pOP8 derives from the mini-binary vector pCB302-3, in which the bar gene in the T-DNA region has been replace with the nptII gene and the multicloning site has been modified to make it compatible with PVX cloning sites.

For screening of the cNbME library and silencing Nb to identify genes involved in Pto-mediated disease resistance, Nb seedlings were agro-infected by pricking the leaves with a toothpick. The chimeric virus infects the plants, spreads and silencing develops after 2½ to 3 weeks. Two plants were infected/clone and two well-silenced leaves/plant were screened and scored for any alterations in:

Pto-mediated HR (co-agro-infiltration of cultures that carry a 35S::Pto and 35S::avrPto plasmid; agro-infiltration of the constitutively active Pto mutant Pto(Y207D);

Chlorosis generated by avrPto-mediated virulence (agro-infiltration of 35S::avrPto); or

Disease response caused by Ps pv tabaci.

In parallel, Nb plants were infected with PVX (empty vector, negative control), PVX::prf (positive control; prf is necessary for the Pto-mediated HR in Nb, its silencing abrogates Pto-mediated HR) and PVX::pds to assess the quality and timing of the silencing process (pds=phytoene desaturase; plants silenced for this clone develop photobleached leaves). The identity of any clone that gave any alteration in the responses listed above compared with the control plant (infected with the empty PVX vector) was determined by sequencing. At least 2,400 clones were screened initially, and approximately 130 clones were initially identified that gave any alteration in any of the responses scored.

A subset of 130 clones were selected (based on clone ID and on % of response inhibition) and cloned into a Tobacco Rattle Virus-based (TRV) silencing vector, in order to confirm and further characterize the phenotype conferred.

Two new assays were added:

Cf9/Avr9 HR (co-agro-infiltration of cultures that carry a 35S::Cf9 and 35S::avr9 plasmid), to determine Pto pathway specificity.

Ps pv tomato T1 (high titer) for non-host HR.

An average of 6-8 independently silenced plants/clone (two leaves/plant) were screened in each experiment and the results of the screening were compared with the controls (empty TRV infected plants and TRV::prf silenced plant). In most of the experiments, partial inhibition in the Pto+avrPto response was observed in the control plants (empty TRV vector infected plant). The % shown in the table for Pto-mediated HR inhibition represents the % of the inhibition above the % in control plants.

Results

1. RNA Profiling Analysis

Table 2 shows the comparisons made between the samples to identify cDNA fragments differentially modulated. Secondary comparisons were made to identify a subset of the cDNA fragments specifically induced in plants expressing the defense response genes (PtoR_T1A). The Genbank EST sequences that were identified to be associated with these fragments were used in competitive PCR to confirm the association of these genes with cDNA fragments identified. The sequences of the cDNA fragments, as confirmed by competitive PCR, are disclosed herein (see Sequence Listing).

TABLE 2
List of primary comparison made to identify cDNA-fragments
differentially modulated

| | | cDNA Fragments | | |
|---|---|---|---|---|
| | | Total | Differentially | |
| Job ID | Primary Comparison* | Assayed | Modulated** | |
| 29612 | PtoR_T1A vs. PtoR_Ctrl | 32995 | 3657 | (11.1%) |
| 29632 | PtoR_T1A vs. pfr3_T1A | 33935 | 2184 | (6.4%) |
| 29652 | PtoR_T1A vs. ptoS_T1A | 34380 | 1845 | (5.4%) |
| 29672 | PtoR_Fn vs. ptoS_Fn | 34779 | 435 | (1.3%) |
| 29692 | PtoR_Fn vs. PtoR_Ctrl | 32488 | 1022 | (3.1%) |
| 32992 | PtoR_T1A vs. PtoR_Fn | 32815 | 3315 | (10.1%) |
| 33012 | PtoR_T1A vs. PtoS_Fn | 33557 | 3319 | (9.9%) |
| 33032 | PtoR_Fn vs. ptoS_T1A | 35073 | 1373 | (3.9%) |
| 33052 | PtoR_Fn vs. prf3_T1A | 34196 | 897 | (2.6%) |
| 33072 | PtoR_Ctrl vs. ptoS_T1A | 34461 | 1524 | (4.4%) |
| 33092 | PtoR_Ctrl vs. prf3_T1A | 34032 | 904 | (2.7%) |
| 33112 | PtoR_Ctrl vs. ptoS_Fn | 34024 | 1400 | (4.1%) |
| 33132 | ptoS_T1A vs. prf3_T1A | 36686 | 293 | (0.8%) |
| 33134 | ptoS_T1A vs. ptoS_Fn | 36725 | 1256 | (3.4%) |
| 33152 | prf3_T1A vs. ptoS_Fn | 35859 | 1474 | (4.1%) |

*Annotations for samples are described in Table 1.
**minimum N-fold = 2, P-value = 0.15, values in the parentheses are percent of the total fragments assayed.

Certain aspects of the invention are published in an article entitled Comprehensive transcript profiling of Pto- and Prf-mediated host defense responses to infection by Pseudomonas syringae pv. tomato, in The Plant Journal (2002) 32, 299-315 (and Supplemental Discussion thereof), the complete disclosure of which is hereby incorporated herein by reference in its entirety. The article details the identification of 432 genes that are modulated in tomato plants, in the resistance response to Pseudomonas syringae pathovar tomato [strain T1(A)].

2. Identification of Functional Classes of Genes

We have identified and disclose herein 227 genes that are induced or up-regulated in the resistance response, specific to the response involving the interaction of AvrPto, Pto, and Prf. The expression data for these genes are provided in the Table 2 of Provisional Application No. 60/348,792, filed Jan. 14, 2002, entitled “PLANT DEFENSE RELATED GENES AND METHODS OF USE,” and are incorporated herein by reference. The sequences for these genes are provided in the Provisional Application as Appendices: Appendix 1 contains 197 sequences that were identified from the public database, Appendix 2 contains 30 sequences that were identified in house through cloning and sequencing, and Appendix 3 contains 16 sequences representing a subset of the 30 in house gene sequences that were extended using ESTs from the public database.

The 227 up-regulated genes were annotated using BLAST results from the public database. These annotations were used to classify the genes based on the proposed functional role of these genes. We have rank ordered the genes based on their functional role: 14 sequences were classified as novel, 35 sequences were classified as unknown, 28 sequences were classified in signaling pathways, 18 sequences were classified as transcription factors, 5 sequences were classified as hormone responsive, 3 sequences were classified in the Jasmonic acid biosynthesis pathway, 2 sequences were classified in the oxidative burst pathway, 24 sequences were classified as general plant-defense-related, 5 sequences were classified as proteases, 1 sequence was classified in the Shikimate pathway, 2 sequences were classified in ubiquination pathway, and 14 sequences were classified in the phenylpropanoid pathway. The remaining 76 sequences were classified in other function roles.

We also identified and disclose herein 129 genes that are down-regulated in the defense response, specific to the response involving the interaction of AvrPto with Pto, and\or Prf. The expression data for these genes are provided in Table 2 of Provisional Application No. 60/390,249, filed Jun. 20, 2002, entitled “PLANT DEFENSE RELATED GENES REGULATED IN RESPONSE TO PLANT-PATHOGEN INTERACTIONS AND METHODS OF USE,” and are incorporated herein by reference. Of the 129 down-regulated genes described, 125 were found in the public database, while the remaining 4 sequences were identified in house, through cloning and sequencing. These genes were annotated using BLAST results from the public database. These annotations were used to classify the genes based on the proposed functional role of the genes. We grouped these genes based on their functional role in Table 2 of the Provisional Application.

Several classes of these genes contribute to our understanding of the resistance response, and are described below.

Cell wall reinforcement: Phenylpropanoids have been proposed to serve as flower pigments (anthocyanin), UV protectants, defense chemicals (phytoalexins, insect repellents), allelopathic agents, and signal molecules in plant-microbe interactions. Furthermore, phenylpropanoids are the building units of polymeric support structures, such as lignin or lignin-like cell wall components. This study revealed that almost 4% of the resistance response genes belong to phenylpropanoid pathway (includes 4 anthocyanin biosynthesis genes) and they are induced during plant defense. Cell wall reinforcement and thickening is associated with plant defense during resistance response. In our study we have shown that, along with phenylpropanoid genes, other cell wall thickening genes are up-regulated and most of the genes encoding cell wall degrading enzymes are down-regulated during the resistance response.

Programmed Cell Death: Programmed cell death (PCD) constitutes the main form of cell death in animals, plants and other organisms. In plants PCD is a typical phenomenon during hypersensitive response triggered by an incompatible plant pathogen interaction. In this study we have shown that 4 PCD related genes are induced during the resistance response. The Arabidopsis LSD1 gene encodes zinc-finger type transcription factor that acts as a negative regulator of hypersensitive induced plant cell death. In this study we have shown that a tomato gene homologous to LSD1 is induced during the resistance response. Thus, it appears that the cells neighboring the infected cell are trying to suppress cell death by up-regulating the anti-death pathway. In contrast, a gene homologous to apoptosis antagonizing transcription factor (AATF) is down-regulated during the resistance response.

Senescence: PCD leads to senescence and we saw that genes encoding senescence related proteins like thiolase, cysteine protease, ascorbate oxidase and certain transcription factors were up-regulated as part of the plant defense mechanism. Overlap between the leaf senescence and pathogen-defense programs have been reported in the literature. Obvious visual symptoms of leaf senescence are loss of chlorophyll pigments due to decline in photosynthesis and chloroplast disintegration. It has been suggested that in cells undergoing HR, a decline in photosynthesis might act as an inducer of senescence. In this study, we show that certain chloroplast-associated transcription factors that are believed to be negative regulators of chloroplast gene expression are induced during the resistance response. These data indicate that the plant is shutting down the chloroplast as part of the defense mechanism, apparently to initiate senescence.

Oxidative burst: The oxidative burst that precedes PCD has been suggested to be a primary event responsible for triggering the cascade of defense responses in various plant species against infection with avirulent pathogens or pathogen-derived elicitors. Here we show that a tomato gp91-phox gene, along with a 2-nitropropane dioxygenase-like gene that is involved during oxidative burst, are induced during Pto mediated disease resistance.

Cell protectants: The oxidative burst may be followed by activation of genes encoding antioxidant enzymes in tissue surrounding the initial infection site to protect the surrounding cells against oxidative damage. We found that several of these cell protectant genes, such as annexin, peptide methionine sulfoxide reductase, glutathione peroxidase and glutathione S-transferase, are induced during the resistance response. Alkaloids are antimicrobial compounds that defend cells against pathogen attack. Using GeneCalling, we have shown that two genes involved in alkaloid biosynthesis are up-regulated during the resistance response.

Ubiquitination pathway: An interesting class of genes that is differentially expressed during plant disease resistance response are the genes involved in the ubiquitination pathway. Much speculation has been made by other research groups about the role of ubiquitination pathway during R gene mediated disease resistance. In animals, protein ubiquitination is not only essential for the normal physiological turnover of proteins, but also appears to have been adapted as part of an intracellular surveillance system that can be activated by altered, damaged, or foreign proteins and organelles. Here we show that certain genes, such as ubiquitin and ubiquitin activating enzyme, involved in ubiquitination-mediated protein degradation, are induced during Pto mediated disease resistance. In addition to induction of the ubiquitination pathway, several proteases are induced that also appear to target the degradation of pathogen effector proteins that enter the plant cell.

Water transport: Water is essential for pathogen multiplication in the plant tissue. One of the Pst disease symptoms is water soaked lesions on tomato leaves. Water is almost certainly essential to produce these symptoms. Here we show that aquaporin 1, aquaporin 2, ZmSIP2-1 and PIP2-1, genes involved in water transport, are suppressed during Pto mediated disease resistance.

Protein synthesis: Plant cell machinery undergoes tremendous stress during a defense response to make necessary plant defense-related proteins. All these extra proteins have to be made in a very short time and hence require efficient translation machinery. Here we show that 2 out of 3 protein synthesis genes, identified to be differentially expressed, are induced during the resistance response.

Signaling: Signaling is an essential part of any biological process. Since plant defense mechanisms are very complicated processes involving numerous protein-protein interactions, signaling plays a vital role. In this study, we have shown that 18 different kinases and 4 phosphatases are induced during the resistance response.

Stress-related genes: Stress-related genes are generally induced in response to any external stimulus. They are induced to activate the plant defense mechanism against any external stimulus. Here we have shown that several general stress-related genes, such as heat shock proteins, are induced during the resistance response.

Transport: Transport of nutrients and ions across cell membranes plays a major role in maintaining cellular homeostasis. Maintaining cellular homeostasis is a real challenge when a plant is under stress. We have found that several proteins involved in nutrient or protein transport are induced during the resistance response.

Transcription factors: The most striking observance among the resistance response genes is the preponderance of transcription-factor-related genes. The rapid induction of these genes suggests that they are integral members of an early-response plant defense network under Pto and Prf control. EREBP transcription factors are members of a large family of transcription factors thought to control expression of many genes during the defense responses induced by a number of biotic and abiotic stresses. It has been suggested that EREBPs regulate the expression of the genes encoding them via interaction with GCC boxes (found in promoters of several PR genes) in their promoters. In this study we have shown that EREBP protein encoding genes Pti4, a Pti4 homolog and Pti6 genes are induced during the resistance response. Two other genes encoding EREBP like proteins also are induced during the resistance response. WRKY proteins have recently been identified as a new family of transcription factors. They have been shown to have regulatory function in response to pathogen infection and other stresses. We identified 4 genes that encode WRKY domain-containing transcription factors, and all of them are induced during the resistance response. SCR is a member of a novel class of transcription factors with similarity to the basic domain of the basic-leucine zipper (bZIP) transcription factors. SCR regulates asymmetric cell division that is essential for generating the radial organization of the Arabidopsis root. Five genes encode SCARECROW (SCR) like transcription factors and all of them are induced during the resistance response. To our knowledge, this is the first report showing the involvement of SCR-like transcription factors in plant defense. NAC domain-containing proteins are a novel class of transcription factors to which belongs the ATAF subfamily of transcription factors. Several NAC domain-containing transcription factors have been implicated to play a role in plant defense. NAC proteins have been shown to directly interact with plant viral proteins to confer resistance against certain viruses. In this study, we have shown that two NAC domain-containing transcription factors are induced during the resistance response. We also identified 4 Zinc-finger type transcription factors that are induced during the resistance response. Zinc-finger type transcription factors are shown to be involved in variety of functions from plant development to senescence. It is interesting to note that we have identified a homolog of zinc-finger protein encoding LSD1 that is induced during the resistance response. LSDI acts as a negative regulator of hypersensitive induced plant cell death in Arabidopsis. Other classes of transcription factors that are differentially modulated during the resistance response include homeodomain-leucine zipper, myb-like, helix-loop-helix and chloroplast- associated transcription factors.

Unknown and Novel genes: Approximately 19% of the disease resistance response genes identified in this study appeared to have unknown functions. Most likely these genes are plant defense-specific genes. Interestingly, a class of genes that contribute to almost 6% of the resistance response genes has no homology to any translated protein or DNA sequence in the public database (thus classified as novel genes).

Conclusions

We have described the discovery of differentially modulated cDNA fragments associated with defense response in tomato by transcription profiling of the genome in resistant and susceptible genotypes treated with the pathogen, Pseudomonas syringae pathovar tomato [strain T1(A)]. We have described the identification of genes that are up or down regulated in the defense response that are specific to the response involving the interaction of Avr-Pto with Pto, and\or Prf. We have described the functional role classifications of these genes and discussed relevant genes in the context of their involvement in the defense response pathways. Our procedure for gene silencing, either in the resistant plant, if the gene is up-regulated or the susceptible plant, if the gene is down regulated, is provided. These results provide targets for inducing the defense response in tomato and other plants and markers for aiding in the identification and selection of resistant or susceptible lines in breeding strategies.

For example, the genes jasmonic acid regulatory protein (SEQ ID No: 1) and 12-oxo-phytodienoate reductase (SEQ ID No: 3) have been implicated to regulate jasmonic acid biosynthesis, which in turn induces a defense response. The gene trans-cinnamate 4-monooxygenase 4-hydroxylase (SEQ ID No: 2 and SEQ ID No: 4) is involved in synthesis of secondary metabolites, which have implicated to be associated with defense response. Further, our profiling experiments clearly show that the aquaporin gene (SEQ ID No: 5) clearly is down-regulated in the defense response.

It will be apparent to those skilled in the art that the polynucleotides of the present invention are useful for creating plants that are resistant to plant pathogens by numerous methods that are well known in the art. In one example, a polynucleotide of the present invention is linked to a pathogen-inducible promoter and a plant is transformed with the construct, such that the plant expresses the polynucleotide sequence upon challenge by a plant pathogen, thereby imparting improved resistance to the plant. In an alternative example, the promoter used is a constitutive promoter. Suitable promoters are numerous and are well known in the art. In yet another alternative example, conventional breeding methods are used to select for a plant having increased expression of a polynucleotide sequence of the invention. The present invention is suitable for the production of pathogen resistant plants from a wide variety of species, including, but not limited to: rice, wheat, barley, rye, cotton, sunflower, peanut, corn, potato, sweet potato, bean, pea, chicory, lettuce, endive, cabbage, cauliflower, broccoli, turnip, radish, spinach, onion, garlic, eggplant, pepper, celery carrot, squash, pumpkin, zucchini, cucumber, apple, pear, melon, strawberry, grape, raspberry, pineapple, soybean, tobacco tomato, sorghum, sugarcane, banana, Arabidopsis thaliana, Saintpaulia, petunia, pelargonium, poinsettia, chrysanthemum, carnation, rose, and zinnia. The plants can be engineered to express the polynucleotides of the invention by any number of well known methods, which will be apparent to those skilled in the art. Further, it will be understood by those skilled in the art that the invention is not limited to tomato genes or to the exact polynucleotide sequences disclosed herein; the invention also encompasses sequences having at least 40% or more nucleotide sequence identity with those sequences expressly disclosed herein.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

References

Bruce, W., Folkerts, O., Gamaat, C., Crasta, O., Roth, B., and Bowen, B. 2000. Expression profiling of the maize flavonoid pathway genes controlled by estradiol-inducible transcription factors CRC and P. Plant Cell 12:65-80

Halterman, Dennis. 1999. Characterization of the Fenthion Response in Tomato and the Identification of Genes that Encode Fen-Interacting Proteins. Ph.D. Thesis, Purdue University.

Jia Y. and G. B. Martin (1999). Rapid transcript accumulation of pathogenesis-related genes during an incompatible interaction in bacterial speck disease resistant tomato plants. Plant Molecular Biology, 40:455-465.

Jia, Y., Loh, Y. -T., Zhou, J. and G. B. Martin (1997). Alleles of Pto and Fen occur in bacterial speck-susceptible and fenthion-insensitive tomato lines and encode functional protein kinases. Plant Cell 9:61-73.

Loh, Y. -T. and G. B. Martin (1995). The disease resistance gene Pto and the fenthion sensitivity gene Fen are closely related, functional protein kinases. Proceedings of the National Academy of Sciences USA 92:4181-4184.

Martin, G. B., A. Frary, T. Wu, S. Brommonschenkel, J. Chunwongse, E. D. Earle, S. D. Tanksley (1994). A member of the Pto gene family confers sensitivity to fenthion resulting in rapid cell death. Plant Cell 6:1543-1552.

Martin, G. B., S. H. Brommonschenkel, J. Chunwongse, A. Frary, M. W. Ganal, R. Spivey, T. Wu, E. D. Earle, and S. D. Tanksley (1993). Map-based cloning of a protein kinase gene conferring disease resistance in tomato. Science 262:1432-1436.

Shimkets, R. A., Lowe, D. G., Tai, J. T., Sehl, P., Jin, H., Yang, R., Predki, P. F., Rothberg, B. E., Murtha, M. T., Roth, M. E., Shenoy, S. G., Windemuth, A., Simpson, J. W., Simons, J. F., Daley, M. P., Gold, S. A., McKenna, M. P., Hillan, K., Went, G. T., and Rothberg, J. M. (1999) Gene expression analysis by transcript profiling coupled to a gene database query. Nat Biotechnol. 17:798-803.

Tang, X. M. Xie, Y. J. Kim, J. Zhou, D. F. Klessig, G. B. Martin (1999). Overexpression of Pto activates defense responses and confers race-nonspecific resistance. Plant Cell, 11:15-29.

Tang, X., R. Frederick, D. Halterman, J. Zhou, and G. B. Martin (1996). Initiation of plant disease resistance by physical interaction of AvrPto with Pto kinase. Science 274:2060-2063.

Zhou, J., X. Tang, and G. B. Martin (1997). The Pto kinase conferring resistance to tomato bacterial speck disease interacts with proteins that bind a cis-element of pathogenesis-related genes. EMBO J. 16:3207-3218.

Zhou, J., Y. -T. Loh, and G. B. Martin (1995). The Pto kinase conferring resistance to bacterial speck disease in tomato physically interacts with and phosphorylates a second kinase, Ptil. Cell 83:925-935.

What is claimed is:

1. A genetically engineered plant, comprising:
a plant engineered to express a recombinant polynucleotide or to manipulate expression of a plant gene, wherein said recombinant polynucleotide or plant gene shows a specific pattern of expression associated with the Avr-Pto mediated defense response.

2. The plant of claim 1, wherein said recombinant polynucleotide is derived from a tomato plant resistant to bacterial speck disease caused by Pseudomonas syringae pathovar tomato.

3. The genetically engineered plant of claim 1, wherein said plant is selected from the group consisting of rice, wheat, barley, rye, cotton, sunflower, peanut, corn, potato, sweet potato, bean, pea, chicory, lettuce, endive, cabbage, cauliflower, broccoli, turnip, radish, spinach, onion, garlic, eggplant, pepper, celery carrot, squash, pumpkin, zucchini, cucumber, apple, pear, melon, strawberry, grape, raspberry, pineapple, soybean, tobacco tomato, sorghum, sugarcane, and banana.

4. The genetically engineered plant of claim 1, wherein said plant is selected from the group consisting of Arabidopsis thaliana, Saintpaulia, petunia, pelargonium, poinsettia, chrysanthemum, carnation, rose, and zinnia.

5. The genetically engineered plant of claim 1, wherein said recombinant polynucleotide is selected from the group consisting of SEQ ID No: 1 through SEQ ID No: 375.

6. A genetically engineered plant, comprising a recombinant polynucleotide
(i) selected from the group consisting of SEQ ID No: 1 through SEQ ID No: 375; or
(ii) hybridizes, under stringency conditions comprising a hybridization medium which includes from 0.9×SSC to 5×SSC, at a temperature of 42° C., to a DNA molecule complementary to a sequence selected from the group consisting of SEQ ID No: 1 through SEQ ID No: 375; or
(iii) comprises a nucleotide sequence which is complementary to the nucleic acid molecules of (i) and (ii).

7. A method of making a transgenic plant cell comprising the steps of providing a DNA molecule according to claim 6 and transforming a plant cell with the DNA molecule under conditions effective to yield transcription of the DNA molecule.

8. A method of making a transgenic plant comprising the steps of transforming a plant cell with a DNA molecule according to claim 6 under conditions effective to yield transcription of the DNA molecule and regenerating a transgenic plant from the transformed plant cell.

9. A method of imparting disease resistance to a plant comprising the steps of:
a) transforming a plant cell with a DNA molecule of claim 1; and
b) regenerating a transgenic plant from the transformed plant cell, wherein the transgenic plant expresses the DNA molecule under conditions effective to impart disease resistance.

10. The method according to claim 9, wherein said recombinant polynucleotide or plant gene shows a specific pattern of expression associated with the Avr-Pto mediated defense response.

11. The method according to claim 9, wherein said under conditions effective to impart disease resistance to the treated plant.

12. The method according to claim 11, wherein said plant is selected from the group consisting of rice, wheat, barley, rye, cotton, sunflower, peanut, corn, potato, sweet potato, bean, pea, chicory, lettuce, endive, cabbage, cauliflower, broccoli, turnip, radish, spinach, onion, garlic, eggplant, pepper, celery carrot, squash, pumpkin, zucchini, cucumber, apple, pear, melon, strawberry, grape, raspberry, pineapple, soybean, tobacco tomato, sorghum, sugarcane, and banana.

13. The method according to claim 11, wherein said plant is selected from the group consisting of Arabidopsis thaliana, Saintpaulia, petunia, pelargonium, poinsettia, chrysanthemum, carnation, rose, and zinnia.

14. The method according to claim 11, wherein said recombinant polynucleotide is selected from the group consisting of SEQ ID No: 1 through SEQ ID No: 375.


SEQUENCE LISTING (395 sequences)

SEQ ID NO: 1 - DNA, 255 bp, Lycopersicon esculentum
aaaagtatctttctatatatcttctaacttgctacacagaaatgtaaaaattacaaaatacatccaaatctggaaatgtatcataaaataaaacaaactaaaagcccagtttcctaaaagaatatacacgcccctgcatatatctcctgtttgtgtacattattttatcgtccacgcagacgtgccttcggcctaatgacttcaaacacaacttgcaaataagaagtgccactcagcctaatgacttcagacaca

SEQ ID NO: 2 - DNA, 255 bp, Lycopersicon esculentum
agcaactatggtgattttatcccaattttgaggcctttcttgaggggttacttgaagatttgtaaggaggttaaggagaagaggttgaaactattcaaagactactttgttgatgaaagaaagaaacttgccaataccaagagcatggacagcaatgctctaaaatgtgccattgatcacattcttgatgctcaacagaaaggagagatcaacgaggataacgttctttacattgttgagaacattaatgttgct

SEQ ID NO: 3 - DNA, 255 bp, Lycopersicon esculentum
tatccaaaaccaagagcaattggaacctatgagatctcacaagttgttgaagattatcgcaggtcggccttgaatgctattgaagcaggtttcgatggtattgaaatccatggagctcacggttacttgattgatcaattcttgaaagatgggatcaatgaccggacagatgagtatggtggatcactagccaaccggtgcaaattcatcacacaggtggttcaagcagtagtctcagcaataggagctgatcgc

SEQ ID NO: 4 - DNA, 255 bp, Lycopersicon esculentum
caagagcatggacagcaatgctctaaaatgtgccattgatcacattcttgatgctcaacagaaaggagagatcaacgaggataacgttctttacattgttgagaacattaatgttgctgcaatcgaaacaacattgtggtcaattgagtggggtatcgcggaactagtcaaccaccctcacatccaaaagaaactccgagacgagattgatacagttcttggaccaggagtgcaagtgagtgagccagacatgcc

SEQ ID NO: 5 - DNA, 255 bp, Lycopersicon esculentum
ggtgtacaatacaaatggcaaaagatatggaggttggtactgaatatgcaccaaaagattaccaagacccacctccagcacccttaattgatcctgaagagttaggaaaatggtcattttacagagctataattgctgaattcatagctactctgttatttctctacataactgtccttactgttattggctacaaaagccagagctccactgatcaatgtggtggagttggtattcttggaatcgcttgggcat

SEQ ID NO: 6 - DNA, 653 bp, Lycopersicon esculentum
ggagaagtagtctcaaatcaggaattagtaaggaatcttatcatgtcgtcttcatctgctgcttttgctccggaccaccacctccaccaccatcacctctctcctccttcaggagaacaactctgttatgtccaatgcaacttttgtgacactgttctcgcggtgagtgttccttgctcaagtttcttcaaaaatgtgacagtaagatgtggtcattgcaccaatcttctctctgtcaacattattcttcccactgctgctaatcagcttcatctcccacattctttcttttcacctcacaatcttctggatgagattcggaataatccaccaagtttgttgatgaatcaacaaattaacccaaatgaatcactcatgccagtccgaggaggagttgatgaaattccaaagccaccagtcgcaaacagacctccggagaagcgacagagagtcccatctgcttacaatcgattcatcaaggacgagatccaacgtatcaaagctggaaatcccgatataagtcacagggaggccttcagtgctgctgcaaagaattgggcccattttccacacattcatttcggacttatgcctgatcaacccgtgaagaaaccaaatgcatgtcaacaggaaagggaggatgttctgatgaa

SEQ ID NO: 7 - DNA, 700 bp, Lycopersicon esculentum
  misc_feature (578)..(578): Unknown nucleotide 578
tacacgaaagaagaaaatggaaggaggaacagaagcctatccagatttaggtagacattgccaactatctgattgccatcaactcgattttctcccttttacctgccatgcctgtttaaaggtattttgtgtggaacatagatcatgcaagtctcatgaatgcccaaaatctgactttaacagccgaatcgttttggtttgcgaaatttgttctatgtccatggaaactaccggctgtaaagttgaagaccacaaagcaatattacaaaaacacgaggaatctggggattgtgaccctaagaagaagaagaagaaacctacctgtcctgtcaaaagatgcaaggggattttgaccttctcaaacactagcacttgcaagatatgccggattcaagtttgcctcagacacaggttccctgctgatcacgcctgtaaccccacttcttcatcatcacagctgttgctaaaggaacccaataacaagtttttgactgctttgcttgcaaggaatgggaaagattgtgggaataaaagtcgtgcctcatctccaagccctgcgaacccttctgtgaaagctngttgatcgtttcacagcacattactattcatttgtcttttgtaattacattattttatgggaaaacaatgttgttctattttcaatttcttcctccttctatgataaaaccacgaaatttaa

SEQ ID NO: 8 - DNA, 574 bp, Lycopersicon esculentum
ttttgccaatcaggcacaacctttgtgaatttgtgaaacccgtttctagtaaagggatatctctccgtgatatccatttcacaccagatgatctagagatgtacattgatcttgccccatttttaaacccatctccgtatgtagtcccagaagacatgtcattaacaaaggtatataatctttttcgccaactaggactgaggcacttacttgttgttccacgtcctgctcgtgttattggcatgatcactcgaaaagatctgatacttgaggataatgatgatccaacggctgtagaactccaatcaactagtatgtgcgcctaatttttccccagcctccagtgacagctgctacacaattacctaaaatgtctaatcatactctgatttctcagaaatgaaaaaaaaaagtctggcagctggtctggacaccatacaacatcaaaatcagtgtagtcccgcaagtggggtctggggagcgtagtgtgtatgcagtcttacccctaccttctgaaggtagagaggttgtttccaaaggaccctcagcccaagtaacacatgagaaacagtat

SEQ ID NO: 9 - DNA, 778 bp, Lycopersicon esculentum
ccaattccatcaagtgttaagcttttgttagtaaggtttttaacttttcaaacgtccgagacaatgactcttgcaattgaaattgagaaaaaacatatcaagaaatcagaaagggtcaagtccttggagatccacccaacagagccatgggttttagtgagcttgtacacaggaactgtacaaatatggaactatcaatctcaaactgtcgagaagaactatcaagtctcagagtcaccagtcaggtcagccaagtttatcgctcgtgaaaaaagtattgttgttggatcagatgatcacttcattcgagtgtacaactacacaacagaggaaaagactaaggaattcaaagcacatgacgattatatcagatcattagctgttcatccttctcttccttatgttctgtcatgttccgatgacacgatgataaagctttgggactgggaaaaagactgggcttgtactcagatttttaaggggcataaccactatgtgatgcaagtggccttcaatccaaaagatcaaaaccagacgtttacaagtgtatcacttgactgcacaatgaagatgtggaatcttagctcctctgatccaattctcgaactagaagcacattcgaaggggatcaattgtgttgatttcttaacttgtaatgataaacaatatctaatcactggatcagatgatcacacagctaaggtctgggacttgcaaaatcaaacttgtgtccagattctagaagtcaccagaacatatttctgctttggcatttgca

SEQ ID NO: 10 - DNA, 587 bp, Lycopersicon esculentum
acagctagctttctcataacattgccattttcgactctccttcttccttggagacattcttattcattttcctctgccatttcaaacaattatcgcgaaaaatggcgttttctgatagaaaatctttgatccctagttatctttacagtacaacatcagctaattcttcaaagaatctcagtagtgcatttgacaatcatgacaatatggataagaagaatttattgattgcagcaccaaaggaaggttcaggttataaggtagaaatgtactcaccggcgttttacgcagcaagcacgataggcggtattttgagctgtggtcttacacacacggctgttactcctcttgatcttgtcaagtgcaacatgcagattgatcctgcaaagttcaagagtatttcctctggtttcggaattttactcaaggaacaaggaattagaggattatttaggggttgggcacctactttacttggttacagtgcacaaggggcatgcaaatatggattctatgaatatttcaagaagtactactcagatcttgctggcgcggaaaatgctgtcagtacaagactttgatttacc

SEQ ID NO: 11 - DNA, 565 bp, Lycopersicon esculentum
gcccaaattggagaagaaagaagaggaagtagaagcagaagtagaagaaaacccaactggcgatgatgaagacactggtgcacaagttgctcccattgttaggctacaagaagttgctgtctccactggtgaagaaaatgaacatgttcttcttgatctgaaatcaaagttataccgatttgacaaggaagggagtcaatggaaagagagaggtgttgggactgtgaagcttctcaagcacaaggaaactggaaaggttaggcttgtgatgaggcaatccaagacgctgaaaatctgtgccaaccacttggtccttcctactatgtcgatccaagaacatgctgggaatgagaagtcttgtgtgtggcatgctgctgactttgctgatggagaactgaaagatgagactttttgcatccgctttgcttcagttgagaattgcaaggctttcaaagagaaggttgaagaaattgctgaatctcaacagacaaagtctggacaaagtgaagaagctggtgctgctgctactgaacttattgaaaagcttagtgttgaaagcaaagat

SEQ ID NO: 12 - DNA, 2118 bp, Lycopersicon esculentum
aattcggcacgagttttcttatataaaaaacatatcatcttcttattgttattttaactaccacatttgaagccggaaaacttagtaacacgatggccggtggaggatttacgacctccggtaacggagcacgcatttcgaggctaaaatcacaccaattggttatcatatcttgtattatggctgctaccggaggtctcatgtttggttatgatgttggagtttctggtggtgttacatcaatggatccatttttaaaaaaattctttccaacagtttataagagaacaaaggagccaggattagacagtaattactgtaaatacgataatcaagggttacaattatttacttcatcgttatacctagctggtttaacggcgacgttttttgcttcttacactacaagaaaacttggccggagattaactatgttaatcgccggttgttttttcattattggagttgtactaaatgctgctgctcaagatttagctatgcttattattggaagaattctccttggttgtggcgttggtttcgccaatcaggctgttccattatttttatcagaaatagcacctacaagaattcgtggaggacttaacattttgttccaacttaacgtaactattggtattctttttgccaatctcgtcaactacggaacagccaagattagtggaggatggggatggagattatcattagggttagctggatttccagcagtattgttgactttgggcgcattatttgttgttgaaaccccaaacagtttgattgaaagaggttacttagaagaaggcaaagaagtacttcgaaaaatcagaggtaccgacaacattgaacctgaattcttggaacttgttgaggctagtcgtgttgctaaacaagtcaaacaccctttcagaaatttactccaacgtaaaaatagacctcaattaatcatctctgttgccctccagatattccaacaatttacaggaatcaacgctattatgttctacgcacctgttttattttcaacactagggtttggaaacagcgcagctctttactcagctgtcatcaccggagcagtcaacgttctctccaccgttgtctcagtctactccgttgacaagctcggacgacgagtcctcctcctggaagctggggtccaaatgttactatctcaaataataatcgctataatcctaggtatcaaagttactgatcattcagacaaccttagccatggttggggaatcttcgtagtagttctgatctgtacatatgtatcggctttcgcatggtcatggggcccacttggatggttaattcctagcgagacgttccctttggaaactcgttcagctggtcaaagtgtaacagtgtgtgttaacttgcttttcacgtttgttatggcacaagcatttctctcaatgctttgtcatttcaagtatgggatattcttgttcttctcggggtggatttttgtgatgtcgttgttcgttttcttcttggtgcctgagacgaagaatgttcctattgaggagatgacggagagggtgtggaagcaacattggctgtggaagaggtttatggtgaatgaagatgatgttgatatgattaaaaagaatggacatgctaacggatatgatcccacttctcggttgtaaaataaaaaataaaagaatattagcggtgttttacgttgattttatcatagtaaagatgcgtagaaataaatcaaacatgcaagagagagatcagagtctactgtctagtctagtgatatcgataagtgtttctcacaaaataggtgtgcatttggattttttttactgcttgttttcctcctttatatcgttcaatgtagtagaaattgtttagccaaagtgtaagagatgcaatatggtgataatttatgtaaccatatagcctatgtatattagagatttggtataattcattttgggaaagcactttgatatgtttaatttcttgttatgtatgtatgacaaggtgtctagttataatgataaggcccaatagaggtttgcatgttgagtattgagacacaaaaatgaaaaaaaattacgctcaatgatcttgaaaaaaaaaaaaaaaaaa

SEQ ID NO: 13 - DNA, 606 bp, Lycopersicon esculentum
  misc_feature (577)..(577): Unknown nucleotide 577
gttctttcaagaacgtgctatattcatgaaggaaagagcttcgggtatgtacacaatctcctcttatttcatggcgcgaattgttggagatctacctatggaccttatcttgcctacattgtttcttaccataacatattggatggccgggctaaagccacaactcttggcattccttttgacactactagtcctgttgagctatgtgatagtgtcacaaggcctcggcctagcccttggcgctataatcatggatgctaaacaagcttcaaccgtgatcactgtcacgatgctagcattcgttctaacaggagggttctatgtacacaaagtcccagcttgtctagcctggatcaagtacatatcaactacattttacagttatcggctacttatcgatgttcaatttggcgaaggcaaggagatatcagacttgctaggctgctcacgtattcaaggaagtgacagagctaattgcaaattcattgaacaagatatcataggccaaattcatccattccatgagcctggaatccttctgataatgtttgttggatatcgattaatcgcttacctngcattgaggcgaattcgagcatagacaaa

SEQ ID NO: 14 - DNA, 572 bp, Lycopersicon esculentum
attccctagctagctggctagctctttcaattcggctcctttagaaacggtttcaattatatatttgtgtatacttcattaagtatctgtgaagaatggagagtgaaggagggaactgtagcaaaagcataaaccaaaatgaattggtattgaaggaagatccgaaatcaaacttttttcaaaagtattatcgatctggtattataaagaaggtgatagcagagataatagcgacgtacctattagtgtttgtgacatgtggtgcagcaagtcttagttggagcgatgaacacaaagtatcaaagcttggagcgtgcgtagctggtggactcattgtaactgacatgatatatgctgttggacatacatctggtgctcatatgaaccctgctgctacctttgcttttgccgccgatagacattacccatggacacaggtgccagtgtatgcagcagcacaagttacaggagcaatatctgctgcattcacgttacgtgtattacttcatccagcaactaagaatgtgggaactacaactccctcaggctcagatattcaagctttaatcatg

SEQ ID NO: 15 - DNA, 465 bp, Lycopersicon esculentum
gtttgtttgtatgatacttttctactttagaaaaaacatggaaacgctaaacaattaccagtattacaagaaaaaatgtcaatactccagagctcaaaagtactgttagtttgtataatactgaaaatgtcaatacatgtataccaattctgagtaccatatatattacaatccgtcctaatctacgcgaattacctaacatttttcccatgtcatagagatcacgattgacagttcagaattcttcatatcaccatcatcatacctatatatgtgttgcttcattttgatcgttcgttttcttatctcttgcgtttggactttagagatgtcacagactgtgaattgctatgccaattgcgttttctttgattgatgaaccagttattgatttgcttcagctgtaatcctgtttcttccactagttttgccttatcatcctcagttggataaggccattttgag

SEQ ID NO: 16 - DNA, 379 bp, Lycopersicon esculentum
gtgcaatacaaacttcaatctccataccaactatcttttcccaccggatttttacttcgaagccggcaataaaggtactctatctttctccgctgttgattcatccaaattcaagttcgagaaagaagataaaattaaaccctttttcgaaacccttgattattggggaattcagaggaaaaggactaagatgatgtgtatgaattgtgggaaggttgtgggctatgtgtatgatgacgggccacccatgactgagagtccgggtcagtttcattttggtcctagtcaggttattcctagagctgcaaggtataggataaaaaataaagctttgaagattacttctgagacttgacatgtgatttgatttttgatgttc

SEQ ID NO: 17 - DNA, 605 bp, Lycopersicon esculentum
aaatcaaattataaaaatatttatttgagcaaatccaacactttaatttcaagggccaccaaaagtctcaatcttgtgagactgaaaaggagcaaactgagacctaactaatataggctgctatagaaaattagcccatcccattttcacgtgcacttactagtcctacctactggctactgttctcccaaaaaaaaagaagacaaatatagtaaacaatatatacaatttataacgacatgtcctagtggctgcctgtctttctctggaacatttcaatgtgtcaacataattttcgttttaattatttatccttagtgttactattattttgcaagaattttcctgatatggcggctgctaaggctgcttgaaaacttggatctttagtcaatgatgaagccatttgttctatcaagaaatgttgaaactctggtctattttgctgttgttgatcagttcctggtgatttacgcttctgaccacttgcactacttgtacttgtaatgctatgaacttttttgatatcattttgtaatgtttttggttctgccaaatcaagagttaatgtttgtcttggcattaccgcagcagcttaagtatta

SEQ ID NO: 18 - DNA, 496 bp, Lycopersicon esculentum
gtcataatcatcccaaacctcaatccactagaagatcatcttcacaatcgattcaaaaccttgcttactccaacttggatgtaacaaaccagccaaacgcgtttcttgaaaatggtcaaagagactcctttgctgttacagacaattcttcagcttcttttggagatgacgatgttgatcaaggctctcctatcagtaaatcaggagaaaatgatgaaaatgaacccgaggcaaagagatggaagggtgacaatgaaaacgaggttatatcatctgcaagtagaacagtacgtgaacctagaatcgtagttcaaaccacaagtgacattgatatacttgatgatggttatagatggagaaaatacggtcaaaaagttgtcaaaggaaatccaaacccaaggagttactacaagtgcacatttactggatgtccggttaggaagcatgtggaacgagcatctcatgatctaagagccgttatcacaacttatgaagg

SEQ ID NO: 19 - DNA, 587 bp, Lycopersicon esculentum
aagtcaagagacataacagtaagttgtatatttcataatctcatctcagtattctacaaagttgtcaaattcaacaacttaaatgcttctaggacaattcagtctcaatctagccaactcagagcaagtccattctgcaggggacccagtatggctgctgttgaaaatttcttaagagtctagtaagatcccattctcacgtgcatctccgagtccaactttgcggtccaaatgttactaacttttcctgtatttacatttgaacaataatttgtgtttcctttttcctcctaagaacttttgatatgagtcaagttggaaaatattgaacaattgtctatacaaagtagttgactgctctgctggacttttgtttaccatctccctgtctgattattatgttggaggatttttcctgatatggcggcagcaagtgctgctttgaaacttggatctttggtcaaggaagtagccatttgttctatcaagaactgttgaaactctggcctattttgatattcacctcctgctgctgatgttgttgttgttgttctaatgctattgccactggtaggactagtagtact

SEQ ID NO: 20 - DNA, 447 bp, Lycopersicon esculentum
gagagttcagataccattgacaatgtgaaagcaaagattcaagacaaggagggtattcccccagaccaacagcgtttgatctttgctggtaaacaacttgaggatgggaggactcttgcagactacaacatccagaaggagtcaactctccatctggtgttgcgtttgagaggagggatgcagatctttgtgaagactcttactgggaagacaatcacattggaggtggagagctctgatactattgacaatgtgaaagcaaagatacaggacaaggaagggatcccaccatatcaacagaggctcatctttgctggtaagcagcttgaggatggccgcacccttgcagactataacatccagaaagagtctactcttcatcttgtcctgaggctccgtggcggtgacttgtgaatgtcctgtgtgctgctgatttcatgatggctg

SEQ ID NO: 21 - DNA, 430 bp, Lycopersicon esculentum
aaagaattcggcacgagtgggaacggaaggtgagcaattgacggagcaggaaactgccatctatgatcgacaaattagggtttggggcgttgatgctcagagaaggctcagcaaatccaatgtgtttgttagtgggctgagaggaactagtgttgagttctgcaagaatattgttttagctggagtaggtagtttgacactaaatgatgaccggttggtgaccgaagatctattatttgctaatttcttaactcctcccgctgaaaatgatttccgggggaagtctgttgctgagctttgctgtgattctttgaaggatttcaatcctatggtttctgtttctgtaaagaaaggtgctctgtccatcttctatgctgatttctttcaaaagtttgatgttgttgttgataattgttgcttcccttttgac

SEQ ID NO: 22 - DNA, 631 bp, Lycopersicon esculentum
tcaatggcactctctgctctatcacctactatgtctacaaacccttatgcatcatcaacctctctatcaaaaacaagatgaatcacccaaaaaaatgccaaatttagtgtctttactacccctaaattcactccaatttcagcatctttctgatccatatgtccttcaaatagctgaaacacttgaagattcattaccttctacctcttctacaccacttcacaagttaagaatcagttcctcagacacccttttgtcaaccccttggccatctcgtaaagatgagccttttagattcactgacacttcttttatcaagaattcaagaattgagccaattcaacctccatctttgagttctttggatgtttctgtagatacccttttgcctacactttcaattgttgatggttatataatggattctttgtctcagttgaatgaatttcctagtggggtttatgtaggtagtctgttgagggtagattctgaggctatattgaaaagggtgtctgaatatgagttcagttctaaaggggatttgttttggttacttaatggtgtatgtaccgctgatgtactactagtgtatgtgccacaagggtgtaaggttgagactccattggtattg

SEQ ID NO: 23 - DNA, 695 bp, Lycopersicon esculentum
gtcaaatcttccaatttgacgccattacagacgcatcacagttctccatcttcgtaactttgcaccaatcgcatcatcaccatcatgattagaggagaaacagtggaatttccttcatattctcatcttaaaaagagattcagaagagtttgtatactcagagagcctcgtgcgtcatgcattaacatcctctaaggctggtctcagatcgcagcacttaggccttcacaaagctttatgtgctttgatgggatggaagagtgcagattctcatgaagatagttgggtttgtgaaaagctatccaatgctgaaactgtggctttgaaggaagatctaatcatctggcctccagttgttatcatccataacagttccataactagcagcaatccggatcagcgtgtggttgtatcagctgaagagctcgagtctaagctgagagatatgggcttcggggagaaggccaaggtttctcgtggtaagcctgcaaatcagagtattcttatggtgaagtttagcgctactctctcaggtttggaagaagcagtgagactgtgtgatatctacgcccatcggaagcatggaagggcggatttccagcgtataagttctggtcagtctggaaacaatgacgatgaaactaaagaaccattgacagacaaagtaggtaaaatcctttatggttaccttggaa

SEQ ID NO: 24 - DNA, 617 bp, Lycopersicon esculentum
  mRNA (1)..(617)
ctatttctgacttggaacttcaagtatgtatccctctctctctcacacacacagattgtaattttcaattgaacggaatggttataaaggggacattaagtagttagggattgaaatggagaaaaattgttatggaggattcatgtagcttataataactagtttgccgtccgatccctttgaaaaattgctcatctttactcatccctttgctctaattccttacatttgaagttggtagatagtagaagacgttgtagagagagaaaaaaagaagcaaaatccgatgatgggaagcgattcaacatgggtaggaagaaaacccatgagacgacttggtggaatgtctgatgctctctctattgctgctgaattgggcttttctgttcctcctcctccttctcaagaggagatccaaaacttatccaccaccactggcgaaaatggtgatgatttgatcagggtcttgaaggaacttactgctgttcaaaggaagattgcagatttacaagtggaacttcaaggtcgaaaggaggacaaaaatgttgctcatctgacacatgtcagtgaaatggaaaagaagattgaaacattgccaaggattactactattttga

SEQ ID NO: 25 - DNA, 643 bp, Lycopersicon esculentum
gaattgcttttttctctttgaagtttcaatctttcttgatatttctgtcactctgtctaaccaatcttgcattttgcttaaagtagagtgttcaagttgatatttttatttgtgggtaagaggaaagaagatgcctagaccaggacccagaccttatgagtgtgttagaagagcttggcatagtgataggcaccaacccatgagaggttctatcatacaacaaattttcagggttgtaagagaaaggcatagtgttgttacaaaaaagaacagagaatggcaacagaagcttcctgttgttgtcttgaaagctgaggaaattatgtactccaaagctaattctgaggctgaatatatggatcctgaaacactttgggatagggtaaatgatgccattgataccatcattaggagagatgagacaacagagattggacaactcttgcctccttgtgttgaagctgctcttaatctgggatgtataccggagagagcgtctaggagccagcggaacagcattccaaggagttacctcagcccaagaactcaggaacctcattgtgtaccttctaaagtttttagtagaagtgccaatgagctgaactctaactcatcgttacctattcgctctacaacccaccac

SEQ ID NO: 26 - DNA, 712 bp, Lycopersicon esculentum
gtataagaaattgccttacgaagagcatattgttccttatttcacttttggttttgcatatgatgaattcgaagatgattacaagatagtgggtattttccctatttataattatgcaagtctttgtcgtgttgaggtcaaaatatatagtttaaagagtgattcttggaaacgtgttaagaattataaaggtgcagagctcttgggtgattctgctaagtttgttaatgggaaatttcattggcttgataagcactggaacattatttctatggatttggcgggtgagaaatgggaagaggttgatcagccctgctgttttaaaggatgtggatttttgaagctaggagtgtttgagagtgatctttctgtgttttgtaattacgcgtggactcatgttgatgtatgggttatgaaggaatatggagtaaaagaatcttggacaaagatgttcaccgtcaagtcacccgaagattctgggggacgtatattttatccaattattttaatgtcaaatgaaggtgaaattttgcttaagtttggatcacgtttcgtgaaatacaatccaaaggatgactcaatcagatatctagatgttactaagttagctccatgtcttgaggtacaaatatatgttaagagcctagtttgccctttttactgatgagatcgcgatttcaccaccaaagcaggctgaaataattctgatgaa

SEQ ID NO: 27 - DNA, 551 bp, Lycopersicon esculentum
ttcaattctaaaattttctcttttcaatttggtaccaaattttcctcttctttaggggttcgaatcagtgtttccctctcttttccatgtcttcatcttctaaatctcatacatcgtaaccctaatttcgtttacaatcaccaatggacggcggcttcactggtggagaaccggtagggagggcgtccacgtcctatgaagctgcaggaggacggcggaggtttacggtggagctcaggccgggtgagaccacaattgtgtcgtggaagaagcttctcaaggacgctaataagtctaatgttaatgttgtcggttctggtccaacgatgatgccggttggagctcctgctagcattcaaactccagtgccgcatcctgctctcgaggctcgattagctccgggacaacctgctgataaagaagtcaaggatgcacctcctggaaatcgtttaaatgctgtgatagaaaagattgaacgcttatatgtgggcaaacaaagtgatgatgaggaagatcttaatgatttcccagatgacgatgaatatgacact

SEQ ID NO: 28 - DNA, 514 bp, Lycopersicon esculentum
gcatgaactcttcattttgccctttgtttcgtccttaattacgattctaccactctggtaaatccatatttctatactgtgatggcatctccgagaagttgccggcgagtaattgaggagaggaatcggaagggaaagttgtcggagaaatccatgtcgtttgaaggatatatcccagggagcacttcagagatatcacgaagaattaggactgttccggacttattctccggcaagggatctgcttccggttttatgccggatttacaggttaggccaaagttgacaaagttgctattgaatgtgacgattcagcgcagtccaggccctgtacaggtgctaatatcaccggaatcaaccgttcacgatctcatcgccaccgctgtacggcaatactcgaaggaaggaagacgtccggcactttgctccacggatttgttcgattacgatcttcactattcgcagtttagcttagccagtctggacagggcagaaaagttgatggcgttgggaa

SEQ ID NO: 29 - DNA, 499 bp, Lycopersicon esculentum
  misc_feature (499)..(499): Unknown nucleotide 499
cagcccgctccaagcctgctgaagaaaggaggcaaatcgagtcagttgtatccactgagttccgcaaaaactccaagcaagttgatcgtaagaatttcatttatatcgagtacttacttcgtcgtggtaacaaacaacttgatcaactcaaaagccctgatactgttggattgtcatcattaagtgttgattcttcacaaacaacaggttcatcctcctcctagattttgtaaagaaaccactctttttttcaactattacaatgagttggttgattatataacatctcttttgctgggattatcatactgagtatactagtattcagaaacatttatatggtgttaaaagaggattttcatcgaggctcgagagatggaatagattacatttacgtttcttcaataaaaattcaagctcgacttgatagtgtaaaaattctttacaagcatatcctatgattgaatgatagtttttgtaatagtagattgtttcattn

SEQ ID NO: 30 - DNA, 565 bp, Lycopersicon esculentum
gacagaatcttggaaacctgatcaaccagagaaggcacaaatgaccattttttatggtggacaagtcattgtttttgatgattttccagctgataaagcaaatgaaatcatgaagttagccaacaaacagaaccccacaaacaatttcacttatcctatgatcaagaatcaaaaaacagctgatcaatctggtgtgagttttggtaacaaattgattcaagaacttcctaaactgtcaatgccacagccttctgttgctgatttaccaatcgcgagacggaattcacttacaagattcttggagaaaaggaaagatagagtaacatcaattgcaccataccaaatctccaacaacaagaaatccaagaatgaggataacaaagcatggttgggattaggtgctcaatttgttaaaactgagcaatatttctagttactattcttgttttttttttgttttgttttcatttttgattattgaagaagggaagtcttggtgtgacgtaactggtaaaagttagtgttatgttggaccagaggtaacaggttcgagctgtggaaaagc

SEQ ID NO: 31 - DNA, 672 bp, Lycopersicon esculentum
tactactattacaaatggcttcatcggagattgtggattccgggagatttgccggtcagaaatcgcatttctctcatacatgtaacttgttgagtcaatacttgaaagagaagaaaggttctttgggagatctcagccttgatatgcatcgcaatttcgattcagctggttctactactatggatttgttgccgatgattgagaaatctggtgagttggttcagaaatcgatgaatctgttccctcaaggtggaatgaaggctgagtcggaaccggaaaaggcacagatgacgatattctatggaggtcaagttattgtgtttaatgattttccggctgataaagctaaggaaatcatgcttatggctagtactagcaagggaaacaatcctgctaaaccattggaatctgctgccgatttggtggttccgagtttcggaaaaacttccatccaggaaaatcaaatgcctaaccagccaattgtttctgatttacctattgcgagaagagcttcattaacaaggtttttggagaagagaaaagataggctaactgcaaaagtaccttaccatagagaggaagcagcagctcctaaaaaggaagaacacaaagcgccatggctgggattgggtggtcaatttgcagtgaaaactgagcaatactagtttaccg

SEQ ID NO: 32 - DNA, 445 bp, Lycopersicon esculentum
ctagtaataataaataaaaccacaaataggtggtctttcaacatatatgatgtatacaagaatatatatcaatctgcaaaaattaaaaaaaaaaaagacaaaatctgaccaaacaacaaaaaacatataaacatacaaaaatgttcaaagtgatatttttaagttatgcctgttgatgagtcccatttccataaggattaacagtaccatttgttccattttgtgtgtgcttgttagagtctgtttttttccttttaatgacaatgaaccaagtaaaagcttctaataaaacagcaatggcacctatggctatgatcacaccagtgtaagccctcttccaattcttctgtccattcaatgcatcaaatcctttgtaaacattcgcgatgctcagacttattaccgcgtatccaacagcatggtggtagatgttccagtataatct

SEQ ID NO: 33 - DNA, 496 bp, Lycopersicon esculentum
aagtggtactcaacgtgtatatatattatacaagaacaaattacaaacaagagaatctaaatctagcaatcaaatatccaaaaatctgatgctaaacatcatagattaatatcaatctcaactaaaacataaataatcatgcctcattatctataaagaattcggaaactatgtaatatgatttattattaaagagaacacatttatttacgcggtctggtcatgcgacctgctggcatatccattagtgccattgccatgaggatatttgtctgagactgatgtttttttccttttaatgacaatgaaccaagtaatggcttccaatagaacagcaatggctcccaagaatatgatcactccgatgtaagcctttttccagtttttctgtccatttaaagcatcaaatccttcaaatacattgatgatgctgagaatgatgacggtatatccagtaacatgatggtagatgttccagtagagtcggaacttgtgg

SEQ ID NO: 34 - DNA, 692 bp, Lycopersicon esculentum
gcaccagatggactcctcttatcaaattgttgccaaggcaaaacaccgccactattactctgcttgttttggacgcccggctgccaccgctcttccaccagttggagttgctacatatcccaatctagccacctccctctacaacacccacctcggcctttttgctctaacttggtcacgcaatctgttcggcagatcttttcatatccatttcctcctcaatggcctttcttctcctcatctatccccttctttccaactccacatcaagccttttgttttctggaagaaacacggatctaacaagctccataacaatgttcacatcttttgggatttttccaaagctaaattcggatctggccctgaacctcgatctggattctacatggctgctaccgttaacggagaaatggttcttctcgtcggcgatttactcaaagaagcttattcaaggacaagagctcgaaacaagtacatgaacatgaacatggtgctgagaagggaacatgtgtactgtgggaacaaactgtacataacaaaagcaaagtttggtggaaaagaaagagaaatctcaattgattgcaggattggggaagatccaaggttgtatttcagtgtggacaacaaaagggtattgcaaattaagcacttgagatggaaattcagagggaatgagagaattgaagtaa

SEQ ID NO: 35 - DNA, 548 bp, Lycopersicon esculentum
gataataattgcctttgctataatagctaaaaccagaatattgatatatagtttacatgtccacaaataaccaccaacagcaacatacaccgtgcaatcccacaaatggagtaatggaatggtacaatgtacgtagatgttaacttctctaacatgcattataagcaggacaactaaacgctgggctgtagaccaagtcctttgcgcgtctttccaacaaatagatccccagattttatcatgcctggaatgcgtacagcgattgacaaacaaggaaattgagccactgcatcttttctccctagagaaactatagccaatgccttggttgctgggttatatacagccaatttgtgatcatcttccactccctttattaacgacgtgatgttcttggctactacctttgcgtgttcttgagccaaatatccttgtttaagttccggaacatcagtgatgttcccaatagcaaagatatttttgcgagccttgatcctcaaatttgaatcaaccattaccttttcacgactgtccaaactatacttcaa

SEQ ID NO: 36 - DNA, 659 bp, Lycopersicon esculentum
gcaccagcacatatctaacctatcaccaccgactaagtcttccgttgtattttcctgttaaagaattttccggccgatctctttctctattttatatcggatttttttctttctctatattatatcggtattctttctgttgaatattcgcatttgcatggcagtaagttttttgaatcgggaggtatctgacctgtgtcttggtaagccggcgttaaagcctattccagctgaagctactgtatcggaggcgttgacattgttgaaaaaatccggcgagactcatgtaagcgtttggagctgtgaccatatcaggaaggttgtagaagaagaagaaggaggcggaggatattctgtttgccgctgtattgggaagatctgtatggtagatgtgatttgcttcctctgtacggaagagaatttggcggatccttctaaggctcttcaaactcctgtggaacagattttgcctaaagggaattccattgtcagacatttagatccaaattctagcttgttggaagcgatagattacattcttgaaggcgctcagaatctggttataccaattcaaaattctaagagcaccccttcgaggaaaaagctttcgagtaaagcgtcatttttgactcccacaaatcataacggagttgaatact

SEQ ID NO: 37 - DNA, 535 bp, Lycopersicon esculentum
gcaccaggtggtttcaattcaataaggaatttacagagtatgagaattttgataaatgtcttgaatacatagaagaatgtatgatcaaacatggaccttcgatggtcttcttggtttctctcagggggcaattctttccggagcattgtcggggttgcaggctaaggaagtgggacttacaaaggcatcaaaaattaaatatctgatcataattggaggtgctaaatttcggaataaatcagtggccgaaaaagcttattcaccagcaattgcatgcccttccgttcattttttaggtgagcaagattacctaaaacagtatgggatagaactgttggaatcatgtgttgatccagtggtgattcatcatcctaggggtcacactataccaagattcgatgagaaaggattagagagtgtgcttagtttcattgagaaggtgctagaagagattaacaagtgatcaagtttgataattttttttcaaaaaaaaaaaatagtggtagtccctagtttgggacatgtagttaccacc

SEQ ID NO: 38 - DNA, 602 bp, Lycopersicon esculentum
gccaagtcaattttcttctatcaatctctaattgaagtatgaaatacaatatttgtctgcagtgtatgctagaaaagcaacattttcatccgtttgcacactaacttaacttaagaaaaacaaatgtgaaaatataagtaataacttgaaagaattacatgaatcaaagttttccctcctttgattgtattaaaggagagaagtaccagcatggttatggtgtagaagattatgagcaatagcatcccttgcaagcctagcattggtcgatctcaattgaatctctggaagcatttcatcatcaatgagctcgaaattgagttctggatccatctcctcattcctcatctggcaaatattatgcaagacgcagcacgcccctagcaccactggcaaatcttgtagtttcacctctgttcttttctgcaaacaagaccatctccttttcaaccttacaaacgcctcctttgccacccgttgaacctcaccaatcttctcattgaaagcatgttgagtccaagttagctgctgctgtgtataaggcactaaaacccaatccattaaagggtatccagaactaccaacaatccaaacgttgttca

SEQ ID NO: 39 - DNA, 583 bp, Lycopersicon esculentum
gaaattattgtagtcttcggcttcattagttctggagagcattctagttcggccgcagaagaagcggagctcggagaaaacaatccggattctcggtgttccatgcgatgctgtttccgtgtttgttcagtttctctcttctttccagtgtaccgaagagcagatgaagaaacatggaattcatcttctagcactttctcatgtgtacttggtaccacagctaaagcagagatgcacaaaggggttagctgatcaattgactattgaaaatgctgtggatatgcttcaattggctaggctctgtgatgcacctgatctatatctgaagtgcttgaaattgatatcaactaatttcaagaaaactgagaagactgagggttggaagttcatcctagatcatgatcctaaactcgaactcgaaattcttcagttcatggatgaggctgattcgaggaagaagaggacaagaagacatagaagggagcagaacttgtatttacagctaagcgaagctatggattgtatagagcacatatgcactgaaggatgcaccagtgtaaggccatgtggtgaggagccctgc

SEQ ID NO: 40 - DNA, 607 bp, Lycopersicon esculentum
cttttgtatgatcttaaggaaaaaactagcagtaactttcctggttctgattatcgctcagatgatcgaaaaaattggatgtcaacactggctctggagaaacttcaagtgaaccagataatttggcccggtacccatgattcagcaacaaacaaaattggaatcccatttatttctcgaccttttgcccgaacacaatctatgtccatttacaatcagctggtaacgggcactcgtgtccttgacatcagagttcaagaagatagtcgtgtctgtcatggcatcctagtttcttaccatgtcgacgttgtgatcaacgatgtgaagaaattcttatctgagacccaatcagagatcataattctggagatccgaacagagtttgggcacgatgatccaccggaattcgacacgtatttagagaatcaattgggggaatttcttattcaccaagacaactctgttttcaacaagacagtagcagagttattgccaaaaagagtaatatgtgtttggaaaccaagaaaatcagctcaaccaaaacatggaagtccgttgtggagcgctgggtatttaaaggacaattggattgacactgatttgcctg

SEQ ID NO: 41 - DNA, 664 bp, Lycopersicon esculentum
  misc_feature (549)..(549): Unknown nucleotide 549
gcagtgaaattttctcttctctccatcatctcactctctgcaaaagcaagatttcatggttgggccttaactcttttgagcaaccacacaccgttcctctatggcgacttctttgaattctgttctgggctttccgtcaaccgcaaactactggaatctatctgggagtgcctctctgcagaaggttgcacacttcccatctacctttcagatgcgggggttaagaagatgtgtaccagttactagacataatttctatcttttagttgctaaaagtgatcgtcttgaaacggaagttagtgataaggactctgaaaatgctgaaaggtcatactccgaagctattacaccaactgtgaatacgtcatcctttgaatcttctaaaatagagggggaagtgcaacccattggtcaaactgattcggccgtgcaagaagccgaagcttcaaatgggtctggaacttctgcaaacttggaagaggaagcatcttcctccagtccaaaaccaacgttgaagagatctccattgacagcaagagagaaacttanggcagccagggttctcagccggtacaatgaatcaaagacctctaaacctcagatgggaagcaaactaattgaggccctacgagaaagtgagaaggggaaaatgagatccggactt

SEQ ID NO: 42 - DNA, 456 bp, Lycopersicon esculentum
gagatcggttagagttaaggttttttgggggttccgattcagtttaccgatcgggttcaagttttattttttcttaattgtttgaattgaatttggtgatactaggaaaaaagaaaaaggaaaaaaaaagggattgaaattggtatttgaagtgaaaggagggtttaggttgaacgatattgaccgccatggttggtttgtgatgtaatatggctctggttggtacagataaagacatgtcgttttacattggtcgcgaggcttcaaagttatggaagagattctgtgcggaggtaacaacagagatcaatcttcttgctgaaaattggaaatatcttcttggtggtttgatttgtcagtacatccatggacttgctgctagaggggtgcattacttttatcggcctggaccaattctttatgacgtctgcttctatcttcttccggagcttggac

SEQ ID NO: 43 - DNA, 551 bp, Lycopersicon esculentum
gataacgaattcagtgtttcagctagctccagttacaaatcagactgcctattagttctatgacttccttttctcttgagctacttttggaaagcctagataagaatatagttgttgaagtatcctcctcatctgttgctatcccattccctgctccacttttcagatatacctgtgaattatgagatcaggaaaaagaggtgcaggacccagagttgcaaagtgacacttgaacttcgttcctgcagccaaagaaattgaccggagagtgcactgtcaggagttagattcgatcttcacggattattctgtggggaagtacttctgaaaagcaagatcctgagtccagattcctctgtcagatcaaaagtacttcacggattgtttccacatgctgcaaaaagagatctcgattttatgcttgttctaaagaggatatgagtattataggggaagttgatgcttgggatgtttatagttcaagtccttctgcattctattatgatggagatgatttgtcacatatacattctcatggtgaagaaaacctt

SEQ ID NO: 44 - DNA, 567 bp, Lycopersicon esculentum
  misc_feature (515)..(549): Unknown nucleotides 515 and 549
aaagattaattacacattctattttaacaaccaaaaaagtcataatattcagttggtaataataaacgatatctaatacaagaaatataatctgtaagctaagctgagctggaaaaataatcttcaaatcactgatctaaatatatattaatactactagctcatgcagtggtggtcaaccaaaaaaaagcttatggaattccaactgcgagagtggagttctggctataacttgaaattgagatcgagctgatcttcgctatccttagaagatgattcaccattagtattcgaagaagatggtaacaacggattgttacgtacaacttggtatggagctctaacagttgccctatctttcctcttctcgagaaacctatgaagtgaagatcttcttgcgataggtagctcggagctgctgctaatttcggcagcctgtgactggatttgcttgtgttgctcctgctctactaagggtgcagatgaaactgatccgagcttaagctgatcagttntagtactactacaaacatggagtggcggcgaatngtgcttgtgctgttgcgg

SEQ ID NO: 45 - DNA, 675 bp, Lycopersicon esculentum
ctactttaagagactctatttagagagaagtggttccaaatccattggaacaacagccaatttgctaattgcagcagcagctggagcttgtacagccataacaactcagccactggatactgcttcctcaaggatgcagacaagcgcatttgggaaatcgaagagcctttggataacacttacagaaggtagttggagtgaagcatttgatggccttgggatttctttactattgacatccaaccctgccattcagtatacagtttttgatcagcttaaacaacgattgctgaaagataaagcgaagacaacagagaaaggcacatcgcctgttgtactttctgcattctctgcttttgtattaggtgcactgtcaaagagcatagctactgttctaacctatccagcgatcaggtgcaaggtcatgattcaagcagcagacgatgatgacgataagaaggctgaacctaaatcaagtaagacaataagtagtgtcatatggtctatctggaagaaagaaggattcttgggtttcttcaagggattacatgctcaaatcctcaaaacagtgttaagctctgcattgcttctaatgatcaaggaaaagatctctgccacaacttgggtcctcatactctcacttaggagaattctgctgctcaggcagagaaagct

SEQ ID NO: 46 - DNA, 665 bp, Lycopersicon esculentum
tttctacacaatccatacattccatcgccggcgtcgtattcctgtcgccggaaaaatgcctcggcagctacttcttcgactgtacttcaacacccgatgttattgtcgatgaacattgacaaattcaaaccactggatttccctaagaactccaccaggaagttgaatcgttctgttactactatcagatcccaattgaattttcccatcatttctccgcaagatcaatggggaacttggactgttcttttcgctaccggcgcttttggcatttggtcggagaagaccaagatcggggctgcactaagtggttcactagtaagtgtactggttggtctagcagcaagtaacttgggcattatagctagtgaagctccagcatataaaatcgtgacaggattcttgcttccacttgctgttcccttgctgttgtttagagctgatatgcgtcgagtattaaagtccactggaacactgctcatggcctttttgcttggatcagttgcaactactattggtactgtggtggcttttttcatagttccgatgcgatcacttggtcaagatggctggaagatagctgccgctcttatgggaagacacataggtggagctgtcaactacgtggctatttccgaagctctgcagacatcacccatcagttg

SEQ ID NO: 47 - DNA, 597 bp, Lycopersicon esculentum
gagattttcgtcgactttgaaaagtaaggcttcgattatcggatttccgaagactcagaagactagtttcatcgattcgaaacggcgaacttgtaaaccggcgaacattaggcttttcccggcgaaaaggtctgaatctacgttgaaatcggcttcggttacggaaccgtcgtcaccgaaggtatcttgtatgggaagagtaagatcgaaacgtagatcacgaagccgacgatcatcggagaaatcaaaaaaatgtgaaaaaaattcactcgagagatcacgaagcaacaatggagagaaacgcaaatccggattctgttcacgattactatcaatattccgatctaatcggagtaacaaaaaaccgtcaaggtccagtactgataaaacgctaggttcaaagtcgacgacggagccgataaatccgctgagaaagagcaaaagtgtgaccgtagggaaaactcgcggaggagctccggttagcgttgaaccggttgggttaggaggaatgaaacggttctcctccggccggcgatcggaatcatggggaggcgatgaaatcaacgcagttgtttaagtgggtcccaatgaaagtgg

SEQ ID NO: 48 - DNA, 650 bp, Lycopersicon esculentum
  misc_feature (612)..(612): Unknown nucleotide 612
ttactgtatccaatgcacactaatatcacaatcgaatttccttaaatcattgtcctaatcctacataatgtacattcaaatgtttaactcattaaggaacacaatcctgctcccaaaagaatactccacactaaaattatacaacaaacagaagacatcggcttcagccactccaacaatcatcaggtgataatgcatctgaatacacaatgtacagcacagaggacagtacttcatcaccttggcagtgttgcattcagtacttgttctactaacaaagtactactgcaactccacagaaaccaaaagtcccaacatctgcaccagagctctcagaacattcaagagaatagaattttgaatgttcagataagtagcagccattttcttgcttttgttcatccacacacctacaaccttaattgagcataatcattctctagtaataatatcgccattatcagctgctaaagttgactccttgatggctaatgaatgaagcatcgattgggcagcaatcaacttttccctgagcatcatattatattcatagaggttttggagatctttctggagattggtcatacttacatgatcagcagcctgcaattntaaaaggggcggaggcggaacagttatgtcattagctg

SEQ ID NO: 49 - DNA, 515 bp, Lycopersicon esculentum
aaaatttcgaggaaatttcaggtcgagtatgaagaagagtgcggtgtcgccgacgtctgtactattcgttgttgccgcaatactaatttcttactactccgatgtctcgtcggcaaaatcagattcagacccaacgccggctccatggcctcatcaattccactcgatactgttcgtcaatttcagcggatctctaagcctaatcgatctctggtacgattggaccaatggccgcaatttcaacatcatacaagaccagctaggtgagcttctctatgatctcgaatggaacaatggcactgtgttccgctacactctagacgatggtaagaagaaatgcagttcagcgctggttgatgttggtattcttcgaccaaattggcttgacggagctacttatcttggtcaacagttcatcgatggatttcactgtaatgtatggcagaaggtggatttcatttggtattatgaagacatcgtcacaaagagacccgtccattgggttttctatactg

SEQ ID NO: 50 - DNA, 537 bp, Lycopersicon esculentum
gagttaattagcaacctaaaaacaaaagttcaagctacttttgccaattacaatagaagcttcactgcccaaatgaatttaccaatgtatatacacacaaaaatgcttttatatacactatataatttttcgacgagacagataactgcttgtaccattttcatgtttatcaccacacaccaacgtcctggcgctcgcatagtccaatcatatcagcagttttcatcagcatatctcgtccaaatgatctcgatatccctaatttgatcagatgatatgaacaatctctcaacatcctgcgatgaattcgaagttttgagtactgctaaagaattatccttcaatacttcatcatcttctggtgctagcaaataaatgccaataaacacagaaatcattccaaaaacgaacatggttgttcttaaagcatcaaacaccttgtattcctcaaagtagacaaatccagtacagattgagaaaaacgtccaagcgatttgaaacataggtacgataagtattgcatcaaatcgcgatagc

SEQ ID NO: 51 - DNA, 444 bp, Lycopersicon esculentum
gcaccaggtaacagtgacggaagagatacctttgtattcttaaacccaacaactccggcagcgaaagtagaggaaaaaattgttcggacagatgaaacgacaacgtcggagaagaaaaagaaggagatctccggcgaagtgaaagtcatcggaaaagtaacgaagaagatgaagaaggttgtgaagaagagtgaaggggttttagctcatgaagcttatatgaagagtaaagctaaagctgaagatcgccggcgatcgtatcttccttatcggccggagctcgtcggatttttcacaaatgtgaacggtggattaacgaagaatgtgcacccattctaagtcaatttttttaaatttaattaattttaatattatagtaatgtaatttgcttagtcaataattagatttgcttgatttagatcatggcctaagggattatgagg

SEQ ID NO: 52 - DNA, 525 bp, Lycopersicon esculentum
gctcaaaaattggctctgcagcattggctagaagctattgatccacgacatcgttatggccacaacttgcacttctattatgatttgtggtttttgagcgaaagctctcagcctttcttctactggttggacgttggagatgggaaagaaataaacctcgagaaatgtccaaggacaaaattgcaccatcaatgcatcaagtatctcggaccaaaggagcgggaatcatatgaagtagtcattgagaatgggaagcttgtatataaacaaagtggtgtatcagttaagtcagtagagggttccaagtcgatatttgttcttagcacaacaaggacattgtatgtaggacagaagcaaaagggcacatttcaacattcgagtttcctatcacgtggtgctatcacagcagctggaagactagttgctcattctggaattctggaagctatttggccatacagtggtcactatcacccaactgaagagaacttcaaggagttcataagctttcttgaagaacacaaa

SEQ ID NO: 53 - DNA, 464 bp, Lycopersicon esculentum
caactccaattccaaccaaactaatctcaaagcaaaaacccttttctcatcactcttcaaatcctcttttgccccaatcaacaatcaaacccttgaggagtttggtggtttcttctgcagctgcaattgaagctgaaacagaaacatctagctcgaattccactccgttgaagtctcttccttttagagtcggccatggatttgaccttcatcgattggaacctggttatcctttgattattggtgggattaatattcctcatgatagaagctgtgaagctcactctgatggtgacgttttgcttcactgtgttgttgatgcaattttgggagctctggggcttcctgatattgggcagattttccctgacactgaccctaaatggaagggtgcgccttcttcggtgttcatggaagaagctgtgcgtctaatgcatgaagcaggttatgaattgggaaaccta

SEQ ID NO: 54 - DNA, 536 bp, Lycopersicon esculentum
  mRNA (1)..(536)
gctttcacaactttagcaatttccaatcttcccactatgccattttcttcattcacttttggccttagtgccacttttatatcctcacttaacatgaccgcgttcattttttgttccgcatagagtggccatgctataagtggtaccccatggactacactctcaagagtcgagttccatccacaatgagtcaagaatccacccgtcgactcatggctcaatattcgagcttgtggggcccaattaggcaacactaaaccaaaccctttggtcctttccaagaacccttttggtaaaaactcaagagggttcgttgaatcttggacattaaaaaaagtagcgttagctattttgtcattagggcatctaactacccacaagaacctttgttcactcatttccaaccctgctgcaacttcaattagttgctcatgagatagggtcccaccactaccgtacgatatgtataacacagagccacgcggctgttcatccagccacgtcatacactccgacccgtcaaccttactacccga

SEQ ID NO: 55 - DNA, 585 bp, Lycopersicon esculentum
  mRNA (1)..(585)
ttttatcttagcaaaactatggataaaagagctgatcaacttcatgtctactttttgccaatgatggctccaggccacatgataccattagtagacatggccaggcaatttgctaggcatggtgtgaaggtaacaatcgttacgactcctctcaacgcgactaaattcaccaaaacaatccaaaaagacagagaggttggcagtgatatttgcatccggacaactgaattcccttgtaaagaagctggattgccagagggatgtgaaaacttagcttctactactacttcagaaatgacaatgaaattcatcaaagccttgtacttgtttgaacagcctgttgaaaagttcatggaggaagatcatccagattgtctagtagcaggaacattcttcgcatgggctgttgatgttgcagccaagctgggaattccaaggctagcttttaatggcactggtttacttcctatgtgtgcttataactgtttgatggaacacaaacctcatttgaaggttgaatccgagacaaaagagttgtcatccctggccttcctgacacaataaagatgtcaagatcaaagcttt

SEQ ID NO: 56 - DNA, 617 bp, Lycopersicon esculentum
  misc_feature (512)..(512): Unknown nucleotide 512
gacctataaatactttattaatttcctttcatttgtaaagacactatctaagacaaacgtccttgaacacaacattcatggcatcgtctaaagattcatatgattctctcctttatttacttatatcataatattcaattcagctggtaactggatttaacaagttgaacaaaatcgtatgttcttgacctcttacaaatttatctatagcggtaaatcataattgaatattcgtacgtaaatcagcataagatttcacatcgattttccaacatttgtctacatattgcctttcttatacttgatcaagaaacattatctagactttaaacccccaatttttggttgttgtgtgacatcttctattagttttgtcaaattaatgtgagatgaacctccttcctccacagcctttgttgccatatctgcaagccctttagctctttctcttctcatttttgcttcctcttcttcacccattaatctttcaataaccatcttaatatcatctntcttcacttgtgcacatcctttttcatcctctaaaaacatcactggattctccataccacccttcactcctgtcttgagtacattcacaataagcttctcattac

SEQ ID NO: 57 - DNA, 530 bp, Lycopersicon esculentum
taatcatgagccgattacaaagttggcccgaaccgattattcgagttcatgaactatccgaaagtggcattaaaaaaattcccgaaaattttgtgaagctcccatcggagaggccaagttcgataaccacgttgtcgtcgtcatcacatgttgagaatattccgttaattgaccttgaaaacttgaattcatcggatgaatccattcgcgatgagacaatggaactcatttccaaggcttgtcgcgaatggggtttcttccaagtggtgaaccacggcgttagccatgaacttatggctaacgcacgtagtgtctggcgagagttctttcatctcccgatggatgagaagcagaagtttgcgaattcgcccattacttatgagggttatggaagtagacttggtgtggtgaaaggtgccaaattggattggtgtgattatttcttccttcattatcttcctgagaagttgaaggatgagaataaatggcctagtcttcctatttcatgcaggcatataattggtgaatat

SEQ ID NO: 58 - DNA, 656 bp, Lycopersicon esculentum
gccatttcgaatcttctatcttctgcgtagaagctacgatttggtttttgatttttttcatcatcgagcaatttcattgctttgatgcaaaaacaagaggcattttcggagggatttggtgctgttttgatctctgggtttagttatgccagagatcttacgtgaggagagcagttcatcttcatcaaccaccgccggcaccatcaccggcgccggcaccggagatttcaagcttaacatatttgggaaatatgaggtcgggaagcttctgggttgtggtgcttttgcaaaggtttatcatgctagggatgtgaggacgagacagagcgtggcgattaagacggtaagcaagacgaagatattgaaaggtggtttcacggagcatgttaagagggagatttctatcatgcgcaggttgcgtcatcctcatatcgtgcgcctccatgaggtgctcgcgacgaagacgaaggtctattacgttatggaattcgctaaaggtggagagctgtttacgaaagttagcaaagggagattcagcgaggatctcagccggagatattttcagcagttgatctccgccgttgattattgtcattctcgtggtgtgtatcatcgagatctgaaactggagaatctgttgcttgatgagaaatggg

SEQ ID NO: 59 - DNA, 522 bp, Lycopersicon esculentum
aactgtgtggggtttgtatgcccagagtatttcaagtctgatttcgtataccggactttgtggttgcaggatagcccatcagaagatattactagtattctctatgatgtttttgactactttgaagatgtcagggagcaacatgggaaggtttttgttcattgctgccaaggggtctctcggtcaacctcgttggttattgcttatcgtatgtggagagaaggacaaagttttgatgatgcctttgagtatgtaaaggcagcaaggggtattgcggatccaaatatgggttttgcttgtcagttattacaatgccaaaaaagggttcatgcttctcctttgagcccaagttcattattaaggatgtacagagttgcacctcattcaccatacgatcctttgcatctcgtcccaaaaatgttaaatgatccctcaccggcagcattagattctagaggtgcatttattatacacataccttcatcggtatatgtatggattggtaagaaatgtgaagcaatc

SEQ ID NO: 60 - DNA, 585 bp, Lycopersicon esculentum
acttgtaaatagaaatattgatcagtatattgaaattgtacaaagtagcaaacatctgtaggaactaaaacaagaagaaaattacatcaaacatagcttattcgcaatgatatgttcattatttctagcacaaaatgcagagacagcaccgccgagtccagactctagagcttaagcaacacccaagagtttcttgatatccttctccatgctagctggagaagtggttggagagtagcgatcaacgacatgtccttctttgtcaatgaggaatttggagaagttccacttgataccatctccaaagaacccacctttgcttgatttcagaaacctatataatggagcagcattatcaccattcacgtcaaccttgtcaaatatggggtactcagccttgaagcgagtgcagaccatttgctggatatcttcaatgtttccaggctcctgtccaccaaactggttgcaagggaaggccagaatctccaaaccttgatccttgtacttcttgtataactccgtcatgtcagtgtaattcgaattagtcaggccacactgtgatgcaacattgacaatgataaggac

SEQ ID NO: 61 - DNA, 458 bp, Lycopersicon esculentum
tatgcagctcttaccaaagctttctcaggacttggtgtggatgagaacttattcatctcaacactggggaaatggaatcggcatcagagggaatcgtacagggtaagcactcctggattcttcagagaagacaaacgacagttccagagatgggatgatcaacatatcctccagcttcgtcaagagtttttgcgtcttaaggatgcagtggttttgtacacaatgcacccatgggaaagagatgctcgtttgttcaaggaagcattattattgcaagtacctaaacatgatgttctcattgagattgcttgcactcgatcctctgaagatcttcttggagccagaaaagcttaccattccctttttgaacactcaattgaggaagacattgccttccatgttcgaagttctgaaagaaagcttctagttgctcttgtaagttcctaccgttatgaagg

SEQ ID NO: 62 - DNA, 631 bp, Lycopersicon esculentum
gcaattgcaaattaaaacaaacatggctgatgaagtgaagctttacaggacatggtcaagtccatttggtttgaggatcgtttgggcgctgcatatcaaagggatcgaatacgaagccatctttgaagatttgagccagaagagccctcaactcctccagtataatcctgttcacaaaaagattcctgtacttgtacacaaaggcaaaccaatctgtgaatcactggtgattctcgagtatatcgacgagacatggaaggaaacagctcctttgctgcctcaggatccttatgagaaagccatggcacgattttgggcaaaatttgtggaagacaagcttctgccatcagtatggagtatttttacagaacaaggatatgacgcgaaaaaggaagcttttgttccagcagtgcaaaacctagaaatcattgaagagcaattgaaagagaagaagttttttggtggagaaagtataggatatgtggatcttgtacttggttggatggcgtatcttcttgatgtttttgaggaggtactcgatttgaagttgtttgatgcagacaaattcccgctcttatcaggatggatgaagaacttctgtgatgctccggcaatcaaacaacacttgcca

SEQ ID NO: 63 - DNA, 580 bp, Lycopersicon esculentum
ctttctccggatgacaattcacacgattgcctgcaagctgctgttcctgataaaaatggagtaaggcatctattgctttgtcgcgtaatactgggaaaaactgaagttgttcatcctggatcaggacagttctatccgagttctgaggaatatgatacaggggttgataatttatcttctcctagaaagtttatcgtgtggaatactcacatgaacagttacgttttcccagaatttatggtcagtttcagagtcagctcacatgctaaagagtctcaaaggaatgcagttcctattcagaacccaaaatcaccttggattacatttcctgcactgatttctgctctatcgaaattcttgccgcctcagaccgtcaaattgattacgaagtatcataatgatcataaggggagaaagatcacaagacgagacttgattcagcaagtgaggaaactagcaggagacgagttgttaactgcaatcatcaaatcctgcaagaataagcaaagcaaaggatcatctgggaactcatccagcacaagctcaattaacttcgagcaacgagatggaggatgctgtg

SEQ ID NO: 64 - DNA, 572 bp, Lycopersicon esculentum
tagcaagtcaaccaccaatccagcattggatccggatctggacagcccggatcagccgggtctggagtttgcccaatttgctgccggctgcttttggggagtcgaattggctttccagagggttggaggagtagtgaagacggaggttgggtactctcaggggaatgtccatgacccgaactacaagcttatttgctccggaacaaccgaacatgccgaggccattcggatccagtttgacccgaatgtctgcccgtattccaatctcctttctctattttggagtcgccatgacccgaccactctaaatcgccaggtatcaaattcctttggtgtttcattttatgtgattaatattaaaaattttttatataaatgtcatgatgatggttgttgctagggtaatgatgtgggaaagcaataccgctcaggaatatattactataatgatgctcaggctcaactggcaagggagtcgttagaagctaagcagaaggaatttatggataagaaaattgtcactgaaattcttcctgctaagagattttatagagctgaagagtatcaccagc

SEQ ID NO: 65 - DNA, 572 bp, Lycopersicon esculentum
gggattctaggtaaattcaatattccgattcaccggaaattgttgaacattggagaattcccggaatgggtggcagcaggtgaccggagactaatgcaggaggttaatcctaccccggacgtgacagtggcgagtgatggttcaggaaatgttacgactattggagaagcagtgaaaatggtgccgaaaaagagcaaaacgagattcgtgatacatgtgaaggaaggtgaatatgtggagacagtgagaatggataagtcgctttggaatgtcatgatgtacggcgatggcaaaactaagaccattatttccggaagcttgaacttcatcgatggaactcccacttttgagacggccacatttgctgttgctggtagaggcttcattgccagagacataacattcaggaacacagctggcgcggcaaagcaccaggcagtggccatgcgttcggggtccgatcaatctgtattttatcgatgttcgtttgatgccttccaggacaccctctatgctcactccaaccgtcaattttaccgagaatgtgacattacaggtacaatcgatttcat

SEQ ID NO: 66 - DNA, 523 bp, Lycopersicon esculentum
gattgaatacaaaagcgtttaaagaagcaatctttagaattcaacacttgaagaggaagggtggaactttgctttacatacctgctggtgtatatttgactggcccatttgatcttactagtcgaatgactctttatttggcccgcggtgctgttatcaaagccactcaggatcccagtcaatggccattagttgatccattgccttcatatgggagaggaagagagcgacctggtgcaaggtatatgagcttactccatggagacgggctccatgatgtgataatcacaggtgagaatggtactattgatggccagggtgatgtttggtggaatatgtggaggcaaagaactcttcagtttacacgacctcatctcgttgaactaatgaactccagaggcatactcatttctaatgtcattttcaagaactctcccttttggaatatccaccctgtttattgcagcaatgttgttattcgatatgtcaccatattggctccagctgattcaccaaataccga

SEQ ID NO: 67 - DNA, 652 bp, Lycopersicon esculentum
cctctttcattttctcctcaaattcgtctctaagagttaaaaaagaagaaagaaaaatggtgaagatttgctgcataggagctggatatgttggtggccccaccatggcagtcattgcactcaaatgccccaagattgaagtggtcgttgttgatatctctgtacctcgcattactgcctggaacagtgacacacttcccatctatgagccaggccttgacgatgtggtcaagcaatgccgaggcaagaatctcttcttcagcacagatgtggagaaacatgtgcacgaggctgatatagtttttgtttctgtcaacactcctaccaagactaggggtcttggagcaggcaaggctgcagatcttacctactgggagagtgcagcccgtatgattgccgatgtctcgaaatctgacaaaatagttgttgagaaatcaactgttccagtaaaaactgctgaggcaattgaaaagattttgacccacaacagcaagggaattaacttccagatcctctcaaaccctgaatttcttgcagagggtactgcaatcgacgacctctttaaacctgacagggttctgattggaggccgggaaactccaggaggccagaaagctatccaagcattgaaggatgtttatgctcattgggt

SEQ ID NO: 68 - DNA, 624 bp, Lycopersicon esculentum
caaacaaatatcacaatatacagaaaagttctagagagagaaagagaagacaatggcttactcgaagattatcgttgttgctttcatgttcgctttggtcgccggatctgcttttgctcaagctccgggagcttctccggtagcttcaccgaaaccgtctccggtaccggtagcaaaacctccaactcctgttgttaccccagtatctgctccttcagtatctcctgctgcaagttcaccttctgattctccagtttcatctccaccagctccaccaaccgttgagactccggcttctgctcctaccgccggtggtgctcttgctccgtccatcggtgctacaccaagcggttctccaacttcctctccaaatgctgcatccttgaacagagtcgctgttgctggatctgccgtcgtagctatcttcgctgcgtctttgctgttttagatctgaggagatatttgcatttggatttgctggagagagggatgtttatgattttaggatttttatatttttagttcatcttactcgttgatgtttattattcgttttgctactttttcttttacctttgggtgatggtgacgattacgtacatgctattgatttgatttttactt

SEQ ID NO: 69 - DNA, 612 bp, Lycopersicon esculentum
tgtgaaacatggtctcctcatagaccctagatttagcaagaacatggcaagttgagtgaagttaactcaatttgcataaattacaatcaacttatacaagaaggaaaggtgaagtttcctaattacgagaaggttttatatcaccaacacaatagaaaatggtggatgcagattgaagttgcatgtctgcatatgaggtgtgagaaattaagctttcaggtacccttttttgtccaagtatgaaataacaatatcggccaaatcattaggagagtcacatagcccttggttctgacgtaaaactatctcagatgttaggggtggctcgtatggatcatcaacaccggtaaaaccttttattttcccagctcgagcaagtttgtacaagcccttaggatcccttgccctttaaatcactactctaattttctataatgttgctgcagctttctcttcttacactagtcttactatcccctgtttccgctgataatttctaccaagacgcggcggtcacgtttggtgaccagcgcgctcagatacaagatggagggcgccttctcacattgtcacttgataaaatttcaagttccggatttcagtctaagaatg

SEQ ID NO: 70 - DNA, 678 bp, Lycopersicon esculentum
cttcaattctatgctgtaacagaactcaatattatacagaggacaaataaaagcctcaagaagaaataataatcaaataatagttgaaaaaggaaaatatagccatcataagttggtaatagaaggaattcaaatgtggtgcatggtttcctgcaggtctagatgatggaacagatgggaacgatgtagcgggttcatttgcacttgcatttcccttggtggcctcgggcaccacactggcaggcgaaggcgcttcggtagaattgcgcttgttcacgggcagagttgttgatctatcgtttgattctccagaatcataacaatcagaggctttccaacccaaaaccatcttctcccgatcaaatattacgtgatagccagtcatgaaattttgtccaatgatgttgacatccccacttttcacaacagctaaacagtatgcgcctgaaccgtccgggagtgagagcattattatcggatcgaaaagataaaattggttgccacctttcattgtcaaatttacgtctggaacttcaaatgtagtttgatttgcacttatcccatagcagtactcaaaacgaattgtgccatcaggttgaatacgtggctgttttgcttcagaatcgaagttttctgtaatgactttgtaaactgtgtcgttcaagtatgtgaatgaag

SEQ ID NO: 71 - DNA, 774 bp, Lycopersicon esculentum
ggtggaggtggattggctgaatatgtggtacctaaggatagtttgactgtttcaagacccgctgaagtatcagctgcagaaggtgcaggtcttgttatcgcaggtcttacagctcttcaggccctcgtcaatcctgctgaggttaagcttgatggaactggaccccggaagaacattctagttacggctgcttcgggtggtgtaggtcactatgctgttcaattggcaaaacttggtaacgcccatgttactgccacctgtggagcccgcaacattgattttgtgaagagcttaggagcagatgagattcttgattacaagactccagagggagctgctcttaagagtccatcaggccaaaaatatgatgcagtgattaactgcacaacaggcatcccttggtctacctttgagcctaatttaagttcaagtggaaaggtgatcgatctaactcctggagctagctccatgttgacctttgcagtaaagaaattaaccttctcaaagaagcagttggtgccatttcttgtaaatgccaataaggaaaacctggaattgcttgttaagcttgtgaaggaagggaagctcaaaacagtgattgactccaaatttcctctgagcaaggctgaagatgcttggagcaagagtatcgacggacatgcgacaggaaagattttggtggagccataaagtagtgatctaccatattatgttattatacataccgtgtgttataaaagatttgtgagggcttgtgcattatgaccagttaaa

SEQ ID NO: 72 - DNA, 653 bp, Lycopersicon esculentum
gagtctacatcaagggatttaggtagaattcagacattgcatacaaggattgttgacaaaaagaatcaaaactctgtttcaagattgggtgaaaaaactgttatttcttcacaatttgagctttcaggaaaactaatggcgacgttggagtcaggggtgagtcatggatcaggggaatatttcatggatgtttttgttggtacacctcctaagcacttctctttgattcttgatactggtagtgatcttaattggattcaatgtgtaccttgttacgattgttttgaacaaaatggtccttattatgatcctaaagattcttcttcttttactaatataacttgtcatgatccaatgtgtcatcttgtttcatctcctgaccctccacagccttgtaagactgagaatcaaacttgcccttattactattggtatggagatagctcaaatacgactggtgatttcgcgcttgagactttctccgttaatctcacaacccctagtgggaactctgagatcaagaaagtggaaaatgtgatgtttggttgtggtcattggaatagaggtttgtttcatggtgctgcaagtttgttaggacttggtaaaggaccactttcgttttcgtctcagctacaatctttatatggacattc

SEQ ID NO: 73 - DNA, 680 bp, Lycopersicon esculentum
aagaataggaactgttttttttttctttttgtacatggctaaattcaagattctaccatcatgaagaacaagttaaattctacaaagacttaaataccaagatttaaagtgaattaagaaaagaaaaaaaaccataaaattggtttgggtcaacctaaccaagatgatcttaacaattatatctagcactaaaaattgatcctctctcaaaagaaacttgtgctagctaattaaaacttttttaattcatgttgttccattgcttgtgtcctggcacccattttgggcattttgggctaaagtagtttgacactactcttgaattcaatagctcaacaattggttcaaatttcacacatcttggtgtcttgtactgatttattgatgcacctaaacttatagcatagtccatcaacttatcaaaagttccactttcaacaatcttgatttccaatggcccaatggatttgtctgaggcacgaccttggcggtatacgctgttgagagactcttcgatggtgagacaacaatcttcgaaaacagatggaggaactggggttgatccattaacatttagctcccagtataacacataatgccctggaatggttgtggttgtatcagcgtaacttgtgtattctgtcacatgtgcatcaaatggcataagatgtgtgactgca

SEQ ID NO: 74 - DNA, 542 bp, Lycopersicon esculentum
gggggaaaatgtcatcggagacagccaaatcagcgaacggtttgacggaaacaaataattccggccttaatttcaatgaaactgagctaactctcggcttacccggcgaatcgcggaagcaaatctccggtgcaaaacgtggaaattccgacggtatggaattaagcctaggaagctcaacttccggtgaacgccgccgtgatatctgcgaagttaatcactcgaaaaacgaaatctctaccggaaacaaacctccagcaaaggcacaagtaattggatggccgccggtgagatcatacagaaaaaacgtgatagaaaagtgcaaatacgtgaaggtagcagttgacggagctccttacttgagaaaagtagatctggagatgtacgacagttaccagaagttgttgaatgctttagaaaacatgtttacttgcctaacaatctgtaattctcaaagtgaaagcaagcttatggaccttacaaatggtgtagaatatgtacctacttatgaagataaagatggagactggatgcttgtagga

SEQ ID NO: 75 - DNA, 722 bp, Lycopersicon esculentum
accccatgaccctacatcaacaacaacaacttcctcacctaccaatctcgttgaccttgtcgatgttgaagttggaaaagagtacgagctcgtcatcacaacatacgccggtttataccgttacagagtcggagacatcctccgagtcaccgggtttcacaactcagccccgcagttccattttgtccggagaaaaaatgttctattgagcattgactctgacaaaactgatgaagctgagttacagggagccgtagagaatgcttcgaaattactcagggaattcaacaccagcgtggttgagtacactagttatgcagatacaaagacaattccaggacattacgtgatatattgggagttattaatgaaggatttgaacaattctcctagcgatgaagtattgaaccaatgttgtttagcgatggaagagtcactcaacacagtttatcgacaagggcgagttgcttgtaactcaattggtccattggaaattcgagtggtgaaaaatggtacatttgaagaattaatggattatgcaatttcaagaggtgcttcaattaaccaatataaggtgcctagatgtgtgaactttgcacctattttggaacttcttgactcaagagtaatgtcaagacactttagcccatccttacctcaatggacacctgaacgacgtcgttgaagaggaggagaagtgtttattaattacacatgcatca

SEQ ID NO: 76 - DNA, 571 bp, Lycopersicon esculentum
ctctgttcatagatctaatggcaactatcaaatccattaaagctcgtcagatctttgacagccgtggtaaccccacagtcgaggttgatgtacatatctcaaatggtgtgtttgccagagctgctgttccaagtggtgcatccaccggaatttacgaagcccttgaattgagggatggaggatctgactacctcggaaagggtgtttcgaaggctgttaacaatgtcaactcaattattggccctgctcttgttggcaaggacccaactgatcagactggtcttgataacttcatggttcatcagttagatggaactcaaaatgagtggggttggtgcaaagaaaagcttggtgcaaatgccatccttgctgtgtcacttgctgtgtgtaaagctggcgctgctgtcaggaatgtgccattgtacaagcacattgctgatttggctggtaacaagaagttggtattaccagttcctgccttcaatgtgattaatggtggatctcatgccggaaacaaactcgcaatgcaggaatttatgatccttcctgttggagctgctaatttcaagga

SEQ ID NO: 77 - DNA, 686 bp, Lycopersicon esculentum
ggaaattattgtcaagggtggtgatatgaaatatcatcggttggctcgagtaccacgcttgatacaagttatcaatttctcgtttgttataaatattatccataatcactaacaagaaagcttacaccaatggaaaataaagtcgttgaagagaaacaagtagacatgatccctctaatgagcccttgtaaaatgggaaagtttgagttatgtcatagagttgtattggcaccattaacaaggcaaagatcttatggttatattcctcaaccacatgctatacttcattactcacaaagaagtacaaatggtggccttctaataggagaggccacagtaatatctgagactggcatagggtacaaagatgtacctggtatatggacaaaagagcaagtggaggcttggaaaccaattgtagatgcagttcatgctaaaggaggaatcttcttttgccaaatttggcatgttggtagagtttccacaaagattttcagcccaatggagaggatcctatctcctgcacagacagaggactaacacctcaaattcgttccaatggcatagatattgcacactttacacgacctagacggttgacaacagatgaaattcctcaaattgttaacgaatttcgagttgctgctagaaacgcaattgaagctggatttgatggggttttgaga

SEQ ID NO: 78 - DNA, 708 bp, Lycopersicon esculentum
aaaagtatctttctatatatcttctaacttgctacacagaaatgtaaaaattacaaaatacatccaaatctggaaatgtatcataaaataaaacaaactaaaagcccagtttcctaaaagaatatacacgcccctgcatatatctcctgtttgtgtacattattttatcgtccacgcagacgtgccttcggcctaatgacttcaaacacaacttgcaaataagaagtgccactcagcctaatgacttcagacacaattgcaagaacttgcaaataacgtccacgacgtctgcccagacgtgccctcgaaggacttaatttccttggcatgatactgtacttgctctttcttcgaatccgttgggccaaggaaatctaaatccaggccttctgtatatatacacagtatactggcattcagatttcgagctacaacttttcttactttccagtatcctctactgcttgaacccgagatttaacgtctgattcggacattggatcccgaattgatccacattgtttgtatacgcttgggaaaatccattgagactctgttggaagtaactcgattggtcaatccgttgatttcttttcccgctttcaacttcttcgtctagattaatactttcaacctttgtgttgcctcggaaatttggaggatgggcgtaaacatcgttgagaaaattcatattgttgttgtcgctgt

SEQ ID NO: 79 - DNA, 662 bp, Lycopersicon esculentum
tatccaaaaccaagagcaattggaacctatgagatctcacaagttgttgaagattatcgcaggtcggccttgaatgctattgaagcaggtttcgatggtattgaaatccatggagctcacggttacttgattgatcaattcttgaaagatgggatcaatgaccggacagatgagtatggtggatcactagccaaccggtgcaaattcatcacacaggtggttcaagcagtagtctcagcaataggagctgatcgcgtaggcgttagagtttcaccagcaatagatcatcttgatgccatggactctaatccactcagccttggcttagcagttgttgaaagactaaacaaaatccaactccattctggttccaagcttgcctatcttcatgtaacacagccacgatacgtagcatatgggcaaactgaagcaggcagacttggcagtgaagaggaagaggctcgtttaatgaggactttgaggaacgcgtatcaggggacattcatttgcagtggtggatacactagggaactaggaattgaggctgtggcacaaggtgatgctgatctcgtgtcatatggtcgtcttttcatctctaatcctgatttggttatgagaatcaagctaaatgcacctctaaataagtataacaggaagacatt

SEQ ID NO: 80 - DNA, 1257 bp, Lycopersicon esculentum
tagctcataaagtaatctaattttttttatatatataaatttttcattacgtaccacactatgccttcaagaatctcagattcttgtagacctcatcactctcaaaaacactttgacttgaactcaattaaagaattaccagaatcccatgcatggacatcatcaaatgatcattatactcaagaaaattcatgtaatttcgagtctattccagtgatcgacctcgataacaacaataacaataataataataatattcttgatcatataggtcatgcatgcaagaaatggggtgcatttcaaattataaaccatagtatatcagagaaattactacaagatattgaagtggctggaaaaaccctattttcacttcctatgcaacaaaaattaaaggctgctcgctctcctgatggtgtcactggctatggcgcagctcgaatttcgtcttttttctcgaagctcatgtggtctgaagggttcaccattgttggttccccaattgaacatgctcgacaactttggcctaaagattacaacaaattctgtgaagtaatagaagaatatgaaaaggaaatggaaaagctagcaggaagattaatgtggcttatacttgggtcattaggaataacaaaagatgatgtaaaatgggccgtaggcccaaaaggtgaaaccaaagaaggatgtgctgccttacaactaaattcatacccggcatgtccggatccgggtcgggccatgggtcttgcagcacatacggattctaccatattaacaatccttcaccaaaacaacacaagtggtttacaagtttaccaagaaggaaatgggtggatcactgtccctccaatacccggggcattagtagttaatataggtgacttattacacatactctcaaacgggtcatacccgagtgttttacatcgggcggtcgtaaaccgaacacgatatcgactttctgtagcttatttatatggtccaccgtcgggggtgagagtctcacccctatcgaaattggtggaccataggcacccacccttgtatagggcagtgacatggagtgagtatttaggtactaaggccaaatattttgacaaggcactttcatccgtacggctttgtgtccctttaaatggatttactgatgccaaagatcataaaggggtacaagttggataaattcactagtgacacttacatttgaattttatttttttttttaaaaaaaaaaaaaaaaaaaaaaaaaaaaaaaaa

SEQ ID NO: 81 - DNA, 613 bp, Lycopersicon esculentum
tcggaatctgagtggatagctaggctgctgatggagcaatcacgtgcagtcaagtgcgccttcaatttcttatcatttagctggcagcaagaagattcagcaagagcttgcaaagcccaatgtacttgaaaggtttctggaaaacaaagatgacattgccaaactacggaaatgctttgcagggttgtggagcttggatgaatccgacattgtcaaggatgcaattgagaggcctgaattgtatgttatgaaaccacaacgagaaggaggaggaaacaatatctatggggaggatgtcagaggtgctcttttgaaactgcaaaaggaaggcactggaagtgatgcagcctacatactaatgcagaggattttcccgaagatctctcactcgatactaatgcgagaaggcatctctcataaagaagaaaccatatcagaacttgggatatatggtacctatttaaggaacaaaacggaagttttaatcaaccaacaggctggttacttgatgcgaacaaaggtctcttcatcagatgaggggggagttgcagccgggtttgcagttttggacagtatatacttggtttgatggaattgtcttgcgcgttgacac

SEQ ID NO: 82 - DNA, 647 bp, Lycopersicon esculentum
gagaactacgaatccagaggaatatatgattaaatttgttcttcaagaaacagagagagcgaaaaatgcctctgcgatcattctcaatacatttgagacactagagagtgaagttcttgaatcactccagactctccttccacctgtctacccaataggccccttgcatttactcgtcaaacatgttgatgacgagaatttgaaggggcttgggtcgagcttatggaaagaagagccagagtgcttacaatggctcgacagcaaagaaccaaactctgttgtttatgtaaactatggaagtattactgttatgactcctgatcaacttattgaatttgcttggggacttgcaaatagtcaacacgagtttttatggatcataagacctgatattgtatcaggttatgaatcgattcttccacctgaattcgtggaagaaactaaaaacaggggaatgcttgcgagttggtgctcacaagaagaagttcttaaccatccggctataggagggttcctgactcacagtggatggaattcgactctggagagcattactagtggggtgcctatgttatgttggccgtttttcgcggaacaacaaacaaactgttggtttagtgagactaaatggggtattggaatggaaa

SEQ ID NO: 83 - DNA, 555 bp, Lycopersicon esculentum
agccagttttcataatacagattgttaccttgcacggaattgctggaacacaaggatccaaaggaggattcaaactagaagtggaaccgcgatatttcactcattaataagtatttggatgccgttaaaagatgaatatggtgtaccaacttcatggtgggttgagaagaataatggaatggttcaacaacaaaatggaacctggattctcgtggatcatgaagatgatgatttttagttctctatcgagcaaaaatatagatcttaaatttatgaacgaacagaaacaaaaatagtaccggttttgattccatgattgattggttcatcatgtacacacgtatgagaggataatttttttttttttatcttttgtgtaatttggaagtagggccaagcgatatacaatatctcagatacgattattggcactgcaacaaaaaataaatgcagtaattatttacttgcattaatatatacaaatgtttattactagtattttatataaatgtcattaggaatgtgataatattaataattgacgttgattgtttt

SEQ ID NO: 84 - DNA, 503 bp, Lycopersicon esculentum
gcggcattggatgcaatggatgcagggaagttatttgttcctgtaaagaacatggacattccagctgcagccgaaattattcgctattatgctggtgcagccgataaaatccatggaacgactctcaaaatgtcgcgtgagatgcaaggatacacgttgctcgaaccaattggtgttgtcggacacattattccttggaatttcccaactcagatgtttcttatgaaggttggccctgcattagcagctggttgcactatgattgtcaaacctgctgaacaaactcctctttccgcgctctattatgctcaattggctaaacaagcaggtgttcccgatggagtgatcaatgtcgttacaggatttggttctactgctggtgctgcactttgctcgcatatggacgtagacaagataagcttcacaggttcgacagaagtaggacgtctagtaatgcaggctgcagcattaagcaatttaaaacctgtctcgctagaattggg

SEQ ID NO: 85 - DNA, 607 bp, Lycopersicon esculentum
atcagtgtagaaagaaataaagaaaacgaatggaggaaatagggtttctagggattggtattatgggaaaagcaatggcggtgaacttgctgcgccatggtttcaaggttactgtttggaatcgcaccctctccaggtgtgatgagctagtgcaacatggagcctctgttggagaaactcctgcagcagtaatcaagaaatgcaagtatacaattgcaatgttgtctgatccagctgcagctctttcagtggtttttgacaaacacggagcacttgagcacgtatgtgccggaaagggctatatagacatgtcaaccgttgatgctgatacttcttcacagattagccaggccattacatcaaagggtggttcattccttgaagctccagtttcagggagcaaaaagccagctgaagatggacaactagtaatcctagcagctggtgacaaggatctgtacgatcaagtagtacctgcttttgatgtcctgtgaaagaaatcttttttcttgggaaagattgggaatggagcaaaaatgaaacttgttgttaatatgataatgggcagtatgatgaatgcgttttcagaaggaattgtactggcttg

SEQ ID NO: 86 - DNA, 614 bp, Lycopersicon esculentum
  misc_feature (511)..(511): Unknown nucleotide 511
gcaccagattatttggacacacttacacaacgagtcaattatcatttaggttatccagtcaacatatgttatgatcactatgcaacgctagcaccacttttgcagtttcacctaaacaattgtggtgatcctttcctacaaaatactgtcgatttccattctaaagactttgaagtggctgttttgaattggtttgcaaaactttgggaaattgaaaaggatcaatattggggatatgttaccaatggtggcaccgaaggcaatctccatggtattttgttagggagagagctacttcctgaaggaatattatatgcatcaaaagactctcattactcagtattcaaagctgcaagaatgtatagaatggattcagaaacaatcaacacatcagtaaatggagagatggattattcagatttaagagcaaagttacttcaaaataaggataaaccagctattataaatgtcacaattggaactacattcaaaggagcaatcgatgacctgnatgttattcttgaaatactcaaagaatgtggctattcacaagatcgattttacattcactgtgatgcagcactatgtggtcttatgaccccttttataaacaa

SEQ ID NO: 87 - DNA, 639 bp, Lycopersicon esculentum
ccgtaatcgaattcatgacattcaacttctccatgcaggcaatagaccatattatcaattctgctgcaaaatcaaactacatgtctgctggtcagatatctgtgcccattgttttcaggggtcctaatggtgctgcagctggtgtcggtgcccaacactctcagtgctatgcggcatggtatggtgcgtgtccaggattaaaggtactagctccatactcttccgaagatgctcgtggcttgctcaaagctgctatcagggatcctgatcctgttgtgtttctggaaaatgaattgctgtatggtgagtctttccctatttccgctgaagctcttgattctagtttctccctccccattggaaaagctaagatagaacgcgaagggaaggatgtgaccatcactgccttctcaaagatggttggctatgcgatcaaggctgctgaacttcttgccaaggaaggaattagtgctgaggttataaatctgcgctcgatccgaccacttgatagacctgccattaatgcatctgtgaggaaaaccaacagacttgtaactgttgaggaaggatttccacaacatggtgttggcgctgaaatctgtgcatctgtggttgaggagagctttgagtatcttgatg

SEQ ID NO: 88 - DNA, 461 bp, Lycopersicon esculentum
accaattcgcggatatgtatcctgcgctattgggtgcccagtcgaaggagcgatttcaccttcaaaagtggcatatgtagcaaaggaacttcatgacatgggatgttttgaaatttcccttggagacacaataggaatcgctactccaggaactgttgttcctatgcttgaagctgtgatggctgtggttcctgttgaaaaacttgctgtgcattttcatgatacctatggacagtctctttcaaatattttggtttccctccaaatgggtattagcacagtcgactcctcagttgctggtctcggaggctgtccatatgccaaaggagcttctggcaatgttgcaacagaagatgttgtctacatgctcaatggtctagggattaaaaccaatgtcgatctacagaaactcctgctagccggggagttcatcagcaagcatttggaccgcccatccggat

SEQ ID NO: 89 - DNA, 434 bp, Lycopersicon esculentum
gagaactacaagctgcaatctgtacgctttcaaatcaatttgttttatagattcaatcaaaatgcatacaattgctccatcatccggtgtaactctcaaatccttagtccagcaaacatcgccaccgccggctcaaatcagacggttcgtcccagctcgactcgtcgtacaatgcgggtaccggtttgattcagacaacacaaacgctagtactgtcaatacaaacagtattccggcgagcaacaatttcgccggccatgtcggagcgtcaagagctgactggcagagctcttgtgctattctggcgagcaaggttgtttctcaccaccaggatactgagaaaacaggtggagctggtgagataactgtagtaaacggacacaaatctctggatctcgttcctcttgataacaatctcccaaagccgctcacaa

SEQ ID NO: 90 - DNA, 415 bp, Lycopersicon esculentum
aaaaacttggccaaataattcccttttcaaactatccaatgctggatttagtttctttaacccccccattgaattattgttttacataaacaaaccacaatgcaaatcaaaactttcactagttgttttctccatcaaattcaacaaactttcatatccttttgggtttataggagttgtttgggccccaagtctttggaacattcttccaatagtatgtgtaataaattgggatccagcagcatacttggcatgttcactacaactcattttaaccattctacaacctttttttacaaaaatggtgataaaattggtgactcttttaattcttgacttttcttggccaattctaactttggcatacataaatggggggcctttccaattggctttaccacttgttggaccaaac

SEQ ID NO: 91 - DNA, 585 bp, Lycopersicon esculentum
actgtctcagaagcatgtagtatatactgagcctcttgatatccaagctggaagcagcgtcacagtttactataatccggccaatacagtacttaatggtaaacctgaaatttggttcagatgttcatttaatcgctggacacaccgcctgggtccattgccacctcagaaaatgttgcctgctgaaaatggcacccatgtcaaagcaactgtcaaggttccattggatgcatatatgatggattttgtattttccgagagagaagatggtggtatttttgacaataagagcggaatggactatcacatacccgtgtttggaggagtcgctaaagaacctccgatgcatattgtccatattgctgtcgaaatggcaccaattgcaaaggtgggaggccttggtgatgttgttactagcctttcccgtgctgttcaagatttaaaccataatgtggacattatcttacctaagtatgactgtttgaagatgaataatgtgaaggactttcggtttcacaaaagctacttttggggtgggactgaaataaaagtatggtttggaaaggtggaaggactctcggtcta

SEQ ID NO: 92 - DNA, 731 bp, Lycopersicon esculentum
gggaagatgatgtacattatcgattgttctcatttgataaggatatgatggacttggataaaaatgggagaattctgtccagaggtcttgccaacattcaccatgtcaatgatactaccatggtgatatcttacttgagaggtcccaatctctttgtgttcaactttcatcctgtcaattcatatgaaagatacattataggtgttgaagaagctggagagtatcaagtcacattaaatacagatgaaaagaagtatggtggtagagcactacttggccacgatcagaatattcaaagaaccattagtagaagagctgatggaatgagattttgcttggaagtgcctctgccaagtagaagtgctcaggtctacaagttgacccacgattctaagagcatgatcactctagtaatcaaagtgcctcatatgatggacacaaaaggaaaggttctacattgccctatactgatcaatattgtggcctttttgaggtgagtttctgtgattcttgagcacaggcttttggctagtcagttatcatgaacttttgccttcagcatccggatactctcttctcctgagcaatgacacttcgacgagaaaaaaactggcttgtgcatgggggtcaaaagcatctgccagattaagatttcaagcctcaaggaaaactatcaccgctttaggatacacacattgacgagtaagctctgataccttctaaagtgccag

SEQ ID NO: 93 - DNA, 555 bp, Lycopersicon esculentum
ctcgtatgtgtattgatgacggtctcgttgtttgcaactttgtcgctcaggctttaaggaaagagcctttgaccgtttatggtgatgggaaacaaacaagaagtttccaatttgtttcagatctggttgaaggcttaatgcggttgatggaaggagaacatgtcggacccttcaacctcggaaaccctggtgaattcacaatgcttgaacttgctaaggtggtccaggaaaccattgacccaaatgcacagattgtacacaaaccaaacacagaagatgacccacacaagcgaaaacctgatatctcaaaggctaaagagttacttggttgggaaccgaaagtggctcttagaaatggtttgcctatgatggttcaagacttcaggcaacgtattttcggtgatcacaaagaagaaagtgcaaagatcaccacagcttaatagaaaagaaaagtgtgttatgtttggatatttgttaaacttggtaaggggtgaaacgcacgcgaagaaaagacgactctcttgtgttgatgttagtttcaagcagaagtgcaag

SEQ ID NO: 94 - DNA, 491 bp, Lycopersicon esculentum
gttatacagttaggttgcaacaattccggatcgacatttatcttcattaagtgattactatattgcagtaattttgatgctgacaagtaccaagcactgagtcacagtggcaaacgtgcatttattttcatacttcatcatggtgacaaaatactatgttttctatagaagtcttttatttgttctggtgtcattagtcatcacttagagagaaaattaagtaaatatagtaggccctgtcttgtttgtacaattagaagttagttttctttaaaaaattattaacacctgcaattcatatacagaagaaagattcatgatttgtggttgagtgatcattactcctttatcttttattaagatataacttgaatgaattcgtgttacgaagtcgtcttcgatagaaacaaagactcaaaaagcagatgatattttcattttccaatagagaatctaatgtgggcctagccctcacttttgatgaatcatat

SEQ ID NO: 95 - DNA, 559 bp, Lycopersicon esculentum
ccatgtggggtttactttttgtgaatgaatatgaattaaagattcaatttttagtacatggtgttgataaagattatattgtgtttttatctttgttgtgtgctatttaatttttttttgaataagttgtgtgaattttatttggcaaagttttaagaaaataaatagaacttttgaatcattatttgtatgaattgtcatttaagttctcttacttagaaaaaatgctgaggttttttcccctttgaattcagttttagtcattggttgaacttaacttttgcaatcccctttcttcatttagactgttgaacttattttttttatcatgttatgcataatttcagcaatatgcttaagttcatgagtacagagtattacagggcctcttgccctcttaataattgtgattctctataaccaagtagtctctggatacgcgctttgtacgtgttacgtattcacgagtatgtaagatttaaaaaaattgcataaatgcaatatagcaatgcttttgacttaattagatatttcatctgtctcaacttatctgacattg

SEQ ID NO: 96 - DNA, 514 bp, Lycopersicon esculentum
cagcctctcatctcctcccttctccttgctgcctccctctcgttgtccagcaatagcgaggcaagcaacgcagcagcaacagttcggcgatggaccagcaacagcttccaacaactccggacggcagcagcagcttcgacgagaaccagcagcaacaacgaagccagcaaccaaacgacgcagcagcgaccagcgcctcccatcagcaaccagaagccgccggctgactgcaacaacgcgcaaccggcaacgcaggcagcaacctcaaccaacaccagcagcgatggaggctgctctgaccggctttctccgtccagcgaggtattcgacaagcttttagctccgagctttgatgggacaatcagaattctatcagacccaaatcgtgactccgacagcaacgaactctcagatctgcttgggaaagtaacgacatggtaacccttttaaattttatatttttttaaaatatatatctattgcttatgaatattgatttgaattttattatttg

SEQ ID NO: 97 - DNA, 325 bp, Lycopersicon esculentum
atcttctttcattatttttttccgggagaaaacttcaaatttttctttattgaggaaaaactataaaaattctcccaattgaaaacatcagttttgttgcagagctctaacgttttgcttgtttctgtttcttgactgcaactgcagtgcagagttactggtgtgtatttttttgctgcttttaaccaatctttgtttctttttttgtccatatatatgtcaagtttgtatcttttctgggtattttcttatagttactgataccctttagattttcaagcttttgagatctgaaatttgaggtatggacaattgggtgttcaat

SEQ ID NO: 98 - DNA, 306 bp, Lycopersicon esculentum
atcatattggattggtaaatttaatttgaacaagtcacaactccgcatacaaattaaaacttacggtacaaacgcggtgttcacactgtacacataataatactcctaaattaatcaatctaaagaaaccaataagttccttctttattgaataaaacatgtacgtataaataatcaaagtcacaccaattgctgatcaaacttgttcagtaattctgacatgtagagatcgaagagaaacttcaacaaccacagcgggtacaacaggtacctgggggtgttggttcacaacacttccacacaatg

SEQ ID NO: 99 - DNA, 601 bp, Lycopersicon esculentum
gcagcacaacaaatcataaaatcatatatatcatttttcttgaattgtcaatgtataacaagtcggttacacaacaattacattcatgggcgaaactagtaattttacaaaacatttacagatgaataacacaactacagatgaagtcaaactggacaagtacaattctctgatgaagtagtctatgccaaaaaaaaacaagatctcttcccagcaaatagataacaaacatcataataagcgcgtcttcaatgatgaataaccatgatcacatcgtttatatacatgcatatggttatacaagaaaccatagcaatatcaaacagaagccattgtctcaacatattgatgtgtatcataccattcaaacaccgaaacatctttccatataacagggatctttgctatctctatatccgcaacctccttgatagtcatacgatgagcatcatatttaacgattacgtctggtgtgggtaccaaaagcctcgctgaattttccttctcattaacaaccacacatacaacattgcgagtaccaccattcacatctagtaaatcaacttcttcactcaacattaatggatgagtaggatgcatg

SEQ ID NO: 100 - DNA, 631 bp, Lycopersicon esculentum
  mRNA (1)..(631)
actactcaattgcacattcgccttagtcgtcacaattcacattttggcaactccgttttcagcccatcgccgagagaataaataataatttgaaaagcgcttcctccacgatctggttgccggaggaggcgattttttgaatgaagagttcacctgaattttctcgaaatggcggaatcgacgacaattcagaggagctcgcctgaaggtgatgatcatgaactgaaggaggagaatattgagaagaaaaaggattttactgcaaatcctgagtttttcagttgtatgcttcagccagtgcctgccgattcagatccaaattacattggaatacgccggttcctcctccatcgcaaagctgaatccggtgtgcttcgtcgcaaggactggagaggcaatggaaaaggatacgtagcttatcgtaattatattagtaggccaaggaattgggagacattgcaaataccaagccgaccaagtaccccagggaacagtgggcgatggatgccttcagcaagtccagtttcccagttactcgagtcggatagctggaattccttaaaggatttacgaagtcccagtcaagcaagtcaagcattgagtcggactactagtttcagttcaaatgtga

SEQ ID NO: 101 - DNA, 516 bp, Lycopersicon esculentum
ggcatttgaacttgcagcaaagttttgttatggaataaattttgagattagcacagaaaacattgccttgctgagatgcgtggcggaatatcttgagatgacagaagactatgcagttgcaaatttggtggaaagaactgaggcctactttaatgaagtagcaattacgagtcttgcgggtgcagttacaattttgcgttcatcagaaaaccttcttcccattgcagagaaagtaaaattggtgagccgatgcatagacacaattgcatatacagcctgcaaggataaccaattctccacatcaggtagagcagaagctggtatcaacggatccacgttttcaaacccgaagcctatagttgactggtggtctgaagatataaccgtccttagaattgattttttccaaagggttctgattgcgatgatggcaaggggattcaagcggcatgcacttggaccaatattaatgctctacgcacagaagtctcttcgaggtttggaaatatttggaaa

SEQ ID NO: 102 - DNA, 604 bp, Lycopersicon esculentum
ggatccccatccgcacatatgatataggtcgctttaagttggttgatggaaagttttcacaggatggaacatcaattgttctttctgatgatgttgggcaaatatatttattaaatacaggccaaggcgagtctcaaaaggatgctaaatatgatcagttcttccttggggattaccggccccttatccaggatgcgcaggggaatgttcttgatcaggagacacagttagctccatatcggaggaacatgcaagatcttctttgtgatgcaagtatgcttccatatcctgaaccatatcagagtacgtaccagcgccgccgcctaggagctctgggtaccgagtggcgtccttcttcaattaaattttctgtaggcacagatggtggtttgggcctaggatatcaggttttaccagtggctgacttggacataatagctgaaccactgccagagtttgtggatacccttttctgggagccagataatgttattctgaatgatgagaccgattcagaatataatatgaatgaagagctttctgctgaagaagagcatgaatggttaagagatggctcttctagtggttcagtatgtagtgaaga

SEQ ID NO: 103 - DNA, 571 bp, Lycopersicon esculentum
tggatccggatctggacagcccggatcagccgggtctggagtttgcccaatttgctgccggctgcttttggggagtcgaattggctttccagagggttggaggagtagtgaagacggaggttgggtactctcaggggaatgtccatgacccgaactacaagcttatttgctccggaacaaccgaacatgccgaggccattcggatccagtttgacccgaatgtctgcccgtattccaatctcctttctctattttggagtcgccatgacccgaccactctaaatcgccagggtaatgatgtgggaaagcaataccgctcaggaatatattactataatgatgctcaggctcaactggcaagggagtcgttagaagctaagcagaaggaatttatggataagaaaattgtcactgaaattcttcctgctaagagattttatagagctgaagagtatcaccagcaatatctagagaagggtgggggcagaggttgtaagcagtcggctgcaaagggctgcaatgacccaataaggagtgacagcacctttctttctccagtcaatagaaagga

SEQ ID NO: 104 - DNA, 624 bp, Lycopersicon esculentum
gcaccaggttaaggagctgttgaatgcgttgttccaccagctccggaaaaacagcaggcacatgctctaaaaaagcatgaagcgtttgctgacgttcgcgcctttcattttcaacatttagaattctttgattaatttgagaaagtctttcagtggtagctggatccatatcttcctttgctttgccaaatagagaaagaagcttctatttataggcgttggaggcccttaaccctttcttattattagtaagataggttgtcttggttccgtaacatggttcaaacctggcttttcatgaatatggaaccccatccactagatttaagtgcgctgaataattgtcctttcctcgtacgcatttgagtactcaaggccccctttcttttggcgggtatcgccacgtggcttaatcccggatcactccttaaggaataggacacaataatatagcgctcattagagaagagagtaccccctttattataagacaggccccccgcgtcctttcttaagagatggagcaatcgtaactcgacagctcaaaactgaccagtacaaaaccatgtgatctggcctcgtttacgtaccccactggcactagcctaacgccctgcctactcg

SEQ ID NO: 105 - DNA, 510 bp, Lycopersicon esculentum
ggtggacattgaagaatttaaatctgctctgtcccaagtggatactgatatgaagaatcttccggcaacagctcaggttgcatctcagcaaggtgtctacttagctaaatgctttaatcgtatggaggaatgtgaaaaaaatccggaaggtcctcttcggtttagaggagaaggccgccatcgctttcacccattcaggtaccgacatttgggacagtttgcacctttgggaggagagcaaacagcagcacaacttccaggagattgggtttcaattggtcatagcagtcagtggctctggtattctgtgtatgctagcaaacaagtcagttggcgtacaagggcattggttgtctctgactgggtaagacggttcatatttggaagagactcaagccagatttaaggtgttacgccctcgacatttcataggagctccagtggatttatcttgaaattccattaggctgttaccaaaagtttcttgatgtgaatcctaacggatttgtt

SEQ ID NO: 106 - DNA, 3134 bp, Lycopersicon esculentum
cgttccaaaaccatacaaagaaccatcaaaatctaattagaaatctcttttttcttcttccaaattccctcattcataattttattttaaaaaaatcaatggcggcatctgtggaaaacagacagtatactcaccttgaaccgggtttatcaggcgtaggccgtactttcaagcctaggcctgattccccggttcgtggttgcaacttccctccttcatccaaccatgaactccctttccaaaaaaaacaaaatcccccaatttaccttgattattcgtctagtgaagacgaggatgacgatgacgaaaaaaatgaatacgttcaaatgatcaaaaaaggtaaaactgaattagaaccatcaattcatgatactagagatgaaggtaccgctgataattggatcgaacgaaacttttccttaatacgtctcaccggtaaacacccattcaactccgaaccacctttatctcgccttatgcatcacgggttcatcactcccgtaccacttcattacgtccgtaaccacggcccagtccccaaagcatcctggtctgactggactgtggaagttacagggctggtaaaacgaccaatgaaattcacaatggatcaattagttaacgaattcccttcacgtgaattccctgtcacacttgtgtgcgcaggcaatcgtcgtaaagagcagaatatggtgaagcagacaattggtttcaattggggtgctgctgccgtttcaaccaccgtatggcgcggagtacctctccgcgccctgttgaaacggtgcggtgttcagagtaagaaaaaaggcgcgcttaatgtctgtttcgaaggttccgatgttttgcctggaggtggtggttcaaagtacggaacgagtataaagaaggaattcgccatggatccatctcgtgatattattgtagcttacatgcaaaacggagaaatgttgtcaccggatcatggttttccggtaaggatgattatccccggattcatcggtggaagaatggtgaaatggttaaagaggattgtggtcactacacaagaatcggaaagctattatcattacaaggacaatagagtcctccctccacacgttgacgcggaacttgccaacgcggaagcttggtggtacaaaccagagtacatcatcaatgagctcaacataaactctgtcattacaactccgtgccatgaagaaattttgcccatcaatgcgtggactactcagagaccttacacgttgagaggctatgcttattctggtggaggtaaaaaggtaactcgagtggaagtgactttggatggaggagagacatggagtgtgtgtacacttgatcacccagagaagccaacaaagtatggcaagtactggtgttggtgcttttggtcactcgaggttgaggtgcttgacttgcttagtgctaaagaaattgctgtacgagctaccgatgagaccctcaacactcaacccgagaagcttatttggaacgtcatgggaatgatgaacaattgttggtttcgagtgaagatgaatgtgtgcaaacctcacaagggagagattggtatagtgtttgagcatccgactcaacctggaaatcaatcgggtggatggatggcaaaggagagacacttggagatatcagcagtggctcctccaacactaaagaagagtatatcaactcctttcatgaacacagcttcgaagatgtattccatgtccgaggtgaggaaacacaactcttcagactctgcttggatcatagtccatggacatatctacgatgcctcacgtttcttgaaagaccatcccggtggtgttgacagcattctgatcaatgctggaactgattgtactgaggaatttgatgcaattcattctgataaggctaagaagctattggaggactttaggattggtgaactcataactactggttacacgtctgattcgtctccaaacagttctgtccatggatcctcttcgatcagtagcttcttagcacctattaaggagcttgttcaaacaccaacaaggagtgtagctctcatcccaagggaaaaaatcccttgcaaactcgtcgacaagcaatccatctcccatgatgttaggaaattcaaatttgcattaccctctgaggatcaagtcttagggttacctgttggcaaacacatattcctctgtgccacagttgatgacaaactctgtatgcgtgcctacacgcctactagcacagtcgatgaagtagggttcttcgagttggttgtcaagatctacttcaaaggtgttcaccctaaattccctaatggaggtcaaatgtcacaacatcttgattctctcccaataggtgcattccttgacgttaaaggtccattaggtcacattgaataccaaggtaagggtaatttcttagtccatggtaaacaaaagtttgccaagaagttagctatgatagcgggtggaacaggtataactccagtatatcaagtaatgcaatcaatattgaaagatcctgaagacgatacagagatgtatgtggtgtatgcaaacagaacggaggatgatattttgctcaaagacgaacttgatgcatgggcagagcaagttccaaatagggttaaagtatggtatgtcgttcaagaatccattacacaaggatggaagtatagtacaggattcgttacagaatcgattcttagagaacatatacctgaaccatctcatacaacattggcattagcatgtggaccacctccaatgatacaatttgctattaatccaaacttggagaaaatgggatatgacattaaggaggaactattggtgttctaaattggatggtgatgatgatagatgatatatctctttggaggaataaattcttttgtattttcagttgtacatattgtgtttttgtttatcatcaaaatgtactacttcttgtagttcttacattttaattttctactcaactttaggtataaaatgttgtactgtgtactagtatgtgttatgtcaaagttaagattgtattcatcatggaattacagtactttgttgtcacaagtcttgttgtatatatttttaccaaattaatgtgtatatatatatagtaaattgaaacatttgtgt

SEQ ID NO: 107 - DNA, 349 bp, Lycopersicon esculentum
  misc_feature (326)..(326): Unknown nucleotide 326
cgtggtcgtgacatagcagtaattgctgctggaggaatagtggatgagcgtggttatgttgctgctctggcccttggtgcacagggtgtttcattaggtaccaggtttcttgcaacagaggaaagctatgctcacccaacgtacaagaggaagctgattgaatttgatcaaacagagtacacagacgtatttggtcgtgcaaggtggccaggggcaccacaacgtgttctggcaacccccttcttcatggaatggaaggcgctcccaagtcatgagaatgagtcaaatcaacctgtcattggccgcacaatcatacatggcagggnaaattgaggtttcgtcgctttgc

SEQ ID NO: 108 - DNA, 659 bp, Lycopersicon esculentum
gctcttattactatgcttcagtcacttcatcatgccaaaaatggtgtagacattgtttctggcacgagagttaagtcacactttgctaagcctaattggagaaacgtctacaaacgcattgctctcaaccacccagaggctaaagttggagtgttctactgtggggcaccggcactaacgaaagagctaaaacaacacgcattggatttttcacacaagacatccaccaagtttgatttccataaagaaaatttttgatccatcaaagaattcctcaaaactgccacggacactaagttgtttttctagaatccaccacctctctattatgtacagataatgatgcacaccaagttgatatatattaattgtggttgtaatattagtctattacaaaaaataagattttttaaaagtatatagataagatgctgaaggaattaagaatgttctcttggagaggcaggcagaagatagtggagttgaacctgttttaatgggtgccacccaccactaaatttggattttacattataattgcatagaggaaggtggttgtcatcattgttttttgttattgtttattttttgtactcttttacgtgggggaagagagagtcaactagtcacctacttagtaattattgaccatgatttgg

SEQ ID NO: 109 - DNA, 493 bp, Lycopersicon esculentum
gctctcaaagatgccgatggataccgaaacaaatcaatcaggagatttcatctttcgatctaagctccctgatatttacatccctaaacatttgcctttacattcttattgctttgaaaatctttcggagttcagttctcgcccctgtttgattgatggttcaaatgatcgaatctacacttatgctgaagttgaacttacttcgagaaaagttgctgttgggcttagcaaattagggatccaacagaaggatacaatcatgatcctcttgcctaattgccctgagtttgtgtttgcgtttattggagcatcgtatctaggagcaattttaacaatggctaatcctctgtttacaccagcagaggtagtaaaacaagccaaagcctcaagcgctaagatcgttatcacacaagcttgttttgcggggaaagtgaaggactacgcaattgaaaacgatttgaaggttatttgcgtcgactccgcacccccgagg

SEQ ID NO: 110 - DNA, 569 bp, Lycopersicon esculentum
gcggaagctggtccagtgctggcgatgtgtttggcatttgcgaaagaaccgtttgatattaaatcaggggcatgtggtaccgttgtgaggaacgcagagatgaaaattgtggatccggatacgggttgctctctgccccgtaaccaacccggtgaaatttgcattagaggtgatcaaatcatgaaaggttacttgaatgaccctgaagctacagctagaacaatagaaaaagaaggatggttacacactggcgatattggatatattgacgatgatgatgagcttttcatcgtggatcgattgaaggaattgatcaaatacaaaggatttcaagtggcgcctgctgaactcgaagcacttcttgtcaaccaccctaacatttctgatgctgctgttgtcccaatgaaagatgaacaagcgggagaagttccagtggcttttgttgttagatcaaatggatctaccattactgaggatgaagtgaaggatttcatctccaagcaggtgatattctataagagaataaagcgtgtatttttcgtggagacggtaccgaaatcttcatcagg

SEQ ID NO: 111 - DNA, 616 bp, Lycopersicon esculentum
acaaataaaagaacctttgcattactaaaaagagaaatacaagatatctagccaaacaatatactgataagatcagaggagaatacacttgcgccattactgaaaatctagaggcactgatatattctcgtgtcccgatttcttgaccaagtagtatgtacgtaagtaaatatcgaaaaacacattcattttattgttgttaataggagaacgaaccgtagatagccgacactttttctacgctccaaatttagggactttggcagttgaaatctgagcaaggaagtgctctgcaacaagtcgtcgttgaattttccctgatgcagtctttggaagagaatcagtcatgaataccttctttgggaccttaaaggctgccaggttcttcttgcaaaatctcataacctctgcttcatcaatgtttgacccttctcttggaataattgcacaatttatctcttcaccatacttgtcatcaggaactccaaaagcaacagcctgagcaatttccggatgagaagctaggactgcatccacttcaataggtgatattttctctcctccacggttgatcaactccttgattctgccaaccaaatgcaagtattcatcagag

SEQ ID NO: 112 - DNA, 575 bp, Lycopersicon esculentum
cgtcaaaataatgcaattgtcagaaagccaaattaccattgtacatcatataggtaaaacttataatgaatctagtctaataagacttttatgaaaaacataaaagagaagaaaattttctgaacaaacaaacaacagtcataattaactgatagtattacaaagtttagaaagatcttggtttcatcacaatggtggaatgcttcaaaatgtggagactgaactgtccacctttctccgaggtgtcgagcttcgactgccctggaggaggcaacatctcaaagttctgcaccaaacgtccaatggtgatgccaagaattggcaatgcaaggataattccggggcaactcctgctgccaacaccgaaaggaagaaatctgaagtcgttgccattggcttcaacgtgtttctcctcttccaagaacctctcaggtctgaactcttcaggtttcttccagtgggcggggttgttagctagccaccaagcgttaactaagattttgctctcagctggaatatcgtatgcagcaagctttgcatcatgaaggttcatgtgtgggactaaaagaggaatt

SEQ ID NO: 113 - DNA, 484 bp, Lycopersicon esculentum
caagagcatggacagcaatgctctaaaatgtgccattgatcacattcttgatgctcaacagaaaggagagatcaacgaggataacgttctttacattgttgagaacattaatgttgctgcaatcgaaacaacattgtggtcaattgagtggggtatcgcggaactagtcaaccaccctcacatccaaaagaaactccgagacgagattgatacagttcttggaccaggagtgcaagtgagtgagccagacatgcccaagcttccataccttcaggctgtgatcaaggagactcttagactccggatggcaattcctcttttagtcccacacatgaaccttcatgatgcaaagcttgctggatacgatattccagctgagagcaaaatcttagttaacgcttggtggctagctaacaaccccgcccactggaagaaacctgaagagttcagacctgagaggttcttggaagaggagaaacacgtt

SEQ ID NO: 114 - DNA, 527 bp, Lycopersicon esculentum
ggaacttggaggttacctgttcgacttcctctttgctgctcaagatgcttctacttctgctctgttatgggcaatcgtgcttctagattctcacccacaagttctggagaaagttcggtcggatgtagcgagattctggtcgccagaatctgaggagccgctgacggcggaaatgctcacggaaatgaagtacctggaagcggtggcgcgtgagataatcagaatcagagctccggcgacaatggtgccacatattgccggcgaagaattccggttaaccgaagattacgttatcccaaaaggaacaattgtgttcccgtcggtttttgattcatcatttcagggttttcctgaaccggagaaatttgaaccggaccggttcatggaggagagacaagaggagcgggtttacaaaaagaactttctagcattaggtgctgggccccatgcgtgtgtcggacagaagtatgctattaaccacttgatgcttatcattgctatgtttacggctcttgttgatttcaaga

SEQ ID NO: 115 - DNA, 664 bp, Lycopersicon esculentum
cctgaggctaataaatctgcttttcaatttggttggtttcatactggagatgtgggatttttggactctgatggatatttgcatttggttggaaggatcaaggagttgatcaaccgtggaggggaaaagatatctccaattgaagtggatgcagtccttgtttctcatccagagattgctcaggctgttgctttcggagttcctgatgacaagtatggtgaagagataaactgtgctgtcattccacgtgaagggtcaaagatagacgaaggagatgtgttagtattctgcaagaaaaatctagcagcctttaaggttcccaaaagggtgttcatgactgattctctaccaaaaactgcaactggaaaaattcagcgacgacttgttgcagagcatttccttgcacagatctcaactgctaaagtgcctaagtttggagcttgaaaatgatacatactatcaatctctcaaataataattattaacatatgatttttactggcaatattattttttctaggcacattagaagttccttttccttgtaaaagtgaacattaaacacagtgatattcttacaatgaatttttagacttgtacaatttaatctgtaagaaaactgcctatagcagacagtagtaataaactttacaacatatcttgc

SEQ ID NO: 116 - DNA, 469 bp, Lycopersicon esculentum
aggcaccaattggtaatagaattcaagaatgcaggtcatatccattgtacaaatttgtgagggaagaattaggtgctaagtatttgacaggtgaaaaagctctatctcctggtgaagtatttgataaagtgtttacagctatgaatgaaggtaagttgatagatccattgcttaattgcttgaaggaatggaatggtgctcctcttccactttgctaagagaaactaggacttatgttgcttggacacttcaaagcgtgcgtgtcggatcctttaaaagtggtatatttttagaggatttgagacagtgcaagcgacatagggaaaacatatttgtcttaatattgtaggtacattatttttcaagtttatttttctagttttgtggaactttttctaatggtttgtatgaatatttgggaatctttgaacttatagatgacactcttaagcaattgtatttgaacttt

SEQ ID NO: 117 - DNA, 566 bp, Lycopersicon esculentum
gcaccagatgaaaggttatttcagtaatgaagaagctactgcttccactctagactctgatggatggttaagaactggagatctatgctatatagatgaagatggtttcatttttgtggttgatagattaaaggagctcatcaaatataaaggttatcaggttcctccagcagagttagaggctttattgctcactcattcagaaatttctgatgctgctgttataccgtttccggataaggaggtcggacagtttcctatggcatatgtagtacgtaaaactgggagtactatatcagagagtgcagtcatggatttcattgcgaaacaggttgctccatataaaagaattcggagagttgcatttgtggcttccataccgaagaatccttctgggaaaatactcaggaaggatttgataaagctagcaacatcaaaactctgagaacaagatttatgatttttgcattttagcaagtgtaacttcctcagcagccatagtcttgttattgtgctaaataatgagtgtgtgctcttgttgattattaatttggtatgattgtgga

SEQ ID NO: 118 - DNA, 539 bp, Lycopersicon esculentum
  misc_feature (432)..(432): Unknown nucleotide 432
caggaaggaatccaagcttggattatggactcaagggagctgaaattgcaatggcttcttactgctcagaacttcaattccttgcaaatccagtgactaaccatgtccaaagtgctgagcaacacaaccaagatgtgaattcattgggcttaatctcagcaaggaaaaccgctgaggctgtagacatcttgaagctaatgtcatcaacctatctcgtggcgctttgccaagctatcgacttaaggcatttggaggaaaacttgaggagtgctgtcaagaacacagtgagccaagtagctaagagaactttgacaatgggtgctaatggtgaacttcatcctgcaagattctgcgagaaggaattgcttcgagttgtggacagggaatacgtgtttgcctatgctgatgatccctgcagctccacctaccctntgatgcagaagctgagacaagtccttgttgatcatgcaatgaagaatggtgaaagtgagaagaatgtgaacagctcaatctttcaaaagattgtagctttcgaggac

SEQ ID NO: 119 - DNA, 509 bp, Lycopersicon esculentum
gcagcctaaggaaggacttgcacttgtgaatggtactgcagttggttctggtatggcatcaatggtactgtttgagtccaacattcttgctgtcatgtctgaagtcttgtcagcgattttcgctgaagtgatgaacggaaagcctgaattcactgactatttgacacacaagttgaagcatcaccctggccagattgaggctgctgctattatggaacacattttggatggcagctcttatgtcaaggcagctcagaagctccatgaaatggatcctcttcaaaaaccaaagcaagatcgttatgctcttcgaacatctccacagtggcttggacctcaaattgaagtcatccgtgcagcaactaagatgattgagagagagattaactcagtgaacgacaacccattgatcgatgtttcaagaaacaaggccttacacggtggaaacttccaaggtacccctattggtgtctccatggataatacaagattggcccttgcatctattg

SEQ ID NO: 120 - DNA, 478 bp, Lycopersicon esculentum
  misc_feature (376)..(457): Unknown nucleotides 376 and 457
tgctataagcctgaagatgctaggatatgcacaaggaagattttcatggagaggtcaaagttggttggttcattccctgatgaaactcttgagttccagaggaaaattcttgagaggtctggccttggtgaatcaacttatctccctgaagctgtgctgagggtaccaccaaatccttgtatggcagaagcacggaaagaagctgagatggttatgtttggtgccattgatgagctccttgctaaaactggtattaagccaaaagatatcggaattctagtagtaaattgcagcttgtttaatccaacaccctctttgtctgcaatgattgtgaaccattacaagctccgtggaaacattgtgagctacaatctangtggaatgggttgcagtgctggtttgatctcgatcgatcttgcaaaagatcttcttcaagtacatcccaatacctatgctntggtgctcaacatggaaaaca

SEQ ID NO: 121 - DNA, 597 bp, Lycopersicon esculentum
  misc_feature (542)..(542): Unknown nucleotide 542
ggcaccagccccatgccaattcttccatttgctcacttcctagtttggctaaacttccaaatgatacatacaacactgagctaattggttgatggtttaaccaatttaggcattcatttgtcattggcttgaacatactaagaccatactctttatcatcatgtagtctcttgtctaagtacattgatggtattgttggtccaattgtctttattggatatatcttcgacatccaatcaattacctacatgacaaataataaactatattatatgattcagttaattcctttaatgttttttacactatgggtggttgagtcaggatttttcactaaggagattcaaaatatgaagaagtaaacatacgaaaccgtagatctggaattcaacatctacataaaaaagaatatgcggaagaatattttacctctttctccaactcatagaagctattgattagaacacaatcaactttgtcaagatttgagaattgatttgctaacatttcaactatcctttctgcttcaggactaataacanaagaaggtacatctgatgcatcgatcgaatttggaaatccaggaattaatatttc

SEQ ID NO: 122 - DNA, 616 bp, Lycopersicon esculentum
gcccctttttcaaagagtatccattttgcccaaattgctgttctttgagtctgaaattcgattcactatggaagaagcaaatgttgttgaagctaaggatggaactatctcagtagcctctgcatttgctggtcaccaagaagctgtacgggacagagaccacaaattcttgacacaagcagtcgaagaagcttacaaaggtgtagaatgtggagatggaggcccatttggtgcagttgttgtttgcaatgatgaagtagttgttagctgccacaacatggttttgaagcatactgaccccacagctcatgcagaggttacggcagtaagagaggcatgtaagaagctcaatcgaattgagcttgcagattgtgagatatatgcttcttgtgagccttgtccaatgtgctttggtgctatccatctatcacgcatcaagaggttggtatatggagccaaagctgaagctgcaattgccattgggtttgacgatttcattgcagacgctctcagaggtactggattctatcagaaagctcagttagagatccagaaagctgatggtaaaggagccctaatagccgagcaagtatttgagaagacccaagaaaagttc

SEQ ID NO: 123 - DNA, 635 bp, Lycopersicon esculentum
gcgacatattaatccagtgactgatgaggatatagtgggttttgggaatgacatagaaaaaatgtttcagtatctgattagaggtacaaatgatctagacgttgtcccaattgtaggcatgggtggacaagggaaaacgacagttgctagaaaggtgtacaatagtgacaacattgtttctcattttgatgttcgagcatggtgcatcgtttcccaaacatataaccggagaaagctattacaagacattttgagtcaagttaccggttccaaggacaaggggtatgaggatgatatccttgctgatgagttgaggaaaagcttaatgggcaagagatatctcattgtcttggatgatatgtgggattgtatggcatgggatgacttaaggctttcctttccagattccggaaatagaagcagaatagtagtaacaactcgacttgagaaagtgggcgaacaagtcaagtgccatactgatccttattctcttccgttcctcacaaaagaagagagttgtgaattgttgcaaaaaaaagtgtttaaaaaggaagatttcccgcctgaactacaagatgtgagtcgagcagttgcagaaaaatgcaaaggactgcccctagtggttgtcttgggtg

SEQ ID NO: 124 - DNA, 649 bp, Lycopersicon esculentum
gggagctttggagtgtgccgtgtgtctgaacgaatttgaagatgatgaaatgttgcgtttgatccctaagtgtgatcacgtttttcatcctgaatgcatcgatgcttggctcaaatctcatgtaacttgccctgtttgccgagctgatcttactcctaaacctgatgaaccggtgccggttcaaactcctgaggtgaaccgggaattggggcaagaacaggagaatacacagcaaaataacgaagttacgattcaaatagatgtagatgagaatttgatgtcacagcaagaggagacctctgtaaaaccagaagttaaacgaaatttgagtttcaacgcggtgaaccggccgccgaggtctttttctctgaagagaccaaagatgttaagcaaattcaggtcacattcgaccggtcactcattagtggtaccaggggagaatttagaccggtatactcttagattgccggagaaggtgaggaaagatgtaatggaccgggctctactaaaccggacaaggagctgtgcagtcacgctaccaagacacggtggcaccacaagaggatacagagccggtccaggagagggaagcaacagaggtgcaaggtcattcaagcgaatcgaccggttcgatccggaaacaaaatca

SEQ ID NO: 125 - DNA, 457 bp, Lycopersicon esculentum
gttaaaagttggcacctcaacaaatattatggttgttcaaacagctcaggctgctgcagagttattcaaaaaccatgatacttccttcgcggatcgaccactggtcgatgtgaattcagcacataattactatcaaggatctccggctttagctcgatatggccccttttggaggtttcaaagacggttatgtactgtggaaatgtttgtacacaaaaagatcagtgaaacagtgcgagtaaggcgaaaatgtgtggatgatatgttgaaatggatagaaaaagcagggtgttctgctgaaaaaggaagtggaattgaggtaacgcgttttgtgttcctagcatcgtttaatatgctggggaatttgattctatcgaaagatttggctgatcctgaatccgaggagggcctcgagttcttccatgccatgaagggaatcatggagtgggcaggggtt

SEQ ID NO: 126 - DNA, 421 bp, Lycopersicon esculentum
cagaatgcccatttggagtttgtagaagttttgaatgtgaaagaacaagttgattctggaatgatgtactatataacacttgtggcaactgatgatggaaataaaaaagattatgaagccaagatttgggtgaaggaatgggaggacttcaagaaagttataagctttaagcttgttggtaaggatagtgcaataattgggggctttaccgatgttccattcccaaacaaacgcgagttccaagatcttacacgttttgctattcaggattataataagaaagaggttatttacaatgacttactcatcttctattttttcctgttaatttcatattcaaatttataatatattaattttgaacccattgcatgagatatctaataatcatttcatgtgaatgaacgtcatatacaattta

SEQ ID NO: 127 - DNA, 549 bp, Lycopersicon esculentum
gtttcgtgtgtgtatttaccactgtcgcactcaagcttagactccattttcatctcttaaactccccatcaccttctaattggacttgcaattttccccctctccacctttaaattctctttccaaatcttcccacttttcacatcatactccataattccaaatttcatacttggtgttttgcgtccactaatctagctagcctctgtttggaactgcaaattcaaaataccaaaatgggaagtggtaaattaggtgattcaggtattattgcgatcacggctaagatcatgatggctgtcgtcgtatttctcttctttacggtgctcttggtattcttccttcacatatactccaaatttttcacgacagaggacgccaaccccaacgacggcacaaggcgccgccggttcgacttcgccggtggttatcaggaagtgaatgcactccggagaggcattgacccttcaatccttaaaaccatacctgtaattccatttgataccaaggattttaaagatggactggaatgctcagtttgtcttaacc

SEQ ID NO: 128 - DNA, 189 bp, Lycopersicon esculentum
aggaggaggccgaggtttgctccggaattggatgggctccactgtttcgaaacaattcttccctattgatttctacattaagtctaaaagattgaaattctttgtacttgtctgattgtaattaactggccatggccatgtaaccgtaaatcacggagattgatgcaatatagtgtagtattaaatctg

SEQ ID NO: 129 - DNA, 499 bp, Lycopersicon esculentum
gttatatatatatagagaaattaaagttcatttctcttatcgatcatcatcgttatatcatcgtatcatcaaatggcattttcggagccaatagttatatgcaagaagcatccacaacacaaacaacaacccggagtatgttcatgttgtttaagagaaaagttatcaaagatcaatggtacaactacaatagctaaaatttcatctattccttcttctatatcctcctccgtttcctcgtccaactgcaactctccacgagctgcaattggacatcgtcgaatcacgtccgatgttgctggcggccactattcgttcgtagcgctagcggccgccaacgctggaggaggaggaggggggttgaagaaaagtaggtcaatagcttttgttgctagagggggtagagttggaattaatggaaaaaagaagaaagaagggttttggtcaaagttgatccggtcaacggggaaacggaccaatcgggttgttgtgcattgat

SEQ ID NO: 130 - DNA, 644 bp, Lycopersicon esculentum
gatttattctcagtatttgaagagatacgatactaatttcaaaatcgtcgaaaacaagtgtgatgggatcagttcaagatgagagtaaggatgaatttaaacaaagtcttacccggggaaaactcaagccttcttcctcatcgtcgttccgtctccgtagcccgagcttgaattctattcgtcttcgtcggatcttcgatgtgttcgatagaaatcacgattgtttgatcagcgttgaagaactcagccaagcacttaacctgcttggattagacgccgatctatcagaaattgaatcgatggtgaaatcatacatcaaaccagaaaacactggtcttagatttgaagatttcgaagccttacatcgatctctgaacgacgttttcttcgggtcaaaatgtgaagataagctcgggttgaattcggatccggcacaggatgaattggatctgaaagaagcgtttgatgtttttgacgagaacggggatggatttatatcggcgaaggaattgcaggtggtgttagagaaattgggattaccggaaggaagtgaaattgatagagtggagatgatgatttcgtcagttgatcaggatcatgatggtcgcgttgatttcttcgagttcaaagatatgatgcgtact

SEQ ID NO: 131 - DNA, 555 bp, Lycopersicon esculentum
cagattaggaaccctaataaacgaatcgggatttactacgattcgatagaagcaagaggcatgtatcaaggtcagagatttgctagtcaaaatttggaacgattttatcaaggacacaaaaatactagctatttgcatccagtgtttaaaggacaaaatttggttttattgggagatagagaaaaaacaaattacaataatgagaagaatttagggatttatgagatggaaatgaagctttatatgcgtataaggctaaagattggttggattaaaactcacaaaattaagcccaaaattgagtgtgattttaaggtacctttggaggccaatggtagatctgttaattttgaggaaactaggtgccatcttgattggtagatttatttattttttaaatcattttcacatttatggagtttatttatttattatcctttttctttcattatatattttcttgattaggagatttatctcctttttgttatttggattatactatggttgttaataaatgatgatttacatttatttatgctgattttctttgtt

SEQ ID NO: 132 - DNA, 602 bp, Lycopersicon esculentum
tcgtatggctgcatccctcattcgtcttcatttccatgattgttttgttcagggttgtgatgcttctctcttgcttgatgaaactccaactattgtcagtgagaagacagcgctgccaaatcttggatcagttagaggctttggtgttatagaacatgcaaaaagagaggttgagaaaatatgttctggagttgtatcgtgtgctgacatactcgcaggtgctgctagagatgcatcaagtctcgttggcggtccatcatggacagtgcaactcggaagaagagactcaaccactgcaagtcatactgttgcggaaactgatcttcctggtccttatgatcctcttagtaggcttatttctggctttgccaacaaaggccttagcacaagggatatggctgctttatcaggatcacattcaattggacaagcacaatgattccttttccgtgatagaatttatagcaacggaacagacatcgatgctggatttgctagcactagaagacgacaatgtcctcaagacgatcaaaatggagacctagctccacttgatttggtaacacctaatcaattggataacaactactttaagaatttga

SEQ ID NO: 133 - DNA, 434 bp, Lycopersicon esculentum
gcaccagattttcctctctttattcttgtaacaacatttagtttcccttatggctgatcaacaaaaaatccatccggttccggagccggagcaagtagccccgccacccaagccctcagctccgttagtgccacgtggctcttcccgttcagagaacggggatcctgagcgtcagcaatcaccaccactattgaaaagatcgttaaccccatattcacccttaaaatcaccaaaaaagaaaagaggctgctgcgtaagatgcctatgttggacatgttgcctatttttcttactaatcgtactcataggcatcgcagcagccgtaatttacctagtcttccaaccaaaattaccaaaatactccatcgatagcatgagaagtacccaatttaacctcaacaccgatacgagcttgtccgcgacatttaacgtga

SEQ ID NO: 134 - DNA, 599 bp, Lycopersicon esculentum
gctaaacacaattatattgtattgctctattgctattgtcatattatcatcaattccgttagctcattgtaacattgcacaaggtcctaaagcagttgaaaattggttccaaaaagtttcccattcaaaaccaaaaataaccaaacttcatttctattttcacgatatagttgctggtaaaaatccaacgtcaataccaattgcccaggctaattctacgtctcattcgcctacggcgtttgggacgctagcaatattagacgatcgattaacaacgggaccagaaaataattcaacgactattggtcgagctcaaggaattgtaggtgcagcttctcttgaagaatttagtttgttgatgagtttgaattttgtgtttactcatgggaaatataatggtagtacacttagtctacttggacgtaacactgttttgaatgaatatcgtgagatgcctattgttggtggttctggtgttttcaggctagctcgtggtgttgcaaccgcaaaaaacacatactttaagtaagaatggtgatgctattgttgaatataatgtcgaagttttaccattattgattaagattatattttgctaa

SEQ ID NO: 135 - DNA, 433 bp, Lycopersicon esculentum
gcatcaatgaagcctatcatctttggtgataatgtggcaacagctcttagaagctggcaccatacagcgaaaaaacgggtgaaacatgggctatcaggacataccacccctgcaaacagcagaccaaccacaccattgcgtggtacctcccctgttcacttattacgcggttatccacaatataatgaggacagtgttcaagcatctcctcggacatccaatgtcgaaaatgaagggtgggctaatgaaaatcaggagggagagatcctgcagcatgcctccactgatcataacaagcaaattgagattacaatgtcagattttacttttggaaacaaataaatgtaaaaacgaattttcttcttcattgttttaagttcattactgtagttcaaatggcaatgattttgtaaaattttatacagaggtactc

SEQ ID NO: 136 - DNA, 613 bp, Lycopersicon esculentum
gcaccagtggagaaccaatgctttaaacatcttagccctgatcagcacaatgaactgtcatccaaactaatggattcttttgacgaggcaatgattacctctgctgtggagaaattggagcaggcgttaagtgatggatctgtctcttatcatgtgctttcagggcgtcacaagagcttgtacagcatttactgcaagatgctaaagaagaagcttagtatggatgaagtccatgacattcatgggttgaggttgattgttgaaaatgaggaggattgctataaagcacttcaagttgttcacgagctatggcgtgaggtacccgggagatacaaggactacatagagaaaccgaaatgcaatgggtaccaatctcttcacacggttgttcttggggaaggcatggtgcctcttgaagtgcagatccgaacaaaagagatgcacttgcaagctgaatatggctttgctgctcattggagatacaaagaaaatgattgcaagcactcctcttttgtacttcaaatggtagagtgggctcgttgggttgtaacaccctggtgcgagacaatgagccgagaccagtcatctgttggccacactgaatcgttaaacc

SEQ ID NO: 137 - DNA, 530 bp, Lycopersicon esculentum
  mRNA (1)..(530)
gagatgatttcaattgaatccattaaatccatagcacctcatatttatattataccaaagtatctgaatgataaatcagaaactcagatattgcatagtatgtgacacaatgaagatacatgataaatgacttattcaaacctttctaaaccaacaacatgcaagaaaaacattaggatgtttccgaaagaactttgcgaacaatttcacgagctttgcatctagccatgagtggctcagcttttggcatagtgacaccttttccttctgtcaaatcgaccttctcatcgcttacccttttccattcaaaacactgcactaaagttcctaaagttaaaccaaccacacgttgagcaagtccggagccagggcaagctctccttcccattccaaaaggcaatagcttccatgattccacttccacaccataaaacctttctggcttgaagctctctggatcctcccaaagtaaaggatccctgtggatggaccaagcattcaccagtagcatcgtgcctcgtggaatgtgg

SEQ ID NO: 138 - DNA, 558 bp, Lycopersicon esculentum
ctgaaactaacacaaagcaggagcaggagcagcaacaaacagagagaagaaaacagaggaagataagaggaaaatttatcgaattcgaatcgagagaaaaggggaagtgaagttgcgaagagtgagaatttcaaaggaaatgaacaaaggagcaaacggaaatcagcaattggagttaccggcgggattcagattccatccgacagacgacgaattggtgcagcactatctctgcaggaaatgcgccggacagtcgattgctgtatcaattatagctgaaattgatctttacaagtttgatccatggcagttgcctgagaaggctttgtacggtgaaaaagagtggtattttttctcaccaagggatagaaaatatccgaacggttcacggccgaaccgagcagcaggaaccggttattggaaggcaaccggagctgataaaccggtgggaaaacccaaaaccttagggataaagaaggcacttgtgttctatgccggaaaagcacccagaggtataaaaacaaattggattatgcacgagtaccgcctcgccaacgt

SEQ ID NO: 139 - DNA, 582 bp, Lycopersicon esculentum
tggttatgctaatgaaagacatgttcgaagtatccacaccaactccacttttgagtttgaagcaagaattcttcacacaagttgttcaagtcttgagggatgagatctctcaaaaggctacaaaagcatcattgcaagtgctagtgcacgcgtgtccatttggtaggaacagagtgaaagcagcagaagcaggagcaattagggttttggttgatcttctgctcgattcatctgagaagagggtttgtgaattgatgttaattttactagatcaaatttgtcagagcgcagaagggagagctgagctattgaatcatcccggaggattagccattgtgtccaagaaaatacttagggtttctaaagtagggagtgaaagggcaatcaagatcttacattcaatctcaaagttttcatcaacaccaagtgttgttcaagagatgttgagtttgggtgtggtagcaaagctttgtttggttcttcaagttgattgtggaagtaaagctaaggaaagagctagagaaattctcaaatttcatgcaaaggcatggaggaattcttcttgtatacctatgaatttat

SEQ ID NO: 140 - DNA, 590 bp, Lycopersicon esculentum
ctctggacaagatttcatttgggttatcagaacagggaatgaagattggctcccagaaggattcgaggaaagaacaaaagaaaaaggtttaatcataagaggatgggcaccccaagtgctgattcttgatcacgaagctattggagcttttgttactcattgtggatggaactcgacactggaaggaatatcagcaggggtaccaatggtgacatggccagtatttgcggaacagtttttcaatgagaagttggtgactgaggtaatgagaagtggagctggtgttggttctaagcaatggaagagaacagctagtgaatgagtgaagagagaagcaatagcaaaggcgataaagagagtaatggcgagtgaagaaacagagggattcagaagcagagcaaaagagtacaaagaaatggcaagagaagctattgaagaaggaggatcatcttacaatggatgggctactttgatacaagacataacttcatatcgttaactagtgatgcaaaaaaaagaaaaaacatgtgtgtttctatattctggcttctgttttgctgatttgatcatattacgtacttcttcatcat

SEQ ID NO: 141 - DNA, 612 bp, Lycopersicon esculentum
aaaattgagaaccaaagttgagagatggcagaagtgaagttgcttggtctaagttatagcccttttaatcatagagttgaatgggctctaaagattaagggagtgaaatatgaatttatagaagaagatttacaaaataagagttctttacttctagaatcaaatccaattcacaagaaaattccagtgctaattcacaatggcaagcccatttgtgagtctatggtcattcttgaatacattgatgaggcatttgaaggcccttccattttgcctaaagacccttatgatcgagctttagcgcgtttttgggctaaatatgtcgatgataaggggtcagcagtgtggaaaagtttcttcttcaaaggagaggaacaagagaaagctaaagaggaagcttatgagatgttgaaaattcttgataatgagttcaaggacaagaagtactttgttggtgacaaatttggatttgctgatattgttgcaaatggtgcagcactttatttgggaattcttgaagaagtatctggagtcgttttagcgacaagtgaaaaatttccaaatttttgtgcttggagagatgaatattgcatccaaaacaaggaatatttt

SEQ ID NO: 142 - DNA, 655 bp, Lycopersicon esculentum
tggagctccatcggttaaatgtgcagtatgccattatattaccaatgttaatgcaggagatggaagagcccatgcacctttgaaccctcctaacggcactgctactactgcttcaggatcctcttcaactgccaaggctcgttcacaaaatcaaactgttgtcgtccaaaacccaatgtctgtcgatgagagtggcaagttggtgagtaatgtagttgttggtgtaacaacaacatcatagctcgtctgctaaacttgctgttcgtggacaacaattgagagttttgctcgcaacaattggaaagaggagaataaagacagccgctgcatataaatatggtagtgtttagtgttaatactacaaccatacccctgtttttcttttgttaacattttgatattatgattgtggggtgattgaagctactactacaagtgttgtgactatttggagtctgtaattgataactacgcgttagatttgtggcgtgctatgcccaaagtcataagtgaagaatgttattatgtaccttatatgccatattattgtatatgactgtataccgtgaaccttactgcctaaacaccttgcttttgagaggcgaattcaggatctagagactagagttagtgagctttgaatgagtgcggtttt

SEQ ID NO: 143 - DNA, 613 bp, Lycopersicon esculentum
caatggcaagtgagaaagtgaaattgcttggatactgggcaagcccttttgctctgaaggttcattgggcactgaaactgaaaggtattgaatatgaataccaagaagaagatctctcaaacaaaagtcctttgctcttgcagtacaatccagttcataagaagattccggttttggttcataatgggaaacctattgcagaatcactagtcatacttgaatacattgaagagacatggaagcacaatccactcctccctgaagatccttatgaaagagccaaggcgcgtttttgggcaaaatttgttgatgacaagtgtgtgccaggaatatttggtacctttgccaaggtcggagtggagcaacagaagatagcaaaagaagctcgtgagaacttgaaaattttagaggatgaactaggcaagaagcacttttttggagaggcaaaaattggcttcatggatgtcacgtctgcttggatcatctgctgggctcagattgttgaggaggttgtggatatcagacttattgatgccgaggaaatgccttcacttgtttcatggtttcagaatgtccttgaagctgctcccattctgaaagaatgtacaccgcc

SEQ ID NO: 144 - DNA, 712 bp, Lycopersicon esculentum
tctataaggacggtcaagttgaacgttatatatatcaaaaaagtatcttattaacgtacgtttccccattacttgacgatccaaaaaataatatctcctctaaagatgttataatttcaccacacgtttctgctagactttaccttcctaaaaacacaacaaattccacccaaaaacatcctattatagtgtattaccatggtggtgcactagttcttggatctgctttttttaaaacgtaccataattatctcaatgtcttggtttctaaatccaacgcaatcgcgatttcagttgagtatcgattagcccctgaacatgatgttaccacaatttacgaagattcttggactgcacttcaatgggtcgcatcacatgctaatgaaaaaaaattcactagtgataacgaagacccgtggataaaaaattacggagattttagtagattgagtatcattggagacagtgctggtgggaatatagtttatcatatggcaatgagagcaggaagagaaggtggtattaatgaaaaggtaactattaatagttcgatttttgtttgtccttactttttggttccaattgagaatattgaagaacatgcatcgtacaagaattggcttattattagctcaccatcagaagctgggctacatagtccattgatcaatccacttgctgagaatgctcctagtttgtcccaattgggttg

SEQ ID NO: 145 - DNA, 700 bp, Lycopersicon esculentum
tcttccttcagttctcagtcactgaaccctcgccggagcatcaaaatggtaacgtctgcaattgctggaacgtcaattgtaccagtctcttcccggcaccaaacgtctttttcgtctctgctttcctctagaagcttaaggaaaaatgtagtttctgttcttcgaagtccatattctgattcatcagatattggattttcaagcaagaagttggggatcccattaaagttcaatgagtacgaatccggtgctcataccaattcaagctatggtgttatcgtagcaaaagagggggctaatccgcccatcatgcccgccgtgatgacaccagtgggcgcgttggatctttctaccgtgttattcaggaatcgaattatcttcattggacaaccagtcaactctgcagttgctcagaaagtaatatcacaacttgtgacccttgcaactatagatgaaaacgcagatattttgatctatcttaactgtcctggtggaagcacatactctgtcttggcaatatatgactgcatgtcatggataaagcctaaggttggtacagtatgttttggagctgctgcaagccaaggagcacttcttcttgctggtggagaaaagggcatgaggtatgcaatgccaaatgcacgtataatgattcatcaacctcaaagtggatgtggagggcacgtggaggatgtgcggc

SEQ ID NO: 146 - DNA, 410 bp, Lycopersicon esculentum
agggtaaatacatgatcaacaatatttttcaatgatcataagagtttatacattgttgaaagaattctgtggcatgtctttacatgtatgaatgggctatatctaaaactatacaaaaaaattaactcatatacagtgagctatatcgtgttgttattaccttacaatacagtacagatgtatcagataaatggatatgctggcttcacctgttggtgactactggatttccgtttgtatcagacaattggatactctggcttgactggttaacgacgacaggatttccagattcatctaaatggatcacagcaacatcaccaggcttgaaatccccagaaagtacagattcacacaggagatcttcaaccatttgagtaacagccctcctcaaagggcgtgcgccatag

SEQ ID NO: 147 - DNA, 709 bp, Lycopersicon esculentum
gtagtggagcgtcatggacagaccctgattccatagttttgatggtcatgcaacaaatgctcggatcatggagcaaaagttcaggaggaggaaagcacatgggttcagagcttgttcagagggtggccatcaatgagctagctgaaagtgtgatggcttttaacaccaactataaagatacagggctgtttggtgtctatgctgaagctaagcctgattgcctgagtgacttggcttatgtcatcatgaatggtatatgcaagttaagttacaaagtgtctgatgctgatgtggttcgcgcacgtaatcagctgaaatcttctttgatgctccacattgatggatcaggccctactgctgaagacattggacggcagttaatcacatatggccgcagaattccatatgctgagttattttccagaattgacagtgtcgatgctggaacgatcaagcgagttgcaaatcgttttatttttgacagggatgttgcaatatcagctaggggaccaattcaagatttgcctgattacaattggttcagacgcaggacatactggctgcggtactagattgctaattctttcctgatgacttaaattcatcttcttttcccagtcggaatcgggacattgggcattataccacacatggtctacaaaacacttttgtcaataaaatgagcgacaaattttgttgatcacttttg

SEQ ID NO: 148 - DNA, 520 bp, Lycopersicon esculentum
tcgacgcgaacaccattcagcatctcataaaacccagatgtggaaacgctgatgtagttaacggtactagtactatgaactccggtaagccaccggcaggttctccgacgatgcacaccgtagctcactactccttctttccgggaagtccacggtggccggcgaacaagagagatctgacatatgcttttgcaccgcagaatggactgacggatgatattaagattgtgttcacacgagcgtttgataggtggtcggaggtgactccattgacgtttactgaaatagcatcgtaccaatcggctgatattaagatcgggtttttcagcggagatcacaacgatggagagccgtttgatggtcctatggggacattagcacacgcgttttccccaccggcggggcattttcacttggacggcgaggagaactgggtgatcgacggtgcgccgatagttgatgggaatttcttttctatattgtcggcggtggaccttgaatcggttgcggttcatgaaat

SEQ ID NO: 149 - DNA, 610 bp, Lycopersicon esculentum
ggcacgagcaaaatcgaaaatggtttcgttgaagctccagaagcggctcgccgctagtgtcctcaagtgtggaagaggcaaagtttggcttgatcctaatgaaggcaatgaaatctccatggccaattcccgtcaaaacatcaggaaattggttaaggatggtttcatcatcaggaaaccaaccaaaattcactcgagatctcgtgctcgccgaatgaaggaggccaagaggaaaggccgtcactctggatatggtaagcgcaaaggtaccagggaggctagactgcctaccaaggtgctttggatgaggagattgagagtcctcaggcgtttgcttcgcaagtatagggaatcaaagaagattgacaaacacatgtatcatgacatgtacatgaaggtgaagggtaatgttttcaagaacaagcgtgtgctgatggaaaatatccacaagacaaaggctgagaaggctagagagaaaaccttgtccgaccaatttgaggctaggagggcaaagaacaaggcaagcagagaaagaaagtttgcaaggagagaagaacgtttggctcagggaccaggaggagagaagccagtacaacctgtagcaccagcc

SEQ ID NO: 150 - DNA, 659 bp, Lycopersicon esculentum
ggcacgagcttttctgtgctttgctcaaaagacttatccggattttggggtctgctgtattgctatcgtcttcaatctttaccaaccctaaccttcggatcaggtctgagcatccaagtttcgagatctgtgtgctcctgttgtcttctactctcagccaagtttcatgtctgatctcgacgtccaaattcctaccgcttttgatccctttgctgaggcaaatgctgataactctggtgctgggtcaaaagattacgtgcacatccgtatacaacaaaggaatggtaggaaaagcctgacaactgtgcaggggttgaagaaagaattcagctataacaagatactgaaggatctcaagaaagaattctgctgcaatggtactgttgtccaggatcctgaactaggccaggttattcaacttcagggtgaccagaggaagaacgtttctacctttcttgtccaggctggaatcgtgaagaaagagcacatcaaaattcatggtttctgattgctctcatcagcctcagctgcacagatattgtacttgttatgtcagtgccagagatatgctatccaaactcaattctactaagcttgctactggttaatttcttctggtttgatataaacactatatttgcttctctatgtttggtctg

SEQ ID NO: 151 - DNA, 638 bp, Lycopersicon esculentum
caaatttgactggcctgtctaaagctgaagcctgcattcaaaagagacggagcaacaactgcagggaattctagccaggtcagtgatggtgcggctgcagtgcttctcatgaagagaagtgtagctatgcagaagggacttccaattcttggtgtattcaggagctttgctgctgttggtgtggatcctgctgttatgggaattggcccagccgttgcaatcccagctgctgttaagtctgctggtcttgagctgaacaatattgatctttttgaaataaatgaggcatttgcatcccaatatgtctattgccaaaagaaactcaatcttgaccctgaaaatgttaatgtgaatggaggggcattggctcttggtcatcctcttggtgctacaggtgcacgttgtgtggcaactctgcttcatgagatgaaacgccgtggcaaagactgtcgttttggtgtgatttccatgtgcataggttctggaatgggggctgccgcggtgtttgaacgaggggatgctgtagatgacttatgcaatgcccgggccaacaataacaacaatttcttgtctaaggatgctaaatgagcatgcttataatatccaaagtctgcttttgttttgagataggaatgtga

SEQ ID NO: 152 - DNA, 807 bp, Lycopersicon esculentum
ttcctggctagcaagctgactgaaaatgctgatgcgtctcgtttggatgatgacagtgaacttcatttgcagccagcttcgtcttctacatctgatcaacatgaactcccattagttggacctgaggatatagcagcagttgcttcactctggacaggcattccccttaagcagcttacagttgatgaaagaatgcttttggttggtcttgatgagcagcttaagaaaagggttgttggtcaggatgaggctgttacatctatttgtcgggctgttaagagatccagaactggcctcaagcacccaaatagaccaatttcggcaatgctcttctgtggtcctactggagttggaaaatctgaactggctaaagctttggcagcatcttattttggatctgaatctgccatgctaagattggatatgagtgaatacatggagcggcatactgtgagcaagttaattggatcgcctcctggttatgtaggctatggagaaggagggactctaactgaagctatccgaagaaagcccttcactgtagtgctgctagatgagattgaaaaggctcatcccgacatattcaatattctccttcagttgtttgaagatggtcacctaacagactctcagggaagaagagtgtcatttaagaacgccttgatagttatgacttcaaatgtgggttcaacagccattgtaaagggtagacagaatactatcggcttcttgcttgctgaagatgagtcagccgcctcctatgctggtatgaaagcaatagtgatggaggagctcaaaacatacttt

SEQ ID NO: 153 - DNA, 494 bp, Lycopersicon esculentum
caaattaaatcaattctttttcttgaagacaaagtaatggtggaattgcaatttcctgctggatttcgattccatccaactgatgaagagcttgtgatgcattatttatgtcgaaaatgcgacgcacagccaattgctgttccgattatagctgaaattgatctctacaagtacaatccatgggatctacctgatttggctttgtatggtgagaaagagtggtatttcttttcgccgagagatcggaagtatccgaacggttcacggccgaaccgagctgcggggagtggatactggaaggcgacgggtgcggataagccgattggtcgtccaaagtcgatgggaattaagaaagctttggtgttttacgcaggaaaagctccgaagggagaaaaaacaaattggattatgcacgaatatcgacttgctcatgttgatcgatctgctcgtaacaaaaacaacagcctaagactcgatgattgggttttatgtcg

SEQ ID NO: 154 - DNA, 596 bp, Lycopersicon esculentum
attcaatgactgtggttcctggcaagacttataggcttagaattggcagcttgactgctctttcagctctaagttttgaaattgaggggcataacatgacagttgttgaggcagacggtcactatgtggagccatttgtggtaaaaaatctgttcatttattctggagaaacatactcagtcctaattaaagctgatcaagaccctacaagaaattattgggcaagtacaaaagttgtcagcaggaataatactactccaaatggtttgggtattttcaattattacccaaaccatcctaggagatatcctcctactgtaccaccctcggggccacgttgggatgacgttgcccccaggatggctcagagtgttgctatcaagtcccacagagatttcatccatgcccctcctctgacatcggacagggtcatagtcatgctcaacacacaaaatagggtgaacgggtatgtacgttggtcagtcaacaacgtctcattcaacatgccccatacgccttacctgatcgcccttaaacacaacttgttacatgctttcgagcaaacccctcctccggacaactatgaccaaaagaat

SEQ ID NO: 155 - DNA, 718 bp, Lycopersicon esculentum
taatatcgaccaatcatggcagctcacagctcaactctcaccatatccatacttctcatgctcatcttctccaccttatcctctgcttccgatatgtcaattattagctacgacgagacacatattcaccgtcgtaccgacgatgaagtctcggcgttgtacgagtcctggctaatcgagcacggaaaatcgtacaacgccttaggagaaaaggataagagatttcagatcttcaaggataacttgagatacatagacgaacagaactctgttccgaatcagagttataagctcggattgacgaaattcgccgatctgactaatgaggagtacaggtcgatttacttgggaacgaagagttccggtgaccggaaaaagttatcgaagaacaaaagtgatcggtatcttcctaaagttggggatagcttgccggaatcaattgactggagagaaaaaggtgtgcttgttggtgtcaaggatcaaggaagctgtgggagttgttgggcattctctgctgttgctgccatggaatcaataaacgcgatagtcactgggaatttgatatcactatcagagcaagagttggtggattgtgataggtcgtacaatgaaggttgcgatggtggtcttatggactacgcctttgaattcgtcattaagaatggaggaatcgacactgaagaagactacccttacaaagaacgcaatggcgtatgtg

SEQ ID NO: 156 - DNA, 574 bp, Lycopersicon esculentum
  misc_feature (530)..(530): Unknown nucleotide 530
gtcaagagcccacaaaacagaatgatcaaaatgctaatggaagatccgaattcaacactgtacagaattttatgcagaataataatgatcagaacaatagtggaaaccaatacaatcagagtataagggagcagaaaaggtcagatgacggatacaattggaggaaatactggctgaaacaagtaaaaggtagtgaaaatccgagaagctactacaagtgtacatacccaaattgtcccaccatgaagaaggttgagagatctttagatggtcaaattactgaaattgtgtacaatggtaatcacaaccatccaaagcctcagtctaccagaagatcgtcatcatccacagcttcatctgcattccaatcttacaatacacaaactaatgaaattccagatcatcaatcctatggttcaaatggacaaatggattccgttgcaacacctgagaattcttcgatttcatttggggatgatgatcatgaacacacttgtcaaaagagtagtaggtcaagaagagatgatctngatgaagaggaaccagactcataaagatggaaaagagaaaacga

SEQ ID NO: 157 - DNA, 608 bp, Lycopersicon esculentum
ctctccataacacaaggtgcccaaacaagaaaattacacttcataaatactcacattttgtgttggttttcttcacaaaatagcaatggctctttcaactaataccactaccaactctcttctttccaacaaatctttgcttcaaaatcaaccccttttgtcttctccttcaaagaatgcatttttcttcaacaaatcaaccaaaacggttagatttgttcaacctatcgcagcggttcattcgtctgattcaaacaaaaaccccattgtttccgataagcccaccaagtcttctccaccggcggccactgcaaccaccgctccggctcccgctgtgaccaaaactgaatgggcggtggatagctggaagtctaagaaggcgctttagctaccggaatacccagatcaagaggagcttagatctgttcttaagacgatcgatgagttccctcctatcgtctttgctggtgaggctagaagccttgaagaacgacttggtgaggctgctatgggaagggcgtttttgttacaaggaggagattgtgcccagagtttcaaggaattcaatgctaataatattagggatactttcagaatccttctttaaa

SEQ ID NO: 158 - DNA, 2829 bp, Lycopersicon esculentum
gagctgaaaattgctaatggcagttggtaactgaacgtttttatacagggaaatgtctggtagacgatcgagttatactttgttgaatcaaattcctaatgataattttttccagccgccggcgccgaagttctctgccggagctggtgtagtgccgtacggtgagtctagttctgctgagaagaacagaggtaaagtatttgatttggacttgatggatcaacgcatgatgcaatcacataaccgggtcggatcatttcgggtaccgggttcgatcgggtcgcagaggcaatcaagcgagggtagcttcggtggtagctcgttatctggggagaactacgtggggacttcctttgggcataagaatgagggctgtggctcgtcggtggcaaggagctgggcgcagcagacggaagagagttatcagctgcagttagctttggcaataaggctctcttcagaagcaacctgtgctgatagtccaaacttcttggatcctgtgactgatgtattggcatcccgagattcagattctactgcatcagcggtaacaatgtcacatcgattgtggataaatggatgtatgtcatactttgacaaagtccctgatggattttattggatttatgggatggatccatatgtttgggctctctgctcagttgtgcaagaaagtggccgtattccatcaattgaatcattgagggcggttgatccctctaaagcaccatctgttgaagtgattttaattgatcgctgtaatgatctcagcttgaaggaactgcagaatagaattcatagcatatcccctagttgcatcaccacaaaagaagctgttgatcagcttgccaagctggtttgcgatcatatggggggtgcagctcctgctggagaagaggaactggtttccatgtcaaaggggtgcagtaatgacctgaaggatcgttttggaactatcgtgcttcccattggtagcctgtctgttgggctttgcaggcatcgtgctttgctttttaaagtgctagctgacatcattgatttaccatgtcgaattgccaagggatgtaaatattgtaatagctctgatgcttcctcatgtctagttcgatttgaacatgacagggagtacttggttgatttgatcggtaagcctggagttttaagcgaaccagattccttgttgaatggtccatcttccatctcaatcccttcacctttgcgctttccgagatacagacaagttgagcctacaactgatttcaggtcattggccaaacagtatttcttggatagtcaatcacttaatctactgtttgatgattcttcagctggagctgcagctgatggagatgcaggacaatcagacagaagttgcattgatagaaacaatgtagtctctagttcaagtaatcgtgatgaaatttctcagttacctctgcctccattaaatgcatggaaaaagggacgagataaagaatctcaactttctaaaatgtataatcctcgaagtatgttaaacccagtgaacatggacgaggaccaggttcttgtgaagcatgttcctccattccgggaagatgctcaatcaccgatgacacgaccagatacagtaaatgatactaggtttcttgctggaggaggtcatgttgtttctgctataccaagtgaagaacttgatctcgatgtagaagagttcaatattccatggaatgatctgattctaatggagaaaattggggcagggtcttttggtactgttcaccgtggtgattggcatggctctgatgttgccgtgaagatcctcatggaacaagattttcatgcagagcgactcaaggaatttttgagggaggttgcaattatgaagcggttgcgacatccaaatattgtactttttatgggtgctgtcattcagccaccaaatttgtccatagtcacggaatatttatcgagaggtagcttatatagacttcttcataaacctggtgcgagagaggtgttggatgaaaggcgtcgcctgtgtatggcttacgatgtggcaaatgggatgaattatcttcacaaacgcaatcctcccattgtgcaccgagatttaaaatctccaaatcttctagtagacaaaaaatatacagtgaagatctgtgattttggtctttctcgtttcaaagcgaatacattcctttcatcaaagactgctgccggaactccggaatggatggcgcctgaagttattcgtgatgaaccatcaaatgagaaatctgatgtatacagctttggtgtcattttgtgggagctggcaactcttcaacaaccatggaataaattgaacccaccacaggttatagcagctgtcggctttaacaggaagaggcttgatattccaagtgacttgaatcctcaagtggcgattattattgaggcttgctgggctaatgagccgtggaaacgcccctccttttccactatcatggatatgctgagacctcatcttaaatctcctctacctccaccaggtcacacagacatgcagttgctctcatgaatacctgtgctctctgcacatattctgccgtcattgcaagccatgtttctgggaggaagaacatttggattatgtcaggcttgcatgatgtacctgttcatattaattcagattggctttggagaaaaccaggttttcttcgcaagctcaagcagtggccactgcctcacagaagatccttcgcactgggcgtgtccatttcctctttgaaccattgtttgagcaactgattttgttcaattccaattttttgtaatttctgcccaggattcttgtataactgtatg

SEQ ID NO: 159 - DNA, 552 bp, Lycopersicon esculentum
ggtaccctgctccacctccttaagctccttttggagatccgaacaaaccacccaacgatagccactcacaaccgactgctccttatggtggataccacgcgccacctcctgctgggccttatggagctagtagcccttttgcatctcttgtgccgtctgcttttccaccgggtaccgatccgaatattgtagcttgtttccagctcgcggatcaggatgggagtgggttaattgatgataaggagctgcagaaggcgctttcttcgtataatcagagcttcagcttgaggactgttcatctgctgaggtatctcttcaccaataccaacactagaaaaatgggtcccaaagaattcacttcagtgttctacagtctccaagaatggaggggaaggttcgagagatttgatagggataggagtggaaaaatagattcatctgaattgagggacgcgttacttagcctgggatatgctgtatcaccttctatcctggaattgctggtctccaagtttgataaaactggtggaaagaatagagccattgaatatg

SEQ ID NO: 160 - DNA, 518 bp, Lycopersicon esculentum
gggatgtgttgtataattttgaacaaaggtggaggaagcagattggaaatagatttatctactcaataaatgagcttgataagtttattattcgtccaacggaagttacagcatcaagggacagagaaacctggaatgttcagatatttcggtctattgatggtggtgctgttaccgacttccctgtaaaacctgacgaagcttctgaggtaggccttgttactggaaaaaacaacgtcattgatcaaagtatccacgatgcatacattagtgccattcgtcgagctaagaatttcatttacattgaaaaccaatactttattggaagttgctatggttggaagcctacaactgatatcaagctcgaggatatcggagctttgcatcttatcccaaaggagatctcactcaagattgtgagcaagattcaagcaggggagagatttacggtctacgttgttcttccaatgtggcctgaaggtataccggaaagtgattctgttcaggcaattctagat

SEQ ID NO: 161 - DNA, 568 bp, Lycopersicon esculentum
gtggtaaagaggttgaaagatgtaggtgctgggaagaaggaatttgagcagcaaatggaggttgtgggaagcataaaacatgaaaatgttgtcgagcttcgagcatattactattcaaaagacgaaaagcttactgtctctgactatttcagtgagggtagtgttgctgcaatgctccatggtaaacggggagaaaacaggatccctttagactgggaaactcgacttcggatagcaattggagcagctagaggtattgctaggatacatactgagaatggtggcaagttagtccacggaaacgtcaaatcctcaaatatctttctgaactctaaacaatacggttgtgtatctgatgttggtctgtcaacaataatgagctcgttagctcatccagttgcacgtgctgctggttttcgcgctccagaagtaacagacacacgtaaagccactcaaccttctgacgtgtacagctttggtgtgttgctattagagctcttgacaggcaagtcccccatccatacaacaaacggagatgaggtcatacacttggtccgatgggttcatt

SEQ ID NO: 162 - DNA, 520 bp, Lycopersicon esculentum
agcagttgtcgaattcgtacaagttaaagtatctcaaatccaatttgtgattctgggtgggggtcaattcatcatatggctgccaatttctggatttcttctcagtacaaagaacttttggaccaagaagaggttgatgtggtgcatcagttggataaagagagaggcatcactctcgatgatttcaagctcatcaagttgcatatgtccaattatgttgcaagattggctcaaaatgtgaaagtgagacaaagggttgttgctaccgcaattacatacatgagacgcgtatatgtcaggagaagtatgaccgagtatgatcctcgtctggttgctccaacttgcttgtatttggcatcaaaagcagaagaaagcactgtgcaagcccgacttcttgtattttacatcaagaaattatattcggatgagaagtataaatatgaaataaaagacatactatacatggagatgaaagttctggaagccctaaactattacttagtcgtatatcatccatatc

SEQ ID NO: 163 - DNA, 544 bp, Lycopersicon esculentum
tgtacttattgacatgagcatacacacagcatatgtgaaggctatccgtgctgctcaacacttcgtttacatcgaaaatcaatatttcattggttcttcatacaactggagtcaacataaagatgttggtgcaaataatctgattcccatggaaattgcacttaaaatagctgaaaagataagagcgcatgagaggtttgcagcatacattgtcctcccaatgtggccagagggtaatccaactggtgctgctacacagagaatactatattggcagaacaagacaatgcaaatgatgtatgagactatttacaaggctttagaggaggttgggcttgagaattcatgctctccggaagattatttgaacttctactgtcttggtaaccgcgaggctggtaaagttgagggaaatgagagtcctagcgcagcaaacactcctcaggcgttcagccaaaaatcccgtagattcatgatttatgttcattcaaaaggcatgattgtggatgatgagtatgtgatattgggatctgcaaacatcaac

SEQ ID NO: 164 - DNA, 630 bp, Lycopersicon esculentum
gttgattggatattgttgggaggaggagcagagacttctcgtttacgagtatatggcaaggggaaacctcgaggatcaactattttcgagatattcgagttgtttgccatggttgaccagaataaaaattatggttggtgctgcaaagggactggctttccttcacggagaagaaaaaccagtaatctaccgcgattttaaggcttctaacatcctcttagactcggattacagagccaaactatctgattttgggctagcaaaggatggaccggaaggcgatgacacacatgtctcaactcgtgtgatgggcactcatggttatgctgctccggagtacatcatgactggtcatttgacaagcaagagcgatgtgtacagcttcggagtagttctgttagaacttataacaggacgacgagccatggacaagaaacgccccctcaaagagcgaatcttggtggattgggcaagaccaatgctaagggatccacacaagcttgacagaataatggacccacggctcgaaggtcagtactcgacacaaggagccaagaaggtagctgcattggcttatcaatgcttaagccaccaccccaggtctaggcctactatgagcaacatagtgga

SEQ ID NO: 165 - DNA, 588 bp, Lycopersicon esculentum
gcacgagggcaggtgaagatgaaggaagacaatcagcagatccctctcggaggagaagtcctgtgccaccactgattacttctggaagtttatccaggcagaaaagtcctattggaaatgattccacgagcaaagaggctatgttgtcaagctccagttttctgggaagacctagtgggtctctgaggcaaggtctagtctctggcagccgtgagactttcaccatgggaaatgactctgaccctacacattctcgcacgcctgaggcaagtccagcaactatgcacaaaatatcaggtggacatagaagctctctacttggtggatcatctgatcctaggtatgcctcatctagcaagaatacttctgggataaagaactatgaatcggcgctcaaaggaattgagactctacattttgatgatgaagagagggctcattgattgcaaatcctttcacatgtaaatttttacttgtttggtagacttcagatatagattgaggccctgaaagcttatttttgtttgaattcgtgggttggaaactagtgaatgaacagttgttattcactctgcccaaagatctaca

SEQ ID NO: 166 - DNA, 540 bp, Lycopersicon esculentum
aattgtaaaataaaaaatttaatttattaagtacaaataatacatatcaaaccaatatgtaaaaagaattgtagactaaaattcataaactgaaaattctagttcttgcctctgatcagacatcagacaacaaagaaaccatgtatttcctcgtgaggaaatgttccatttccaactcataaagtatgtgctaaatgttacaattgaaatacatctttctatgcttttcttacactattcaagcaagtactatacaagtaaaaatgcactagtacacatatccaatgcttaaacaacatggatctgaatgaaatgttaattgcgcgtgggtgcaatattttggtttactaccttccagacatgaggtttatcatgccttgatttccatagaaagatccaacatcccaatcttctgttgacgaagagagggtcatcaacgcaaccactattgatctcatacttggtcttattaggggattttcatgggtgcaagctttggcaagttgtgccatcttccggactgaatcaaggtgatagtca

SEQ ID NO: 167 - DNA, 336 bp, Lycopersicon esculentum
  misc_feature (306)..(329): Unknown nucleotides 306 and 329
atttacagatctggattgcctcaaccgtctaatttcccctttcttaaatcccttaatcttcgatccatcatatatttgtgccctgagccttatcctgaagagaatttggagtttctgacaattaacagtatcaagcttttccagtttggtattgatggcactaaggagccatcagctatgtctagcagtgctataacagaggctctgaaagtgattactgatgtcaggaatcaccccgttctcatccattgcaagcgcggaaagcaccgtactggttgcctcgttggttgcctgaggaaattgcangaactggtgtatgttctgctgtnggtggag

SEQ ID NO: 168 - DNA, 568 bp, Lycopersicon esculentum
cagcccactttacatgtcgccggaaatagtcacagccggtgaacaggacactcctgccgatatctgggcacttgggtgtgtaatttctgagatggctaccggagttccagtctggggatactcagagttagcccaactgttgatgaatattggagttggtaatgagttgcctgaatttccgacgatgttatcggaagaaggtaaagattttctggaaaaatgcttggtgaaggactcgaaaaagagatggacggctgagatgcttctattacacccgtttgttgctgatgaagatgacactgttttattgaatgacgaaagatgcaacagtggcagcccttcaacatctcctaggtgcccatttgatttcccagattgggtatctgatcactcttctgaatcatcagtaacatgtccaattacatctttgccctcgccggcaattcaagaattgcggaatttgaacggcgggtcatggtccacaacgccggctgaaaggctgcgggcactgttaagtggtgcaatcagacctgaatctgactggtcggctgctgatggttgggtcaac

SEQ ID NO: 169 - DNA, 432 bp, Lycopersicon esculentum
ggaccgaattacacaaaatatcttatactacttccgttttataatcttcgcaatgcagtcaccgtcttcacattaaaatgattgataacgttaaaggtatgccgccggcaaccgagaaaggttgccggttaacggcgttgatagattccggtggactagcagaagtagatctgagtgagaacgagcaaaattctactcgacgtaggcgattggatgaacgtctgttgaaatcgacgactgagctaccggaaaatttctatgtcttcgcagatgattacaggcattgtaagaggaaaaaaagtactgatactgatactgatgatcatcgagttcaactatcgctatctagtgaagtgaaaaaagtaatggagagcttggtgacgtgctgttcacatgggtcgatatagatgatcggccggagaacggaaatgg

SEQ ID NO: 170 - DNA, 532 bp, Lycopersicon esculentum
tttaagcttgtctaatgctcacacctcttctgcagtagaactagattgtactcttttaaacaaaaatgaaacaaaaatacatgtattcccgcccaaaaattagagtttgttacactcatatttcctatacttagtaagcttgcaaaaagaatgaagatcagcatcctgcggtcaactgtgtagaaccaactgcattcctgtgctgaagatgcaaagcagccaaaaggccttgccatgtttctccaggtcctccagccacttgaaaatctaggagcttcctctgtttcctgtagtattcttctagcgggttgacctgctctgcatgcacatgcaggttcttcctcgagacatcttccatgatacttgaaggcctaaaatgaccaccctctggctgccgactggtactaattccagaagcccccatgcggaggaattccagtggtgcaaaagaggatctcgaaaacatcgaatcctatgtgaacaaattgaaaacaatagccaattctctagcagaaatcaacagtcccatctc

SEQ ID NO: 171 - DNA, 442 bp, Lycopersicon esculentum
ttttctggctgcaggtggtattatgctattcgtgatgatcgccggttacttcccgttctacgatcagaatctgatgttaatgtacaaaaaatctacaaaggcgaattccggtgcccgaaatggatctctccggacggtaagcggattctatctcgtcttctggatgtaaatccggcgaccagaattacgatcgaagaaatcatcagagatccatggttcagaaagggattgaaatttatcaaattctcagaagaggaagaaaatagtaagataaattcgttaacaaattctctgaatgcgtttgatataatttcgttctcccagggattagacatttctggattgttcaaatcgaacaatccggtggatgatttggagaggatagtggtggaagagttcgcggagagtgtaattgagagaattgaagaggttgggaagaagg

SEQ ID NO: 172 - DNA, 571 bp, Lycopersicon esculentum
aagaaattaacacactccacaaccacaagagagactatcacctgagttcttatttattattattatttagaagcaacaacacaaactacttctcccaaaaatattattacaacagaaataatgggaaattgttggccaaaacctgtggatgttgttcctcccacctctaatactcctcctccagtgatcatgaagaaaccaattaatacatctccgggaagtaacacaaggagtgtggtggcgcagcctagcggagatggcggtgattcgaggaaaatggaagttccggctagcggaaaaataattacgccaaatttaaaaatgtttacgctagcggaattaaagagcgcgacgcgtaattttcggcccgatacggtgctaggtgagggaggatttggtacagtatttaaaggatgggtcgatgacaaaacatttgcaccttctagagttggagttggtatgcctgttgctgttaaaaaatcaaatgcagacagtgaacaaggtcttaaagaatggcagatccatttcggtgttgcagccatgcctgtggcagcttcagctgtttactttc

SEQ ID NO: 173 - DNA, 598 bp, Lycopersicon esculentum
accatttcaagacgaaaacatcatggcgatgtataagaagatttaccggggtgatttcaaatgtccaccttggttttcatctgaagctaggaggttgatcacgaagatgttggatccgaatcctaattcaagaatcactacttcaaagatcatggattcgtcatggttcaaaaaatcgatcccaaagacgttaaggaacaaagatgaggaagaatttgcatttgcatttgcatcagataagtctagtaaacaagttgagacaatgaacgcgtttcatatcatttctttatctgaagggtttgatttgtcaccattgttcgaagagaataaaaggaatgagaaagaacagatgagattcgcgactacaatgtcagcaagtagtgtgatatctaagcttgaggaggtagcaaagacaacgaatttcatcgtaaaaaagagtgattcctgtgtaaaactacaaggacaagtggtggggagaaaagggaaattaggaattgctgctgatatatttgctgtgacaaattcatttctagttgtggaagtgaataaagcaagtggtgatacattggagtataatcaattctgcagcaaagagcta

SEQ ID NO: 174 - DNA, 623 bp, Lycopersicon esculentum
gcaccagggaagttatgaggagcaaggagcagccacaagtcctgcctccaatcagtgaaccaggtcaaacatctgaatcaggctccgatagtgtaaaaatacctactgatggatctgatgactgggaaattgatatccgcctgctgaaatttgaaaataaagtagcatctgggtcatttggagatctgtataaaggtacatactgtagccaggaagtggctataaaagttctcaagcctgaaaatttgaatatggatatggtaaaagagttctctcaagaggtctttatcatgaggaaaattcggcacaaaaatgttgttcaatttataggggcatgcacacgacctcccaacctgtgcatcgtgacagagttcatgaccagaggaagcatctatacttttttgcacaagcaaagaggtgcttttaagcttcctactctcctgaaggttgcaattgatgtttctaagggaatgagttatctgcaccaaaataacataattcatcgtgatctgaagactgccaatcttctgatggatgaacatggagttgttaaagttggtgactttggggttgctagagtgcagactcaaactggggtgatgacagctgaaactggaacgtat

SEQ ID NO: 175 - DNA, 379 bp, Lycopersicon esculentum
tttttttttttttttttttagatacgtttgattgatcataatcacatagagcaactgtaacacaaatttgctcgcaactcttttcaaagtagactgctaaatacaaactgaagtgagatataaacttacgggaaaaaaaaatactgccctttcaagtcctggcaatccttttggagccttgcaacttgtccatacatattcaaacgtgtccccggaagctttctggtcaatttactactaacaaacatgtacaaaaacatatttccttttcactagttggctactaagatgggaagactttgaagagtggaacaaaaaagataatacaaagtacagaactaaaatgcactgcagccccattacattgtccttttcgacc

SEQ ID NO: 176 - DNA, 427 bp, Lycopersicon esculentum
  mRNA (1)..(427)
atactatttatccagtggttatatggctccggagtatgcaatagacggactattctcaattaaatcagatgtatttagtttcggaatagttatactcgagatcataagtggaagaagaaatactggattttttcatcaagaagaagcgtcgaatctcttaggccttgcatggagactttggacagaagataagtcaatggacttagtagatcaaaaactacatgaatcatgcaacaaagaagaagcaattaaattgataaatattgggcttttatgtgtacaagaagatccaaaagataggcccaatacatcaaatataattatgatgcttggtagtgaaaattccaatattatttcacttccaagaccaaatcaaccagcttttatgacaagaaaatgtggtaataataataacacaacttcttct

SEQ ID NO: 177 - DNA, 617 bp, Lycopersicon esculentum
  mRNA (1)..(617)
gttcaccgtgatgtatcattgaataacattttgcttgaatcagaattcgggccacgactctctgactttggcacagcaaagttgttagcttccgactcatcaaattggaccacagttgctggttcttatggctatatggcaccagagcttgcacttaccatgcgtgttacagaaaagtgtgatgtctatagttttggagtcgtagcgatggagactatgatgggaaggcatccaggggagcttttaacttcattatcagcatcaacaacattgtctccggaaatacttttgaaggatgttcttgaccaaagacttcctcctcccaccggccacttggcagaggcagtggtttttgtgatcacgattgccttggcatgtacacgtaccacgcctgaatcgcgaccaaccatgcgttctgtagcacaagaattgtctgttcagactttgccttaccttccacagccactggggacaatagaaatgagcaaactaacaagttttcagaaataatgtacaggttgtagagttttagtgttgtaatggatctattcaaataagtatgttttagatgcacttttccctagaaagaaagaaataatatcatcttgttaatcatg

SEQ ID NO: 178 - DNA, 505 bp, Lycopersicon esculentum
tacaattacaggcatgtcgtgattgtataagtaatgcaactcgtaagattctagaggtatgtccttacaagagatgggccttaggttattatgatcactgtatgctacgatattcaaatgagtccattataggcaaccttgcaactcaaccagaaagaatcttatttaacatagcaaatgcctcgagtccggatgagtttatgcaagatctaagaaccatgctagaaaacttacgaagtgaagcttcacagggcggtatgcagaagtatgctactaatagtactcagggccctgattttcagactatatatgcacttgtccagtgcactgcggatttaacagctcaagattgctttcaattgtttagacagtggctttagaagtttacctagctgctcctgttataggaagcagggtctcaactttatgatgcccagctgcaattttcggtatgaaatttactcctttttcgaccagcgaccaacccaagctccaccacccttat

SEQ ID NO: 179 - DNA, 651 bp, Lycopersicon esculentum
tttatattagtcatcatgaatccagaatacgactatctgttcaagcttttgcttattggagattctggtgttggtaaatcgtgtcttctcttgagatttgctgatgattcatatcttgagagttacatcagcaccatcggtgtggactttaaaatccgcacagttgatcaggatgggaaaaccattaaacttcaaatttgggatactgctggtcaagaacgttttaggacaattaccagcagttactatcgtggtgcacatggcataattgtggtttatgatgtaaccgatcaagagagcttcaataatgtcaagcaatggttgagtgagatagatcgatatgcaagtgataatgtgaacaaacttcttgttggaaacaagtgcgatctcacagctcagaaggtggtttccacagagactgctcaggcttttgctgatgagatcggcatccctttcatggaaactagtgcaaaaaatgccaccaacgtagagcaggctttcatggctatggctgcttcaatcaagaacagaatggcaagccaaccggcatcaaacaatgcacgacctccaaccgtgcagatccgaagacaacctgtcaaccaaaagagcggttgctgctcatcttgaaaggaccaaactcgatgatgattc

SEQ ID NO: 180 - DNA, 552 bp, Lycopersicon esculentum
tgcggcatgaatggagaccaatttcagaaaccggagatgattcagagattgaaaccctagtttcttcatcaatgttccaatcgttaatttctgtttgattttaatcttcggcgtgcaaaaactccgattctgttggtcaatttcatagcaattggattttacctcgattcgatctgaggacttttgggtctttttttgcaaatcgaagcggtgaggaatatttaggcaggtgattgtagaggataattttgtttgaaaaaatgccgtcgcacgcggatgtagatcggcagatcgagcaattgatggagtgcaagccgttatcggaggcggaggtgaagacgttgtgtgaacaagcaagggcgatactagtggaggaatggaatgtgcaaccggtgaaatgtccggtgacggtgtgcggtgatatccatggacagttttacgatctgattgagctttttcgaatcggcggcaacgctcctgatactaattatctcttcatgggagattatgttgaccgtggatactattctgtcgagactgtctcactattgg

SEQ ID NO: 181 - DNA, 554 bp, Lycopersicon esculentum
gagaagagagtgaagcattaatctcgaagaaatttcacccgctaatactaactgtggtagagcaactaaaccacttatcattcacagaattcgtcgaaacaggtaatttagatttcatcaattgtgcatcatcattttctgattcagggtcctgtgggacttctacattatcaacgacgttaatctcttctttcttctggttttcaaactcgccattctgaattgcctttagaatttccaacacttcatccatcgttggtctcatttccttctcgagttggatacattgaaaagctagctctgcaactgaagtagtcattctcctaacttcagcattggactcataaccaagagatggatcgatcaactcctcgaatgcacatttcaaaatcttgcttactgcaaagttagccaaactaatttcatccctatcccttcttatatctacagcaaggagcgacgatatgagctcaataaggacaactccaaagctatacacatcacttttacttgttagctgatagcaagcatgataatccggatcaacatatcct

SEQ ID NO: 182 - DNA, 498 bp, Lycopersicon esculentum
gcacgagctatcatcaacaattagcatattgtgttgtacagttcatagaaaaggaacaaaatctggctagcatagtgataacgagattgttgagatattggccagtgacaagtagccagaaacaattgatgttcatcagtgagttggaagagattttagaaatgatcagtattccagagtttgagaagatcatggtgccactgtttcgtcgcataggctattgccttaacagctcccatttccaggtggctgaaagagcacatttcttgtggaacaacgataacgtccttaacctcgtgatgcataacaggcatgtaatcatgccaattattatctcagctttagaatggaacagccagaaccactggaataaagcagttcttaatctcagtcaaaacgtgaggaaagtcttctccgagatggatgaagagcttgtacttgagtgccaacacaagcttgaagaagaaaatgcaaagtcaaatgtagcagcggagagac

SEQ ID NO: 183 - DNA, 618 bp, Lycopersicon esculentum
ggagttgctgctttcttcttcaacttccaagaggaatttattgcagaaagcagcagcaatggctttagatgctgtataaagtgctttaactaaacatgaacttgaacaccctttgccgaaaacagccgacccacgagtccagatttctgggaattttgctccggtaccggaaaatccagtctgtcaatctcttccggtcaccggaaaaatacccaaatgtgttcaaggcgtttacgttcgaaacggagctaaccctctttttgaaccaaccgccggacaccatttcttcgacggcgacggtatggttcacgccgttcaattcaaaaatgggtcggctagttacgcttgccgtttcactgaaacagagaggcttgttcaagaaaaagctttgggtcgccctgttttccctaaagccattggtgaattacatggtcactctggaattgcaaggcttatgctgttttacgctcgtgggctcttcggacttgttgatcacagtaaaggaactggtgttgcaaacgccggtttagtctatttcaataaccgattacttgctatgtctgaagatgatttgccttaccatgtaaaggtaacacccaccggcgatcttaaaacaga

SEQ ID NO: 184 - DNA, 332 bp, Lycopersicon esculentum
cacataatcaaccatcacacactgatgcaaccaacgccgccggcgacaaggctaagaaaattgttagctattggggggtggatcctcccaaaatctccaaggaagatggtacaccatggaagtggaacagcttcaggccatgggagacgtactcagctgatatttcaattgatgtggagaagcaccatatgcctaccaattttatggacaagtttgcttattggacagtccaatctttaaagtaccccacctacttgtttttccagagacgtcacatgtgtcatgctatgttactagaaacggttgcagccgtgccaggcatggtaggtggt

SEQ ID NO: 185 - DNA, 512 bp, Lycopersicon esculentum
ggagtatccaacattagacataaatgttgtctccggaaaagatctcaacaaggtcaatctcatcacaaaaatggatgtatacgtcgtcgtttcaatctccggcgccgatgacgacagatctaatcagaaaaccaaaacccacgttgatcatgacggtgacaataaccctacctggaatttccccatcaaattcaccatagatgatagccctgctgttcaaaaccgtttgcatcttgttttcaagcttcgatgtcaaagggcgcttggagataaagatattggacaagttgaggtacccatcaaagagcttctcgaatcttccaataccactaaacaatttgtaagttatcaaatcagaaaaccttccggtaaacccaaaggtcaactcactttttcctatcaatttagtgataagatcaccggtaatactgtcgttgattctaaaattgaaaaccccgttactgcttatccggcggcaaatccaaccccgatggtgggatccacttctgtgt

SEQ ID NO: 186 - DNA, 547 bp, Lycopersicon esculentum
cttcaatcgtttctattgacgtattttaacagatcgaaagatgtttggaagagcaccgaagaatagcgataatacaaagtattatgagatcttaggagttcctaaggctgcttctcaggaagatctcaaaaaggcttatcgtaaagctgctatcaaaaatcaccctgataagggaggcgatcctgagaagtttaaagagcttgctcaagcttatgaggtcttgagtgacccggagaagcgtgagatatatgatcagtatggagaggatgctctagaggatgttatccacccgctcaaggtttctttagaggatctttacaatgggacatcaaagaagctttcactatctcgcaatgtgctgagctcaaagtgcatgggtgaagggtccaagccaggtgcttcaatgaaatgttctggctgtcaagggtctggaatgaaagtttctatcagacagctcggtccatccatgatccagcagatgcagcacccttgcaatgagtgtaagggtactggtgagatgatcagcgacaaagataggcgccctcag

SEQ ID NO: 187 - DNA, 691 bp, Lycopersicon esculentum
ggtcatttggtgcaacggctaattacacccaccgcttttccacgaaatcccacgggcgtatcggagccagaatcggaagtactgcacttgaactagaattgggaggtggaagaaaagtatctgaattcagtaccattcggatgctgtactctataggaattcaggggatcttttggaagtttgagttacatcgtggggggcaaaagttgattgttcctgtattgctttctaggcatttgaaccctatttttgccaccggagcatttctggtaccgacctcactgtattttgtactgaagagatttgttgtgaaacctttttatcttcagcgcgaaaagcagaaggcatcagaaaatatggataaaactcttggccaggttcgagaagcaaggacggctgctaagaaagctcaacaactattgcaaaatgtggccaataggaaaagaagtagacagcttgaagcaggtggactgggttgttacaaaagcactgtatggcagtcataaagctctgaagaacagaaatcaaatagaggaagtgaaagatgaagtggcttctcaaatcattgatgtcactctacctttaaattttcttgtgagcgactcagggcaactaaagctgcacgagggtgtaaagaaatcaggaaattatgggattttgtgatcccttgtcctggagaacccaagcaatt

SEQ ID NO: 188 - DNA, 514 bp, Lycopersicon esculentum
gcacgaggtgaggattttgataacagaatggtgaaccattttgtccaggaattcaagagaaagcacaagaaggatatcactggtaacccaagagctctaagaagattaaggacagcttgtgagagggctaagagaactctctcttgcactgctcaaacaacaattgaaattgattccctgtatgagggagttgatttctattctaccattacccgtgccagattcgaggagttgaacatggatctcttcaggaagagtatggaaccagttgagaaatgtttaagggatgccaagatggacaagagcaccgctcatgatgttgttcttgtcggtggatccactagaattcccaaggtgcagcagttgctgcaagacttcttcaacggcaaggaactctgcaagagcatcaacccagatgaggctgttgcctatggtgctgctgtgcaagctgccattctcagtggagaaggtaatgagaaggtccaagacttgctgcttttggacgtgacaccac

SEQ ID NO: 189 - DNA, 569 bp, Lycopersicon esculentum
tgcaagccaaaacgcaaatctcacacaattttgcataatccaaaaaaaattaaccaaaaagaaggcaatggaaacggttatggaagacgaaaagtacaattggagcgaagtgaagcttcattctctcatacccatagtgccggagccggagctcgatagggaaaccggcgaacggcgtcgcggccgtgatatagtgatcgctatagatcacggtcctaatagcaaacacgcttttgattgggccgttactcacctttgccgtctcgccgatacgcttcatctcgttcacgccgtctccagtctgaaaaatgagatagtttatgaagctacgcaaggactcatggagaagcttgctgttgaggctttttcggttgctatggtgaagacagtggctaggattgtggaaggagatgctggaaaggtaatttgcaaggaagcagaaaggttgaagccttcagctgtggttatgggaaccaggggacgtggcataattcaaagtgtactgcaaggaagtgtcgctgagtattgcttacacaactgtaagatagcaccggtcatcatagttcctg

SEQ ID NO: 190 - DNA, 536 bp, Lycopersicon esculentum
gcaccaggagatattcgtttagcacaaaacaggcatactattcaattccctttcgttccagaagcatggatctaattgacaaggcgaagaattttgtgtcggagaagatagccaacatggagaaaccggaggcaaccatcaccgacgtcgatcttaaggggatcggtttcgacggccttgcttttcacgctaaagtctccgttaagaacccttactctgttcctattccaatcatggagatcgattacgtcctcaaaagcgccaccagggtaatcgcatcaggaagaattccagacccagggagcataaaggcaaatgactcaaccatgttagatgtgccagtgaaggttcctcacagtgtgctagtgagtttggttagggacattggaggagattgggacgtcgattataccctggaattgggtctcattattgatattccggtcattggcaacatcaccattcccctctcttatagcggggagtataagcttcctacattgtcagatttatgggaaggtggaaaagaagaagag

SEQ ID NO: 191 - DNA, 486 bp, Lycopersicon esculentum
gcaccaggataactctgaacgaacaatttgaattaaattgatatcgaaatggctcttgaagctttgaattctccaactggtactacttcaaatccgcaaacgtttcaatttgagagcaaaggccagcagcagcttcggtaccttgagaattggactaaagggaagagatctaagaggtcacggagtatggatcgtcaaccgactgaagaggaatatttggcgctttgtttgattatgcttgcaaggagcgatggctctgttaatcacgtacggtctctaccaccgccggtgccagtgatgaaaattcacgagacggcggagaagatgttgtataggtgttcggtttgtggtaagggattcggatcttatcaagctttaggaggacataaagctagtcaccggaaactcatcgccggcggagatgatcagtcgactacctctactactacgaacgctaacggaactacaagctccggtaacggtaac

SEQ ID NO: 192 - DNA, 627 bp, Lycopersicon esculentum
  misc_feature (624)..(624): Unknown nucleotide 624
cagagcaattattcaggtgtagcgagtgtcctaaagtttttacttcttatcaagcacttggtgggcataaagcaagtcaccgtaaaattaacgttcccgccaccggagacgatgatagcaatccgtcgacgtctacttccactagcggcggcggcgttaatatctctgctctgaatccgagtggccgttctcacgtgtgctctatttgtcaaaaggcttttccaactggacaagctttgggtggacacaaacgccgccactacgaaggaaaactcggcggtaacaatcgttatatctgcggtggcggagaaggcggaggcgttcatagtggaagcgttgtgactacatcagacggcggaagcggcaacggcggcggcggcgcgtcgactccgattgcgcgtgactttgacctaaacatgcctgcctcgccgggatggcaattggatctgacaatcgactgcggaggtagaactcaacatccgattgaacaagaagtggaaagtccaatgcctgctaagaagccgcgtttatttttcgagtgaaaatttgattgaattctgttttaagtcaaattagttaattggttttacatgtgatgatattattttaatatatacattttanggg

SEQ ID NO: 193 - DNA, 437 bp, Lycopersicon esculentum
cttagacattatagaggcgttagacagcgtccgtgggggaaatttgcggcggagattagagatccggcgaagaacggagctagggtttggcttggaacgtacgaaacagctgaagaagctgcaattgcttatgataaagctgcttatagaatgagaggatcaaaagcacatttgaatttcccgcaccggatcggtttgaatgaaccggaaccggttcgagttacggcgaaaaggcgagcatcgccggaaccggcaagctcgtcgggaaacggttccatgaaacggagaagaaaagccgttcagaaatgtgatggagaaatggcgagtagatcaagtgtcatgcaagttggatgtcaaattgaacaattgacaggtgtccatcaactattggtcatttaaaaagccgaatatttctccgaacgcaaaatactatatga

SEQ ID NO: 194 - DNA, 629 bp, Lycopersicon esculentum
caaaatcaaacgaaaaatttcaaacatgcattggttaaataaaagatttagacaagaagcaggaatgaattcgaattcgaattccctccaaaataacaatcaatttcaacagcagcaaccaaggcttactggagatgaagagtactctgttatggttgcaactctgaaaaatgtgatcaatggtaatattccaacgcaaaattatcaagaattcaatgtcttttcgccatataattattctactgccaccacaaccacgaatgttacttcttcttcttcgccttctactagtatgtctactagtttcgaacaagtattgggtgtctctgctgaacaagaaccttgtcaattttgcagaattcaaggttgtttaggctgtgacatttttggtaccacattgtcttcttcttcttcagcgcctgctgctgtggctgctcctgttgctgataataagaagaagagtagtagtagtagtacagctacagtcgcaatcgcgaagaaaaagaagaagaattacagaggagtgagacagaggccatgggggaaatgggcagcagaaattcgtgatcctcgaaaagctgcacgtgtttggctgggaactttcactacggcagaagaagcagctagag

SEQ ID NO: 195 - DNA, 608 bp, Lycopersicon esculentum
  misc_feature (577)..(577): Unknown nucleotide 577
acaacaaattacaaaacaaaatatactaaaatctgtatttttagcttcttttcaccatctaatctcacaaaaatatcatctttccctgttttatcgagtaaataaagattagttccaactggattttgttcccttttccccttctctccataacgaacctaatttgcagttctgtatttgggggttttcttcttttggaagaacagaaaaaaaggtgtcttttgttttacttttggagttattaagatatggcaatgaccaaaaccttatctatgatctcaagaacaggaagagacttgcagagatacaatgatggatgtcgtcaagttgttggatgcattccgtacagatatagaaaaaccaatcaatcatctacagttcaagggacccaaattgatgatttggaattcttattgatcagctcacagaagagtccaaagtggatgttccctaagggtggttgggaaactgatgaggcattagaagatgctgctttaagagagacttttgaggaatctggtgtagttggtgatgttggggttcaggagtatttaggtacttggagttttaagagcanaagtcaagggacatttcatgaggggcatatg

SEQ ID NO: 196 - DNA, 645 bp, Lycopersicon esculentum
ggaaaacgggtgtggttgggtacttatgacaccccagaagaagcagctgtcgtttacgataaagctgcagttaagctcaaaggtcctgacgccgttaccaattttccggtatcaacaacggcggaggtaacggtgacggttacggaaaccgaaaccgagtctgttgccgacggtggagataaaagcgaaaacgatgtcgctttgtcacccacctcagttctctgtgacaatgattttgcgccgtttgacaatctagggttctgcgaagtggatgcttttggtttcgacgttgattcacttttccggctgccggattttgctatgacggagaaatactacggcgatgaattcggcgaatttgactttgacgattttgcccttgaagctcgatagtgtacgaggggctatttcgtccatttttgcaaatgggttcactggttagttgactagtgacgtggcatttttggcgggaatatatatatagtgattagcagtctctattcatacgaagactttgtgagagatttttgtttttatttttctgttaattgtgggtgaatattgtaatatgaaaaattttgtatggtgaaattgaattaattaacgatgaagataaggagagtgaaggggggatgtgtgtatttt

SEQ ID NO: 197 - DNA, 681 bp, Lycopersicon esculentum
gcaccaggaggaaagggccttggactatggaagaagatttaattctcattaactacattgctaatcatggtgaaggtgtttggaactctctagctaaatctgctggtctcaaacgtactggaaaaagttgtagactccgatggctaaattatcttcgacctgatgtcaggaggggtaatattacacctgaagaacaacttttgattatggaactacatgctaagtggggaaacaagtggtcaaaaattgcgaagcatttgcctggaagaacggataacgagataaaaaactactggaggactaggattcagaagcacattaagcaaggagagaacatgaatggacaaggaagttcagagcaaaatattgatcatcaagaaggaagtagtagtcaaatatcatcagttggtcaagcagataacatagagacttactctccaacttcatacaatggaaatttggacactaattttcaagcctcaaattttctcaatgaaacaaatgacaacatgtggagcatggaggatatctggtcaatgcaattgcttaatggagattaaatcaagtatgtttggaatatttaatttgagtaattaattaaaacttagattgaaaccattagtaacgtattatgaatattaagctgttagatattacagctagcctatattatatagtgct

SEQ ID NO: 198 - DNA, 553 bp, Lycopersicon esculentum
  mRNA (1)..(553)
gcagctatatatcaggcacccttccattccaatggatccaagaaaagaagttatacaaaatggactcaataatcatcctagttttgatcaagattacttttcaaatcatgttgttggagtgggagattcttctcctcctcctcaagaggaaggggaaaaggactattctgatgcaatgtacaaattcttaagtcagatgcttatggaagaagatgatttggagaataagccatgtatgtttcatgactgcatggctctccaagctaaagagagatacttatctgatgtacttcatggatccgaaaacaactattccccacaatctgtcattatcaatccacacgattcctctagttttctcagcaactattcacctgattctattgagtcacctcaatgggatcttaatttcgaatcccctgcttccatgtccactttatctaatcatgactctttcttcacttccttcggcaatggccattttgaagaaggagctgtcaatgtgttacaaagtggcagcagcagcaattcaccaactggtctgagggaaaag

SEQ ID NO: 199 - DNA, 707 bp, Lycopersicon esculentum
gatgactctcttacaagctcttgcagcacgacccggtggtgccccctacgtacgtattacaggaattgatgatccagtttcacagtacgctcggggagatggattggctgcagttgccagacggctatcagcaatttctgaggaattcaacattgctgttgagtttcatgcagtgccagtttttgctccggaaatcacttgggatatgcttgatgtaaggcctggtgaggctctggctgtaaactttcctttgcaacttcaccacacccctgatgagagcgttgacgtgaataaccctagagatggtctcattaggatgataaagtcgcttagccccaagatagtcactttggtggagcaagaatcaaacacgaacacagctccatttctccctaggtttgtagaagctctagattactaccatgcaatgtttgagtccatagatgtgaccctactaagggacatgaaggagcggatcaatgtggaacagcactgtttggctagggatatagtgaatgtcatagcatgtgagggcaaggaaagagtagaacgtcacgagctattggggaagtggaaatccaggttcatgatggcaggttttcagcaatatcctttgagctcttatgtgaattcagtgattaaggacctcatgaagcgttactcggagcattatacactggtggagaaagacggagctatgctgttgg

SEQ ID NO: 200 - DNA, 591 bp, Lycopersicon esculentum
ctttgacacttcaccacacacctgatgagagcgttgacgtgactaatccaagggatgagcttctaaggatggtgaagtcattttctccaaaggtggtcacgttggtggaacaagaatcaaacacaaacaccgacccctttttccctcgatttcaagaagctctagactactattctgcaatgttcgagtccatagatgtgacattagaaagggacaggaaggaaagaatcaacgtggagcagcattgtttggcaagggatatagttaacgtcatagcatgtgagggcatggaaagagtggaacgccacgagctattagggaaatggaaattgaggttcacaatggcagggttccaccagtatcctctgagctcttacgtgaattcagtgataaagagcctcatgaggtgctactcagaacattatacactggtggagaaagatggagctatgttgttgggatggaaaaagaggaacctaatctctgcatcagcttggcactgaagaaaattacgaaaggacagcctggtggactaagctctcggggtctagcgaagatctagtttagcaaataactaagtatgtaagttca

SEQ ID NO: 201 - DNA, 518 bp, Lycopersicon esculentum
caggctgcttatcgcgtgtgccagagctcttgctgagaacaacttggatgattttgaaaaattgattgccaaggcaagaagtgctgtgtccattactggtgatccaatccagcgtctcggtgcttacatcgtagaagggctagtagcaagaaaagaagcatccggaaccaacatatacagggctctcagatgtaaggaaccagcaggctgggacttgctttcctacatgcatatcttatatgagatatgcccttatctaaagttcggttacatggctgcaaatggtgcaatagcagatgcgtgtagaaatgaaaatcgcattcacattatagacttccaaattgcacaagggactcaatggctgactcttcttcaagctcttgcggccaggccaagcggtgcaccttatgtacgtattacaggtattgatgatccagtttcaaaatacgcccgaggagatggattggccgtagtgggaaaaaagctcgcagcaatttctgagaaattcaacattcctg

SEQ ID NO: 202 - DNA, 666 bp, Lycopersicon esculentum
caacggtaaaccaaagagaccagcttctgcggatggtgaagagcttaaatccaaagctggtaacagttgttgagcaagacatgaacactaacactgctccatttctacaaaggtttgcagaagtctacaactactattgtgccgtatttgagtctctcgatgcaactctttcaagggatagccaagagagggtcaatgtagaacggcaatgtctcgcacgtgatatcatcaacattgttgcctgtgaaggattggagcgaatagagcgttatgaggttgcagggaagtggagagctaggatgatgatggctggtttcactccatccccaattagccgaaatgtctatgaatcgatccggaatcttatcaagcaatatagcgaaaggtacaaagcagaggaggaggcaggtgcactttatttcgggtgggaagacaaaaccttgactgttgcttcagcttggaggtagaaacaacatatgctctctcagaaacaaacaatgctgtgcattggagatcaacttgtagcgaaaaccgactgtgtgataatatttcagtgtttatgatcagaatgcatggcattgtatactttgatgctattcttagatacgattaacttctgtgctgatgtaggttgatgtgaaactagtagataaaactgtttttttt

SEQ ID NO: 203 - DNA, 388 bp, Lycopersicon esculentum
rgatcctaaaggcttcatcacttaccttggtctcaaggttagaagggattatatgtgtctgtattgcaacgatagatgtcgtccttttagtagtttggaagcagttcggaagcatatggatgcaaaaaaccattgcaaggtacactatggtgatggcggtgatgatgaagaggcggaattagaagagttttacgactatagcagcagctatgtggatacaactgggaagcagcttgtttcatctggggattttaacaacaatgtagagctgggaagtggtggatctgaactcataataacaacaagaactgatgatcgattgtctgttaaatcaatcgggtcaagagagtttttgagatactatcgccagaaaccaaagcctacgcgt

SEQ ID NO: 204 - DNA, 428 bp, Lycopersicon esculentum
yggccatgcactaaaagcaagcgctccaaaaatcggaactgagcaattgcaacatcacttgacataacagcctgcattccttcgacgccactgattccaactccgatatcagcttcttgaagcattcctacatcattagctccatctccaactgccaatgtgatctttccggtaccgtttttgacaagtcttgttaccagtgccttctgttttggtgaagaacgacagcaaataacagaggcacatttgattgcaaggtctaaaaacatatctttcacttcatcatctagggcatatgtaagagatttcccatcaatgatcaaagcaaatgcctctgtgcttgaagcagtaagtagagcctttccttcagtaatttgctgtgtaacacttccctttgaagccctggcaatggcattcttttctccgga

SEQ ID NO: 205 - DNA, 300 bp, Lycopersicon esculentum
ggatccttgagtagttcaccatgtgtacctttttcaacaacttttccccgatgtatgactgcaatcatatcagcatttcttactgtggtcaatcgatgtgcaactatgacggttgttctgttgatcataattctatccaatgcttcttgtacaactctctcagattctgcatctaatgcacttgttgcttcatctaacagtaatatccttggatcttttaaaattgctctggcaatggcaattctttgcttttgcccaccagaaagttgagttccatgttctccaaccatggtgtctaga

SEQ ID NO: 206 - DNA, 361 bp, Lycopersicon esculentum
cctaggagaaagacgaatcttcgggctaggtcttggtgaggggatagggccatacatgttgaaacctccgttagcaactcgtggtgacaagttaacttgttcaagtgctcggaactgtagctccgttggatattccctaacacaaccaatacgaggcccgtttcctgttgaccattttcgagacaaacgtttagcaaagctaaacgatgtgtcttcttgtttcttgtcttcgcgttcatcatctgttgtattaacctctctcttttcctctgttttttcctctgtacttctcttaggtgattctttagtaatgagtgattgatgattcgatccctcgttgttaactctaaacgaatgatca

SEQ ID NO: 207 - DNA, 404 bp, Lycopersicon esculentum
cctaggttcaatctgcagcatcaatgcatcaagtatctaggacctaaagaacgtgaatcgtatgaagtagttattcaggacgggaaacttgtctataaacaaaacggtgtctttgtggagacagtagagggttcaaaatggatctttgttcttagcacaacaagaacattgtacgtaggaaaaaagaagaaaggtgtgtttcaacactcgagtttcctatcgggtggtgcaactactgctgctggtagactcgttgctcatggtggcgttttgaaggctatttggccatacagcggacactatcaccccacggaagagaacttccgggaattcattagtttcctcgaggagcatagcgtagatcttacaaacgtcaagagatgttctgttgacgatgatgatca

SEQ ID NO: 208 - DNA, 367 bp, Lycopersicon esculentum
gtgcacggatcgcctacttcattgccttgttcctttacttctcactggctgttcggattaatttcttccgaggcttcaggttctcattggcttggtgggcttacacttttccaatgaccggagctgctattgccacgatcaagtactcgatcatggttaacacggttgtgacaaagtgcctggttgttatactttgcaccctttctacattcacagtgacaggactgcttgtgacaaccatcatccatgcctttgttctaagagacctctttccaaatgacatttctattgcaattagtgaaagaaaacctaaaacacatagacgatggtatcatcatagacgagctggaagcacagatattgatca

SEQ ID NO: 209 - DNA, 386 bp, Lycopersicon esculentum
caattggcagaggaattgcagaagcaatttgttattgcagagtccttacaaattttcgcaaggtatgaaagggggttgattgagtcagctattccgaatcgagagaaattatactgtccttataaaaaatgtgcaaaattgctgagccatgatcctgatgacgatgaagagatcgttatgaaggggaaatgtccttggtgcgcaggactgttgtgcgcgcggtgcagagtaccttggcacactgggcgtgactgtcaacagtttcaggaagaagaaaaagacagagaagatgacttaagggttaagctacttgctgaaaatcacaaatggaagaattgccctcattgcaattctcttgttgataaagttgacgatggctgtgtaca

SEQ ID NO: 210 - DNA, 335 bp, Lycopersicon esculentum
acgcgttactttaaggcaaaatcagactacgtgtatcagccaatcccagagggaggtctcgatggaatatggagattcttctttgaagatgaagctcaatcatctcaagtgatcttaagtccttatggtgggaggatggatgagatttcaccatccgctataccgttcccccatagagctgggaatttatacaaaatccaacatttggtgtattgggatgaagaaggggaagaagtagctgagaggcatataagttggataagaagactttattcttacatggctccttttgtttctaagtcaccaagagctgcttatatcaactatagggatcy

SEQ ID NO: 211 - DNA, 163 bp, Lycopersicon esculentum
gggcccacaaaatagtgtcacgtaactcccaaatggtaggaaattattaataaaataagttcagagataaaagaccttagtatagtatgagtgtgtctctgagatttcatgctaggttgagagggtatttgtgcattatcccaaaagaaaaactgtttctaga

SEQ ID NO: 212 - DNA, 387 bp, Lycopersicon esculentum
gggcccaatgaggtgcacctggactaaagtgcactgaaattaccctggagtttaataattccgctataggtgtaaaattaacacacctaggagccttatactgatttatagatgcacctctagaaattgcatagtccattagctcttcaaatgtaccattttccactactctaatttcaagtggtccaatcgagttatctgcaactcggcactgtcgataaaccgagttcaaacattcttccatagccaaacaacattgattcaaagcctcgtggctcggcgaattggttgggtccttaataagtaattcacagtaaataacataatgtcctggaatggttttggtgtcggcataactagtgtactcgaccacgctagtattgtaca

SEQ ID NO: 213 - DNA, 178 bp, Lycopersicon esculentum
gctagcttcatttcagaaagatgaagatggatttgatcaccaagaagcaaaaatgcctgatctgcctgatccagaaacgcttttatcaatggaagatttgagagaaatgcgcaagactgatccccggcttcccaggacataccgaaataaggtttctactgcaaaatttgttccatgg

SEQ ID NO: 214 - DNA, 124 bp, Lycopersicon esculentum
gaattctatcccaaaagaggcagcataccaaataattaatgatgagttgatgcttgatggcaatccaagtttgaatttggcatcatttgtgacaacatggatggagccagaatgtgataagctt

SEQ ID NO: 215 - DNA, 221 bp, Lycopersicon esculentum
tgatcatcgctgctgctttgatgtagaggtgacacgtgtactaacacgtgtcttgttcttttgatggacggacgagatttgttttttctaatactttaaattactttttgtaattttgtgatttatttgtttttggtacattgtgggttttttatttttatttttattttgttaattatatttttcattttagagtgaatcattcatcatccttgtgtaca

SEQ ID NO: 216 - DNA, 268 bp, Lycopersicon esculentum
ggtaccaaactcaccaaatctatattaatagagcaaccatttcagtgattttattaagagaaaaactgaatactgcctattttggagagagttcatatggagaagaacagcaacatgtcaaagaaagaagaaggattctttgaacaaaaccaagaaggtgacaatcatcgtcagtacattatgcatagccaagtgaagaagattaaggaggaagagtctgagagaaatgtaattgaccggccggttttagcaagggaggtcatcatga

SEQ ID NO: 217 - DNA, 114 bp, Lycopersicon esculentum
gggcccagtaagttctgttggtagtttgaaatggtcttgtgtaatcatgagaaaagctggcatttgtctgtattcagacaggagatagtcaacatcaacagaaaaaaggaattc

SEQ ID NO: 218 - DNA, 235 bp, Lycopersicon esculentum
agatctaatcactggttctgtaaaaacccgatcagactcgtggtcaaacttttatgtgtttaactaaaaacaatggtaaatcgatagaattacatatacattttaaaatctaaattctgaactccggcctaacgtgtagacataattaaaagtaaacaaatgaaaggtaggcaaagagagccatagctatcgaagacatgcacaatggggtggttgatgatgagttgttgctagc

SEQ ID NO: 219 - DNA, 138 bp, Lycopersicon esculentum
gaattcagtgagattattgaatttctcatttataattattaatttcatgtttatgacacttttcaaatttagttttggacaaggcatggctccttcttctgctccttctccttccaatgatggcacaacaattgatca

SEQ ID NO: 220 - DNA, 333 bp, Lycopersicon esculentum
tccggaaacaaaatcagaccggtggtttctcaagattgcaccgccattctttaccagaggttcatcaatgaagtcaccaaaagtaagagctgataacgaagaagcatccacatcacgcagcaatatgaaagtagcagtaaaattgccgtccttccaatgcctggagccgaaaggtgatgaaccgggtttattggcaaatgactcggctcgatctccggtttaaagaatgttataattactgactcgggtttaattaagcgtggtggaaaggtaaaactttttgttcgccggcaaatatggagaacgcgtggtcgaagctaatgggctgggccc

SEQ ID NO: 221 - DNA, 340 bp, Lycopersicon esculentum
ccatggaaatctattggattttgaagtaattgcgagtattcccaataatctctagccatatctgagctctgaaataactgttgttgctgattctgatgctgctgatcagattgacgcggaggttgattatatgattgaaaaaaaaatggtgatggtgatggtgatcgcgttgatgctgcagacattaattggaatcgcggagctgcggttgatgatgaggttgatatggctaatcttgttgtgggttgttgttgttgttgtggtaataaccgaacattttcagggaaattgagtttagctctgtttcctctgaatctaagagcggcgtcatcataagctt

SEQ ID NO: 222 - DNA, 245 bp, Lycopersicon esculentum
  misc_feature (81)..(89): Unknown nucleotides 81 and 89
gctagcgctgtggtctcatgtccaccgaagaagaacgttttacattcatccaccatttccctcgtactcaacatcctcccngttccccngctaacaacagactcagaagatcgtgctctgtacgatcaacgctttttcttttccgctcctcaataatcgaaatgaggagcgaatcgatctccttgcctagattatgggcttttaacgtctcttttagacacattatttggttcccataaggtacc

SEQ ID NO: 223 - DNA, 282 bp, Lycopersicon esculentum
gtgcaccagaaaaaccggtggagttgcccgtgtttcctctacttatgggaaggaagctagtggctggaagttgcataggagggatgaaagagactcaagaaatgttggactttgcggcaaagcataacataacaccagatattgaagtggtgccaatggagtacgttaacaccgcgttggaacgtcttttgaaatcggacgtgaaatatcgttttgtgctagacattggtaatacattgaacaagaaatgagaatagggaggggatcaattatgtagctagc

SEQ ID NO: 224 - DNA, 238 bp, Lycopersicon esculentum
gaattcttcacaaagctctcggccttggagctatcaagtgatccctgatcctgagcaatagtgtgaagggtccgatggacatccctggccatgcccttagcatcaccacagacataaacataacctccctgagaaatcatgttccagatgtcgtccgccttctgtgtcattttatgttgcacatattctttgttaggtccttcacgtgagaaggcaacaactagattagaaagtgcac

SEQ ID NO: 225 - DNA, 61 bp, Lycopersicon esculentum
rgatcctccactcaaaagaacttgatccgattgaagaaggccttttctttgcctcaaatty

SEQ ID NO: 226 - DNA, 233 bp, Lycopersicon esculentum
tccggaaattttccatgtcaataggtacaggtttccctgttacattagtgaacctttgtgaaaatgtacttttatctgatgtcaccactcccttaaagtaaccaagactttcaccatttaaacccgtcaatgaccatgaatcttcattatccaaccactccaacctcccaacgcctaccccaaacctactttgaactccaggctcctggcataacgatttacaaattgaattc

SEQ ID NO: 227 - DNA, 83 bp, Lycopersicon esculentum
agatctatttcatgcttccggtctccaagcttaaatatccactcactgcctctgacatggcagctcttgccctaaaggctagc

SEQ ID NO: 228 - DNA, 380 bp, Lycopersicon esculentum
ccatggatgagttcagattgcatattgagaaagacaaggcctttgctcgaaggttccagcccatcttggttaatgaaccaagtcaggcggatgctgtccagatactattggggttgcgtgaaaaatatgagtcgcatcataagtgtaggtacagtctggaagccatcaatgctgctgtggaactatcatcaagatatataccggataggtatcttcctgacaaagctattgatcttattgatgaggctggtagtaaatctcgtatgcaagctcacaaaagaagaaaggaacagcagatatctgtactttcacaatcacctagtgattattggcaggaaattagagctgttcaaaccatgcatgaagtgatcctggctagc

SEQ ID NO: 229 - DNA, 157 bp, Lycopersicon esculentum
  mRNA (1)..(157)
cctagggaaataaccgacgcaccacctttgtacgcttcggtggtgctccaccacgacgcaacggaaggtggtgacttaattttcttggtcttagggctaagagattggatgaaatcgacgataatcgaacgctgtacggaagtgaattttccgtacc

SEQ ID NO: 230 - DNA, 229 bp, Lycopersicon esculentum
gtgcacataatttggaggctcatttgcacttgcttgacggatatcatggaaaagggagaagatttgtgcttgaaaaaggcagagacatagccttagtgaacaaagcatgtgattttttgaaagatcattattgtgttcctcctaattggagacaagatgaaaataaaggtatgattagagacaaagatggacgttgggttcaacctgaacgtcctagacttgatgatca

SEQ ID NO: 231 - DNA, 382 bp, Lycopersicon esculentum
ggatccatccttcatcagtttcaaactgagactattatatcgttctcgtgaatactgtcttgatgccttcctccaatctgttccagccttcatcatagtagagaattcatcgtaacttatccgtccatccttgtcagtgtctacatcttgcataatagcattgataacttcttcactgttggtttcaatctcgtcatctaaggcttcccttaattcctcaatctctatatatccattttggttcctatcaaaaaaatcaaatgccttcttcaggtgctcctcatttgccatctttcttagatggactgaaattgctacaaattccccgtagtccaaaaatccatctttatcgacatcaccaacatccataagaatctgtaca

SEQ ID NO: 232 - DNA, 169 bp, Lycopersicon esculentum
cctagggttcaccctagccggctcgggcacacccgagccgattaaatgaggaaaatttagcctagctttcgagccacgaatcttgaatgcggcttggtcataagccaatgctgcatcctccggtgtctcataagtccctaaccataatctcgcatttttcttatccgga

SEQ ID NO: 233 - DNA, 1519 bp, Lycopersicon esculentum
ccttgttggtgctggcacagttggacggggaaataagggctctggtcttgacattgctaatttgctaaaaccaacacttggccgtggtgaactgcagtgtattgcatctaccaccatggatgagttcagattgcatattgagaaagacaaggcctttgctcgaaggttccagcccatcttggttaatgaaccaagtcaggcggatgctgtccagatactattggggttgcgtgaaaaatatgagtcgcatcataagtgtaggtacagtctggaagccatcaatgctgctgtggaactatcatcaagatatataccggataggtatcttcctgacaaagctattgatcttattgatgaggctggtagtaaatctcgtatgcaagctcacaaaagaagaaaggaacagcagatatctgtactttcacaatcacctagtgattattggcaggaaattagagctgttcaaaccatgcatgaagtgatcctggctagcaagctgactgaaaatgctgatgcgtctcgtttggatgatgacagtgaacttcatttgcagccagcttcgtcttctacatctgatcaacatgaactcccattagttggacctgaggatatagcagcagttgcttcactctggacaggcattccccttaagcagcttacagttgatgaaagaatgcttttggttggtcttgatgagcagcttaagaaaagggttgttggtcaggatgaggctgttacatctatttgtcgggctgttaagagatccagaactggcctcaagcacccaaatagaccaatttcggcaatgctcttctgtggtcctactggagttggaaaatctgaactggctaaagctttggcagcatcttattttggatctgaatctgccatgctaagattggatatgagtgaatacatggagcggcatactgtgagcaagttaattggatcgcctcctggttatgtaggctatggagaaggagggactctaactgaagctatccgaagaaagcccttcactgtagtgctgctagatgagattgaaaaggctcatcccgacatattcaatattctccttcagttgtttgaagatggtcacctaacagactctcagggaagaagagtgtcatttaagaacgccttgatagttatgacttcaaatgtgggttcaacagccattgtaaagggtagacagaatactatcggcttcttgcttgctgaagatgagtcagccgcctcctatgctggtatgaaagcaatagtgatggaggagctcaaaacatactttcgtccagagttactgaataggatagatgaagtagtggtattccgtccactagagaagcctcagatgctcgagatattaaacctgatgttgcaggaggtaagagctaggcttgtttcattaggaataagtttggaggtatcagaagcagtaatggacctcatatgccaaccaggatttgacagaaactatggcgcacgccctctgagggaggctggtactcaaatggttgaa

SEQ ID NO: 234 - DNA, 631 bp, Lycopersicon esculentum
  misc_feature (467)..(475): Unknown nucleotides 467 and 475
ttcctccaaacccaaaaggcataaatcccatcttatgtttacatccaccatggatgtggtccttaaacctctttggtttgaactcattcacatcttcaccccaaagtgtcttatcatgaggcatggacaccacatcaatccacatatttgttccattaggtatgaccaaatcatccactttaatgtcttctctagcttgcctttgcacatttggtgctggtgagtatagtcttagtacttcattcatcacccatcccatcttcctaagaccaacaagcttagtgacatctatttcaccatcatcacccatcacttgtttgatttcctctctgagttgattttgccactctggatgctgtgccaacaacaacaaagtccaggccagtgctagcgctgtggtctcatgtccaccgaagaagaacgttttacattcatccaccatttccctcgtactcaacatcctcccngttccccngctaacaacagactcagaagatcgtgctctgtacgatcaacgctttttcttttccgctcctcaataatcgaaatgaggagcgaatcgatctccttgcctagattatgggcttttaacgtctcttttagacacattatttggttcccataaggtacc

SEQ ID NO: 235 - DNA, 658 bp, Lycopersicon esculentum
ttccattacttatgttgatgaaatctcatggtaagcttgttatggttggtgcaccagaaaaaccggtggagttgcccgtgtttcctctacttatgggaaggaagctagtggctggaagttgcataggagggatgaaagagactcaagaaatgttggactttgcggcaaagcataacataacaccagatattgaagtggtgccaatggagtacgttaacaccgcgttggaacgtcttttgaaatcggacgtgaaatatcgttttgtgctagacattggtaatacattgaacaagaaatgagaatagggaggggatcaattatgtagctagcagtgtttcctccatcatctatttatactgcagtaactgagatttagtgaataccttacagaataacaaccctttgcctttactctattaactaagtgaaatccgtatatctctgatacatcttttcacatgaaaatcatacaacaaagtcaaattctgaacttagtactcaagaagacagcttgcactaatgaattatgacggaacaccaagatatgatgcaagttgcaagatgacactgtactttttcctcaaaaaaatgacacagagtactccacaagctgtcactttgatttaaaaagatacatctatttcttttctttccaact

SEQ ID NO: 236 - DNA, 584 bp, Lycopersicon esculentum
agtatcaaacccaattttctcattcaccttataacaatagaaagaaagaaagagatcgattgttaaagatgaagaacgttataaaagttgtgtttgtgctttctatcatcatgtgtgtcctccttacaccaaccattgctagggtgagtgcgagtcgtggcagttcaaggaccccagcagtccgtgctgctggtcgcgctagcaacaactcatcatcaaccaccccattgtgcatgtcttcgatagctatggctctctttgcctacctttcatttgtttacttttaattatgtctacacgttaggccggagttcagaatttagattttaaaatgtatatgtaattctatcgatttaccattgtttttagttaaacacataaaagtttgaccacgagtctgatcgggtttttacagaaccagtgattagatctgaaactgttatatttctatttttttaaaaaaatgcatttgttgttctttatattactgcatgcatggagtactgaggccatcagccatatcacaattataatgtaatctgttgtctgttgtagttttgaataaaaaaattgcttttgatact

SEQ ID NO: 237 - DNA, 792 bp, Lycopersicon esculentum
cttcctcctcgaatatcgtcgatcaaaagcagctgaccagctacgtcatctcaattggtggagagctccgtcaatatagcgttcccatcaacgtctctcaagttcttcaatctgacatctcttccaccttcctatgcagttccgaccgtctttactttgaccatttcataccttcacttgattcccaataccagcttcttcccaatcagatctatttcatgcttccggcctccaagcttaaatatccactcactgcctctgacatggcagctcttgccctaaaggcaagcactgctctggaagaactcaacaaaaacaaaagccgaaagtctaggaagatcacgagtaagaaccgaatttcacccatgcttcttccactagaacaagaaaccaataattattacaagtccactaccgtaggacttgggatttccatgagatctgcatcggtcagaaaattgcagcgattatcttcacgccgggcaaagatggctgtgcgatccttccgtaagcttatcaccattcaagaagggttcgttcttttttagttaatgatatattctttttggttgccggtaaacatgcgatgcgagttagtgctcttctctcgctctacacgtgtatgtgtatatatatatatatataggaaatacaaaacaaatatatattttacatatatacgattaaatttccttgtatgtaaatggtacttaattaattagcttgtacaacaacaacaaccatgtattacttattagtacagtttaaccaaaaaaatctacacttgacgat

SEQ ID NO: 238 - DNA, 688 bp, Lycopersicon esculentum
tttgggtaccaaactcaccaaatctatattaatagagcaaccatttcagtgattttattaagagaaaaactgaatactgcctattttggagagagttcatatggagaagaacagcaacatgtcaaagaaagaagaaggattctttgaacaaaaccaagaaggtgacaatcatcgtcagtacattatgcatagccaagtgaagaagattaaggaggaagagtctgagagaaatgtaattgaccggccggttttagcaagggaggtcatcatgaccaggcaacattctagatcacgccttgggttaatatctggccaacctatatctgttggcgactcttagaagtattgagtcaaaattggttgttttcaattacaagaagcctatatagatcacttaggttctaccatctttcttgctctaccattttaagagggttttaggctttgtttctatgtacgtaattatgcctccagtacagaatattaaatggttatgaaacctttagaagagtttatctcagttttccttttctatctagattgcttacttgtttagtcttttttcttctcctgtttatgtaattttcatctggatggttaatggagaagtcttatcttatccatgtttgaccactttttgcgaacatcaaaagctaatgatgaaaattgagctgacaattttacattc

SEQ ID NO: 239 - DNA, 856 bp, Lycopersicon esculentum
cagattaaccattataatagctcagaatgtctctgcattatctgacatcagatgcgaaattggcagaggaattgcagaagcaatttgttattgcagagtccttacaaattttcgcaaggtatgaaagggggttgattgagtcagctattccgaatcgagagaaattatactgtccttataaaaaatgtgcaaaattgctgagccatgatcctgatgacgatgaagagatcgttatgaaggggaaatgtccttggtgcgcaggactgttgtgcgcgcggtgcagagtaccttggcacactgggcgtgactgtcaacagtttcaggaagaagaaaaagacagagaagatgacttaagggttaagctacttgctgaaaatcacaaatggaagaattgccctcattgcaattctcttgttgataaagttgacgatggctgtgtacacatcacttgcaggtgtaatgaggaattttgctacgcatgtggagcaacttggagcaaaaggcattggaattgccagactcgttaatccaattggaacatgaagatggacaaatacgactacaaacaataaaaatttatcaatgaaacctgtcgtaatatttcattatatatgattttttaaaaatacgataccatatttatttcatgttaattaaagtgagttgatgatacaaaagttacttaatcatgtcgttaatttgatgcgccattcaagttgtagtattattattacaaacttgatcagccttgtttagcagtcactcgttgagttgttgttatgcttaatatgtcgttaatttgttgttttgcttattacttgttcattttaaagtaacatatttattgagtaccctg

SEQ ID NO: 240 - DNA, 1079 bp, Lycopersicon esculentum
tgaagagtttaatttgcaattgttgtaataaggaattttttgacgaaaatgaacaaaaaattcattacaaatcagaatggcacacttataatcttaaacgcaaggttgctggagttcctggattgacagaagcactctataaagccagacagtctgcacttttagaaaccccattgctatatagctgtgaacactgtggcaaagaatttacaacctctaaagctcatgctcaacatctcaaatctaagaagactcacttagcacgagcttcgcaagagatagctcatcatcacgagtgggaggaggttgatcaagaggaagagatcgcgtgtgaagacaatgactcattgaaggaactaaagatgaacaggagcagtactagtagtggtgttaaggatgaggagtttgatccgcgttgctgtttcatgtgtgatatgaaacacgatacagtagaaaactgcacgatccatatgcacaagaagcacgggttcttcatacctgatatcgagtatttgaaggatcctaaaggcttcatcacttaccttggtctcaaggttagaagggattatatgtgtctgtattgcaacgatagatgtcgtccttttagtagtttggaagcagttcggaagcatatggatgcaaaaaaccattgcaaggtacactatggtgatggcggtgatgatgaagaggcggaattagaagagttttacgactatagcagcagctatgtggatacaactgggaagcagcttgtttcatctggggattttaacaacaatgtagagctgggaagtggtggatctgaactcataataacaacaagaactgatgatcgattgtctgttaaatcaatcgggtcaagagagtttttgagatactatcgccagaaaccaaagcctacgcgtactaatgatacagctataagctctgcctagcttcaaggtatcgaagcacgagactagccaccatgtagagggagcacaatgctatgatggaagtgttgaaggccatgaatagatgtggtgtggatgctgcactctgggattggcaggaaaagcaatgttattcgagacctccct

SEQ ID NO: 241 - DNA, 1985 bp, Lycopersicon esculentum
cttggcaacatatggagatggtgaaccaactgataatgctgccagattctataaatggtttgaagaggggaaagagaggggtgactgctttaagaatcttcagtatggagtatttgggcttggcaacagacaatacgagcattttaacaagattgctaaagttgtcgatgagcttctggctgagcaaggtgggcagaggcttgttccagtgggtcttggagatgatgatcaatgcattgaagatgattttgctgcatggcgtgagttagtgtggcctgaattagataagttgcttcttgacggggatgatgcaactgctacaactccatatactgctgctgttttggaatatagggttgttacctatgacaagtccaactttgataacgacttgaccaacacaaatggtcatgcaaatggacatgtcatcgttgatgctcaacatcctgtcagagctaatgttgctgtgaggaaggagcttcatactccagcttctgatcgttcttgcactcatctggagtttgacatttcttgcactggacttacgtacgaaactggtgatcatgttggtgtgtactgtgaaaattttgttgaaaccgtggaggaagctgaaaggctactgaatatatcaccggatactttcttttccattcacaccgataaagaggatggcacaccacttggtggaagttcattgccatctccattccctccttgcactttgagaacagcattgactcggtatgctgatgttttgagttctcctaaaaagtcttctttacttgctttagcggcatgttcttctgatccaaatgaagctgatcgattaagatatctagcatcacctgctggaaaggaagaatatgctcagtggatagttgcaagtcagagaagccttcttgaagtcatggctgaatttccttcagccaagccttcaatcggtgttttctttgcttctgttgctcctcgcctacaaccaagattctactccatctcatcatctcctaggatggcggcatctagaattcatgtcacttgtgcactggtttacgacaaaatgccaactggacgaattcacaagggtgtctgctcaacatggatgaagaatgctattcctctagaagaaagcctttcctgcagtacggcacctatttttgttcggcaatcaaactttaaactgccagctgataacaaggttccaatcataatgattggccctggtactggattggcaccattcaggggttttctccaggaaagaatggctttgaaggaggaaggagctgaccttggtcctgcagtgttattttttggatgcaggaaccgccaaatggactacatctatcaggatgagttagataacttccttgaggccggtgcactttctaatctagttgttgccttctcacgtgaaggacctaacaaagaatatgtgcaacataaaatgacacagaaggcggacgacatctggaacatgatttctcagggaggttatgtttatgtctgtggtgatgctaagggcatggccagggatgtccatcggacccttcacactattgctcaggatcagggatcacttgatagctccaaggccgagagctttgtgaagaatttgcaaacgaccggaagatatctgcgtgatgtgtggtaattgtctcatggcaagcctattgtgattagagaaatatttaggaaggcgaggacttgcaaagaacaatcagccttccaatagggtgggtaaaacaagtgtacgatttgtcgtcactgctctactatttgtatgattctttttttacttttggataaaagattttttggttgatgtagtttatatatatcattaatatatatgtacaactgtgggacaaataattatgctagtattaattttaggcaatatttattcaagatcttgtgccttgtaatttccctaatgaaccagcaatatggttcagccaacattgtatgctgacattttaaggcca

SEQ ID NO: 242 - DNA, 999 bp, Lycopersicon esculentum
tcatgatcatgggctggacagaaaagaagacgtgatcaagaagaaagtgttactggagaacaagctcaaaggggttatggaggtatcggagaatttaaaaatggagaatcatcttcctctgttaagctcgaagaagatacaagcctggcaacaccacaaacgagcagcatatcgacaactactgcgtcgcagacaccaccgcaagcatcggaagtaacaggtgaagaaacaggggagaggaagaggagatacagaggagtaagacagagaccatggggtaaatgggcagcagagataagagatccacataaagcagcaagagtttggttaggcacattcgatacagcagaagcagccgctatagcttatgatgacgccgctcttagattcagaggaaacagagctaaactcaatttccctgaaaatgttcggttattaccacaacaacaacaacaacccacaacaagattagccatatcaacctcatcatcaaccgcagctccgcgattccaattaatgtctgcagcatcaacgcgatcaccatcaccatcaccatttttttttcaatcatataatcaacctccgcgtcaatctgatcagcagcatcagaatcagcaacaacagttatttcagagctcagatatggctagagattattgggaatactcgcaattacttcaaaatccaatagatttccatggaggacaacaatcatcatctttgttagaacaaatgttactagcttcatcattgggagtgttacattcacatacatttccttcttcgtcatcttcttcattagcaacttctgcagcttcttccactacttcccctgcatatcccctgttttactctgctcaacaatcacgcttctttcagccacaaactcatcaaaatcaaagcaatagcagtagcaacagctcaaattttcctccacctttttggactagttccggccactatccaccttcttctagcttatttttcatactacttccc

SEQ ID NO: 243 - DNA, 743 bp, Lycopersicon esculentum
  mRNA (1)..(743)
aaaataatcctcttacaatattcttatcaatggaatcctctaagcctatcgtaactattttcatcttattattttcactctttgttcttttccctaatccttctttagccaattcaagaatacttgctctagttaaagaaaagccccttgtcctaaaataccataaaggggctctattaaaaggaaacattacgattaatcttatatggtacggaaaattcacttccgtacagcgttcgattatcgtcgatttcatccaatctcttagccctaagaccaagaaaattaagtcaccaccttccgttgcgtcgtggtggagcaccaccgaagcgtacaaaggtggtgcgtcggttatttccctaggaaaacaaattttcgacgaaaaatattctctagggaaatatctgaaagaccctcaactcgaatctttggcttctaaggctactcgggtcaactccattgctgtcatattgacggcgaacggatgtgggggtcgacgacttttgcatgaaccgatgtggcatgcacgggtcgacccggttgaagaaagcgggtaaatttgcttatgcatgggtgggtaactccgcgagtcagtgtccgggtcaatgcgcgtggccattccaaaagccgattgtgggaccacagattacgccgttagttgcacctaacgagacgtggggagttgacggaatgattattaacttggcgactgttttagcgggtaccgttacaaatccgtttga

SEQ ID NO: 244 - DNA, 818 bp, Lycopersicon esculentum
gctcattacacggttctgtttgtaactctctaccaaagacttccaacaaatgagacactcccaaaggagctacatcctgtgttctttctatttgttgctgcccctagtgttgcttctatggcatgggcaaatatccaagggtcctttgattttggtgcacggatcgcctacttcattgccttgttcctttacttctcactggctgttcggattaatttcttccgaggcttcaggttctcattggcttggtgggcttacacttttccaatgaccggagctgctattgccacgatcaagtactcgatcatggttaacacggttgtgacaaagtgcctggttgttatactttgcaccctttctacattcacagtgacaggactgcttgtgacaaccatcatccatgcctttgttctaagagacctctttccaaatgacatttctattgcaattagtgaaagaaaacctaaaacacatagacgatggtatcatcatagacgagctggaagcacagatattgatcaattccttaagtacgctgattctgctgaagccaaagatatcgaagcagctctatctggcagcgtagagttaataagcgcctctgctccaaaagaagttagtcaagattgagtcgttctgtttgaggatcctgcgttaaaatagcttgcattcgcttgtaaagccttatacttttaatcaagtgaggaacaccaaagagtttggtgatcctgtgaacttgatatagaagaaggcacatgaaaggtcttgtaaaagaaggatgcaagaagagaacgagtgtcgttccaagtttaaaagtt

SEQ ID NO: 245 - DNA, 655 bp, Lycopersicon esculentum
tggcttcttattttagagtttttcttttcttagccttttttgttgcttcttctatagctcaatctccggctccggcacctaaaatttctccggcagcaactccaactccggcgccggcaccagtattatcaccgccgaccgcaactccaactccatctccaactccgacaccaactccggcaccttctaccgctcctactacttcaccaactccatctccggctgcttccgcaacttctccatcgccgggaccatccggtgccggcgtcgtttcaccaccagcacctactcctaccggtggtgcaccggcggatcagccttctgccgacaacacaccgtcgtcgcctaacgccggtaacagagccgtcattggcggtgctgcgttcgctggcgtgatcatcgctgctgctttgatgtagaggtgacacgtgtactaacacgtgtcttgttcttttgatggacggacgagatttgttttttctaatactttaaattactttttgtaattttgtgatttatttgtttttggtacattgtgggttttttatttttatttttattttgttaattatatttttcattttagagtgaatcattcatcatccttgtgtacattttaagtgaaataaatttgagtttttattctttttgtttc

SEQ ID NO: 246 - DNA, 576 bp, Lycopersicon esculentum
  mRNA (1)..(576)
gaatatcaacacattccaataaatattcaacacataggattgcgatttcaaaccaatcaagacaactaaaacacttctcatgagtaaattgactaattaatatcaacgttgaatattcaactctactcacaaagatacaaaaagaaaaaaaaaagattttattccacttatgtggtctcgatcatgacttgaattaacttcatcattaaaattgtaaacgctgtcgttctggggcccaatgaggtgcacctggactaaagtgcactgaaattaccctggagtttaataattccgctataggtgtaaaattaacacacctaggagccttatactgatttatagatgcacctctagaaattgcatagtccattagctcttcaaatgtaccattttccactactctaatttcaagtggtccaatcgagttatctgcaactcggcactgtcgataaaccgagttcaaacattcttccatagccaaacaacattgattcaaagcctcgtggctcggcgaattggttgggtccttaataagtaattcacagtaaataacataatgtcctggaatggttttgg

SEQ ID NO: 247 - DNA, 720 bp, Lycopersicon esculentum
gatgtcgataaagatggatttttggactacggggaatttgtagcaatttcagtccatctaagaaagatggcaaatgaggagcacctgaagaaggcatttgatttttttgataggaaccaaaatggatatatagagattgaggaattaagggaagccttagatgacgagattgaaaccaacagtgaagaagttatcaatgctattatgcaagatgtagacactgacaaggatggacggataagttacgatgaattctctactatgatgaaggctggaacagattggaggaaggcatcaagacagtattcacgagaacgatataatagtctcagtttgaaactgatgaaggatggatccttacaaagctaattaagtaaagccaaaatatgacatcttgagtaagtactagaactcagcttttggctttctaaattcgtcctttctttcctgttcacccatcgatctgccattttttatttcgtctctgtattttttcttctgagggtggatcaattaacttggggacgactgcaacattcatgtgttctttgagttgtaatcatgttgtgtcattttctttttgcttttcgtttggacaatccgagaaaatatggtgaagggtagaaaggtgattattgtggttgtatataggttggcatcgtatgcttcaactgttcagttatggataccagttatttaatgatacaacatttcagattc

SEQ ID NO: 248 - DNA, 839 bp, Lycopersicon esculentum
  misc_feature (839)..(839): Unknown nucleotide 839
caggcacgattgattgacgcgaatggaaggattcttgatagggcttccatgggagaagatctattttgggcaattagaggaggtggtggtaatagttttggacttgtccttgcatggaaggtcaaattagtagatgttcctgagatagtaactgttttcacacttgacaaaacattggaacaaaatgccactaagcttgttcataagtggcaatatgtggctccaagattccatcaagatttgttcattaggatcttggttagtagattgaactcaagtaatcagggagatgataacaaccaacaaacaatcgtagcttcattcaactccatattccttggtggtattgatcgattacttcccatcatgcaagaaaatttccctgaattatggttgagaagagcagattgcattgagatgagctggatagagtctatattgtactttgcagggtttccaacagatggacctcttgatgtcttgctaagtaaggttcaactctcaacgcgttactttaaggcaaaatcagactacgtgtatcagccaatcccagagggaggtctcgatggaatatggagattcttctttgaagatgaagctcaatcatctcaagtgatcttaagtccttatggtgggaggatggatgagatttcaccatccgctataccgttcccccatagagctgggaatttatacaaaatccaacatttggtgtattgggatgaagaaggggaagaagtagctgagaggcatataagttggataagaagactttattcttacatggctccttttgtttctaagtcaccaagagctgcttatatcaactatagggatcn

SEQ ID NO: 249 - DNA, 255 bp, Lycopersicon esculentum
raatttcactgaaatacacacaacttacatacatattacataaacccaactccggcgccggcgccggcagtgaacagtggcaaattaagaaaaaaaaaactgaaatataacatcagccataattttaaaaaaagttaattaaactctaattacttcttgcttaagaaatcaccagccatcttaagataatcagctccttcactctttttttcctcctccttatctccttcaacaaccggctccggcgccggatcc

SEQ ID NO: 250 - DNA, 169 bp, Lycopersicon esculentum
agatcttcgagggtggttggcttccactagtttttgcgtcttggttcctctgtgtgatgtatatttggaactatgggagtgtgttgaagtaccagagtgaggtcaagcagaagatttcaatggatttcatgcatgagcttggctcttctcttgggacggtaagggtacc

SEQ ID NO: 251 - DNA, 323 bp, Lycopersicon esculentum
raattcggaggagacaccccatacagttttatgtttggaccagatatatgtggtacacagacaaagaaacttcatgttatactttcctaccaaggacagaattaccccatcaagaaggaattagaatgtgaaacggacaaattaacacatttctacaccttcattcttagacctgatgtatcatacagcatctggattgatggtagagaaagagattctggaagtatgtacacagattgggacatacttcctccccgcaaaattaaggctgtaaatgcgaaaaagcctactgactgggatgatagggaatatatagaggatcc

SEQ ID NO: 252 - DNA, 268 bp, Lycopersicon esculentum
gtgcactaattttctagaccaaagtggtgcatgtaaatgtaacaggatcatacaaagcaggaaccaagtatttccttccatttgtaccatgcgcattgtagcttgctcctgtagttttgtccaccaataaatctccgggatagcctgggtatgcgcccttagcgtacacaccagggcatgcagatgcagcttccaatggcgcatctgcttcgccctggtagtaaccgtttccaaatgggtttgtagcggttccagctaataagctagc

SEQ ID NO: 253 - DNA, 536 bp, Lycopersicon esculentum
gctttataataataccaacctaaaccatgggctatccaacaactccatcaattttataaagagctctccaaaaacactaacttttaggcaaaaacatcttcatttcatttcatttctcataaatcccaatttgaactaaatttcttcaatcatgataggcaatagatctaagccggtgatcggaaaactcaccggtgcagctttttccggtagccggacgatggatggtgctaccagcccaagaagtccactagatttcaagtttccatcaccaagaggcttaaaaagctttgatttcggtggtgttggattagctatagtagctgcactagaaaagtccggtggtaaaaacggcgaaattccggcgaacaaagccgtttacaaccggaattcaaacagatcccttccgattccggtgatttctccgtcgaaaaattcagctagatttagaccagaaattgaggaaacagagatggatagttttgaagaatatacaatagttacttgccgtgcaccaggtaataaaccctacacaa

SEQ ID NO: 254 - DNA, 763 bp, Lycopersicon esculentum
gtagaaaggtggtttggagataagaggccactctcattactctactcatcttggagtaatcaatcatcaaatgaaggtaaatcaaaagcacctcacttggagaactgcaagctaagtgcccaaacaaatgaacgacttgatacccgacttgtaaatcataccttccctccatggaagggacacttttcctccacacctcaaggagcttatcctccctggatcaaggggtcagatgaagaaaattaccccttaacgaggaaggtgcaacgcgacatatggctccatcaacatcctttaaactgcagtgatggaaatgttaagtttctgatagctgactgggagagaataccagggtttggtattggtgcacagatagctggaatgtctggtcttcttgcaatagcaatcattcagaaaagaattcttgttaccagctattataaccgtgctgaccacaatggttgtaaaggttcatcacgctccagttggtcttgctacttccttccagaaacatctccggaatgcagagatcgtgcttttcagatcatgaaacaaaaagaagcatgggaaaaaggggatcataaccataaaagaaaactatacgtctaaagagatatggtctggacgcactccaaggtcatggggaaaaccttggagttatttgcagccaacaacagatattaatgggaagtttaattgcttatcatcgcaaatggataggaggtgggtggcgtgctcaggcagtgcgctacctaatgcggtt

SEQ ID NO: 255 - DNA, 570 bp, Lycopersicon esculentum
ccctgctcaagtctacaatcgcgagaatgggcatgttggttttatgctatcttgctatgatgctgagctaagctatagtcgaagaaccgacactttccaggccagatatcctccacatggaaggagggcaatggcaatagagacaggtgtaacatgggatagattaagagtacctcctcttgatacctctccacatgatctttatatctctgattgcttgactgaattatgccctggtgatcacattgagattcaatggagaagaaacaaagagttcccttatggctggtggtatggtgttgtaggccacttggaaacgtgtgatggaaatgagtattactgtcgttgtcatgaaaatgatacagtagtgctagagttcaaccagtatactcatggatcgcggtggaggacaacacacattgataggaaagatcatcgcgaggaagggaatgaggctgatggattttatggaggaatccggaaacttcatagcaatgaagagatttctatgtggagacgcctgtggccatccgatgtcttggagtaagttgcaagctcaccattgttggc

SEQ ID NO: 256 - DNA, 312 bp, Lycopersicon esculentum
tcaaaagatgctggaagaactcccaggtcaaaaagcatgtttgttcgtatgtttagcgagaaatctttcaagaatagaaatggcaagagtgatcaagaggctcaactggtagatactgatagagatgttgaaattcaatgataagatccgcatgaattctatccactagcatgctttgcttaatctgtctgtgcactttcagtcaacaggtttagattgaagactattttgaaagcttttattagttcttcctgctagtaatgaattttatatttcttggacaaagtttatgaatatgtgcatcagttcatc

SEQ ID NO: 257 - DNA, 635 bp, Lycopersicon esculentum
tgctcgagaaaatcagaggaaaagccgaccctcctatttatacgattcagttatgtattcccaagcatgctcttagtttgatcttctcattcacaaatggaatcgaatgggatggtccctatagactacaatttcaagtccccaaggcttggagaaataaaccaactgaattcttcactgagggtcttacacaggagttgagccaggagggtgcctgtgacagggcaatcttcccggactcgagtatcttgatcacaagatgtgctatggttggtaacttgaatgtggatggaggtgatcgttgcaatcttgatctagtaccgggatgcattgatccagattctccccagtataatccactagccaatgtagatgatggatcttgtccaccatattcagattctgaggattaactttcagtacatatatacgactcgtgatgtttatagtttagtgttatcgatggaagtctttcacacgtgcgtgtattcgatttcaactatttacctttgtgataaagaaaatgtgtagaattactaaaaggaatgaatctccaaccttatactgtgaagtatctagttatggtgtctttactagttggtttgtaatttgtatagaactttgatacttcactt

SEQ ID NO: 258 - DNA, 397 bp, Lycopersicon esculentum
tttcagtcataacaacttgtctggacccattgctccggagattagccagtgcaagttgctgacttatgttgatctcagcaggaaccaactttcgggtgagattcctactgagatcacaggtatgagaatactcaactacttgaatttatcgcgaaaccacttagttgggagtattcctgcccctatttctagtatgcagagtttaacttctgttgatttctcgtataacaacttttctggtttagttcctggaaccgggcaatttagttatttcaattacacctcatttctaggcaatccagatctttgcggaccctatttgggcccttgcaaagagggcgttgttgatggggttagtcaacctcaccaacgaggagccttaacgccttcgatgaag

SEQ ID NO: 259 - DNA, 457 bp, Lycopersicon esculentum
aaagtaatgttgttgatgacatggttgctagcaacgccattctctatgaatctggcgagcaccctgaccatgttgtcgtgatcaagtatgttccatatgttggagacagcaagagggcaatggatgagtacatgtcagagattttcatgggcggaaagagcactatagttatgcacaacacttgtgaggactctcttttggcagctccaattatcttggatttggtccttctcgctgaactcagcactcgcattcagctcaaagctgaaggggaggggaagttccactccttccaccctgtggctactattctcagctacctcaccaaggctcccctggtaccaccaggtactccagtggtgaatgccctttcaaagcagagggcaatgcttgagaatataatgagagcttgtgttggtttggcaccagagaacaacatgattttggaatacaaatg

SEQ ID NO: 260 - DNA, 539 bp, Lycopersicon esculentum
tctggagatgattgtgttgcagttaaaagtggttgggatgaatatggtataaattatgggatgccaacaagtcaactgatcatcaggcgcctcacctgcatttctccttatagtgctgcaatagcattaggaagtgaaatgtcaggtgggattcaagacgttagagcagaggacatcacggctatcaacactgaatcaggggtgaggatcaagactggtgttggccgaggcggttttgttagagatgtatatgtcaaagggatgacattgcacaccatgaaatgggtattttggatgacaggcaactatggacaacatgcagacactcactgggatcctaatgcattaccagagataaaagggatcaattaccgggatgtagtggctgagaacgtgtcaatggctgctcgtcttgaggggatttctggggatccattcaccggtatctgcatttcaaatgtgactataagtatggcaaagaaggctaagaagtatccatggacttgtactgatattgaaggaattactagtggtgtgca

SEQ ID NO: 261 - DNA, 245 bp, Lycopersicon esculentum
cagaaaagtttggaactccggagcttgtttctattttgaaagaagccggagctacccattccaaagatcatggaaagccagccggtcctgcaaagcagctcaagcagactgtcagtgacataaaacatgacgtgcactctcagctccaacagagtcggcagactggcttcaaagtacgaaaaattgcaatgaaggtgaagaagctccacataagtggtcttaacaacgccatcaataatgcaacg

SEQ ID NO: 262 - DNA, 685 bp, Lycopersicon esculentum
gaggtcatttttagcacaacttgatcaagacgacagtagtggcaatgatgacaattacaattatcattaccaaagtgaaaagaaaagacgattgttaccaaaacaagttgagtatcttgagaaaagtttcgaggtagaaaacaagcttgaacctgaaaggaaagtgcaattagctaaagaaactgtattgcagcctatacaagttgctatttggtttcaaaatcgacgagcacgatgcagaacgaaacaaattgagaaggattatgatgtccttaaagctagctttgatgaattgaagactcaatatgattgactcttcaaagagaatggaagcttgagaaatgaggtacatttactcaaagagaagttgatcaacagagtaaaagggaaggaaattttagaatttattagtgcacttgatcatgtagaacctcaaaatccttctattggagttgattcaaatttagcaatggcgaactgcaagcaagaagatgcaaaaagtgatgttcttgattcggacagtccgcgttttactgacggagattacacttcatatttggagcctgctgattcttcaaatgtatctcagacagaacactctaatttttctcgagaaaatgacacattatgctttccaaaacttggagaacatcatcatcacgatgatttacctgttaattcttgt

SEQ ID NO: 263 - DNA, 572 bp, Lycopersicon esculentum
tggccgacgttgaatagaggtgcttcgtcggtggtctggcatgggccaccaccgaccaaacactttcggaagctttttctcagtacggcgaagtggtcgaatccaaggtctgttgcagggcagagatcggatccgagcctttggcttcgatgaccctctgttactgaattgtgatctgttactattctctggtttacttgactgttactgttactctatgttactgttactatttgttactattattccaattccccgaaacggtacgttccgtcttcctctgtttttacacgagatgaagatagatcgcttttatgtttctccccctcatgcttttttctttccgatctgctttgttttggttgttaatttgtagatctgtttggatctgacgcaattgttatgttctttttttaaaattttttcagatcatcaatgaccgggaaactggtagatctagaggatttggattcgttaccttcaaggatgagcaagccatgagggatgctattgaagggatgaacggccaggatcttgatggtcgcaacatcaccgtgaacgaagctcaatca

SEQ ID NO: 264 - DNA, 698 bp, Lycopersicon esculentum
tgctgtggctgcaaagtttgttgaagtatcaaaatgcatctgttcaccactaataccaaatgccatgtcatctacaacagttcttccagtacatgtggctcctccttcaacaagtgtggtgaccacggtggacgtctgactagcaggttcatgagtccttgaccagtctgggttcccaaaccctgtcacaaatccattgacatcgaatacatcggagatggctccgagtcggaggagcaaatcacagccatgctactcaggacctctatgactctattgcagctggaaattatcctgaatggaagctcttcattcagattatggatccagaacatgaagacaaatttgactttgatccacttgatgtgacaaaaacttggccagaggacatcttgcctttgcagccggtgggaagattagttctgaacaagaacattgataacttctttaatgagaatgagcagctagctttctgcccttctattgtggttccaggtgtttattactcagatgataagatgcttcaaactcgtattttctcctactctgatacccagaggtatcgacttggaccaaactatttgcaacttcctgctaatgctccaaagtgtgctcatcacaacaatcactatgatggctctatgaattttatgcacagggatgaggagatcgactacttcccttcaaagtatgatcaa

SEQ ID NO: 265 - DNA, 405 bp, Lycopersicon esculentum
ttaccggtagccacataataatgccacttggccacacaattgagcatgaagaaatgcttgaagttattagacttgaagggcactctattggacaggaagatgcttttatgccaagagatattcaccttttacagatgtgtagtggcactgatgagaatgcagttggagcctgttctgaactagtttttgcccctattgatgagatgtttccagatgatgcacctttgcttccctccggatttcgcattattcctctcgaatcaaaatcaggtgatgcccaggatacgttgaacgcacatagaacattagatctagcatcaagtcttgaagtgggcccagcaacaaactcgactactggagatgcagcttcttgttacagtgcacgatctgtactgacaatcgctt

SEQ ID NO: 266 - DNA, 503 bp, Lycopersicon esculentum
  misc_feature (501)..(501): Unknown nucleotide 501
acgagaggggcttaattggcatataccatgactggtgtgaaggcttctctacatacccgagaacgtatgaccttcttcatgctaatcgcttgttcactctgtaccaggacaagtgtgaatttgaagatattctcctagaaatggaccgtgttctgcgtcctgaaggcgcggtcattcttcgagatggagttgaagtcttgaacaaggttaggaaaatcgccgcgggcttgagatgggacactaaattattagatcatgaagatggacccttggtacctgagaaaatctttgttgctgtcaaacaatatttcgttgaaggcgatgaagaccaaagcacgccaaatgacgattgagtgatgccaatattcgattacatcgagagaaacctgagaattctgaattgggaaggtaagatctccaggcagagatatgaaggttttttttcttctcaaatatggcatacaacaagtagtgcaagttcccatgtagagcaaatttagnta

SEQ ID NO: 267 - DNA, 605 bp, Lycopersicon esculentum
tcagaatcttcccagatcctctggtttaccactgagaagtttcacaggcaactcaagaaagctttattccactgttgtttcactggggatgacaaaatttgctgatcagcgtctaagtattgtggcacaggttgtatctggtccagaatctactacagagaaggatgaggaaaaatccgatacatacagtcatgacatgactgcagctatgggagctgtcttgacttataggcatgagttaggaatgaactacaacttcatccgtccagatttgattgttgggtcatgtctacagactcctgaggacgtggacaagcttcggagcattggagtaaaaactatattttgcttgcaacaaaatccagatcttgaatactttggggttgatatcaatgctattcgtgaatacgccaacaaatgtggtgctattgaacacttgcgtgccgaaataagggattttgatgcatttgatttgaggttgcggcttccagctgtaataagcatattgaacaaagccatcaatagaaatggaggtgtgacttacatacactgcacagctggacttggcagagctcccgcagttgcgttgacgtatatgttctggg

SEQ ID NO: 268 - DNA, 556 bp, Lycopersicon esculentum
ggcacgagctttatttaacttcaaaattaatcaatcatgagttactttaaccgcgtttgggtagccactaccgtcgccgtcgtaaacagccacgccgatcacggccagaaattgaaatccggcatccaatctctaaaccacggtaaaaaaagattctcctcttctgtcgcttccggaaccgacgttgccggtctccggccgatgtccggtatattaggatctgacgtcggggggttcatcggaggcggaaaaggcgaggagaagaggaaacaaactgatgagtcactccggcaagttatgtacctaagctgctggggaccaagctaatgtatggtggtagatcggaagtctcccgttcgattttaggtggctataagtcgccggcgacgggaacgcggagtgagttgaccgagttgactcgtattagagggcgaatgaacttagttaactcggcctatcccgttatgcactaattgcagttttaatactcaaaactacgacctctaattgtaaattttgtgtatatcctagtttaactattgtaaatttaactaat

SEQ ID NO: 269 - DNA, 435 bp, Lycopersicon esculentum
tagtatccttgcaggacgtataatttcccttttctcttcttctccccttaacgtgagccgattcgctaacctgcaccatgagcttacttgacaagctctgggacgacaccgttgccggtccccggccagatagtggcctcgggaagctccggaagtattctacttttagtcagcgttcaaattccggcaaggaatcagaagtttcaacaccgagatcctttactgaggaagcaagtgaggacgcggtgaaggtgacgagaagtatcatgatcgtaaagccttccgggagtcagaatagagattcacctcccgtttctccggccggtactactcctccggtatctcctttttctggttcttctggaagagaagcatttcggttccgtcggcgatcagcgtcatttgcatacgagaatgccagtggggttggaccca

SEQ ID NO: 270 - DNA, 529 bp, Lycopersicon esculentum
  misc_feature (394)..(394): Unknown nucleotide 394
aaccaatctttcctatcggccaccggaaaatgtccaacgcgcctccgcttttctcatgcctataacagatctgacttttggacgaatctatactgccaatctgatccgattctgtcaccctagaattctatcaagccgtaatttcgctgcactcaaaggtccgatggccgaggaaacgaatctcaaaagcaccgtagagtcaacgatggataacaagatcaccgttgagtcaacgacgaataacaaaacggaatcggagaagaaacaggaagaatcaaaggttaccataccaccgccgccggagaagccgttgccggacgattgttgcgggagtggatgtgttagatgcgtgtgggacatgtattatgaagaactcgaggaatacaataagctntacaagagcaatccagatgttaaaccttcttagagaatctagaattcatccgtgttcatatgaattggtgtttgctacattcatgattaaaatattgaagacagatttacgaacaggcagattctttgtaaactt

SEQ ID NO: 271 - DNA, 588 bp, Lycopersicon esculentum
gggaaacaccggtgtaggattggcattcatggctgctgctaaaggctacaaactcatcattacgatgccttcttcaatgagtcttgagagaagaattattctgcgtgctttcggtgctgagttggtgcttactgacccggcaaaagggatgaaaggtgcaatttcgaaggccgaagagataaggggcaaaacacctaactcctatattcttcagcaatttgaaaaccctgctaacccaaagatacactatgaaaccactggtcctgagatctggaaaggctcaaacgggaaagtggatgctctagtctctggaattggaacaggaggcacaataacaggttcaggcaagtatttgagagagcagaaccccaacatcaagctgtatggcgtggaaccagttgaaagtgctatcctttctggtggaaagcctggtccacataagattcaggggattggtgctggtttcgttcctggtgttttggaagttaaccttattgacgatgtagttcaggtttcaagtgatgaatccatagaaatggctaagcttcttgcattgaaggaaggattgctagtgggaatatcatctgg

SEQ ID NO: 272 - DNA, 336 bp, Lycopersicon esculentum
tttttttttttttttttttcaaaggaaaatacttcattaaactaacataaacattaaaacaaagagacaatgaaaatttttacatactagtggcacaaaaaatatgtaacatggagttcatgtcatttttttcttttttatctaaatctttttatacaatgcaagactgaactccaattttcctccacacctagttatgattcgtcttgtgctggtggtgctggcactcaacaggggcaacagggaacctagcgctatccgtgcaataatcgtaaaccatatggttcgccctgacccatatcagctggtgactttggtgcaaattcaactcggaca

SEQ ID NO: 273 - DNA, 450 bp, Lycopersicon esculentum
gaacaaatctgcaggtaatgccaagaaacaagaagcagataggagaagtaccagcagtaatgctgaaagtgaacaagagaaactgaacagagttggatgggctactcaaaagagtaagtccctggatccacactctttgtactcagctcctaaggtccagcctagtttcagaagcctcactactagtccagaaagaacacaccttaagtctaagaaacatcagggagagacactacaacgcaagtcatcagacccacaggtatatttatcagttcatatgggccagtcttctggagcggacttgcatgcccctttgttgagccacaatgaacagctagagagtcaccgcatgagcacagaggaaaacatgcaaagctactcaccttcatcttctcattcactgagtatgctggaggaacaaaatagttctacagaagatgagatgaagcc

SEQ ID NO: 274 - DNA, 715 bp, Lycopersicon esculentum
ggtaataccagttcttgggttttggtgggtttgcttcaatcattgctatatatccatctcactcactttttatactactaattgggatctagtaaagagagataaaaagggtgagacccattttggaattttgggggttttttttggggggggtttcaaagtgaaaagagaagttctttatatttttttgaatgacaaagttatgaggattttctcaggcttttggaaggatttgagtgtttgcagctctaaaagtagctttggtgttgctaaaaaggaggagaaattgtaaaaaaaggatttaatagattgtgagatcaaagtgtacaacaaatggcgatgtgttgaccagaatggatcctcaggcttttatcaggctgtcaatagggtcgttggggttgagactctctgggacaactttaaatggtacaaaatcagggataagtgcactctcttctccctgtttatgcgagatccgtcttcgaggtttccctgtacagacatcatctgttccctttatttcctcacctgaagctaccgtggatattcacaatgttgcatccagcttttatctcgaagaatctgatttaaaagccttacttgaacctgggtgtttttatgcgcctcacgcatgtctagagatagttgtattcacaggacacaaaggtggccactgtggagttggtattaagagacagcaagttgggacttttaa

SEQ ID NO: 275 - DNA, 706 bp, Lycopersicon esculentum
ggttaagagatagacgtgttcgcctttctgtcaatactgctatacagttctatgatttgcaagaccgtctcggctgtgatcagcccagcaaggctgtcgaatggctgctaaaagcggccgctccttctattgctgagcttccacctcttgaggcatttccagacacactgcagctgagtgatgagaaaaagtcaagtgagcagggttttgattcagctgatgtggaaatggatgatgatcttcattataatcagcagcaacaaccttgttgtagcaattcagagactagcaagggttctggattgtcactttccagatctgatagtcggctcaaggcaagggagcgagcaaaggaaagggccacagagaaagaaaaggaaaaagaaaacaagtcttgtattgttggtcaacaccaccaaaacatgcaccctggctcgtctttcactgagctattgacgggtggtatgagcgataacaacacgagtcctaatggtgggtccatccaccaaaatacagcaaggcaatggtctacaaatccgttggagtattttacatcaagattactaggcccatcagctactcgtggaatagacaaccaaatatatttgggaaatcctctacagccattaagaccaatgtttagtattacgggtgagcatagagcggagctgcagaatttcccatttggtggtgacaacctagttcc

SEQ ID NO: 276 - DNA, 785 bp, Lycopersicon esculentum
taccaacaggcacaaacaagtaaaccacagcatcggcaagttggtggtgaaagtagccgagcaattccaatggatgttgaaaagcgtcaagtttctacaggcaacaatggaccttatgttgctgaggtgactgatcgtctaaggccgaataacccttccagtcctggaattcgtatgcaatttaagtcaccaacaaacaggaatttaacttctgccaatcttgagagaaatgtattaagtagtgctcatatgccatcttcttcattggctcctgttttaccaagagcaaatgctacaaaacctgttttacccactgaaacaacacatgatggtgataacggaggcagcagctggatgtcctccttgcgacgtatttcttctgcaaagtgataaaaaggataagatgaagatgaagttgaagttggggctagtgagccttcaaattgggcagctgatacttactttatgattacaagctggttcggatgactatggtggctgatcatggcccgctgcaattttatgagttcaagagggtggtgtgaattgagcaggacatgaaagcagatggcagctgtttgtctttctatgggagctcgggaatcattccatttgatgatatgcccgtgagatgttgtttctgtcggtgtaatgattccccttgaattcaatcaactccagaagaagattaactagtgattgcattgtttggtttgttgattgtgcgaagggatcagtattcactggttccttggtaccatcttctgcctgagctatattcctgc

SEQ ID NO: 277 - DNA, 340 bp, Lycopersicon esculentum
ccagagcatgcaccaccaagcacaagatagaggaagagatagccgcagtagctgcagttggtgctggtggatttgcattccatgaacatcatcagaagaaagaagcaaaagaagaagaggaggaagctgagggaaagaagaagcatcatttcttctaatgaattctctttgtcataacatatagtatttgttgtgtgctgatcaatctatcctttaattaatacgtactcagcatgcacctaaataatttggtatgtgtgttacactactcttcttttgatgttgtgcacctactttaatttgcatgagtgtgaataatattacgtatattgtttttctt

SEQ ID NO: 278 - DNA, 654 bp, Lycopersicon esculentum
ggttttggaaatatgggtatctacagacagcaaaaattggcatttttctttgtggctttattgggctttattcacatccgtctgaataatgctcaaagttccacaacacttgttccggcaatcattacatttggcgattccgctgtggatgtaggaaataatgactacattcatactctctttaaggctaattatccaccttatggaagggattttgtcactcgtcaacccaccggtagattctgcaatggaaaattagctactgatatcactgctgatacacttgggttcactacttacccggctgcatatttgagccctgaagcatcaggcaagaatctacttattggtactaatttcgcttctgctgctgctggatttgatgataaaaccgccatcctcaaccatgcaattccattttcaacacagatgcaatactacaaagagtatcaatccaagctagcaaaagttgcaggcagtcaaaaagcggcatccatcctaaaggatgcactctacataataagtgcaggaagcagtgatttcttacagaattactacatcaatccgtatcttaacaaactctacactcctgatcagtatggttcttatcttgtcggcatattcactagctccgtcaaggatttgtatgggcaa

SEQ ID NO: 279 - DNA, 315 bp, Lycopersicon esculentum
gcttcaatccgttgcatcattcgctgctcggaaagagatcacgaagagatcaattcttgatttgtattcaataatatcagtgatgcaggtaaccaggatcaacacgcagcgctttgtttatccagattgagctccaaagtctgttgtgagcctccttgcattccggaggacatctatttcattttctttagtatttttgttttaggatgttgtaaggtaggcttcatagactgttagtagtttagtttggagttattttacttgttgagaatcaagtgccttttggccagattaatttatgaagtatttgttttt

SEQ ID NO: 280 - DNA, 397 bp, Lycopersicon esculentum
gcaggaatcaaagaagatggtaagctagaccaagctcagttagtcgtagattatttgaagttgtgtgagagacatccagtgccatggagaatgatccgttctcatgtgcacaagatgctgggagagtggtttaggatcgagccaagcgttagagaggatttcaaccagcaatcccaactcaccttcgaattcctttatggcttggtaaatcgattaagggaacttggagtgagtgtaccactttatgtgaaggagactcaagaggaagctgcatctccaaatggagacggggcataaattatcttctgtaagttcgatttgattccacttagttgtacaatcaaaacatagattgtcattcttgctggcaaaaaggaaagttagatcgtcattcttt

SEQ ID NO: 281 - DNA, 369 bp, Lycopersicon esculentum
tttttttttttttttttatcgactagcaggtcagaactgcataaataatccttcaagtaaaagtctattcatgtactgaaaaccaacacttgctatttctgccaaaaaaattatatgaattagggatgcttccttccacaaatctaaattttgatgcttctcaaagtatactaatgttttggagaactatgaagacaaaaaagctgttggcaacatggcttgtactgaaggtgtattgtttgtccttcatgaattcaacttccagcttattcccatgagtattgctcatttctcaagctttgctgattgctagggtaagctctttcctgccgaggagtttgtctataataaccatttccccttgatctg

SEQ ID NO: 282 - DNA, 660 bp, Lycopersicon esculentum
taatgttagaattggtactggcaatacagttggtgctcgtacctccaatatatttgatgatctagacaaatttcagagcagatatcaaggtgccttcaacaaaaccaatcctaaggcttcagaagaggcacgaatattagcatttcaagcaatccgagctatatcaaattttttctactggcaatcaccaattggaggagatgcagaagacatccatgctttggtaccagttaggagaaggctaattgaacccaacgttaagaagcttagacatgttgtagaagctatgaaacaagtcaaaaaaatcaacagttaatcaaagtagcaacgatgagtattggtctccatcaactcctacacttgcggacaccagtttggtagttggtgaagttgattccttccccaaattaccaatggaattctatgatgaagctttctcaccaagggagaaacatcaaggtagtcccagactcgggatagtgccattcacaacagcaacagcagcagcaattacagtttggataaaacttggagaaaaacgagtgttagactcaaagatcatattggtggttctctcgcattagacattgtgaacagcccatggctcaaggtcattctggcaggtattacatgtatatgatttggtatggcactagtgga

SEQ ID NO: 283 - DNA, 3818 bp, Lycopersicon esculentum
ctgggatttttagttgaattttagtgctaggagatggtggggttttaatttttgaagcttttcttgaaaaggggttgtttttttatgattaaactgctgaaagctgtttgcttttttgctcttttgatagaaaaagtttgtctttagccttccatctgatattttgttttagtaatggattaagcatttgttgaatttttgttctggggcttggatttgagttcttgattgtgtttcagagcttaaagtttgatttttttttgctaagtttgatcatttgtagaatcccttttggatgaaatggcagtttctttgactcgtttgtcatggtggtggtggggtgggaaagagaaggaaccagtgtctaatggatcttcaatgaatcccttgcaagattttggatttggattgaaggaacaaagtgatagtttgaagtttaagtcagtgaggggggctaatctagcttcaactactacgaaagtgaagaggaaatggaagagtagagaggagaggaagagggttgatgagaaatatgatgtggtattggtgccatctgatggtgtttgtttatcaggttccgaatccgatgactctgactggtcgatcgggtggctggagccacatgcccctgatttccagaatgatgatgagggagatgacagttttgctgtgttggttccttgctacaggcatgattgtagagagataggagagccaaacaatcagtttttaagtgccattgagaatctctcaaatgggtatcctgctgtagaaaggaagatcaagaattggttgaaaatgttacaaagagctgcaactaatgcttattcatggtgggctgctagccatattaggactaaacaatccaaatggctagaacaaagccttcaagatatggaagagaaggtagaagatgtgatcaagctcattgaagaagatggagattcatttgctaaaagagcagagatgtactacaagaaaagaccagagctaataaactttgtggaagaatcttatcgtgcctatcgagcgttggctgaacgatatgacaagttatcgagggagttgcagagtgccaacaacaccattgcaacactttttccagaacagattcaactcgcgatggatgaagaagatgattatggtacaccaagaatgccaaagaacttccctcaagtaccaacaactggtgcaaatataccaaacgttcctccaaaggctcctgttaaagacatgaaaggtttgttgaaaacaggcacactgcaatttctaggcaagaaaacagctaacaaagcaagagatgcaaacaaagaaacaccaaaatctggtttgactaaagacgaggcacttgaagagattgacaagctacagagagatatactgtctttgcaaactgtgaaagagtttgtgaaaagctcttaccaaagtgggattgctaagacgatggaaatcgagcaccagattgttgagaagcatcaaaagatttgcagtttggaggatgaatttggtgaggctcgggttattgaggatgatgaggctaggactttgatggcggaagcagcattaaaatcgtgtcaagaaacgttggctcagctccaggagaaacaagaacagtccaccaacgaagcaaaagaggagtttaagaagatagaagaagctcgtaagaaactcaagtcattcaggcaaaagtacttaggtgatccagctgatgaaacagaaccagatgaaaaagatgatgaatctgctggagtaacagatgtatcatcacaatgcacgagcctagaagaaatcggtgagaaaatggagtcgttgcatggcaagatgaacgagcaatttgatgcaagctctatgtcatctctaacagtgacacaattggcagagaagattgatgagcttgtgaataaggtagtcagcttagaagcagcagtttcaacacaaactgttcttattgagagattaagaacagaagcctgtggactccacacacacattcggactttggaagacgataaggcagctactctgacaggggatacacacaatctgaacgttagagtgaccatgctagaggaaaagttgaagggaattcaggatctaaacaaagacgttgaaactcaaaacagcagccttaagactcactttgctgaagctcataacagtcttggccagttatcgttcaaattgacgagtgtgaaaccagatgaagaagttgatgagacggattcatcacaagatgaagaggaggatcttgctgatatcaggttacaaaatgagccagaaaagaaagaaaatcatgttagtgcaagtgaagctaagaaagaacaagatcctgttagtgcaagtgaaattaagaaagagcaagatcctgtttcagttgttagtgataaagaagttcaggaagatacgaaatgccttaagaaacatgtcgaacctctagagccaacagtagctgaaaaaggtgaagagagagtctcatctaaatctgaaagtagtgttcatcatgagcaaaaaccactggaagacgaggacaaggacgatgaccttacatggcagcaaatgctgttgactggtttggaagataaagagaagatcctcttaacagaatatacaacaattctcaggaactacaaggaattaaagaggaaactaattgaaatggagaagaaagaaagagacactgaattcgaagtcacgttgcagataagagagctaaaaagtactatttcaaagaaggatgaagagatagaaagtttacgtctaaagttaagtctggtacaaggaaatgcttctgaaagttcagagtcgaaagaagaaaaacatcaagatctcaatccttctgatgatagaagcttaaaacctgaggacacgcctaaaaatgaagagaaggatgaacaggatgccaagatcattttgattgatcaatgttcatccctctcgccagttgaagagaaactccggatgggcattgatgcattactagacgaaaacttgaatttctggctaagatttagctcagcatttcatcagattcaaaagttcaagaccacagcccaagatttgcagggcgaaataacaacactcaaagaaaaggaaacaaaagagggcagctccaaaacagacatgaaatcagaaatcaggcccatatataaacacctgagggagattcagaatgagctaacagtatggttagaacaaagcttgtcactaaaagatgaactgaaacgtagattctcgtctttatgcagcattcaggaggaaatcacaaaagcactaaaggacggcgttgaagaagatgaaatcagattcagtagccatcaagctgcaaagttccaaggtgaagttttgaatatgaaacaagaaaacaacaaagttagagaggaactagaagctggtgtagagcatgtaactacacttcaagtagatgttgagaagacattaagaaaactagatcatcagtttgatgttggtggaaatcaaccacaattaacaaactcagcaagtagatcaaggatacctttgagagcattcatctttggaacgaaagtaaagaagagtaaacgttcgttttttcatcacaataggaaataccaggtcctgaaaggtggagtacctttatagttgtctttttttttccctttttccggttttcccctttgttctcattcttctccaggtattttttcatgtatctggataggcaagaatgttggtcatacttttttttttctttttttttgagtgagaaggtttggggattgtatgtatggtcttgatgtgtaagttagtgtaaacagataatttatataacagtaccattc

SEQ ID NO: 284 - DNA, 561 bp, Lycopersicon esculentum
taccatcaatccatcaacttatggtgaactggaaaatccttctgctgttcccttggtaatttcaagcaatagtcatccaaggtccggaagatctggttctcggcaccgttcttctcgctctcggatgatccctgttgctggagctggtgatggcgctgctcctgttaatgttatgcatgatgagagtgattcacaaatttctattggtcgcatccagtcagagatagctacttcacttgcagctgcagcggctgctgaattaccttgcactgtgaaacttaggatttggccttatgatattaaggttccatgtgcagcccttgatgctgaaagatgtcgcttaataatacctcatgccgtactttgcagtgaaatgggagcccatttttcaccatgtgggcgatttttagcagcttgtgttgcatgtattctgccgaacttagactctgatcctgggtttcatggtcatcttcatcatgatactatgacagcttcgacttctccaaccagacatccagttgctgcccaccaggttatgtatgagctacggatatattccct

SEQ ID NO: 285 - DNA, 750 bp, Lycopersicon esculentum
tctccggccaaatcctgccggaaaactctaatttttaagcgacttttgcatgtagacttcgaaatcaaggactgctattacttgtcagcgaattgaagcaatagaaggaaagatgatgcaacacaaaagaagtccaatttccctcgagcatagcagcagcctcacatctcttacaccaaaacgactaaaggccgatatgctcatttcctccaaggagaagaaggagaagtttggtgaacgtatagttgctcttcaacagctggtgtcaccatatggcaagacagatacggcttctgttctccttgaggcaatgggatacataaaattcctccatgaacaagtgaaggtgttgagtgcaccatacctcggaactatgccaatgtcgaagacacaggaatcacaaccttacaatttgagaagccaaggcttatgtcttgtgccggtatcatatactgtgtggtgtggcaagcagcaatggtgctgatatttgggctcccattaagacctcacaaaagttctagtccagagaacgatgtgtgatttcaccatcactaacgtccatggtaaggtcttcgatttacaataaccacaccagtgtttcaaaaaaagcagccgaaaggatgcatttttcaacatggagatgcagggtatgtgatgattctcctggcagggtccacttgattttttgtggttcaaacgtctaaggagacgtagagcttgttgttctgataatgctagtagccagatcta

SEQ ID NO: 286 - DNA, 601 bp, Lycopersicon esculentum
ggatctaaaaaagttgatgtttttctgaataaatcagcaaaatttaaggatggaaaatgggtttactgcagaagatctgtcgaagattggaggtattgctaccgtatcactgcttcattccttcatcccaactcattggttgccattttccattgtgggtcgtgctcaaaaatggactctttcaagaacccttttcgtcactgcatttggagcagtattgcacgtgttatccacctcacttcttggcataacggcaataacgatatcaaacacaattgctggggaggaaacagtgcacaaactggcttccttactgcttgtagttcttggaggtagctatgtcatcttacatctctttggaaagggtggtcatagccattcacacaatcaaccaatggagaagatggctgtggcagggcttatacttgtccctgctttgtctccttgcgcaaccactctccctgtatttcttgctggtggaaattcgtcttccatgatggtgcttgccatcatagttatacttttcagcacgagtactgttatgacctcattggtggctctatcattttatggtgcaagtcaactcaagtttcatggggtg

SEQ ID NO: 287 - DNA, 702 bp, Lycopersicon esculentum
gctcaattgcatgtatgtcgatgtttggaactctagagcttcaaccagaagttaatgaagtagttgagtccatggtggagcgcttaagaactttgagctcaaagtcaaaaggacagtttgttgcagtggatctgagggttgatatattggagaagaagagttgccaaggtgacagttctcttaaatcaaagagctgctatggtccagaggagattggtatgttcttgagaaaaattggttttaacaaggatacaactctatatctgactcaatcaaggtgggatagcagccttgatgcattgaaggatctctttcctaaaacatatacaaaggaaagcataatgcccattgacaagaaggcaaaatttctcgactcagagtcttctgaactggagaaagtaatcgacttctacatgtgttctgagagtgatgtttttgtaccagcaatctcaggcctcttttatgccaatgtggctggtaagagaattgcttccggaaagacacagattcttgttccagctgatattcctggctcttctgctgcttcgactgattacatatcccattatgtctcaaagaagaaccacttcgcctattcatgtttctgctagctccctgtgaagaaaggctgaaaatgcagatggttgagattgcggatttcttctacgttattccttgcacttatgtttgtaagaacacagg

SEQ ID NO: 288 - DNA, 1887 bp, Lycopersicon esculentum
ggcacgagattgatcctcattctgcataagaaaattccggtacatttcatgccgtgtcggtgtcagcaaggtacaccaaaagtagtaaacagggtggcaaaaaagaggaggatttagcaatttgttgatttttccttctttctttctttcttgtcaacttcaaaaccccatcggctctctttcaaggttcccctttctttctctattaacgcctctctctgagttcttgttgatatttccaaagaccccatttttaacactgctcacaatatggctggcgaatccatttcaggcaatttaaaagacctttccttgaatagaaatggtgcggtgtcaaagaagtctcatttagttgtttgctttggggagatgctcattgacttcatcccaactgttgctggagtttcacttgcggaagctcctgcctttgaaaaagctcccggtggtgcacctgctaatgttgctgtgtgcatctcaaagttagggggttcatctgcttttattggaaaggttggtgacgatgagttcggccgtatgttggctgacattttgaagcaaaacaatgttgacaattccggcatgcggtttgatcatgatgcaaggactgcactggccttcattacactcacagctgaaggtgagcgggagttcgtgtttttccgtaatcctagtgctgatatgcttcttcgggagtcagaactcgatgtagatcttattaaaaaggccaccatcttccattatggttcaattagtttgatcgacgaaccttgtaggtcaacacaccttgctgcaatggacattgccaaaagatcaggtagcatactgtcgtatgatccaaacctgagattgcctttatggccttcagaagatgctgctcgaagtggaataatgagtgtatggaacctagcagatattattaagataagtgaggatgaaatttcattcttgactggagccgacgatccaaatgatgacgaggtggtgttgaagaggcttttccatcctaatctgaagcttttgcttgtaactgaaggttcagctggttgcagatattacaccaaggaattcaagggaagagtaaattcgatcaaggtaaaagctgttgatacaactggtgctggtgatgcatttactggtggagttctcaagtgtctagcttctgatgctagtctttatcaggatgaaaagcggttaagggaggctatcttttttgccaatgtttgtgctgccctgacagtgacaggaagaggtggaatcccttcccttcctacacaagatgcagttcgacaaactcttgccgaggtcactgcatgagaaggcagaacaaagttttgttctcttcacactgtatctgcattattctagatttattttcacaatgatcgatttattttgttttcgtctctggcatctgttggtcggttcctctctttggaaagaagttgcagccaacgagacatgcagggaaaaatagggtagcgcgtcttctgtcagtcatgcaaggaaatgctggaaagcctttttcgctaagtcaaaatacaagctgttatgtctccgttacatatctgatccttgttacggatccatcagaagccaagatagtgaaggttgttaacattggttattgagatttactgcgtgtagagagaagaacaaaaggtggacatgcatttaacgactatcagcttttgtttgttttaatatgtttccttttcaagaacctttctgtttttgtttcctttaaagtgtctgtattataaggtgacttcaatgctgtcttgattagaaatcagcagaacaaaaaatattacttatgcagttatgtggtttgatgtactactcagaaatcagaataatatgagtctcatactgttgatctcttccatc

SEQ ID NO: 289 - DNA, 532 bp, Lycopersicon esculentum
gcactctggtggtggaacagggtacctcgctttatcagtgcaatagttataaacagtgtatttttgacgaacccaacgaagtctcctatactgtaatgcatctaaatcttggaaggccttttgatcccaccatttcatgcctttagtgttacaaacttggacttcttgtggcgtggcagcttcacatccatccacgtggaacgatgtgtatgacgcggtgaatggggcgttggcccaattggttttctcaagcccacctcttgtggcccaatcatctgcgtcccatagactcgagtatatcttcatgggctgattgaatggaaatttcacaccaagatctttcgaatttttgaatgctctaattggaaagtcgtccacaaagatcacaatgaggtatgtattccaaagaacagaataagaatggtagcccttggttggatcaaaccaaagatatattctctgttctctgtttccttttcctcctgtgaatacatttgtctgcaatatgtatggctgcccagttctgttcccc

SEQ ID NO: 290 - DNA, 553 bp, Lycopersicon esculentum
ggggagctaaagttccagatctgagcgatctcagtagcgctgatgttgtttcgttgttgcagtaccatgctattccgagttacactcctattggaaccttgaaaacgacgaaggatccgatttcaactctagctactaatggcgctagcaagtatgatctggcagtttcaacagaaggtgaccaggtgacacttgacacaggtgtcgattcgtccagaatcgcttgcacagtcattgactctactcccttctgcattttcaccgtcgacagtgtgcttctacctgtggaattgtacggaaaagctccatccccagcaccaggaccagctccggagacttcttcaacaccagcaccagcccctgcttcggaagccagctctcctactccaatgatgtctcctcctgctccaccgacttcatctacagctggttctccggcggatagtcccactgcagattcggagaacagtacggcgaagaagaacgccggtaacgttaacactccggcgctactgaaagctctactcacagtgtcagtttcggtaattgtgtc

SEQ ID NO: 291 - DNA, 559 bp, Lycopersicon esculentum
gccaacacattgttgatcatagtaaggcttccaccgggataagtattggcttttccagtttgctaattttctctcacacttctctgccactccgctccaaatgctaattgacttactttttgcacctaatgtattgacatgcccaagtagatggtgggtagctcccctatctggccccctagtgtattggctagctcctcaatttgtaccacttcattaattggatagcgtagcttgccccccagtttatatgaaggcctgacacagcttcaaaaataatgaaaatgatcctcaaaatcagaaaatggtccctcactgcttcagaatactgtaggtgagtgatttctaggtggttacttgccctgttttgctcttggaaccctctgatgcagtctttggcctttgttgtgttgatcaggctactcatcccctccattgctagaatgaacaagaaaggtgagagcggatctacctgcctcaaccccctttgagaagagaaaaaaccagttctattgatcataactgaaaatttgactgtgcctatacaatatttgatcca

SEQ ID NO: 292 - DNA, 438 bp, Lycopersicon esculentum
gcaaaactgtaacatctatgctcgaaagccgatggctaatcaaaagaacactatcacagcccaaaacaggaaagaccctaaccaaaacactggcatttcaatccatgcttgcaaaatcgtggcaacacctgatcttcaggcgtctaaaggaagcttctctacatatctaggtcgaccatggaagatgtattcgcgaacagtttacatgttatctaacatgggtgatcatatacacccacatggttggcttgagtggaatggtgatttcgcgctgaacactttgtattatggtgaatacatgaactttgggccaggagcagcagtagggcaacgcgtcacgtggccagggtatcgggtgattaagtctgtcgaagaggccagcaagttcactgtatcaaagtttatttatggatcatcttggttgccttcaactggggt

SEQ ID NO: 293 - DNA, 526 bp, Lycopersicon esculentum
atggtagcattttacaatgttagagcagtaatgaaattataattttttttttctagattttttcatgcatgtttcaacatacgttttgtagtgacagttaaaaagtgcattgttaagccaactgattgtcttatctctcatgctgcgatgaattagaagcttgactcgtttgttgttggtctcctcaagtaatagcgcaagctgtttgcaagaacctgttgaaggggtttgatgagagctttgttcttgtagcttacatgaaattttgccttagctagcagtccctcggtgtcaaattctcctgatataacatcaatgtttatttcagtaatgatcttgacgagatcaactattaatccaggtcgatctgctgcctctacacacagtaagcttcgttcaggaccatcatcatagacatggatatgagttgctatgtccacatctagcttttgacttggttgaaaagcaccaaaagcttcacccatagctaactgagagctagattccggatgaaactccatcaaat

SEQ ID NO: 294 - DNA, 489 bp, Lycopersicon esculentum
tttagcacatgaatctctctccgttagcctcgaaacagcccaatcaacatccgctatgatgttgaaattgtcacacagtcaaggcatgacgcctagagaagtaggcgccatgcaagactgtgtggaggaattaagtgacacagtaagtgaactcagaaaatctttaggagaaatgaagcaattgaggggaagagattttgacatgaaaatgagtgatattcaaacatgggtaagtgctgccttaaccaacgaagacacgtgtacggaaggattcgccggaaaagcgatgaacggaaaagttaagacagcaataaggggaaagattgttgaagttgcacatatgacaagcaatgctctggctttgatcaacacccttgatgcccttcatggctaattatatatatttgcatgtgttatatattaaaattaatcttcttttaaaaagtgtgtactagttcattgctgtaatataatagataagatatcttc

SEQ ID NO: 295 - DNA, 452 bp, Lycopersicon esculentum
gagaatggaagaattagtgatcgagaggcaaaagaggattgcagaaagaagtgcagctaagggttcgtccccagcagcttccaagaaagggccagcaggaagtaaaacagcttcaaagatctcaccttcatccaaacttcacacatttccagtgcaggtcaagcattaaggagcaatccatcaaggaaaagtatgatccgaccttaaaatatatttgattgctttgccgcgaatatggaacatccacaattcactgctttgtgcacattggattatccacaaatatatcttggagaaataaaatattttcgatgaaaattatagatttttttctttactaggacagctctgcttgctggcttgatgcaagttattccttctcatttatttagacattcatgtcccatcataaggttttgccagtatagttgcttgaggctttattattattt

SEQ ID NO: 296 - DNA, 498 bp, Lycopersicon esculentum
gcacgaggttgagaaggagaagagtagtggaccaaggccaaggaagaacaagtacagaggaataagacagaggccatggggaaaatgggctgctgagattcgcgatccacagaagggtgtacgcgtttggcttggtacattcaacacagcagaagatgctgctagagcctatgatgaggctgctaagcgcattcgtggtgataaggctaaactcaacttttcagccccatcaccaccagctaagcgacagtgcactagcactgtcgctgctgctgatacaccaccagcactactccttgagagttctgacaactctcctttgatgaactttggatatgatgtccagtatcagagccaaactccctactaccccatggaaatgcccatagttagtgaagattatgaactgaaggaacagatttccaatttggaatcgttcctggaattggagccatctgatcaattttcagggatcgtcgattctgatcctcttaaatg

SEQ ID NO: 297 - DNA, 545 bp, Lycopersicon esculentum
ttactgttcgacgctatgaagatcaaatggtgccaagcatgacgtcatctttgttcagagctgaagatgaagagaactccgtggacattaggaattcagttgtggaaagtagggcagaagttccaacagacatcaatgtagctgaagttcataggagagacacgcgtgaccaagccgtcatgcaaactgctcgaaccaatgctaccccacattttaattttgcggtaggtaggaggaattctgaagattcaacagcagaatcttccagtagtactagagaaagggatgggggtgtagtgcctacatggtctccctcaagtagtaactattcggaccaatatgttggtgatgataatggtattgggaccagttcatcttacttgcaaaggcatccacaatctcaccagctaatgaactggcagcggctttctcaaacagggtgatcatttatgtggagttacgatagcaacaaggcggagaagcaagtggctaatacagacatttggctcagcacatttagtacagcattcatctactctttctct

SEQ ID NO: 298 - DNA, 434 bp, Lycopersicon esculentum
ggaggatacaaagctggtgcacttaaagaaggattaaaacataattatgttaaagattgcgaatacgttgtcattttcgatgccgattttcgacccgaaccggattttctccgtaggtcgattccgttccttatacataaccccgaaatagctcttgttcagggtcgatggagattcgtgaattcaaatgagtgtttattgacaagaatgcaagagatgtcattagattatcattttacagtggagcaagaagttggctcatctactcatgcattctttggttttaatgggactggtggtatatggagaatagcagccattgatgaagctggtggatggaaagatagaacaactgttgaagatatggatcttgctgttagagctagtcttaaaggatggaagtttgtttatcttggtgacctccaggtgaaaag

SEQ ID NO: 299 - DNA, 546 bp, Lycopersicon esculentum
atttagactaaaaatgggctatattgaacaagggcctatgatcctttcctaattatctgttacaacttacttttagctggactaccaaggatagttctagctcagtacaaatagtggactattcaaatcttcctcacactcacttgacatctcattattctaccagattattcagacatttagctagcattttagacttagggtcatgcagtggtgtatatctatgtcatgcttagagcgacacatttatcaattactatgcctagtcatgcattattttcatttcatcaggatatcattttacccctcatgcttcagagttaaactatgtacacaagatataaacatgtcggtttaacagtaaaaataatcagtctcttggggaaaacaaacacatgcaagaaaatagtaaaagagcatagtgaattgccatacttagagttcaccctagcactcacattccatttaccccacccccaacaaaaagtcatgcaattgttcgcaattcataaaaagaattgaaatacgagagtggtaggtgaagca

SEQ ID NO: 300 - DNA, 643 bp, Lycopersicon esculentum
caaatggctgtctcatccatagccaatctcttctccttcttcaacccttccaaacccccaacacccaaagctccaacccttcaattctctccctccgccgttgattctctcacttcttcaagacctaacaatgattcttcgtcaatggtcgtttcatccaatgacaatcacaaatacccaatattaccagtatcgtcttctgattccgatgttatggccgtaacttgtccctcacttgcttatgcgaacaccctttacttcagatcggcttataatgttcaggttattgtgggtgataatgaaccggaagaaaagcttctcggccgatttaggagggaggttatgagagctggggtaatccaggagtgcaagcgtaggagatattttgagaactcgcaagatgagaagaagcgcaggacgcgtgacgctgctaggcgcaatcgtagaagacgtggcccaccaagaaacttctcagatgataagcaagaaacgactaagagtaagagggatgacgatggggaagataactgggaacttcctgatggaggtgttgccttttggattattgtagtttctttttcctttttttggtccttctgtagaaagtggtttatttcccactttggagagaattctattcatg

SEQ ID NO: 301 - DNA, 366 bp, Lycopersicon esculentum
tagtcctctttcttcaacatacaaaccaagaaaatacagcacattcttccctgtacatagcactaaaatcttgtgcaaacacaacccagatgacccacaagactcgttgaggaaaagtgaaaataaatttgaaaagttggctttagctgcagtggcagatggggttttgacactgggctcagtggatccagcatcagctgctaagactggtggcagaattggtggtcaggcttttcgaccatcatctcctcctcgctcttgctcaccaaggatcaataattcatgaacaaatatatatgtgaatccaccagttgctcctcctctagtaggtggatatggatatggctatggtattccattctatgg

SEQ ID NO: 302 - DNA, 563 bp, Lycopersicon esculentum
atcaacagagaaattaaaggtcaatcgacatctgacctacaccggcaggcaactcgtgaccttgagtctgctgtatctgcatggcactcgagttattgtcgcctgataaaatttcaaagggactttatccgatcccttcatggttggttcaagctcacccttgtatctgtcaatactgagccaactaatggcaacagggagttctcagattcttttatgttttgcgatgagtggaagctagcacttgaccgtattcctgacactgttgcatcagaagctatcaaaagcttcataaatgttgttcattcgatatatgtgaaacaaacggaggagttgaagatcaaaaagcggactgaatctgcatcaaaggagcttgagaaaaaggcttcgagtgtcaggaacattgaaaggaagtattacaattcattctccatggttggcattggacttcctgatgggggacctgataatggtaatattttaaatgtacgagaccctcttgctgagaaaaagctgagcttgcagctagccaacggcgtgtggaagatgagatggtgaagcat

SEQ ID NO: 303 - DNA, 489 bp, Lycopersicon esculentum
tttaagagagaaacaaggttcacatctaaatgctcggccaatgaaattatcagtaagattgaagaagctgcaaagcccctcggttttgatgttcacaaaaagaactacaagatgagacttgaaaatgtcaaagctggaagaaaagggaaccttaatgttgccactgaggtatttcaagttgccccttctcttcatatggttgaagtccgaaaggcaaaaggagatactttggaattccacaagttctacaagaatctttcgactagtctagaggatgtagtgtggaaaactgaagaggacatgcaagctaggtagtattactaccatgaactgcaattgcataagcttttgctggtcccacaatctgtgtaaaaaactttgcaacttggttctttctttcccttttttttgtcatttggtctgttctttgtagtgggacttttttcttccatgagctaatggccaactggtctatttcatcatggagta

SEQ ID NO: 304 - DNA, 900 bp, Lycopersicon esculentum
  misc_feature (804)..(863): Unknown nucleotides 804, 847, and 863
gaatatgctcatctggggagaaagaaactaagtcagtcatgtccctttttgatgactttataaagagttgggataaaaggcatcctggacaaccgaaacctcggctctttacagttggcagacttgatgttgccacgactggcttgattattgtgaccaatgatggggagttcacgcaccagatttcacatccttcatctaatttgtcaaaggaatacattgcaactatcgacggtgaagttcataagcgacacttgatagccattagtgagggaacaattattgacggtgtccattgcaccccagataatgttgaactactaccagggcagcctgacttatcaagacctcgtctgcgcattgtggttcatgaaggacggaatcatgaagttcgtgaacttgtgaaaaatgctggacttcagttgcgtgcactgaagcgtatacgtataggtggtttcaggctccccgcagatcttgcgcttggcgagcatgttgagttaaatcaagctaatctgaaggcattgggttggaagagttaaagttggatgagatcagaaccaagaacagcatgcatgcatcgaatcgacataagaaccggggattatgaagaaaaattagacagtgactggttagtagcactcacttatgcatgttctgttccaaatcttgcagatctgctaaggtataagggtttaatcccgtgcaaaacgaagtgccctcatatgtctgactgtcaacaagtaaatattcccaaaaattggggtgctttaattaccctccattggattaccatcttcttttggnctattttgcccaatttaacaagccaaccagggatcttggtaanattgtaccctggcccngggcggggccggttcgaaaatttccagcacaacttgg

SEQ ID NO: 305 - DNA, 551 bp, Lycopersicon esculentum
ccggtccggaattcccgggtcgacccacgcgtccgcacccgccgaaggccagagagaaatatctcatcttctctccaccggatcggttcaatcgagagcgagggagaaagggaggtggtgcgtcgaggggggccgagattttgccggtttggagccagataggggtatgagctgcttcgcgcaccgccgccgctccgacgtcgcccgccgccaggacggcctcctcgtcttcgtcttcttcctctcgattcccatacgtaccagtagttttaatttaagtagtagtaattgtagtagcaatctcttgtaaactgcgctcgcctttgcgtgttcgtggtccttttgcgctttctcgtccctctttctctcaatctctccttattcttctccttccacggcgaagccggcggctggtatggatcccaatggcctcaaaactggaggccttttgctcccgaccatcgagaggcggtgcacgtcgcctccatccgtcattgtcatcggcggtggcatctcgggcgttgcagctgcccgtgccctctccaattctt

SEQ ID NO: 306 - DNA, 565 bp, Lycopersicon esculentum
ggtatccttttctcttaactctcaccttcttctttccaccgtttctttctcagcttaaatatttctcttcttttctttttctccctttgcttgcttctctttatctcctctccttcaagatggcagataacaacaccacaactcaagattttgatggttcctctcagactacttctactggtaacaaatatggaggacttgctccaaagaagaagcctttgatttcaaatgatcatgaacgtgcctttttagactccgcagactgggctctatgcaagcaaggtgcaggagttgatcaaaaatcaacggtggctatagaaacattgcgtcccaaattacagagaacaactcatcaacaattgcctcctagaagacctgcttgtacatcttgattcacatgtggtaccttcaaaatgctgattttgttatgttatttgctggctaagtagacataatatgtcactataaatacatggttcttttcttcgatatgggacttggatgtaatcaccaataactctttaagaacttttgttgcttaagcatttataaattattatttccc

SEQ ID NO: 307 - DNA, 623 bp, Lycopersicon esculentum
tttgatcacataacaaactatttacttgctttagttaatgtgttgaacgcaacatttggtgcacgaaatagaggtgtaatttccagggtgaatgtatcgtatctgatgaaaaattcaaccaatattaacctacccaataggtgcaccaaatctttgcctggacattgcttgttatctggtgctggactctctgtttcccttccattagaccataggacatgtttcagcattttctcaccatcattcatgaacctatcaggtacaaactcatccgggtttgcgaaaatcttatggtccctactagccatgggctgatatccaactacgaattgtcctttattgatcttgtaagatgcatcgtggctttgaaccatgaactctttcttcgccttaccatactgtaatggtactggtgggcgaagtctcaatgtctcgtacactaccgatttgaccaaactcattttgttaatcgctgataatgtgattgcaccaccttcttctttaataacactcctgatttctttaacaagttgtgtgtgtagactagcccctgcttcgcccacaaacctaattaatcacatgagagatgaaaagtacattttagatgagatggttacacactt

SEQ ID NO: 308 - DNA, 524 bp, Lycopersicon esculentum
atcgaatacggccccaagacaattcaagtgatcactaaatcagcggtgaattgtccgatgaggaaaccattgccggagaatttgaactatccatcaatagcagcactgttttcaaccgcaacaaaaggggtatcaagcaagacattcttcagaacagtaacaaacgtaggcgacgcaaatgcagtgtacagagtgaaaattgaggcaccgaaaggggtgaccgtgagtgtaaagccggcgaaattgggattctcagagaagataaggaaattgagttactatgtaacaatcacagtggacagtaagaatctggtattgaatgattcgggtgcagttttcgggtcactttcttgggttgatggaaagcatgtggttcggagccccatagtggtaacccaatgagcccattgtaaatagtaaactaatcttgatcctgtttgtttaaatcagaaattatgggagataatatgggatagatgtgctaatcacattaaacaagtagattctacacttttttagagtgg

SEQ ID NO: 309 - DNA, 591 bp, Lycopersicon esculentum
taccgtttacccaattccctcttcgtcgtcccttcttacttccctctcaggggagaagaagtcaattcactgcattcgctgcaaagaagttcacgtcagggaagaacaagagaagttcaatttcaactaaagagccagtaaaagaagaagaagaagaattccagacgatcaatattcgtattgatagtcaagatagggtttctgcaaccacaacaactggtgatgcttttgtgatgccgaagcttcctggagaggaaactgatttctgggaaggacctcaatgggatggatttggattctttgttcagtatatgtgggcttttggcgttctcttttcgctgattgcatgtggtattgctgtggccacatataacgatggagctacagatttcaaggctactcctgtgtataaggaagcaatggaatcacaagaacttcttgaagaaccagaggcttctaactcagatgtgtttgagtcaaatccaactgaagaagcacctagtttggaatagataagtttgctaagtgcacttctaatgaagaattctgtcttcatattgtcataattacgggtaaaacagaagtccagct

SEQ ID NO: 310 - DNA, 527 bp, Lycopersicon esculentum
aatgagttcatgaataaatctcaagtgagtttctgatgatataattcaattaaactcacaaagatgaattactgtgttgttcctgatttcaaaatggatgatgattattatgatattccttctgctattttcaacaagaaatccacaatagcagatgaagaaatcatggagctagtatggcaaaatggtggagtaattatgcaaagccaaaatcaaagatctgtaaggaaatctaacctttttcccgagcaatcagctgtagaacaaacggtagcagtttcagctgcactgtatatgcaagaggatgaaatgaactcatggcttcaatctccacttgatgattcctccttcgatgattttctaaatactacaccgagttgtgacgccgtgacctgcgccgctgcagcaccgccgggagaaattggtacttcgaaggtggagatccgtccgcctctggttccgccgtgttcgcggccgattagatgtacagagggtgaattaccacatcgattacagaattttgggca

SEQ ID NO: 311 - DNA, 659 bp, Lycopersicon esculentum
aaaaaacttcaaagaattttgtctactacatcaagaaaacaagattttacacaaatactcttttcttttcttttttgtgacccacacaaatgatatagtactcaatctaaaccaagcttgcaaatcatttcaactttgcttatttcccaaatctacaaaacatagggaagagaaggagaaatctatctaactatatataccatttgagaagcaagaccccacccaccctggaattgtttctttaagtgccaaagttggaaacttttttgaggtatcaatcaagggttattggcttatcgatccattcgttatcagcagcaatcctaccggagccacgaccagtcattttgccaaaagaacttactgctgataaccccttgaaactactgctgccctcgctgaagtttcgtgatctcctagtctttcgaagttttgaggagtggagatcagaatctgtgtcattgttctttgtgaccttgatagcttgtaactgtacaatgttgcttctgcagaatgggcacacgggttctggtggactagtcgtggtcgggtttggcttgttgtggcagcataaggccagtacacaatgtgcacacatctggtgcccacagtcctggacttcaattgtgcataattgatcaaaacagatgcaacatact

SEQ ID NO: 312 - DNA, 546 bp, Lycopersicon esculentum
aaatgagctgcttccgacattgcttcctaatcttctttctactttggttcgtgttcagactctatgtgaggaatgttctgacccggagaagactccgggtaagctgttgatgcagtcggaccttgatatggcttctgggtggttgtctaagcagatccatcatctagaccttctttgccgttctggtgtgcttcgtcagtctactgctattgttctctctcacccttcgtcgaactctaccaaagacgatttagttctttatattagagacgtgtttactagaattcagatcggcggagttgagttcaagagaaaggctttggaatcgttgattcagctgctttctgaggatgaaaaatcagctggtcttgttgcaaaagaaggacaagttgggtacttgattaatttacttgaccttaatacggatccttctatacgtgaacaggcagttcttgctgtatcaatgcttgtttccatgagcgaacaggcaagaaaatgcgtgtttgaagagggtgctttgggtcctttactgagaatcatcgagtc

SEQ ID NO: 313 - DNA, 603 bp, Lycopersicon esculentum
gagaacttgaaacctggccctatgtcggagcgtaactacggcctgttcaagccggacggtacaccgtcttatcctttaggattctccggaatcaactccggcggcagcaccaatagcagctccggtaatagacctgcaactggtggtggttcttcaacaccggcatcgtggtcaccacaggacggaagttcctcttccggctatatgtccattacttctaattcggggagggttctattttgctggaaatcctggattctgcaactttacatcacagtcggttcaatttcagtgctattttctcaactgtaaactccattggagttgctgtgaagtttcgccattgttgagtactgttgtaaaagttaatttgggcgtttccggcatagctagccgttttattctttgattatagtgttattcttttgttaaacgaataatcggcggcttgatttgctctgctgatgagattcgtcggtacgccggaaagtccaaaagctgacagctgctctccactttcttcgctggcttgatgtccttattttctgcatgataagattagatcaagttgtaatttggtaacaataattttgtatatttatc

SEQ ID NO: 314 - DNA, 249 bp, Lycopersicon esculentum
ctgcattgttatgcttcttgcttattttggttatcttgtgttccaattgtggactcaccgacaacttttcgacgcacaagcagaagaaaatgaaggtgatgatatcgaagatgaagaagcagtgcttagtttttggagtgcatttgcatggttggttggaatgactattcttattgctctattatctgaatatgttggtggtaccattgaggcagcatcaaattcttggggaatttcagtgagtttcat

SEQ ID NO: 315 - DNA, 518 bp, Lycopersicon esculentum
  misc_feature (344)..(362): Unknown nucleotides 344 and 362
aagtttgtgagtgtaaaaatggtgcaaccccatgtcctattggtgacatttcctgcacaaggtcatatcaatccatctcttcaatttgccaagaggctaatcgaaatgggcattgaggtgacattcaccactagtgttttcgcccaccgtcgtatggcgaaaatcgcggcttccacggcacccaagggcttaaacttggcggcattctctgatggatttgatgatggtttcaagtctaacgtggatgattctaaacgttacatgtcagagataagaagtcgtgggtcccaaacattgagggatgtcattttgaagagttcagacgaaggacgtcctgtcacgtncctcgtctacactcttcntgctaccttggcagctgaggtagcgcgtgaactccatatcccatccgcgttactatggattcaaccagcaactgtgctagacatatactactattacttcaattgctatgaggatgaaatgaagtgtagtagctcaaatgatccaaattggagtat

SEQ ID NO: 316 - DNA, 500 bp, Lycopersicon esculentum
ttagtagtgaagatgagtatattaggagacatcatagacatgatgttagagataatcagtgtagttcatcacttgtaaagcatattaaagctcctgttcatcttgtttggtcattagtgaggaggtttgatcaaccacaaaggtacaaaccatttgttagcaggtgcatcgtgcaaggggaccttgaaatcggaagtgttagggaagtgaatgttaagtccggactcccagctaccacaagcaaggagaggttagagcttctagatgatgacgagcacatttttggtgtaaagatcgtaggtggagatcacaggcttaggaactactcatcaattatcactgttcacccgaaggtcattgacggatggcctggaacaatggtagtcgagtcattcgtggtggatgtaccagatgggaatactaaggatgaaacttgttactttgtcgaggccctcatccggtgtaacctcaaatcactagctgatgtatccgagcgctta

SEQ ID NO: 317 - DNA, 462 bp, Lycopersicon esculentum
gcacgaggccagatggtttcatgatcaaaatgagagatggaaggcatttgaaatgtgtgcataataatccacaaggaggtcatctaccggattatgctccacatccagcgattgtattaagaatggaagacgggacagggcttcttctccctattattgttttggagatgccaagtgcattgctcatggcagctgtgcgtaatgtccaaattgcccgaccaacaatgtatcaagttgtgaatgacatggttgagaagatgggttacacggttaaacttgttcgagttacaaagagggtgcacgaggcttattatgctcagttatatttaacaaagtccgacaatgatgccgagagtattagctttgatcttcgtccgtctgatgcaatcaacattgctgtgagaagcaaggtgccaatacaagtcaacaagtacttggcttatagtgatggtatgagaattg

SEQ ID NO: 318 - DNA, 455 bp, Lycopersicon esculentum
  mRNA (1)..(455)
ccatcacgagttgaattggtgattgcttttctatggagggctctgatcaatgtttatcgatgtaaaagtaatggacgtttgagaccttgtttattgagcgtccctgtgaatttacgtggtaaaattgactttccaagatacgaaaactcctttgggaattttgctattgaagtcccggtcaaattcatcccgggagaaacaggaatggagttaaaagacattttactattgatcaaggacgtcatacaaaaaatcaacgtatcatttgcgaaatctagtgatgatatatattcgctagcattcaagtttcataaagaaatacaataatgggaagaaaatgaacaagtggatgtgtgcatggcatcaagtttatgtaggttccctattaatgaagctgattttggttggagtaaaccttgtttgctaagttttggattgagacgtagagatatgtt

SEQ ID NO: 319 - DNA, 510 bp, Lycopersicon esculentum
gcacgaggaaagatcggagatttcatcaagtgctagcagtaaagtccatcagaaagtagctcaagagacggattgtagaactattatagatcaagacagtgatgagtctgatacagaggttttcagagtgaagagacgtcctcgagcagaacatagaagtgtacatgattcaatgtctatcaacgttgaaaatcagagttttaaaagattaaagaaacatcaatcaggacgattggggtcattgtgtttgccagagcattcctcgacttgtgatataaatcataggtccgttgctatttcaagccaatcgaaggaagcattggacttccatcccagagacaagtcagtgagaggtggtacagttccagtctgtattaagttgaagaaaggggttggttatgaacaagatgagcacaagagagatgataggttaccgtttgaattggggcagagtaagaggagggaaccagggagaacagagagtgggtcgaagcgtttgaaagtcagagg

SEQ ID NO: 320 - DNA, 532 bp, Lycopersicon esculentum
gggagtatctagttctcttgattgaacttttcggatgaatatctttagtccgtgtaatgcatgatgccaccactgatagtgcatcactatagaagtgcattcgatgataatttatgttgctgtaggtacatgatttcctccaacattggtgaggtcgcctctattttccttactgctgccctaggcattcctgaaggtcttattccagttcagcttctgtgggtcaacctggtcactgatggaccacctgctacagcattgggatttaatccaccggataaagatataatgaagaaacaaccaaggagaagtgatgattcattgatcagtgcatggattttatttcgctatctggtttgcactctaattatttatttaattttccacttcctacttctgtttgtgagaatgaaatctagctcatcaatttctaaatgcttgatgccaggtaattgggttgtatgttggtgtagcaactgtgggtatttttatcatctggttcactcatgactccttccttggcattgattta

SEQ ID NO: 321 - DNA, 633 bp, Lycopersicon esculentum
gggttgcagtgtcaaatactgttgcctttacaaatccaaatgcaccattatacccgcggttagcacagggaaagacatggaatgaaatgactgttacatggacaagtggatatgatatcaatgaagcagagccctttgtggagtggggcccacaacgtggacaacagactcgttcaccggcagggactttgactatcgaccgtagtagcttgtgtggtgcaccagcaagaactgttggatggcgcgatcctggattcattcacactagttttctgaaggagttgtggcccaatttagtatatacctacaagctggggcataaactgtttaatggcacgtatatctggagtcagatgtacaaatttaaagcatcgccctatcctggccaaagctctcttcagcgagtggttatttttggtgacatgggaaaggaagaagctgatggctcaattgtgtacaatcaataccaacctggctcccttaacactaccaagcaaatcattgaggacttaaagaacattgacatagtctttcacattggtgatattgtttacgccaatggatatctttctcagtgggatcaatttacttctcaagtagagccaattacttcaagagtaccatacatgattg

SEQ ID NO: 322 - DNA, 494 bp, Lycopersicon esculentum
ccctccaaaaaaaaaatgatgatgatcggccgatcacctcccaccgtccaaatccctccttgggacgtcaccgatgatccaacggttgatgtttattccccattctccgttagccctgattctgctttcgatgttttgacggcgttgcagcgttacctgccatcaaatggtaacgatgtgatttttgactctgatgggttggatattccggtggatgcattttcatgtgataatttcaggatgtatgagtttaaggtgaggaagtgtgcacgtggaaggtcacatgactggacggagtgtccgtacgctcatcccggtgagaaagctcgccggagggatccaagaaagtatcattactcaggtacttcttgtcctgattttcgtaaaggtcagtgtaaaaaaggtgatgcatgtgagtacgctcacggcgtatttgagtgttggctacaccctgctcgctatcgtacgcagccttgtaaagatgggacccactg

SEQ ID NO: 323 - DNA, 501 bp, Lycopersicon esculentum
  misc_feature (481)..(481): Unknown nucleotide 481
gcttgctcttacttccaagaaaccaggaaagacacaatgcatcaaccatttccggataaacgatagctggcatcttgtggatttaccaggttacgggtatgcagctgcaccacatgaagtacgaacagattgggctaagttcaccaaagattatttcataaaccggccaaccctcgtctcagtcttcctcctcatagatgctagcattcctgcaaaaaagattgatcttgattatgcaagttggttagcagagaataagatcccaatgacaatagttttcaccaagtgtgataagcggaaaaagaaaaagactggaggtaaaaagcccgaagagaacttgcaggattttctggagttgatacagaagttcttccaaactgcaccaccttggattatgaccagtagtgtcaccaaccaaggccgagatgagatactgttgcacatgtcccagctgcgaaattattggcttaagcattgattntgcatcatatgctttattgg

SEQ ID NO: 324 - DNA, 508 bp, Lycopersicon esculentum
  misc_feature (263)..(473): Unknown 263, 293, and 473
aagaatggtagtactacattttatcaaacattaataattaataactcaacagaagacaatccaagcaacctgttattttcactctacaaacataagtcttgtcatcaacttggcaagtacttaccttaggtcatattgagaccaaggttgatttgtactactagaatttgtacagcaagactaaattgcttatcactctattgtctcgggccgaagggagagctagaacggacagcactgcgatgatcaggtgtccctactcngtctgattgacctgatcctggctttggttngttcataaagttaccaagtgatctccaactcttctctgaatttgtatctgcatccacaagtccatcaacatactttgtcgaaccaattttctcaaactgcactctttgcctgggtgcactatgcgacctaagtttcgcaaggtatgattctgtattagacatatagttggggtgacctangtagtccccaaacaagcttcgtgagcaatcacttt

SEQ ID NO: 325 - DNA, 574 bp, Lycopersicon esculentum
cagttctcgaatatcatggagttccggtttatcaatcgatgtcgagtttgagaacgagtaatggtttgatgaacactttagctactgatggagctaataactacgatttcgttgttcaaaatgacggacaagatgtgacattgaagactaaaatagttacagggaaaatcactggaactgtatttgataaagagccagttgcgattttctcgattgatgaagttttagagccaatcgaattgttcaaattagctccaactcctgcaccagcaccagcaccgacacctgaagcagatgccgagtctccgaaaccggccgggaagaaacacaagtcaccaccagcgccggcttctccggcggattctccggttgctagtcgcaaaggcaaagttgctgattcgacggcggataagaacggtgggtttagattcaacggcggagctatggtaacccctgttttgttcatttggtttgcccatttgttactgtaatttttattttttattttagatttggtgttatgttttagatctgaaataagttggccaaaataaaatggctctgaattgtaact

SEQ ID NO: 326 - DNA, 545 bp, Lycopersicon esculentum
agatgtttattggacttctctaaggcctgtttatatttatgggccacttaactacaatcctgttgaagagtggttcttccaccgattgaaggctggtcgcccaattccgattcctaactcagggatgcaaataactcaacttggacatgtgaaggatcttgcaaaggcttttgttcaggttcttggaaatgagaaagcaagcaagcaagtatttaacatatccggagagaaatatttcacatttgatggattaactaaggcttgcgccaaggctggtggtttcccggaacccgagcttgttcattacaaccctaaggagttcgattttggcaagaagaaagcattcccattccgtgaccagcatttctttacatccatcgagaagtcaaaggctgagctaggatggaaacctgattttgatctggtggaaggtcttacagactcttacaacctagatttcggacgaggaacattcaggaaagcagctgatttctctacagatgatatgattcttgaaaagacactagttccccagagctaatatt

SEQ ID NO: 327 - DNA, 465 bp, Lycopersicon esculentum
gcacgagctaatttatgtctaatcctcactagatctatcatacagttcttcattcattctttctatttttacaactatttctttaagcattgctccaatggggaatgttgcactttttcatttgatttgtggaattttcattttttggagtgtttctctggtgaaagcagaggatacttataaatatttcacatggactgctacttatggaactctttatcctcttggtgttcctcaacaggtgatcctcattaatggtcaatttcctggtccaagacttgaccttgttacaaatgacaatgtaattctcaatctgattaataagttggatgaacctcttctgttgacatggaatggaatcaaacaaagaaagaattcttggcaagatggagttttgggaacaaactgtcccattcctataaattcaaattacacttacaagttccaaacaaaagaccagattgg

SEQ ID NO: 328 - DNA, 559 bp, Lycopersicon esculentum
ccaactttatggaaatcttggtcccaaggatatttgtactgaagatcaccaagagctagctcgtgaagctgcaagacaaggaatcgtcttgcttaaaaacacggcaggttcattgcctctatcccccaaatccatcaagtcattagcagtcattggccctaatgccaatcttgcctataccatggttggaagttatgaaggtaaattggatactatgttctattcactgacggtataaagattttttaacacatgcagtgtttatataacttcaaaacaccagttactaagagtcttttctcgcattttcttgaatatttacaggtagtccatgcaagtatacaactcctttggatggcctaggtgcatcagtttcaacagtttatcaacaaggctgtgatatagcttgtgccactgcacaagttgataatgccaagaaagtagcagctgcagctgatgccgtagtgttggtaatgggatcagatcaaactattgaaagagagagcaaagacagatttaatataactctccctggacaacaatcacttctagtaacaga

SEQ ID NO: 329 - DNA, 615 bp, Lycopersicon esculentum
ttaaatcttaagaatgggtatcataattttcatccttgttcttctttttgtagactcatgtttcaacattgttgaaggaagaatccctggtgtttactctggtggttcatgggaaactgcacatgctacattttacggcggaagtgatgcttctggaacaatgggcggtgcgtgtggttatggaaatttatacagccaaggatacggagttaacacagcagcactgagtactgctttgtttaacaatggattaagttgtggagcctgttttgaacttaaatgtacaaatactcctaattggaaatggtgtcttcctggaaacccttccattttaatcacagctaccaatttctgcccaccaaattacgcgttgccaaatgacaatggtggctggtgtaaccctcctcgccctcactttgacctcgctatgcctatgtttctcaaacttgctcagtaccgcgctggcattgttcctgtaacttatcgcaggatcccatgccgaaagcaaggaggaatcagatttaccatcaatggattccgttacttcaacttagtgttgatcacgaatgtagcaggtgcaggggatattattaaggtttgggtaaaaggaacaaa

SEQ ID NO: 330 - DNA, 581 bp, Lycopersicon esculentum
ctatgacagtgatgccggatcttccaccggttaaaacaaagctcgataaaccaaatccgacagctgaatccagagaaacaccggttgcgagtgcgactgaaaccggtgaaaggacggttcaacagcaacaacagagttatccaagtagtccactatggcattacccagcaccaccggtgcaaaccatacccgtttactatgtccctggttcggttcagccaggaaatatcccggttcaacccattacattaagggctccatatattcatccttttccagttcctccgaatcaattacccgtcgggtatccacccgcttcaagtatgggccaagtatatagtggattgaggccggtaatgagtgttgacccgaatgaattaagagttgttactgaaggaatgaattactatgcagttaggaattcaggagttgttccgggttacggaggtccagttagcgaagaaatgcagggatccggaggtgatggaatgtcgggtcgggtctcccaatgatcaaaaggtttgattgagaagcatattttgtgtgctaccgtttgtgttaatcattttgttttaatttga

SEQ ID NO: 331 - DNA, 611 bp, Lycopersicon esculentum
gcaaagtctctggagctctaagctttgctggcaagctaaatcatccaccagaaatcttgctgctggacgttttcttccaaatcaacaaagccttgggatgcagctcgcacaattaggtcgagatgcttctgcagcaggtgctgctcgcaaattagttactcagagaaagaacatagaggttgttgaatctgttggtattgctgggaaatctgatccggacaggaaagtgaagtactttagattggagttgcttgataagaaacaggaagaagaagacgaggatttagattttagagctctcgaaaatggatttgtggcagtcactccagtatctcttgtaatgcatgtggaaacagatgttaatgcagctgcatcaaagtggatttcttctgcattagaagtggaacaatgaaaagatgaagctaatattcagtattgtttgttgtacatctttagatgactttgttcaggtcatattgcaaaacttacatgggccatagttgtgatgcctcaattatttatttggtttttcaaccttttaatttgtgtcccgcgaagccaatgcttccactgtttacttgaaaggccagcatgttgtaaaagtgccatac

SEQ ID NO: 332 - DNA, 380 bp, Lycopersicon esculentum
cggaactgaattggtacggattgcttcgagatgtctccagtatgagcctcgtgagcggccgaatctgaagtcattagtttctgcattatatcctcttcaaaaagaagctgaggttccctctcttgtattaatgggtatatcatctgatggcgaaactatgcctctatctccacttggtgaagcttgtttgaaaacggatttgactgctatccatgagatcttagaagcacttggatacaaagatgacgggggagcagctactgagctttcatttcagatgtggacagatcagatgctggaaacgttgaactcaaagaaaaagggtgatgttgctttcaaaaataaagattttagtgctgccattgaatgctatacgcagt

SEQ ID NO: 333 - DNA, 227 bp, Lycopersicon esculentum
aggtttcgatctcttatattatatagcattagatcttttctcccttgtgtgtaaaaatgttcaagcagagagcagtttccatggttttaggttccattgtagtatcaaagttaatgtaagatagggtgaagtgtgatccatatccaaagttaaatttattttatatggtatcaaatagtaaacattatttggattcaatgctttttagtttagtgtttggttgtcac

SEQ ID NO: 334 - DNA, 501 bp, Lycopersicon esculentum
cttttccgatcaccggagctaccaccggagtaaacaccaccatgtcagtcaaggagtgcactcaccacaaagacaaaaaacgaaaacgcgtccgacgactcttcgccggaatcctaattttcctcttcgtcgtcctgttaaccatcttactagtctgggcaattctacaaccgaaaaaacccagattcattctccaagacgccaccatcttcaatttcaacgtctctgctccgaatatcttctccacatcgattcaagtcactatatttgcccggaaccctaacgataaagtcggggtttactacgataagatgaaaacatacgcaaactatcacaatcaacaaattacatattatacccaaattccttcagtttatcaaggtcataaagacgttaacatatggtcgccgttcgttttcagtaacaatgttccgatttcaccttataatggaccagatctgaaggaagaccagcaaaacggcggcgtttggctcgatttca

SEQ ID NO: 335 - DNA, 547 bp, Lycopersicon esculentum
tcgaagtcgtgctaatactgatccacatccttttctccaggttgatgctcttggaagagttgctttctcttctattcgcaggccccgaccgcttcttgaagttgatccaccaaaatcatgtgctactggatgcattgaacagaaaatgtctgagaaacccctggagcaagagccaatgctagcagctagggtgactattgaggatggacttagtcttcttctcgatgttgatgatattgaccggttcctacagtttaaccagcttccagatggaggagatcagttgaaacgcaggcggcaggttctgcttgaggatttggcctcatcacttcagctagttgaccctcttggcaaaagtgtccacactgtgaatttggctgctaaagatgatgttgtcttcttgaggatcgtctcactttccaagggccggaaactgctagtacggtaccttcagcttcttttccctggaagtgaactgaccagggtggtttgcatggctattttccgtcatctaaggttcttgtttggtgggattccaagtgatcac

SEQ ID NO: 336 - DNA, 448 bp, Lycopersicon esculentum
  misc_feature (442)..(442): Unknown nucleotide 442
gcaacaattacggttggtggttcaaaccatggttttaccaacacgctcaaaccgcactaaaaagaggggaatttgttgagtacattccaactagggactactaccacagacacacgagatcgttgtattgggaaggtaaactaattcttccattcggtgatcagttctggtttaggttcctcttaggatggctcatgccaccaaagattgctctgctcaaagccacacaaagtgaggctattagaaactattaccatgaccatcatgtcattcaagatctccttgttcctctttacaaggtcggtgattgtctcgagtgggttcatcgcgagatggaggtatatccaatttggctgtgcccacacagaatttacaagctgccagtgagaccaatgatctaccctgaaccaggattcgagaaacacaaaaggcagggtgacanccgaat

SEQ ID NO: 337 - DNA, 543 bp, Lycopersicon esculentum
attaattaggcctaaccttagcttattgacatggcttccaaaaatcaggcctctattacccttttcttatcacttaatctcctcttctttgctcttgtaagtgcagactgttcaactgatattttgaaatttggggcatgtgctaatatacttaatgatttggtgggtgtaattatcgggactactccaacttcgtcatgctgcagtttgattgacggactggtggacctagacgccgcggtttgcttgtgcacagccctaaaagcagatgtgctgggaattaatttggatatatcactctctctaaacatccttctcaacgtctgtggaaaaaaatatcctactggttacacttgttgattgaaaaattattatgtgtttgtgaaccatcgatctacctcttgttcagaataagtaagctttgcatacatacataaatgcagcttcatcacttcactttacttacttactagttctgtcatgttcatgtatctttacctttttggacatgtcaattcaataaatggaatgttgtttctattc

SEQ ID NO: 338 - DNA, 598 bp, Lycopersicon esculentum
ttgcaatggtccttccacagtctctgttgctatagctgcctactgccaccactcacacactgccaccaaaatacccaaatgacaactctctcctacaccttccctagcaccaccagcagcagcagcagcactaggaggttcttgattactagcaatgggtcttcttgccggcggcgccccaatatttccactgaagaccaaccctgttcctcttcagcctgttatcaaggggcagtgcagtcttatggtctgcagaggcaacgagtacaattaaatggcatatcaaaggctgtgagaacgagaagacttgattcagttggaagacaaaagaaaggagcaattagatgtaacattgaacttcaggacttcataggtggagatctcattaaacctgatttgggtcaatggttatcagatgtggaagaacacaaagccatagcaatttactctcctcatgaagggggctatgaaggacgctaccttacacgtcttaagtaccagggatatcatttcttggacctttcggctcgtggcctcggtgaccctgaaaccactcttaccaaattccaccctgtctgccctgctcatgttggtaagc

SEQ ID NO: 339 - DNA, 596 bp, Lycopersicon esculentum
ggtgacgcagaaatcaacctatcactccccggcgatgggttcgatgtagtcgtacgcgccaaaacggcgcttgaacttgcttgccccggcgttgtctcttgctccgacattctcgccgtcgctgcccggaatctcgttgttcaaaccggaggcccgttttatccagttaatttgggccgtaaagattcttttacttctaaagcttcgttggttgaaggaaatctgccccgacccacaatgtcgatggatcaaatcatacaaattttcgggtcaagagggttttcggtaccggaaatggtagcgttatccggtgctcacacaattgggttttcccattgtaaagagttcagctcaaatctttacaattacaacaaaacttcccaatatgatccttcttacaatcccagatttgctcaagctttgagaaatgcttgtaataatttccagaaagatccaacattgtccgttttcaacgacatcatgactccgaataagtttgacaacatgtattaccagaacttacccaagggtttgggtctgttgtcttcggaccggggtctgttttcagatccgagggacagagttcatgttgaaga

SEQ ID NO: 340 - DNA, 558 bp, Lycopersicon esculentum
aattaactgagagattataatatttcacaatttctcaacaatacacttaaaaaagagaacatatagtgctaattaaaattattaataatcatatacatattaatcaacatcttttgccctttttgcaaccaagtgcaccagcattcattgtaatagaattaaccaaagtagatggccttgcacttaaacttgatgcttttgcataacttgaagaagctccagcaccagatcttatgaaaaatgctccattaagcattaaatcaccttctgatctccaattccaattcttccattcactttctgctgcatcctcatgttttgtcacctccttgttgaagatatcattaggagcaaggaatctattgccttgactattgatagttggagaagcacttcctccaattgcatacatctcccaatgagtgtagtcattattcaccacatggaaatatccatgtctacatcttggcatcctttgcacaagcccttcaccaaaatgattaaaagcaatggtaacttgcatattcttgtctctagtgaatgaatcactatgtccca

SEQ ID NO: 341 - DNA, 523 bp, Lycopersicon esculentum
tctttttcttttccaaatccaaacagccccaaaatttccaattttttcttcctcatataagccaataatcaagaacagattcttcaactttaaacacaccctcaaattttcttggatcagtgtattataaatctcaacaaaagggacattttttttagtacccttttttcctttgtgttttctcttttctcctacatcacttgtctctcttgaggaaatcaagaaactatgagcagaattcaagaaaacatgtcttacacatgtgatgaggctgctcacattagatcttctttatctcaaatcctattaacaagtagtaccaacactttagactcaatattttctcattaccaagaaccaaaagatcaaatgactacaactagtcctgttcttgaaccactatgttcttcagtctatctcagacaaaaagatttgctccaaaaattttgggaacaaaataggacaaatatcacaattccaacaacttccacacaaacccaacttcaagaatctttacattctc

SEQ ID NO: 342 - DNA, 516 bp, Lycopersicon esculentum
  mRNA (1)..(516)
cgctcctttctctgatggctatgatggcaatttcaaaggttcatttaatgagtttcatttatattattcttctataaagtctcatgggtcagaatttattttcaatttaattaaatcaaatgcaaaaaattgtactccttttacacatgttatctacactgttgttatggaatggactgctttggtagcaaaagaattaaatactccatctacattattttggatccaaccagccacggtttttgatatttattattatagatatactgattattcggattatttcaagaattgtgattctaatgataaaataatagagttaccaggattgcctccacttagtccaattgattttccttcttttgtatttgataacgtggagtgcaacaattgggcagctgagtctattaaaacacaaattgatttattaaatagtgaaaaaaatccaaaaattctcgtgaatacttttgatgatttagaatggatgctctgagatttgacaatggactatgatga

SEQ ID NO: 343 - DNA, 586 bp, Lycopersicon esculentum
ggcaaagtttgacatagctgagttgaacacaattaaggttgcaaagtcatgctttgttcctgttttactgggtcatggtgttgatgatgattttatacagccccatcattcagatcgtgtatttgatgcttacatgggagacaagaatattataaaatttgagggtgatcacaactctccgcgcccacagttttattttgattctataagtatcttcttcaacaatgttttgcaaccaccaatggatgaagctggtgctgcattctttgacatgccacaagattattttggcaagggcagttggagtacagtccatgaactggactatatggatgatgtccatgatgttgcagctgcaccaactagtagcaccgaagatgtaataaagcaggttcgctccaaaagacccatgagtcggatagaggtcccttctgacatatctgctcaggataaccaaactgcagaccaggaagaaggtactggaacctactctgccccatcatcttcaaaaatgatcagctttgaactttccaatggtgatccatttggtcccaatgttcctgcatcaatagatgatgatgactat

SEQ ID NO: 344 - DNA, 542 bp, Lycopersicon esculentum
tgaaaatcatgagaatgacacacaattaattgatagggaaaccaatgatgatattgcaaatgctagtaatatagcagctcaaaaacattttcaacaagttcaacacatcaaaggggcttgagattatcaagtaaaaattaaaaatgatctatgttgcccgtcggacgtaagttaaattaattttaaaaagattttattgagcgtgatcaaaagttccggaggcagtctcttgttttttgtaatttttttaaattctaaaagcatatgcatatcaagtgtaatatcataaactataaatagtttctctttgtattaattttttctattttctcttaggttgttatgtagctagtgcaagacttctgatatttattaatatggttttgattaattgaagcagacttttgtttaatgagctcatgaatttgatcattgtacttttcataatccttttgttcaaatttattgtcgtgtttacatcaatttaatgacatgtagtatgtgatattcttattattaagctatcgttaatgaactcgttt

SEQ ID NO: 345 - DNA, 552 bp, Lycopersicon esculentum
gaaagatcgtgaaggtgatgtccaagtcttaaaggagcgaagtgagattattgaggaagttgtggatgaagtagtgcatgcctaccatggtggcttcaataaagctattcaaaattattctcagattttgcggttattcagtgaatccacccaaagtattggggttttaaaaggtgacttggcggaggcaaagaagcttcttggtgcccgtaacaagcagttgcatcagttgtggtatcggtctgttacattgcgacatattatctccttgttagatcaaatagaaggcatagctaaggttcctgctcgtattgaaaagctgatcaatgaaaagcaattctatgctgccgtgcagttgcatgttcaatcagctcttatgcttgaacgagagggtcttcaaacggttggtgctttacaagatgtcagatctgagttgacgaagctaagaggagttctcttctataaagtcttggaggatctgcatgcccatctttataataagggtgaatacagttcaactctcttcagtatcagtgaaagagatgatgaagt

SEQ ID NO: 346 - DNA, 475 bp, Lycopersicon esculentum
caggtgcagctggtccgacagggttggccgaggacgacagacgtgttgagtttaatattatttcgggcacgtctatgtcgtgccctcacgttagtggtttggctgctttgcttaaaggagcgcaccctgattggagcccagcagctatccgctcagctcttatgaccacggcttatacagtgtacaagaaaggaggtgcactccaagatgttgtgacaggaaaaccatccacgccatttgatcatggtgcaggacatgtagatcctgttgcagcactaaaccccggacttgtttacgacttgaaggctgatgattatctgaatttcctctgtgccttgaactacacatcaatccagatcaatagtgttgcaagaagaccctttagttgtgcaacaaataagaagtttcgtgtcgctgatttgaattacccttcatttgctgttgtatttccagaacaaatgactgcaagcagcgg

SEQ ID NO: 347 - DNA, 474 bp, Lycopersicon esculentum
gtgaataaataagcagaaataagctcaaagtttcgtatggtaaagaggacttctctgaatctcgtaagctagaaaagcagatcgaagacccccttatggcttcctaattccatcattctatctaagcagtagctgtgcttttgaagtttttagtcatgtctcgttcatatctctcagttaacgagtcgatgacatatacctcagttaggatcctctggaagttgttgctcttgctgcactctcttgctcttctccgcgtgatcttgtcaagttgcaaccatttgctgttttccttgtctaatggcagcaattgtagcggcgatgctgtctttcttttagctgttactttaggtacatgttgctcttgttccttaacaagtttgtatattttttcgttcaatatgctactagtttcattccatgtatgttttgtttattcgaaatggctaatgtaaactggattttgtgtgtttt

SEQ ID NO: 348 - DNA, 661 bp, Lycopersicon esculentum
tacagagtgatagtactggattttgttatagagagaggagaaatgagtctcaccggagtaacattgttggtcacactcatggtggtggccgccgtcactttaccggcaaatgcctgtctgccggcggaccaagcagcattgatggatatcaaagcagctcttaaggagccatatttgggcattttcaacacatggaccggcagcaactgttgccagggctggcatggtgttagttgtgacccgactactcaacgggttgcagacattgttcttcggggagaatccgaagatccgatttacgagaaagccggccggtctggttacatgacaggttcactctccccttctctctgcaaactagacaagctcactacactcatcgtcgccgactggaaagatatctccggcgaaattccggcttgtgtcacttcattaccagatctccgaatccttgagctcatcggcaacaaaatcaccggtcaaataccggaaaacatcggtcagctgagtaaactcactgttctaaacctcgccgataataaaatctgcggttcaattcctgcctccattgtcaacctcggaaaactaaagcacctcgaactcagcaacaatcaactcaccggcgaaatcccgtccgatatccgaaaactcggaatgatga

SEQ ID NO: 349 - DNA, 485 bp, Lycopersicon esculentum
agcacctactaatattgcttctattatatactatatactccaaacgtagttttacattacaaaaactactatgcttaaaagctcacaaatttcttatcaatatatccacgttgaaactcgattgattttgaattttcacgataaaaacgaggatatgtaacattctatttcattttcccgaactcgaactcaaaatttcatttcagatctccacgcaacatccggattgtacaaatcctgaaaattcatcaggaacatttatctttgttaatttactgttttgtccttgttttttgttgttttctacgaaaaagaacccatgcggggcaccttgataggccgacccggtaaccaaggtccgataaccaagatccacaccacatgctccgactttcacattcgggtcggattctgcccaacaagttaaatttccatttttacccacccccacacatgccctaggacactggctcgaaccccatgca

SEQ ID NO: 350 - DNA, 624 bp, Lycopersicon esculentum
  mRNA (1)..(624)
ttaaaactcaagatcaagccttttcatctagatttgctcctaatgctcttcaagcgcacaaccattacaaattttctgtggcatggcttcctgtttgtcctcaatggcgaacgcttcgaagaatattgaacactaacctcttatcttccaataagcttgatgcaaatcaacatctaaggtctcaaaaggtgaaacaattgattgattattgtgacaaatgtagtcaacaaggtgaagctttggatataggtcaagttgttttcaagactagtcttaatttgttgtcaaacacacttttctccaaggatttggctgatcctttttcagattcaaaggtagagttgaaggaagttatatggggtatcatggctgaggtagggaagcctaatttagtagattttttccccatacttgaaaaaattgatcttcaaggaataagacgtcgcgctatcattcattttggtaagttgtttaaactttttgatgatttgatcaacgaacgattggaggaaaagaaaagaccaggattcatggaaaagagcgatgttcttgaaatatttctcaatattactgaaaaaaatcctcaagagattgatcataatcacatcaagtccatgttcatgg

SEQ ID NO: 351 - DNA, 638 bp, Lycopersicon esculentum
  misc_feature (474)..(474): Unknown nucleotide 474
gcacgagattgacgttgggagacttttagaagcagatatagtctcaaatggccaaaaagtctcactgacaactactgctcctatcgatccagcatctggattggggagttatgtcgagacacttttccggaagtcgaacattgaattcagcgtggttattgcacagatgaatggacggaactattcatcacgttctgcacactgctttcatgtcgggaaaacaaagctgaagttgggcaaaggatggattacaaaagctagagattcatactctaagtctatgcagctatgtggatttagaggaggcggaaattctgcggcaaagtcattgttttggctaccaaggaaaggacattcctttgtgttagtatttgaatcagagaaagaaagaaacggagcccttatggctgctcggaaatatgccatggattgcaatgtgataacaactagtgtcactaaagtatagtgagttgntgctcagactcttgaaaaatgctacgacactcgtgttggatcctcgtaaaatgcactacttttggagtatctgacacacatccgtcaacattttggagagtccgagcaacacagaatgagtgtaattctagctggacctgatgatgatgctttgctttaaacat

SEQ ID NO: 352 - DNA, 516 bp, Lycopersicon esculentum
tgttgctggggaagaggactcggccaccgatgaagagaacaacaagtatgacagaattcactttagatctcaatctcaataatcctgaccctaattcttctcttagtaattacaataatccattcgatcctcacaatcctttttcacctgcaaacacccagcagcaacacctagatcaacaacgtttattgacagcagcgaaaaccgtttccgtcagaaaccgccgtaaatctgctgattttctagaaactgctaattttttgaaagcttgcttcctctgtaaacgccggttaatccctggccgggatatctacatgtacagaggagatagtgctttttgcagtctagagtgtagacagcagcagatgaatctagatgagaagaaggacaagtgttcgttggttgcatctagaaaggactacatgaagaactccgtcgctgccgccgtagccggcgggaagggatctgacgtttccgccacaagcgagactgtcgccgccgtttaggtagtggaaa

SEQ ID NO: 353 - DNA, 545 bp, Lycopersicon esculentum
ggctaaatctgaaagaagcagcagatatttatgttcttggcttccaggaaatagtccctctaaagcctaaaactgtaattggagcagaagatccaacagaagctactaactggaatgtgctcgtcggtaaaactctaaatagcaagtatggtggtgcctggttaacacccatggtcaatccaatcaccaatgataattatcgatatgatcaagccactgaatcagatagtagactgaggaatgactacgaaatagtttcagcaagaggtcagtctagaactgaatataagctctcagattgtgttggtagttacaaattgatggctagcaagaagatggttggtgtcttcattagtgtgtggatgagaaggacattgctaaagaagtattgtgtctccgaagtaaaggtctcttcagtagcttgcggcattatgggctatttgggaaacaaaggatccgtctctgtgagcatgtccataggaggaactagtttttgctttgtggcagctcacttagcctcaggagaaaagaaaagtgatggaagg

SEQ ID NO: 354 - DNA, 731 bp, Lycopersicon esculentum
gcagcgctttattcatcaaagctgatagctgatactgataagttggagtcaaagtctcattctcaattttcacttagcagatcgcatatgactgattatgctattccaaatgctaatccagaaaggctggagctgcctatagaaagtggtaccatcagcatctctagtgcctattctcaagggctattgaacaccttgacacaagcgctgcatagttctggagtagatttgacccaagccaatatctctgtgcaaattgatcttggaaagagagcaaatgggagagtaaattcatcagcttccactgttaagggtgacaacgtttcaacaagcaatcaaccaattccaaaatctagagttacaaccacaagggaggagcctgatcacgccttcaaaaggcggaaaacaagctaatgctaatttgaaacactagtgttctttcttcttttttttccattggttaaatttaattgttttcctctttcccttccccttctcaaccaatatcgatgtccacacctgatacagaaaatctcccaaatgagagatcacaagccactgtatatgtaatgaactagtactactactatttttctgccccttcaaattgttgggctgcacaaatttacctgactagagtgtgatattttattactttggtagtaaactactgtattaatttattactaaggctttcaatgatgatgatgagacagctgcattttccaggt

SEQ ID NO: 355 - DNA, 648 bp, Lycopersicon esculentum
  misc_feature (620)..(620): Unknown nucleotide 620
caaggatccaagtgccaagaaacctgaagattgggatgacaaggaattcattgatgatcccgaggataagaagccagagggctatgatgacattccagaggagataactgatcctgaagccaagaagccagaggactgggatgatgaagaagatggtgaatggacagccccaaccatccccaatccagagtacaagggcccatggaaggcaaagaaaatcaagaaccccaactacaagggaaagtggaaggctcctatgattgacaacccagacttcaaggatgacccagatctatatgttttcccaaaattgaagtatgttggagtggagctgtggcaagtgaaatctggaactttgtttgacaatgttgtgatatgcgatgatccagagtttgccaagtcaattgcagaggaaacatggggaaagcagaaagatgctgaaaaggctgcttttgaggaagcagaaaagaagagagaggaggaggaatcaaagaatgctccagctgaatctgatgccgatgaagatgacgaggctgatgaggctgattcagatgatgctgatgacaagtctgactccaaggatgaggacacacatgatgaactgtaagagccaagcattntgattcaactgacaaagaagcttacttg

SEQ ID NO: 356 - DNA, 624 bp, Lycopersicon esculentum
aattaatcaagtattatgatattggtttagctagtattaaggctagaaatggatatgatgcttttcatgttgctgctaaacaaggtgattttgaaatggtgaaggtactattggaggcatttcctcagctgtcagttacatttgatcagtcaaattccacagcattgcacactgcatcagcacaaggtcacattgatgttgtcaatttcctcttggagactaatagcagtttagccaccataccgaagaataatgggaagacggcgcttcattcttcagcaagaaatggtcatgttgcagttgtgaaggcacttctaagcaaagaagaagggattctaaattggagagataagaagggacaaacagctcttcatatggcagtcaaaggccaaagtgttgatgtcgttaacgagcttattctatcggatcctactttggctaccattattgatggaaagggtaatactgctctgcatattgcaacgcgtaaaggccgtgtagagatcgttcaagcagtcgtaaaggacaaacgaatgaagtgggatgccatcaataaatctggtgaaactgctcttgacgttgctgagaaagcgcgaaattcagagatagcagcgattctaaagg

SEQ ID NO: 357 - DNA, 599 bp, Lycopersicon esculentum
  misc_feature (563)..(563): Unknown nucleotide 563
gaagaagtcagccatggcttctgagaaagaagctgctcttcttgctgttccttcagattctcctaccatatttgacaagatcattaacaaggaaatcccagcagatattgtatttgaggatgacaaggttttagctttcagagacataaatccccaagctcctgtgcacattctgcttattcccaaggtcagggatggcttaaccggactttccaaggctgaagaaaagaactgtgatgttcttggtcgacttctctacacagcaaagcttgtagccaaacaggaaggtcttctagagaatggattcagactcgtgatcaacgatgggcctgatggatgccaatctgtttaccatcttcaccttcaccttctcgggggacgacagatgaactggccacctggctaaaaggacgtcgagatgaattccagatttcatcctatcatatatgtcatgtgtgcttaaaaaaacccttataatctatgtgtagcatttgaagaaacttgtgctatgctcctatggagacaaataagactactctcgacaattatgatatttatcattntatcactaacacagattctctgcactcttgcctaat

SEQ ID NO: 358 - DNA, 2534 bp, Lycopersicon esculentum
tgaaaatttcacaaacttcattttattgtatcaaaaggggcttagtttatcagtaagtttttatctgataaactaaccagcattgcccattttttctcttacttgtagttacttgtatacaaaaacaacagatctgcacagtgaagtaatttttacccacaaaaaaatgaagggttttgttggttctctggtttttgtgattcttcttgttgttccagtagcattagctgggcataattatggtgaagctttgagtaagagctttttattttacgaagctcagagatctgggtaccttcctcgtaaccagagagttcaatggaggggtaattctggacttaatgatggaaaagctagtgggattgatttggttggagggtattacgatgctggtgataatgtgaaatttggtttgcccatggcatttactgttacaatgttgtcatggagtattttggaatatggtagacaaatggctgctagcggtgaacttagccatgctatggatgctgttaagtggggtactgattatctactcaaagctcatcccgaaccttatgttctttacggagaggttggtgatggtaatacagaccattactgttggcaaagaccagaagatatgactacatcaagagctgcttatcgaatcgacccgaaccatcccgggtcggatcttgccggagaaacagccgccgccatggcggctgcttccatcgtcttccgtcgttacaacccgggatattctaatgagctccttaatcatgcacatcagctgttcgagtttgctgataagtataggggcaaatatgatagcagtattactgtcgcccagaagtactaccgatctgtcagtggatacgcggatgaattactatggggagctgcatggttatacaaggcatcaaacaatcaattttacttgaattatttgggaagaaatggtgatgctcttggtggaactggttggtctatgactgaatttggttgggatgtcaagtatgctggtgtccaaacacttgttgctcagttcttgatgtctggtaaggctggtcataatgcacctgtatttgagaagtaccagcagaaagcagagaactttatgtgttcaatgcttggaaagggtaatagaaatacccagaaaactcctggaggtctcatctataggcaaagatggaacaatatgcagtttgttactagtgctgcattccttgccactacttattctgactatttggcttctgctggcaaatatctcaagtgttcttccggttttgtttctccgaatgagctcctctcattcgctaagtcacaggtggactacattcttggtgacaatccgagagcaacaagttacatggtgggatatggaaacaattacccgagacaagtacaccacagagcttcttccattgtctcgtttaaggttaatcctagttttgttagctgtcggggaggctatgctacctggtatagtagaaaggcgagcgatcctaatctccttactggtgctcttgttggtggacctgatgcctatgacaactttgctgatcaaagagataactatgagcaaactgagcctgccacttacaacaatgctccgttgatcggagtgttagcaagacttcatgctggtcacagcggttacaatcagctccttccagttgttcctgacccgaagccaacgccaaagccagctccaagaactaaagtaactccagctccaaggccaagagtacttccagtcccagctaatgctcatgttactattcaacaaagggcaactagttcatgggctctgaatgggaagacttactacagatactcagcagttgtaaccaacaagtccggaaagacggttaagaacttgaaactctccatagtcaagctctatggtcctctctggggtctaacaaagtacggtaactcgttcatcttcccagcttggctcaactctttaccagctggtaaaagcctagagtttgtgtacattcacactgcttcacctgcaatcgtctccgtttcaagctacactctagtctaaaagacatcaacaagttgaaaaagaaaaaaatgaagtggaaatgaaatgctcacacatttttcttgtggttctaatgtaagaattgtgcagctatactactactactactagtactaatcggcggggtcaagcaagtccctttcatcgtttctttttcgtttgtctgtaagttttcaacgtcttgttggtttgattttggttaagacttttgataacgaggtgttaggttgataatgtggagaagtcggaggaagaaaggagtgaagagacgagtttatacaagtgctcatcctctttctcctccactttatatatatacacatagtgtttcagaaacagagaaaaagagtgaaaggagtagttgtaaagcaaagaaagttttgcttttgacttctctctatatttattatcaatctataactcttcattctcctttgtaatttcaatgcaagcttgaaaatattcctaattaattttgtaaaaaaaaaaa

SEQ ID NO: 359 - DNA, 2802 bp, Lycopersicon esculentum
tttttttttttgtattagttgatcggaaaattgaagatttttggccggaaaatggaaagaagaagtgggtattgtttatcagttataatgttagtgtttggagtggttttcttgcattgtttggtgatgacgtcatttgccgctaatgtgacgtatgatcaccgggcattggttgttgacggaaggagaagagttttgatctccggttcaatacattatcctcggagtactcctgatatgtggccagaccttatacagaaatcaaaagatggaggattggatgttatagagacttatgttttctggaaccttcatgaacctgttcgaaatcagtatgattttgaaggaaggaaagatttgattaattttgtgaagttggtggagagagctggcttatttgttcatataaggattgggccttatgtttgtgcagaatggaactatggtgggtttcctctttggttgcatttcattcctggaattgaatttcgaaccgacaatgaaccgttcaaggcagaaatgaagcgattcacagctaaaattgttgacatgatcaagcaagaaaatctatatgcatcccagggtgggccggttatcttgtctcagatagaaaatgagtatggcaatggtgatattgagtctcgttatggtcctcgtgccaaaccttacgtgaactgggcagcatcaatggctacgtctttaaatacgggagtgccatgggttatgtgtcagcaaccagatgcccctccttccgttattaacacttgcaatggattttattgtgaccaattcaagcaaaattccgataaaacacccaagatgtggactgagaattggaccggatggtttctgtcgtttggtggtcctgtcccttacagaccagtggaagacatcgctttcgctgtggctcgatttttccagcgaggcggaactttccagaactattacatgtaccacgggggaactaactttgggagaaccagtggtggaccgtttattgcaactagctatgactatgatgctcctttagatgagtacgggcttataaatcaaccaaaatggggtcacttgaaagatctccataaagccataaagctttgtgaggctgcaatggtggcaacagaaccaaacgtcacttctctgggctctaatatagaggtcagtgtttataaaactgattcacagtgtgctgcatttctcgccaatactgctacgcaatctgatgcggctgtgagcttcaatggaaactcatatcatttacctccatggtccgtcagcattttacctgactgcaagaatgttgcttttagtactgcaaagattaattctgcatcaaccatctcaacatttgttactcggtcttcggaagctgatgcatctggtggatccctatcaggttggacttcggtgaatgagcctgtaggcatctcaaatgaaaatgcattcacgagaatggggttgttggaacagataaatactacagctgataaaagcgattatctctggtactccctgagtgtcaatataaaaaatgatgagcctttccttcaagatggatctgcaacggtacttcatgtgaaaacacttggccatgttcttcatgcttatattaatggaaggctatcaggaagtggaaaaggcaacagtagacattctaactttacaattgaagttccagtcacacttgtgcctggagaaaacaaaattgacctgttgagtgcgactgtgggacttcagaactatggagcattctttgatctcaagggagcagggattacgggtccagtgcaattgaaaggtttcaaaaatggctctactactgatctttcatcaaagcagtggacatatcaggttggattgaaaggagaagatttgggcttatctaatggaggttctacactttggaagtcacaaactgcattgcctacaaaccaaccattaatatggtataaggcaagttttgatgctcctgctggtgatacccctctttcgatggattttactggaatggggaagggtgaggcatgggtgaatgggcaaagcatcggtcgattttggcctgcctatattgcaccgaatgacggttgtactgacccctgcaattatagaggaggatacaatgctgaaaaatgtctcaaaaattgtggaaaaccatcccagctgctataccacgttcctcgttcatggctgaaatccagtggaaatgtcttggtgttgtttgaggaaatgggcggggatcctacgaagctgtcttttgcaacaagagagatacaaagtgtatgctcaagaacatctgatgctcatccacttcctattgatatgtgggcttcggaagacgatgcacgaaaaaagtcagggccaactctatctcttgagtgccctcatcctaatcaagtcatctcatcaatcaaatttgcaagctttggcactcctcaaggaacatgcgggagctttatccatggtcgatgcagtagcagcaatgctctttcaattgtaaagaaggcttgcattggatcaaaaagctgtagtcttggagtttcaataaatgcatttggtgatccatgtaaaggggtcgcaaaaagtttagctgtagaagcttcttgtacatgatgcctcacgaaactaatgaaccgagaaggcacgttccttaagtgttaaagtagtataaggtcagataaaccattactaagttacagcttcagaaagatagtgcttagctacatatacatcatggagaggaagaccactttggcttccctcaccgatttgatgaaattataataaaagaaaactagcttcaat

SEQ ID NO: 360 - DNA, 772 bp, Lycopersicon esculentum
gcaaaatggaccaaactttgccccgtcaacttgcacttgaaggctccaggaaggaagctggacacaaacagctcaagttggatataaacttagagctccctgctccaactgataatatggaggcaagttcttccagaactgaaagtttcgacttgggatcaatactggcccgtgctgaacaacccagttcatcaagaactgatttctgtcccgaagggcttcttggacatgacccaggtagtagatgggttaagcgcctcaaaatgagtgcttgcggttctttggcttttggtactaaaagctccagtttagtaggagaaacatctaatgagaagtcacacaagttcctcaatcaaattcctaaaggaaccatagctaactctgaattagcatcatctagtaaacgtcatggtaaagaattgatggttcacgacaacactgcaagcttagcaatgaactctagtactgcttctatgagtgttatcaagaaagatctagaggctttgacttctcattcttggattcaaaggttgcttcatgataggactaccaatgcaccaaagaggccccaaccggtggtggtttgtgagcctcaaacctcaaagttggagctcgatgatttccagaagaagcaacttccaagccttggtgccatggccctcatgggtaaggcaatgaatggttttcagccatgtgaatatcatagaaaaggccctttagttgtttggacactaagagtttctgaaaagtcaatagttggcgctaggatgga

SEQ ID NO: 361 - DNA, 702 bp, Lycopersicon esculentum
tgcttcttcttctcttaaaatttgcgtcttttcttatctttccatttccgtgattctctttggttacttcctttactagtatactatttcactaagtgaaaaagacgcaaactttttttccttttatacagcttttgttcaatttttcaatattcttgaactgatatggctgtggctatggcttcttgtgggagcatttctggggtttatactaatttaaatgcaattgagaaaatacccatttcaagttcagttctcttcagaggttcttttggtttggatcctgtacagagaatatgccttgttcccaagagattagctttttctgagagtacaattattccaaaagcatcttcaactgcagctgttgagggtggaagttcccaagatactgctgtcccaacgcccaaagtcataatagatttggattcggatccagaaacaactgtagtggaggttacctttggtgatcgccttggggctcttcttgacacaatgactgcactaaaaaatcttggactgaatgttgtcaaggctaatgtctatctagattcatcagggaagcacaataaattatgcatcacacatgcttctacaggtagaaaggttgaggatccagagctgctagaagcaattcgattgacaattatcaacaatttgatggagtttcatccggaatctagctctcagttagctatgggt

SEQ ID NO: 362 - DNA, 559 bp, Lycopersicon esculentum
gaggaactagtctcgagttttatcttctgtttcacacacttctagagagaaatagggtgattggcgtagtttttattttcttttctgtggttatttttgaattcttaggaaatgttgggtgtatttagcagttccattgtttctccgcccgacgagctcgtcgccgccggtagccggactccttcacctaagatcacgtcggatgctctggtgaaccggtttgttcaaagcaattcgtcggcgatttctatgcagatcggcgattttgtccaactcgcttactctcactccaaagagtctgctgtactccccaggtcatttgctgttaaggatgacatattttgcttgtttgagggatcacttgacaacttagggagtctgaggcaacaatatggccttgccaagcctgcaaataaggggatcctggggattgaaccatccaaggcacttcgtgacagagcaccttaccctccaaatcatgttgttggtcaccttgaaggcagtttcgctttcattgtttttgacaagtctacttccaccttgtttgtggctaccgatc

SEQ ID NO: 363 - DNA, 580 bp, Lycopersicon esculentum
gaacaaataaataaactaagcaccaaataaagcatcatcttggtgtaacatctctgccttggtttgggatccagggcagagatctttttaatctctgtctctattccggactcgaatccaaaatggcgtttcaagattttgatcaaatttcagagcggaggaagctcgagagacagcaaaagatgaagaagaggataatgattggtgttgtattgctactcctcattctcattgctgttgctgcttctgttgtctattttgttgtgtttaaaaacaagagcggtgattcctcctccgctagtaaatctaataagaacgcgtcaacgcctcctccaaaagaaactgaaacaccgactcctaaggctgatgcatcacctcctccacctgaggctgctgatacagcaccaccttctgatgaaggtggtgctgctgcccctgcaccaaatgtggacggagctgcatcacccgctggagatgcagcacctgctgcaccatcacccgacggtggcaacgcatcagcagacccagccccctcaccacaaggtgaaggagctatgggagcaatatgcgcgggaacaga

SEQ ID NO: 364 - DNA, 596 bp, Lycopersicon esculentum
  mRNA (1)..(596)
cacaagggacagctgatcaggaaagaaagaattctactaaggacagctcaggatcagccaaagttagtgatggggctagcagtattgcaaaaacaagtggaagtgccaaaattagtgatcgagcagattttcttgagagtggtaagagcagcatctgtagggggagcacaggaactgacgtaagtgaggagagtacttctagcagcttgagcagcagcgttaacaaaccccacaaagctaatgactcaagatgggaagccattcaagctatccggactaaagatggggcattggatttgaggcacttccggttgctgaagaagttgggaagtggtgatattggaagtgtgtatctgtctgagttgtgtggaacaaagtgttattttgccatgaaagttatggataaggcgtcgttagctagtcgtaagaaattgcttcgtgcacagacagaaagagagattttgcagtccctggaccatccatttctcccaacattatatagccattttgaaacagaaaagttctcatgtctagtgatggagttctgcccaggaggagacctgcacacactcagacaaagacaaccaggggaagca

SEQ ID NO: 365 - DNA, 533 bp, Lycopersicon esculentum
  mRNA (1)..(533)
acgtatcgttttgcttttgcaacaatcgagattcaaaaaaccatgtttacagaagttgaacaacaacagcatatagagaaactcgaaatattcaagatccaaggcagagataaacgtggccgcaaaattttgcgcatcatcggaaaattctttcctgctagaaatctgagtgttgaagttgtgaacaagtatttagcggagaagatctttccggaacttgagaaaaggccgttcgccgtggtgtatgttcacaccgatgtggagaaaagcgagaatttccccggagtatcagctttacgatcgttctacgatgcgattccggtgaaagtccgagagaatctggaggctatttactttcttcaccctggtcttcaagccaggctcttccttgccacatttggtcgcttcatcttcagcggagggttatatgggaaattgaggtatgtgagcagggttgataatctgtgggagcatgtgaggaggaatgagattgggatgccagaaaattgtgtacgatcatgatgaagatcctg

SEQ ID NO: 366 - DNA, 492 bp, Lycopersicon esculentum
ggtgaaatggcttaaaatgaacgctacatgtccactatgcaagtacaacatcctgaagggaaatgatcaggtttaaagcagggagaatgaaaaacattgtacattgtttcgggttattaaagccacttatggaagcttgaaacactacttgctaaatccatatcagaacagaaatctgctttccaactagtcaagcttcctctcatgttcgcttagcatattgagatcttctctgtagtgaatcgtcttctgtgattaaaaaaaatgtagcaccccgcgaagaattaaaaacgaagaagagttggtccttccccccagctcatgtaaattatctttggttccatttgtttccaataggtatacaggatccaactcttgtaagtatatgctattttcttgtgtgtggtttatgtccagttttactgtgctcttttctctaagaaaacagaaaagtgggtgataatttgatatctaagttttactcaatttgtt

SEQ ID NO: 367 - DNA, 549 bp, Lycopersicon esculentum
tggagttgaagggccattgggtgatagaatgttcgatttggtcacccagaagcgtaaggatcaaaagcttacatatgaggaccttgtgattgctaaaggaacctatgaaaaagggacaaatgctgatattgaagagtttatctatcagttgttagatgtctttgatgatggcgttgttggaagatcagatctggaagcagtcttgactgcagtactaagaaatatatcttctcatgagagccatcaatctgaacctagtccggacagggaaattctcgacattttcctcaatgctgcaaatttgacaacggatgatacaaaatgcgctgaaagttgtatgtcttttgaggaatttaaaagttggtgtgcccgtcttccatcagtaaggaagtttctcggaactctgttgtctacacgtgattcaggctctgaggttcctatgctagtatacccagagaacattgatcctgctgtaatattgttaagaaaggaatatgcttggcatataggtggggccctcccacaagacgagctgcatgaatggagact

SEQ ID NO: 368 - DNA, 462 bp, Lycopersicon esculentum
  misc_feature (424)..(424): Unknown nucleotide 424
ctctacacaaccttccttcttccacagccggtttcagatccggagtcacatttccccgaaggaataatcgaacagcagcggtatatgccgccgccgttgcggaagcaccgccggaattgcaaaggagaacaccggcgagtctctacgatgtactaagggtaaaatcaaatgcgtcacccaaagagataaaaacagcttaccggaatttagctaagctataccatcccgatgctgcgtctctgtcggaggaatcttccgacggacggcatttcattgagattcacgaagcttatgctacgttatcagatcctgtttctagagatctctatgatctcaagctaaacatgagctccggccgacgaggttttgggagctccggcgatggaattcgtaggaatgggtcagagttttacccgaccccaangtgggaaacggatcagtgttggtgacgtcagatgatta

SEQ ID NO: 369 - DNA, 746 bp, Lycopersicon esculentum
gggcagagagagagagagagagagagaagagaaatggagaaggaaagagaaaaacaggtttacttggctaggctggctgagcaagctgagagatatgatgaaatggttgaagcaatgaaggcgattgctaagatggatgtcgaactgactgttgaagagaggaatttggtgtcggttgggtataagaatgtaatcggagcaagaagggcttcatggcggatcttgtcttcgattgaacagaaggaggagagtaagggtcatgagcagaatgttaagagaataaagacttacagacagagagttgaagacgagcttacaaaaatatgcagcgacattttgtcagtgatcgatgagcacctcgttccttcgtccactactggagaatctactgtcttctactacaagatgaagggagattactatcgctatttagcagagttcaaagcaggggatgatcgtaaagaggcgtctgaacagtctcttaaagcttatgaggctgctactgccactgctagttcagatcttgctccgactcatccaatcagacttggactcgcattgaatttctcagttttctactatgagattctgaattcacccgagagggcatgccacttggctaagcaggcatttgatgaagctattgccgaacttgatagcctcagtgaagaatcctacaaggacagcacccttatcatgcagcttctaagggataatctcactttgtggacatcagatcttgaaga

SEQ ID NO: 370 - DNA, 546 bp, Lycopersicon esculentum
  mRNA (1)..(546)
gctctgtttctcaactttctgctgtgaaaatgcgtgagattcttcacattcaaggtggccaatgcggtaaccaaatcggatccaagttctgggaagttgtgtgtgcggagcatgggattgattcaaccggaaggtaccagggagacacagatctacaacttgagagggtgaatgtgtattacaatgaagctagtggtggacggtttgttcctagggctgtgcttatggatctggaaccagggaccatggacagtatcagatctgggacttatggccagatctttcgtcctgataactttgtttttggccaatctggtgctgggaacaactgggctaagggtcattacactgagggtgctgagctgattgattctgttctcgatgttgtgcgtaaagaagctgaaaactgtgactgccttcaagggtttcaggtgtgccactccctgggaggagggactgggtccggaatgggaacccttttgatttcaaagatcagagaggagtacccagatagaatgatgcttaccttctctgttttcccatctc

SEQ ID NO: 371 - DNA, 510 bp, Lycopersicon esculentum
ctcgagagaggtcagagttggtggttttgggccccacctggcactaaaatggcacgtatttggggtcgtactaattgcaactttgatggtgctggaagaggttggtgccagactggtgattgtggtggagtcctggattgcaaaggatggggtaaaccaccaaacaccttagctgaatacgctttgaaccagtttggtaacctagatttttgggatatttctgttattgatggattcaacatccctatgtcttttggcccaactaagcctggccctggaaaatgtcatccaattcaatgcacagccaatataaacggtgaatgccctggttcacttagggtacctggaggatgtaacaacccttgtacaacattcggaggacaacaatattgttgtaatcacggtccatgtggtcctactgaattgtcaagatttttcaaacaaagatgtcctgatgcctatagttaccctcaagacgatccaactagtacttttacttgtcagagttgg

SEQ ID NO: 372 - DNA, 700 bp, Lycopersicon esculentum
  misc_feature (626)..(626): Unknown nucleotide 626
ttgagctccgttctcatttgagtgtgtcctctttcaaaactattgacgggcgaggcaaaaaaatcaaactcactgggaaagggttgaggttgaaagaatgtgaacatgttattatctgcaatctagagtttgagggaggtagaggacatgatgttgatggcattcagattaaaccaaaatcgaggcatatttggatagatcgttgtagccttagcgactatgacgatggtttaattgacatcacaaaagagagcacagatattactgtttctaggtgtcacttttcaaagcatgacaaaacaatgcttattggaggagattcttctaattgtggtgacagatgtatgagggtcaccattcaccattgtttttttgacggaactagacagcggcatcctcgtgttaggtttgccaaagtacatttgtacaataactacactaggaactggggaatttatgctgtttgtgctagtgtagaatcacagatatattcacaatgcaatatatatgaagctggacaaaaaaaggtggccttcaaatatctaacagagaaggcagctgataaggaagaagcatgtactggtagcattaaatctgaagatgacttgtttgtttgtggaactcangctggtttattgtctacttgcagcgaaaataacgtcttcaatccatctgagttctaccaaacctggactgttga

SEQ ID NO: 373 - DNA, 566 bp, Lycopersicon esculentum
tttcacaatccttctatttcttcaaactcttcatatcaatttcgatacaaagttcgattattttatttgtgaagtcgtgatttaatatgtctggaaagaacagttacaccgcgtataactcgaacgagacatcatgggcagatcaatgggatccagaaccagcaagttatacaagttttgacaagaaaactggaaataataatagttctaagatttcaagtaaagttggagatactttaggaaaaactaaatatgttgcttcaactggggttaaaaaggtaaaatcaagtgctactgctggcttccaatggattaaagacaagtgcaataagcctaagtaaaattttattattattattattaatattgtgttttttttttgtatatagtgttttttgatgtattaataatacttttgtttgttctttcgttcatttttagttaatttaatgaataaataagtgattacatgattcatttttcatgattagtattaatattgggtttttctttttgtatatagcgtttttgatgtattaataatacttttgtttgttctttcgttc

SEQ ID NO: 374 - DNA, 676 bp, Lycopersicon esculentum
gaaaactgaaactgaaactctgaaagtgaaaccttcttgacttcttttttgaaacaccaaacacgtccacagtccagccgccgcagcagcactcgccagtcgccggctcaccacgggccaccactctcattcatctctcaactctcatctctcgttttcgtcactcatggcacaaactggaagtaaaggaagtgaaagtggagcttcaaatgagagcataccttgtacctcatctccaagtgagggtcaagttgatgttgatacttcaaagaaaagaaaaaccatggaacctagagctaattgttggaagcattttgaaaagttcaccgatgaaaatggagctagtaaatccaaatgcaagtattgtgcaaaaacttatgcagcttctacatcatctaatggtacgtcatcaatgaatactcatttgagaacatgtcctaaatttcctcgtgacactgtagataaaggtcaaaatttaattaattttctaccatcttcaacgggatcaaaggaaggggtcattagcacttggaaatttgaccaagcacaaagtaggaaagctttagccaaaatgataattgttgacgagctaccatttagttttattgagaaagagggttttaaaaacttcatgagagttacaatgccccaatttcatattccttctcgtaaaact

SEQ ID NO: 375 - DNA, 545 bp, Lycopersicon esculentum
gcaattcactcagagcagtggctctgaccaaatggcaggttgaatggtttaactggatgtatcatgtcgcaggatgaattcaaggaattggctcgctgttatttgtgtggtaagacttggaacatgaccttgaacgtcagatggggaggcaaacagaggtctttcaaagtcatgaccaccaagaactcgagggaatcacttgtaaaataccaagaagttcagataaccatatcagaaatgaggtagtgcctggactgctctttcgcagtctgtatggacaatcctgccaggacttcaagtcactcattcgttaaagtatataattgaactgattgtgaaagatggtttccgcaatcatatttagtagaatcaacatatgattctttaaataggcctgggtcatcctggtctgtaggtgcacacttctctttaatgtccataagacgtgtcttttatatgtttttcttttctctggtttgatctccttagggttaggggatcaatatccaggtcttcatctccatctatgaactgctattttctta

SEQ ID NO: 376 - DNA, 547 bp, Lycopersicon esculentum
ggtaatacaaattagttgttgtataacatctcaaaccatcatggactggttcaaggctcaaggccttgcatgtcattggcaagttttaaaaaataaatgcctaatacagttagcccttgtaactaggataaaagattgcaggctggtttcagtcaaaagaaatgctgatctataactcttatccacttactttacaataggaggctgagctaattgttgtagctgatggacgacgtgtgacatcctcggacgcattgatggtacatgttgtgtacaagaataaacaagatcaacaactttttggatgactccaacttcaggaatctgagaagaggaagaagatgatatgagaggatccaagagttcaacatatcgatgagcttgcaccagaggcgtggcccattcaaatatactttgccatccaacagaatctactgcctgtgctggtcttcgtccactaattatttcaagcaagaggactccaaagctgtagacgtcactctttgtagtaagctcatttctgtaaacaaactccggagcaagatac

SEQ ID NO: 377 - DNA, 608 bp, Lycopersicon esculentum
ggcactaagaaatactctccaatcttcttcttcttctctctcaactgtaaaacaacaaaaaatggcggcttctgcacggcgatcaaccggaccggtgctccgttcattatctcccgccggcagattttactcatcgtcatctagaacttcgtcgtcttctgcttcatcgatccggtttacactggaccgtgaaacttccccaagccggtctatttccgtaatgaaccggcagaaaacactggcttcttctcagaagaagaggtgtatgtgttcgccgacgaatcatccaggttcgttccgttgtagtatgcataagaaaatggatattcgacggagtagtagtactagccaaacggcgtcgaattcgattcaattgcatatgaggcgatcggcgatgacgaattcgttagtgcgaatcggaactgttgaaggtgagcttgttaaaagagctttagcggctttgattcgtccttcttcgcatcaacagcgtcgccgttcggatttccaacgccgacctagtcgactttccgttatttccagttccggtgactcataagtcagttattctttctctagaaatttcattcaacaacgcgtgctgcgccgat

SEQ ID NO: 378 - DNA, 293 bp, Lycopersicon esculentum
tatcatattggattggtaaatttaatttgaacaagtcacaactccgcatacaaattaaaacttacggtacaaacgcggtgttcacactgtacacataataatactcctaaattaatcaatctaaagaaaccaataagttccttctttattgaataaaacatgtacgtataaataatcaaagtcacaccaattgctgatcaaacttgttcagtaattctgacatgtagagatcgaagagaaacttcaacaaccacagcgggtacaacaggtacctgggggtgttggttcacaac

SEQ ID NO: 379 - DNA, 492 bp, Lycopersicon esculentum
gatgacgataagaaggctgaacctatatcaagtaagacaataagtagtgtcatatggtctatctggaagaaagaaggattcttgggtttcttcaagggattacatgctcaaatcctcaaaacagcgttaagctctgcattgcttctaatgatcaaggaaaagatctctgccacaacttgggtcctcatactctcacttaggagaattctgctgctcaggcacagaaagctaaaaagtgcttaaattgtgatcatttcagctcaagtcctgagtaaaacaggcttattatatgagaaatcagaagaacgagagttcattgcaaggttgcagtctaagtagttacttccaattacagtctgcaaggcgctctagcagaatgatgaatttaggagaacaatgtggtagctcttggtcttcaaagaagagaataattcgacaaagaaatatttatttttttaacaactttagaataatgtggtgaaagaatctctg

SEQ ID NO: 380 - DNA, 275 bp, Lycopersicon esculentum
attccagctgagagcaaaatcttagttaacgcttggtggctagctaacaaccccgcccactggaagaaacctgcagagttcagacctgagaggttcttggaagaggagaaacacgttgaagccaatggcaacgacttcagatttcttcctttcggggttggcaggaggagttgccccggaattatccttgcattgccaattcttggcatcaccattggacgtttggtgcagaactttgagatgttgcctcctccagggcagtcgaagctcgacac

SEQ ID NO: 381 - DNA, 325 bp, Lycopersicon esculentum
  mRNA (1)..(325)
ggaagaatctggagcagttgtcgaattcgtacaagttaaagtagctcaaatccaatttgtgattctgggtgtttctcaattcatgatatggctgccaatttctggatttcttctcagtacaaagaacttttggaccaagaagaggttgatgtggcgcatccgttggataaagagagaggcatcgctctcgatgattttaagctcatcaagttgcatatgtccaattatgttgcaagattggctcaaaatgtgaaagtgagacaaagggttgttgctaccgcacttacatacatgagacgcgtatatgtcaggagaagtatgaccg

SEQ ID NO: 382 - DNA, 740 bp, Lycopersicon esculentum
gctcaattgcacattcgccttagtcgtcacaattcacattttggcaactccgttttcagcccatcgccgagagaataaataataatttgaaaagcgcttcctccacgatctggttgccggaggaggcgattttttgaatgaagagttcacctgaattttctcgaaatggcggaatcgacgacaattcagaggagctcgcctgaaggtgatgatcatgaactgaaggaggagaatattgagaagaaaaaggattttactgcaaatcctgagtttttcagttgtatgcttcagccagtgcctgccgattcagatccaaattacattggaatacgccggttcctcctccatcgcaaagctgaatccggtgtgcttcgtcgcaaggactggagaggcaatggaaaaggatacgtagcttatcgtaattatattagtaggccaaggaattgggagacattgcaaataccaagccgaccaagtaccccagggaacagtgggcgatggatgccttcagcaagtccagtttcccagttactcgagtcggatagctggaattccttaaaggatttacgaagtcccagtcaagcaagtcaagcattgagtcggactactagttttcagttcaaatggtgagtgacactgaatgatcgtcccacgtagaaaggtcggacctgcatattcctttgtggggatgcattgcattttttgatcaatgcaagccatggttacggttataaagtttg

SEQ ID NO: 383 - DNA, 456 bp, Lycopersicon esculentum
tcgattcttagagaacatatacctgaaccatctcatacaacattggcattagcatgtggaccacctccaatgatacaatttgctattaatccaaacttggagaaaatgggatatgacattaaggaggaactattggtgttctaaattggatggtgatgatgatagatgatatatctctttggaggaataaattcttttgtattttcagttgtacatattgtgtttttgtttatcatcaaaatgtactacttcttgtagttcttacattttaattttctactcaactttaggtataaaatgttgtactgtgtactagtatgtgtatgtcaaagttaagattgtattcatcatggaattacagtaactttgttgtcacaagtcttgttgtagagatttttaccaaattaatgtgtatatatatatagtaaattgaaacaccctctataataaacccct

SEQ ID NO: 384 - DNA, 512 bp, Lycopersicon esculentum
ctgaagctaagcctgattgcctgagtgacttggcttatgtcatcatgaatggtatatgcaagttaagttacaaagtgtctgatgctgatgtggttcgcgcacgtaatcagctgaaatcttctttgatgctccacattgatggatcaggccctactgctgaagacattggacggcagttaatcacatatggccgcagaattccatatgctgagttattttccagaattgacagtgtcgatgctggaacgatcaagcgagttgcaaatcgttttatttttgacagggatgttgcaatatcagctaggggaccaattcaagatttgcctgattacaattggttcagacgcaggacatactggctgcggtactagattgctaattctttcctgatgacttaaattcatcttcttttcccagtcgtaatcgtgacattgggcattataccacacatggtctacaaaacacttttgtcaataaaatgagcgacaaattttgttggacacttttgtttt

SEQ ID NO: 385 - DNA, 530 bp, Lycopersicon esculentum
tttctacacaatccatacattccatcgccggcgtcgtattcctgtcgccggaaaaatgcctcggcagctacttcttcgactgtacttcaacacccgatgttattgtcgatgaacattgacaaattcaaaccactggatttccctaagaactccaccaggaagttgaatcgttctgttactactatcagatcccaattgaattttcccatcatttctccgcaagatcaatggggaacttggactgttcttttcgctaccggcgcttttggcatttggtcggagaagaccaagatcggggctgcactaagtggttcactagtaagtgtactggttggtctagcagcaagtaacttgggcattatagctagtgaagctccagcatataaaatcgtgacaggattcttgcttccacttgctgttcccttgctgttgtttagagctgatatgcgtcgagtattaaagtccactggaacactgctcatggcctttttgcttggatcagttgcaactactattggtactgtggtggc

SEQ ID NO: 386 - DNA, 599 bp, Lycopersicon esculentum
gcagcacaacaaatcataaaatcatatatatcatttaccttgaattgtcaatgtataacaagtcgtttacacaacaattacatccatgggcgaaactagtaattttacaaaacatttacagatgaataacacaactacagatgaagtcaaactgcacaagtacaattctctgatgaagtagtctatgccaaaaaaaaacaagatctcttcccagcaaatagataacaaacatcataataagcgcgtcttcaatgatgaataaccatgatcacatcgtttatatacatgcatatggttatacaagaaaccatagcaatatcaaacagaagccattgtctcaacatattgatgtgtatcataccattcaaacaccgaaacatcttcccatataacagggatctttgctatctctatatccgcaacctccttgatagtcatacgatgagcatcatattcaacgataacgtctggtgtggttaccaaaagcctcgttgtatcttccttctcattaacaaccacacatacaacattgcaagtaccaccattcacatctagtaaatcaacttcttcactcaacattaatggatgagtagtatgca

SEQ ID NO: 387 - DNA, 543 bp, Lycopersicon esculentum
cgacatattaatccagtgactgatgaggatatagtgggttttgggaatgacatagaaaaaatgtttcagtatctgattagaggtacaaatgatctagacgttgtcccaattgtaggcatgggtggacaagggaaaacgacagttgctagaaaggtgtacaatagtgacaacattgtttctcattttgatgttcgagcatggtgcatcgtttcccaaacatataaccggagaaagctattacaagacattttgagtcaagttaccggttccaaggacaaggggtatgaggatgatatccttgctgatgagttgaggaaaagcttaatgggcaagagatatctcattgtcttggatgatatgtgggattgtatggcatgggatgacttaaggctttcctttccagattccggaaatagaagcagaatagtagtaacaactcgacttgagaaagtgggcgaacaagtcaagtgccatactgatccttattctcttccgttcctcacaaaagaagagagttgtgaattgttgcaaaaaaaagtgttt

SEQ ID NO: 388 - DNA, 523 bp, Lycopersicon esculentum
tcagttcaagatgagagtaaggatgaatttaaacaaagtcttacccggggaaaactcaagccttcttcctcatcgtcgttccgtctccgtagcccgagcttgaattctattcgtcttcgtcggatcttcgatgtgttcgatagaaatcacgattgtttgatcagcgttgaagaactcagccaagcacttaacctgcttggattagacgccgatctatcagaaattgaatcgatggtgaaatcatacatcaaaccagaaaacactggtcttagatttgaagatttcgaagccttacatcgatctctgaacgacgttttcttcgggtcaaaatgtgaagataagctcgggttgaattcggatccggcacaggatgaattggatctgaaagaagcgtttgatgtttttgacgagaacggggatggatttatatcggcgaaggaattgcaggtggtgttagagaaattgggattaccggaaggaagtgaaattgatagagtggagatgatgatttcgtcagttgatc

SEQ ID NO: 389 - DNA, 491 bp, Lycopersicon esculentum; identical to SEQ ID NO: 94

SEQ ID NO: 390 - DNA, 478 bp, Lycopersicon esculentum; identical to SEQ ID NO: 120
  misc_feature (376)..(457): Unknown nucleotides 376 and 457

SEQ ID NO: 391 - DNA, 445 bp, Lycopersicon esculentum; identical to SEQ ID NO: 32

SEQ ID NO: 392 - DNA, 344 bp, Lycopersicon esculentum
tggctgcttcaatcaagaacagaatggcaagccaaccggcatcaaacaatgcacgacctccaaccgtgcagatccgaggacaacctgtcaaccaaaagagcggttgctgctcatcttgaaaggacgaagctcgatgatgattctgatgtggaatgtaaaattagtctctcttgatggcttgataggaagactcttcactgttattactttctttcaactctttatcgacactcaattcttttcaatctatttggattaacaatatgtagaaaatctaggaatattggtgaatactagtatttgctttctattctttggaccttcatcaaaatgattttatctgc

SEQ ID NO: 393 - DNA, 340 bp, Lycopersicon esculentum
  misc_feature (248)..(248): Unknown nucleotide 248
gcaagagcttgcaaagcccaatgtacttgaaaggtttctggaaaacaaagatgacattgccaaactacggaaatgctttgcagggttgtggagcttggatgaatccgacattgtcaaggatgcaattgagaggcctgaattgtatgttatgaaaccacaacgagaaggaggaggaaacaatatctatggggaggatgtcagaagtgctcttttgaaactgcaaaaggaaggcactggaagtgatgcancctacatactaatgcagaggattttcccgaagatctctcactcgatactaatgcgagaaggcatctctcataaagaagaaaccatatcagaa

SEQ ID NO: 394 - DNA, 318 bp, Lycopersicon esculentum
tgaggtcatacacttggtccgatgggttcattcggttgtccgagaggagtggactgctgaagtattcgacttggaactattgaggtacccaaacatagaggaagaaatggtggaaatgttgcagatagccatgtcttgtgtggtaaggatgtcagatcaaagaccaaaaatgtttgaagttgttaaaatgatcgagaacgttcgtcccacgagcttagagaatcagctttcatctgaaggaaaagcagaaacttcaaccccaagagaagaagccacacctctgcctgatacacaatctgattctcaatgagtttgcca

SEQ ID NO: 395 - DNA, 597 bp, Lycopersicon esculentum
tactactattacaaatggcttcatcggagattgtggattccgggagatttgccggtcagaaatcgcatttctctcatacatgtaacttgttgagtcaatacttgaaagagaagaaaggttctttgggagatctcagccttgatatgcatcgcaatttcgattcagctggttctactactatggatttgttgccgatgattgagaaatctggtgagttggttcagaaatcgatgaatctgttccctcaaggtggaatgaaggctgagtcggaaccggaaaaggcacagatgacgatattctatggaggtcaagttattgtgtttaatgattttccggctgataaagctaaggaaatcatgcttatggctagtactagcaagggaaacaatcctgctaaaccattggaatctgctgccgatttggtggttccgagtttcggaaaaacttccatccaggaaaatcaaatgcctaaccagccaattgtttctgatttacctattgcgagaagagcttcattaacaaggtttttggagaagagaaaagataggctaactgcaaaagtaccttaccatagagaggaagcagcagctcctaaaa